(19) United States
(12) Patent Application Publication
(10) Pub. No.: US 2026/0021053 A1
(43) Pub. Date: Jan. 22, 2026

(54) MANUFACTURING SYSTEMS AND METHODS FOR CELLULAR THERAPEUTIC PLATFORMS

(71) Applicants: CYTONUS THERAPEUTICS, INC., Carlsbad, CA (US); THE REGENTS OF THE UNIVERSITY OF CALIFORNIA, Oakland, CA (US)

(72) Inventors: Remo MOOMIAIE, Carlsbad, CA (US); Richard KLEMKE, Oakland, CA (US)

(21) Appl. No.: 19/168,506

(22) PCT Filed: Apr. 11, 2024
(86) PCT No.: PCT/US2024/024058

§ 371 Date: Sep. 24, 2025

Related U.S. Application Data
(60) Provisional application No. 63/495,716, filed on Apr. 12, 2023.

(51) Int. Cl. A61K 9/50; A61K 35/766; A61K 38/20; A61K 38/21

(52) U.S. Cl. CPC A61K 9/5068; A61K 35/766; A61K 38/208; A61K 38/215; A61K 38/217

(57) ABSTRACT

Described herein are methods for obtaining enucleated cells from nucleated cells. Also described herein are methods for cell processing, including providing a composition containing nucleated cells and enucleating at least a portion of the nucleated cells to produce an enucleated cell fraction. Also described herein are methods for cell processing, including expressing the heterologous gene product. Also provided are pharmaceuticals compositions comprising an enucleated cell.

### CROSS-REFERENCE

This application claims the benefit of U.S. Provisional Application Ser. No. 63/495,716 filed on Apr. 12, 2023, the entirety of which is hereby incorporated by reference herein.

### STATEMENT AS TO FEDERALLY SPONSORED RESEARCH

The disclosure was made with the support of the United States government support under grant number 1 R43 HL158351-01 awarded by the National Institutes of Health. Accordingly, the government has certain rights in this disclosure.

### SEQUENCE LISTING

The instant application contains a Sequence Listing which has been submitted electronically in XML format and is hereby incorporated by reference in its entirety. Said XML copy, created on Apr. 10, 2024 is named 53712-731_601_SL.xml and is 42,803 bytes in size.

### SUMMARY

Described herein, in some aspects, is a method of delivering a therapeutic agent to a target cell of a subject, the method comprising introducing a plurality of enucleated cells comprising the therapeutic agent to the subject or a sample of the subject in vivo or ex vivo under conditions sufficient to deliver the therapeutic agent to the target cell of the subject, wherein the plurality of enucleated cells is obtained from a cryopreserved composition or a cryohibernated composition, and wherein the therapeutic agent is delivered to the target cell in an amount that is greater than or equal to about an amount of the therapeutic agent delivered to an otherwise comparable target cell of the subject by otherwise comparable enucleated cells that were not cryopreserved or not cryohibernated. In some embodiments, the method further comprises preparing a fluid composition comprising the plurality of enucleated cells from the cryopreserved composition. In some embodiments, the cryopreserved composition is cryopreserved in liquid nitrogen. In some embodiments, the cryopreserved composition is cryopreserved for at least about 24 hours, for at least about 48 hours, at least about 72 hours, at least 96 about hours, at least about 5 days, at least about 6 days, at least about 7 days, at least about 10 days, at least about 15 days, at least about one month, at least about one month, or at least about one year. In some embodiments, the cryopreserved composition is stored at at most about −80° C. prior to cryopreserving the cryopreserved composition. In some embodiments, the cryopreserved composition is stored at a temperature no higher than about −80° C. for at least about 24 hours. In some embodiments, the method further comprises preparing a fluid composition comprising the plurality of enucleated cells from the cryohibernated composition. In some embodiments, the cryohibernated composition is stored at a temperature no higher than about 4° C. In some embodiments, the cryohibernated composition is cryohibernated for at least about 24 hours, for at least about 48 hours, at least about 72 hours, at least 96 about hours, at least about 5 days, at least about 6 days, at least about 7 days, at least about 10 days, at least about 15 days, at least about one month, at least about one month, or at least about one year. In some embodiments, the plurality of enucleated cells from the cryopreserved composition are suspended in a xeno-free media. In some embodiments, the plurality of enucleated cells from the cryopreserved composition are suspended in a freezing media. In some embodiments, the freezing media comprises at least 2%, at least 5%, or at least 10% DMSO. In some embodiments, the freezing media comprises CryoStor® media. In some embodiments, the CryoStor® media is CryoStor® CS5 or CryoStor® CS10. In some embodiments, the freezing media comprises DMSO, sucrose, sodium hydroxide, potassium hydroxide, or a combination thereof. In some embodiments, the freezing media comprises about 2% to about 15% DMSO. In some embodiments, the freezing media comprises about 0.5% to about 2% sucrose. In some embodiments, the freezing media comprises about 1% sucrose. In some embodiments, the freezing media comprises about 0.5% to about 1% sodium hydroxide. In some embodiments, the freezing media comprises about 0.6% sodium hydroxide. In some embodiments, the freezing media comprises about 0.05% to about 0.5% potassium hydroxide. In some embodiments, the freezing media comprises about 0.1% potassium hydroxide. In some embodiments, the preparing the fluid composition comprises thawing the cryopreserved composition. In some embodiments, the thawing the cryopreserved composition is performed at room temperature or at 37° C. In some embodiments, the method further comprises reconstituting the plurality of enucleated cells from the cryopreserved composition subsequent to the thawing. In some embodiments, the reconstituting the plurality of enucleated cells from the cryopreserved composition uses phosphate buffer solution (PBS). In some embodiments, the reconstituting the plurality of enucleated cells from the cryopreserved composition uses sodium lactate solution. In some embodiments, the reconstituting the plurality of enucleated cells from the cryopreserved composition uses saline solution. In some embodiments, the therapeutic agent comprises a virus, an exogenous DNA molecule, an exogenous RNA molecule, an exogenous protein, an exogenous peptide, or any combination thereof. In some embodiments, the therapeutic agent comprises the virus. In some embodiments, the virus is an adeno-associated virus (AAV), an adenovirus, a reovirus, a coxsackie virus, a retrovirus, a poxvirus, a baculovirus, or a herpes virus. In some embodiments, the virus comprises an oncolytic virus. In some embodiments, the oncolytic virus is an adenovirus, a human immunodeficiency virus, a Maraba virus, a Measles virus, a Newcastle disease virus, a poliovirus, a Seneca Valley virus, a parvovirus, a Semliki Forest virus, a Vesicular Stomatitis virus, a Sindbis virus, or any combination thereof. In some embodiments, the amount of the virus delivered to the subject is measured in viral titers in the target cell. In some embodiments, the viral titers measured in the target cell are greater than the viral titers measured in the otherwise comparable target cell. In some embodiments, the viral titers measured in the target cell are equal to about the viral titers measured in the otherwise comparable target cell. In some embodiments, the exogenous protein comprises a cytokine or a cytokine receptor-binding fragment thereof. In some embodiments, the amount of the cytokine or the cytokine receptor-binding fragment thereof delivered to the subject is measured by the secretion of the cytokine or the cytokine receptor-binding fragment thereof from the plurality of enucleated cells. In some embodiments, the secretion of the cytokine or the cytokine receptor-binding fragment thereof measured is greater than or equal to about the secretion of the cytokine or the cytokine receptor-binding fragment thereof by an otherwise comparable enucleated cell that was not cryopreserved. In some embodiments, the secretion of the cytokine or the cytokine receptor-binding fragment thereof measured is greater than or equal to about the secretion of the cytokine or the cytokine receptor-binding fragment thereof by an otherwise comparable nucleated cell that was cryopreserved. In some embodiments, the exogenous protein comprises an immune checkpoint inhibitor. In some embodiments, the immune checkpoint inhibitor comprises an inhibitor specific to PD-L1, PD-1, or a combination thereof. In some embodiments, the exogenous protein comprises an antigen. In some embodiments, the exogenous protein comprises an immunomodulatory protein. In some embodiments, the therapeutic agent comprises the exogenous RNA molecule. In some embodiments, the exogenous RNA molecule encodes a cytokine or the cytokine receptor-binding fragment thereof, a chemokine, or any combination thereof. In some embodiments, the exogenous RNA molecule encodes the cytokine or the cytokine receptor-binding fragment thereof. In some embodiments, the cytokine or the cytokine receptor-binding fragment thereof comprises interleukin-12 (IL-12), interferon-α (IFN-α), interferon-β (IFN-β), interferon-γ (IFN-γ), interleukin-7 (IL-7), interleukin-21 (IL-21), tumor necrosis factor α (TNF-α), granulocyte-macrophage colony-stimulating factor (GM-CSF), interleukin-15 (IL-15), or any combination thereof. In some embodiments, the exogenous RNA molecule encodes the chemokine. In some embodiments, the chemokine comprises stromal cell-derived factor-1α (SDF1α), C-C motif chemokine ligand 2 (CCL2), C-C motif chemokine ligand 3 (CCL3), C-C motif chemokine ligand 5 (CCL5), C-C motif chemokine ligand 8 (CCL8), C-C motif chemokine ligand 1 (CCL1), CXC motif chemokine ligand 9 (CXCL9), CXC motif chemokine ligand 10 (CXCL10), C-C motif chemokine ligand 11 (CCL11), CXC motif chemokine ligand 12 (CXCL12), or any combination thereof. In some embodiments, the exogenous RNA molecule encodes an immune checkpoint inhibitor, an antigen, or an immunomodulatory protein. In some embodiments, the immune checkpoint inhibitor comprises an inhibitor specific to PD-L1, PD-1, or a combination thereof. In some embodiments, the method further comprises treating a disease or a condition in the subject. In some embodiments, the disease is a cancer. In some embodiments, the cancer comprises a solid tumor. In some embodiments, the cancer is a lung cancer, a cancer metastasis in lung tissue, a liver cancer, or a cancer metastases in liver tissue. In some embodiments, the liver cancer is a hepatocellular carcinoma or a cholangiocarcinoma. In some embodiments, the cancer is the lung cancer. In some embodiments, the lung cancer is a small cell lung cancer, a non-small lung cancer, or a bronchial carcinoid. In some embodiments, the lung cancer is the small cell lung cancer. In some embodiments, the lung cancer is the bronchial carcinoids. In some embodiments, the lung cancer is the non-small cell lung cancer. In some embodiments, the non-small cell lung cancer is an adenocarcinoma, squamous cell carcinoma, or large cell carcinoma. In some embodiments, the method further comprises administering the plurality of enucleated cells to the subject intravenously. In some embodiments, the target cell of the subject comprises a cancer cell. In some embodiments, the target cell of the subject comprises a solid tumor cell. In some embodiments, the target cell of the subject comprises a lung cell. In some embodiments, the target cell of the subject comprises a liver cell.

Described herein, in some aspects, is a composition, comprising: a plurality of enucleated cells formulated from a cryopreserved composition or a cryohibernated composition, wherein the cryopreserved composition or the cryohibernated composition comprises the plurality of enucleated cells that are cryopreserved or cryohibernated, wherein at least a subset of the plurality of enucleated cells comprises (i) a therapeutic agent, and (ii) intracellular organelles sufficient to release the therapeutic agent in vivo or ex vivo in an amount that is greater than or equal to about an amount of the therapeutic agent released by otherwise identical enucleated cells that were not cryopreserved or not cryohibernated. In some embodiments, the plurality of enucleated cells comprises a diameter comprising less than or equal to about 70% of an average diameter of a nucleated parent cell. In some embodiments, the plurality of enucleated cells comprises a diameter comprising between about 1 micrometer (μm) to about 100 μm. In some embodiments, the plurality of enucleated cells comprises a diameter comprising between about 5 μm to about 25 μm. In some embodiments, the plurality of enucleated cells comprises a diameter comprising about 8 μm. In some embodiments, the therapeutic agent comprises a virus, an exogenous DNA molecule, an exogenous RNA molecule, an exogenous protein, or an exogenous peptide, or any combination thereof. In some embodiments, the therapeutic agent comprises the virus. In some embodiments, the virus is an adeno-associated virus (AAV), an adenovirus, a reovirus, a coxsackie virus, a retrovirus, a poxvirus, a baculovirus, or a herpes virus. In some embodiments, the virus comprises an oncolytic virus. In some embodiments, the oncolytic virus is an adenovirus, a human immunodeficiency disease, a Maraba virus, a Measles virus, a Newcastle disease virus, a poliovirus, a Seneca Valley virus, a parvovirus, a Semliki Forest virus, a Vesicular Stomatitis virus, a Sindbis virus, or any combination thereof. In some embodiments, the amount of the virus released is measured in viral titers in a target cell. In some embodiments, the viral titers measured in the target cell are greater than the viral titers measured in an otherwise comparable target cell. In some embodiments, the viral titers measured in the target cell are equal to about the viral titers measured in an otherwise comparable target cell. In some embodiments, the therapeutic agent comprises a cytokine or cytokine receptor-binding fragment thereof. In some embodiments, the amount of the cytokine or the cytokine receptor-binding fragment released in vivo or ex vivo is a measurement of the secretion of the cytokine or the cytokine receptor-binding fragment thereof from the plurality of enucleated cells. In some embodiments, the amount of the cytokine or the cytokine receptor-binding fragment thereof measured is greater than or equal to about the secretion of the cytokine or the cytokine receptor-binding fragment thereof by the otherwise comparable enucleated cells that were not cryopreserved or not cryohibernated. In some embodiments, the amount of the cytokine or the cytokine receptor-binding fragment thereof measured is greater than or equal to about the secretion of the cytokine or the cytokine receptor-binding fragment thereof by the otherwise comparable nucleated cells that were cryopreserved or cryohibernated. In some embodiments, the exogenous protein comprises an immune checkpoint inhibitor. In some embodiments, the exogenous protein comprises an antigen. In some embodiments, the exogenous protein comprises an immunomodulatory protein. In some embodiments, the immune checkpoint inhibitor comprises an inhibitor specific to PD-L1, PD-1, or a combination thereof. In some embodiments, the therapeutic agent comprises an exogenous RNA molecule. In some embodiments, the exogenous RNA molecule encodes a cytokine or the cytokine receptor-binding fragment thereof, a chemokine, or any combination thereof. In some embodiments, the exogenous RNA molecule encodes a cytokine or the cytokine receptor-binding fragment thereof. In some embodiments, the cytokine or the cytokine receptor-binding fragment thereof comprises IL-12, IFN-α, IFN-β, IFN-γ, IL-7, IL-21, TNF-α, GM-CSF, IL-15, or any combination thereof. In some embodiments, the exogenous RNA molecule encodes a chemokine. In some embodiments, the chemokine comprises SDF1α, CCL2, CCL3, CCL5, CCL8, CCL1, CXCL9, CXCL10, CCL11, CXCL12, or combination thereof. In some embodiments, the exogenous RNA molecule encodes an immune checkpoint inhibitor, an antigen, or an immunomodulatory protein. In some embodiments, the immune checkpoint inhibitor comprises an inhibitor specific to PD-L1, PD-1, or a combination thereof. In some embodiments, each enucleated cell of the plurality of enucleated cells lacks a nucleus and comprises one or more structural features of a nucleated cell. In some embodiments, the one or more structural features comprises one or more tunneling nanotubes. In some embodiments, the intracellular organelles comprise a Golgi apparatus, an endoplasmic reticulum, or any combination thereof.

Described herein, in some aspects, is a pharmaceutical composition, comprising: a composition described herein, and a pharmaceutically acceptable: excipient, diluent, or carrier. In some embodiments, the pharmaceutical composition is in a unit dose form. In some embodiments, the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesicaly, intravaginally, intrarectally, orally, sublingually, transdermally, by inhalation, by inhaled nebulized form, by intraluminal-GI route, or any combination thereof, to a subject. In some embodiments, the pharmaceutical composition is formulated for administering intravenously. In some embodiments, the pharmaceutical composition further comprises at least one additional active agent. In some embodiments, the at least one additional active agent comprises a cytokine, a growth factor, a hormone, an enzyme, a small molecule, a compound, or any combination thereof.

Described herein, in some aspects, is a kit, comprising: a composition described herein; or a pharmaceutical composition described herein; and a container storing the composition or the pharmaceutical composition. In some embodiments, the kit further comprises a resuspension buffer. In some embodiments, the resuspension buffer comprises PBS. In some embodiments, the resuspension buffer comprises saline solution. In some embodiments, the resuspension buffer comprises sodium lactate solution. In some embodiments, the kit further comprises instructions comprising a method for delivering the composition or the pharmaceutical composition to a target cell of a subject, wherein the method comprises: introducing the composition or the pharmaceutical composition to the target cell of a subject in vivo or ex vivo under conditions sufficient to deliver the therapeutic agent to the target cell. In some embodiments, the method further comprises treating a disease or a condition of the subject by administering the therapeutic agent to the target cell of the subject. In some embodiments, the disease or the condition comprises cancer. In some embodiments, the cancer comprises solid tumor. In some embodiments, the cancer is a lung cancer, a cancer metastases in lung tissue, a liver cancer, or a cancer metastases in liver tissue. In some embodiments, the introducing the composition or the pharmaceutical composition to the target cell of a subject comprises administering the composition or the pharmaceutical composition to the subject intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesicaly, intravaginally, intrarectally, orally, sublingually, transdermally, by inhalation, by inhaled nebulized form, by intraluminal-GI route, or any combination thereof. In some embodiments, the kit further comprises at least one additional active agent, wherein the at least one additional active agent comprises a cytokine, a growth factor, a hormone, an enzyme, a small molecule, a compound, or any combination thereof.

Aspects disclosed herein provide methods of delivering a therapeutic agent to a target cell of a subject, the method comprising: a) preparing a fluid formulation comprising a plurality of enucleated cells from a cryopreserved composition, wherein the cryopreserved composition comprises the plurality of enucleated cells that are cryopreserved, wherein at least a subset of the plurality of enucleated cells comprises a therapeutic agent; and b) introducing the fluid formulation to the subject or a sample of the subject under conditions sufficient to deliver the therapeutic agent to the target cell of the subject in vivo or ex vivo in an amount that is greater than or equal to about an amount of the therapeutic agent delivered to an otherwise identical target cell of the subject by otherwise identical enucleated cells that were not cryopreserved. In some embodiments, the cryopreserved composition is cryopreserved in liquid nitrogen. In some embodiments, the cryopreserved composition is cryopreserved for at least about 24 hours. In some embodiments, the cryopreserved composition is cryopreserved for at least about 7 days. In some embodiments, the cryopreserved composition is cryopreserved for at least about one month. In some embodiments, the cryopreserved composition is cryopreserved for at least about one year. In some embodiments, the cryopreserved composition is placed at most about −80° C. prior to cryopreserving the cryopreserved composition. In some embodiments, the cryopreserved composition is placed at most about −80° C. for at least about 24 hours. In some embodiments, the plurality of enucleated cells from the cryopreserved composition are suspended in Xeno-free media. In some embodiments, the plurality of enucleated cells from the cryopreserved composition are suspended in CryoStor® media. In some embodiments, the CryoStor® media is CryoStor® CS10 media. In some embodiments, the preparing the fluid composition comprises thawing the cryopreserved composition. In some embodiments, the thawing the cryopreserved composition is performed at room temperature. In some embodiments, the thawing the cryopreserved composition is performed at 37° C. In some embodiments, the thawing the cryopreserved composition is performed at 37° C. in a water bath. In some embodiments, the method further comprises: reconstituting the plurality of enucleated cells from the cryopreserved composition subsequent to the thawing. In some embodiments, the reconstituting the plurality of enucleated cells from the cryopreserved composition uses phosphate buffer solution (PBS). In some embodiments, the reconstituting the plurality of enucleated cells from the cryopreserved composition uses sodium lactate solution. In some embodiments, the therapeutic agent comprises a virus, an exogenous DNA molecule, an exogenous RNA molecule, an exogenous protein, an exogenous peptide, or a combination thereof. In some embodiments, the therapeutic agent comprises a virus. In some embodiments, the virus is an adeno-associated virus (AAV), an adenovirus, a reovirus, a coxsackie virus, a retrovirus, a poxvirus, a baculovirus, or a herpes virus. In some embodiments, the virus comprises an oncolytic virus. In some embodiments, the oncolytic virus is an adenovirus, a human immunodeficiency virus, a Maraba virus, a Measles virus, a Newcastle disease virus, a poliovirus, a Seneca Valley virus, a parvovirus, a Semliki Forest virus, a Vesicular Stomatitis virus, a Sindbis virus, or a combination thereof. In some embodiments, the amount of the virus delivered to the subject in vivo or ex vivo is a measurement of viral titers in the target cell. In some embodiments, the viral titers measured in the target cell are greater than the viral titers measured in the otherwise identical target cell. In some embodiments, the viral titers measured in the target cell are equal to about the viral titers measured in the otherwise identical target cell. In some embodiments, the therapeutic agent comprises a cytokine or cytokine receptor-binding fragment thereof. In some embodiments, the amount of the cytokine or the cytokine receptor-binding fragment thereof delivered to the subject in vivo or ex vivo is a measurement of the secretion of the cytokine or the cytokine receptor-binding fragment thereof from the plurality of enucleated cells. In some embodiments, the secretion of the cytokine or the cytokine receptor-binding fragment thereof measured is greater than or equal to about the secretion of the cytokine or the cytokine receptor-binding fragment thereof by an otherwise identical enucleated cell that was not cryopreserved. In some embodiments, the secretion of the cytokine or the cytokine receptor-binding fragment thereof measured is greater than or equal to about the secretion of the cytokine or the cytokine receptor-binding fragment thereof by an otherwise identical nucleated cell that was cryopreserved. In some embodiments, the therapeutic agent comprises the exogenous RNA molecule. In some embodiments, the exogenous RNA molecule encodes a cytokine or the cytokine receptor-binding fragment thereof, a chemokine, or a combination thereof. In some embodiments, the exogenous RNA molecule encodes a cytokine or the cytokine receptor-binding fragment thereof. In some embodiments, the cytokine or cytokine receptor-binding fragment thereof comprises interleukin-12 (IL-12), interferon-α (IFN-α), interferon-β (IFN-β), interferon-Y (IFN-γ), interleukin-7 (IL-7), interleukin-21 (IL-21), tumor necrosis factor α (TNF-α), granulocyte-macrophage colony-stimulating factor (GM-CSF), interleukin-15 (IL-15), or a combination thereof. In some embodiments, the exogenous RNA molecule encodes a chemokine. In some embodiments, the chemokine comprises stromal cell-derived factor-1α (SDF1α), C-C motif chemokine ligand 2 (CCL2), C-C motif chemokine ligand 3 (CCL3), C-C motif chemokine ligand 5 (CCL5), C-C motif chemokine ligand 8 (CCL8), C-C motif chemokine ligand 1 (CCL1), CXC motif chemokine ligand 9 (CXCL9), CXC motif chemokine ligand 10 (CXCL10), C-C motif chemokine ligand 11 (CCL11), CXC motif chemokine ligand 12) CXCL12, or a combination thereof. In some embodiments, the method further comprises treating a disease or a condition in the subject. In some embodiments, the disease is cancer. In some embodiments, the cancer is lung cancer, cancer metastases in lung tissue, liver cancer, or cancer metastases in liver tissue. In some embodiments, the liver cancer is hepatocellular carcinoma or cholangiocarcinoma. In some embodiments, the cancer is lung cancer. In some embodiments, the lung cancer is small cell lung cancer, non-small lung cancer, or bronchial carcinoids. In some embodiments, the lung cancer is small cell lung cancer. In some embodiments, the lung cancer is bronchial carcinoids. In some embodiments, the lung cancer is non-small cell lung cancer. In some embodiments, the non-small cell lung cancer is adenocarcinomas, squamous cell carcinomas, or large cell carcinomas. In some embodiments, the treating the disease or the condition in the subject comprises administering the fluid formulation to the subject intravenously. In some embodiments, the target cell of the subject comprises a lung cell. In some embodiments, the target cell of the subject comprises a liver cell.

Aspects disclosed herein provide formulations comprising: a plurality of enucleated cells formulated from a cryopreserved composition, wherein the cryopreserved composition comprises the plurality of enucleated cells that are cryopreserved, wherein at least a subset of the plurality of enucleated cells comprises (i) a therapeutic agent, and (ii) intracellular organelles sufficient to release the therapeutic agent in vivo or ex vivo in an amount that is greater than or equal to about an amount of the therapeutic agent released by otherwise identical enucleated cells that were not cryopreserved. In some embodiments, each enucleated cell of the plurality of enucleated cells comprises a diameter comprising less than or equal to about 70% of an average diameter of a nucleated parent cell. In some embodiments, each enucleated cell of the plurality of enucleated cells comprises a diameter comprising between about 1 micrometer (μm) to about 100 μm. In some embodiments, each enucleated cell of the plurality of enucleated cells comprises a diameter comprising between about 5 μm to about 25 μm. In some embodiments, each nucleated cell of the plurality of enucleated cells comprises a diameter comprising about 8 μm. In some embodiments, the therapeutic agent comprises a virus, an exogenous DNA molecule, an exogenous RNA molecule, an exogenous protein, or an exogenous peptide, or any combination thereof. In some embodiments, the therapeutic agent comprises a virus. In some embodiments, the virus is an adeno-associated virus (AAV), an adenovirus, a reovirus, a coxsackie virus, a retrovirus, a poxvirus, a baculovirus, or a herpes virus. In some embodiments, the virus comprises an oncolytic virus. In some embodiments, the oncolytic virus is an adenovirus delta 24, a human immunodeficiency disease, a Maraba virus, a Measles virus, a Newcastle disease virus, a poliovirus, a Seneca Valley virus, a parvovirus, a Semliki Forest virus, a Vesicular Stomatitis virus, a Sindbis virus, or a combination thereof. In some embodiments, the amount of the virus released in vivo or ex vivo is a measurement of viral titers in a target cell. In some embodiments, the viral titer measured in the target cell are greater than the viral titers measured in an otherwise identical target cell. In some embodiments, the viral titers measured in the target cell are equal to about the viral titers measured in an otherwise identical target cell. In some embodiments, the therapeutic agent comprises a cytokine or cytokine receptor-binding fragment thereof. In some embodiments, the amount of the cytokine or the cytokine receptor-binding fragment released in vivo or ex vivo is a measurement of the secretion of the cytokine or the cytokine receptor-binding fragment thereof from the plurality of enucleated cells. In some embodiments, the amount of the cytokine or the cytokine receptor-binding fragment thereof measured is greater than or equal to about the secretion of the cytokine or the cytokine receptor-binding fragment thereof by the otherwise identical enucleated cells that was not cryopreserved. In some embodiments, the amount of the cytokine or the cytokine receptor-binding fragment thereof measured is greater than or equal to about the secretion of the cytokine or the cytokine receptor-binding fragment thereof by the otherwise identical nucleated cells that was not cryopreserved. In some embodiments, the therapeutic agent comprises an exogenous RNA molecule. In some embodiments, the exogenous RNA molecule encodes a cytokine or the cytokine receptor-binding fragment thereof, a chemokine, or a combination thereof. In some embodiments, the exogenous RNA molecule encodes a cytokine or the cytokine receptor-binding fragment thereof. In some embodiments, the cytokine or the cytokine receptor-binding fragment thereof comprises IL-12, IFN-α, IFN-β, IFN-γ, IL-7, IL-21, TNF-α, GM-CSF, IL-15, or a combination thereof. In some embodiments, the exogenous RNA molecule encodes a chemokine. In some embodiments, the chemokine comprises SDF1α, CCL2, CCL3, CCL5, CCL8, CCL1, CXCL9, CXCL10, CCL11, CXCL 12, or combination thereof. In some embodiments, each enucleated cell of the plurality of enucleated cells lacks a nucleus and comprise one or more structural features of a nucleated cell. In some embodiments, the one or more structural features comprise one or more tunneling nanotubes. In some embodiments, the intracellular organelles comprise a Golgi apparatus, an endoplasmic reticulum, or a combination thereof.

Aspects disclosed herein provide pharmaceutical formulations, comprising: a) a formulation of any one of preceding embodiments, and b) a pharmaceutically acceptable: excipient, diluent, or carrier. In some embodiments, the pharmaceutical formulation is in an unit dose form. In some embodiments, the pharmaceutical formulation is formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesicaly, intravaginally, intrarectally, orally, sublingually, transdermally, by inhalation, by inhaled nebulized form, by intraluminal-GI route, or a combination thereof, to a subject. In some embodiments, the pharmaceutical formulation is formulated for administering intravenously. In some embodiments, the pharmaceutical formulation further comprises at least one additional active agent. In some embodiments, the at least one additional active agent comprises a cytokine, a growth factor, a hormone, an enzyme, a small molecule, a compound, or any combination thereof.

Aspects disclosed herein provide kits, comprising: the formulation of any one of the preceding embodiments or the pharmaceutical formulation of any one of the preceding embodiments; and b) a container storing the formulation or the pharmaceutical formulation. In some embodiments, the kit further comprises a resuspension buffer. In some embodiments, the resuspension buffer comprises Phosphate-buffered saline (PBS). In some embodiments, the resuspension buffer comprises sodium lactate solution. In some embodiments, the kit further comprises instructions comprising a method for delivering the formulation or the pharmaceutical formulation to a target cell of a subject, wherein the method comprising: introducing the formulation or the pharmaceutical formulation to the target cell of a subject under conditions sufficient to deliver the therapeutic agent to the target cell in vivo or ex vivo. In some embodiments, the instructions further comprise a method for delivering the formulation or the pharmaceutical formulation to a target cell of a subject, wherein the method comprising: introducing the formulation or the pharmaceutical formulation to the target cell of a subject under conditions sufficient to deliver the therapeutic agent to the target cell in vivo or ex vivo. In some embodiments, the method further comprises treating a disease or a condition of the subject by administering the therapeutic agent to the target cell of the subject in vivo. In some embodiments, the disease or the condition comprises cancer. In some embodiments, the cancer is lung cancer, cancer metastases in lung tissue, liver cancer, or cancer metastases in liver tissue. In some embodiments of any one of preceding kits, the introducing the formulation or the pharmaceutical formulation to the target cell of a subject comprises administering the formulation or the pharmaceutical formulation to the subject intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesicaly, intravaginally, intrarectally, orally, sublingually, transdermally, by inhalation, by inhaled nebulized form, by intraluminal-GI route, or a combination thereof. In some embodiments of any one of the preceding kits, the kit further comprises at least one additional active agent, wherein the at least one additional active agent comprises a cytokine, a growth factor, a hormone, an enzyme, a small molecule, a compound, or any combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Some novel features of the inventive concepts disclosed herein are set forth in the present disclosure. A better understanding of the features and advantages of the inventive concepts disclosed herein will be obtained by reference to the following detailed description that sets forth non-limiting illustrative embodiments, in which the principles of the disclosed inventive concepts are utilized, and the accompanying drawings of which:

FIG. 1 illustrates a flow chart showing non-limiting steps of a process for composition or pharmaceutical composition of enucleated cells for delivery of therapeutics, according to an embodiment of the present disclosure.

FIG. 2 illustrates a timeline for production of the enucleated cells for the delivery of the single-domain antibody according to various embodiments, as compared to a typical biological drug development timeline.

FIG. 3A illustrates the workflow for an experiment evaluating the adherence of fresh enucleated cells and cryopreserved enucleated cells to fibronectin-coated plates.

FIG. 3B are images of fresh enucleated cells (left; e.g., pre-freeze) and cryopreserved enucleated cells (right; e.g., post-thaw) 24 hours post plating. Images acquired by Nikon Eclipse Ti microscope.

FIG. 4A illustrates the workflow for an experiment evaluating the secretion of IL-12 by fresh enucleated cells and cryopreserved enucleated cells. The enucleated cells are plated in triplicate.

FIG. 4B illustrates the level of IL-12 (in nanogram per milliliter) secreted by fresh enucleated cells, enucleated cells cryopreserved in 90% FBS+10% DMSO, and enucleated cells cryopreserved in CryoStor® CS10. The data shown is the mean of three samples per group of enucleated cells tested and the error bar depicts the standard deviation of the data.

FIG. 5A illustrates the workflow for experiments evaluating the functional abilities of fresh enucleated cells and cryopreserved enucleated cells in vivo.

FIG. 5B illustrates the level of IL-12 (ng/mL) in supernatant from cultures with either fresh enucleated cells or cryopreserved enucleated cells

FIG. 5C illustrates the level of IL-12 picograms/mL (pg/mL) in plasma from mice injected with either fresh enucleated cells or cryopreserved enucleated cells on day 1, day 2, and day 3 post injection. The data shown is the mean of three mice per group of enucleated cells tested and the error bars depict the standard error of the mean of the data.

FIG. 5D illustrates the level of IFNγ (pg/mL) in plasma from mice injected with either fresh enucleated cells or cryopreserved enucleated cells on day 1, day 2, and day 3 post injection. The data shown is the mean of three mice per group of enucleated cells tested and the error bars depict the standard error of the mean of the data.

FIG. 5E illustrates the fold change of expression of IL-12 mRNA in the lungs of mice injected with either fresh enucleated cells or cryopreserved enucleated cells on day 1, day 2, and day 3 post injection. Fold change is calculated using the delta-delta Ct method comparing the target gene expression level to the house keeping gene hypoxanthine phosphoribosyltransferase (HPRT) expression level. The data shown is the mean of three mice per group of enucleated cells tested and the error bars depict the standard error of the mean of the data.

FIG. 5F illustrates the fold change of expression of IFN-γ in the lungs of mice injected with either fresh enucleated cells or cryopreserved enucleated cell on day 1, day 2, and day 3 post injection. Fold change is calculated using the delta-delta Ct method comparing the target gene expression level to the house keeping gene hypoxanthine phosphoribosyltransferase (HPRT) expression level. The data shown is the mean of three mice per group of enucleated cells tested and the error bars depict the standard error of the mean of the data.

FIG. 5G illustrates the fold change of expression of IL-12 mRNA in the livers of mice injected with either fresh enucleated cells or cryopreserved enucleated cells on day 1, day 2, and day 3 post injection. Fold change is calculated using the delta-delta Ct method comparing the target gene expression level to the house keeping gene hypoxanthine phosphoribosyltransferase (HPRT) expression level. The data shown is the mean of three mice per group of enucleated cells tested and the error bars depict the standard error of the mean of the data.

FIG. 5H illustrates the fold change of expression of IFN-γ in the livers of mice injected with either fresh enucleated cells or cryopreserved enucleated cells on day 1, day 2, and day 3 post injection. Fold change is calculated using the delta-delta Ct method comparing the target gene expression level to the house keeping gene hypoxanthine phosphoribosyltransferase (HPRT) expression level. The data shown is the mean of three mice per group of enucleated cells tested and the error bars depict the standard error of the mean of the data.

FIG. 5I illustrates the percentage of single DiD-labeled enucleated cells present in the lungs of mice injected with either fresh enucleated cells or cryopreserved enucleated cells on day 1, day 2, and day 3 post injection. The data is presented as the total number of DiD+ events as a frequency of single cells. The data shown is the mean of three mice per group of enucleated cells tested and the error bars depict the standard error of the mean of the data.

FIG. 6A illustrates the workflow for an experiment to evaluate the delivery of VSV to the lungs of mice by fresh enucleated cells or cryopreserved enucleated cells. Five mice are injected with either fresh enucleated cells or cryopreserved enucleated cells.

FIG. 6B illustrates the titers of VSV (plaque-forming units per gram of lung tissue) found in the lung of mice injected with either fresh VSV-infected enucleated cells or cryopreserved VSV-infected enucleated cells. The data shown in the mean of five mice per group of enucleated cells tested and the error bars depict the standard error of the mean of the data.

FIG. 6C illustrates the titers of VSV (PFU/mL) of supernatant collected 48 hours after either fresh VSV-infected enucleated cells (e.g., pre-freeze) or cryopreserved VSV-infected enucleated cells (e.g., post-thaw) were plated.

FIG. 7A illustrates human Wharton's Jelly (mesenchymal stem cells) MSCs transfected with mouse IL-12 mRNA and seeded as fresh or after freezing and thawing process. The secreted mouse IL-12 was analyzed from the conditioned media of each condition by ELISA. n=3.

FIG. 7B illustrates human umbilical cord MSCs transfected with mouse IL-12 mRNA and seeded as fresh or after freezing and thawing process. The secreted mouse IL-12 was analyzed from the conditioned media of each condition by ELISA. n=3.

FIG. 8A and FIG. 8B illustrate C57BL/6 mice inoculated subcutaneously with 1×10⁶ EO771 tumor cells. After 12 days the mice were stratified by tumor volume. Every 3 days the mice were treated intratumorally (i.t.) with cryopreserved human bone marrow enucleated cell transfected or not transfected with mouse IL-12 mRNA alongside intraperitoneal (i.p.) injections of anti PD1 antibody. FIG. 8A: Tumor volumes were measured 3 times a week and mice were sacrificed if not passing health criteria. FIG. 8B: Kaplan-Meier curve of the same mice as in FIG. 8A. n is indicated in the figure.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

Quality control of enucleated cell platforms for biomedical applications is a challenge, which become magnified by large scale manufacturing. Some of the many benefits of the enucleated cells disclosed herein are attributed to an absence of a nucleus, such as unwanted gene transfer in vivo, limited lifespan in vivo, and so forth. However, existing large scale manufacturing techniques result in a portion of nucleated parent cells in the resulting therapeutic composition, obviating the benefits of the enucleated cell platform.

In addition to the advances in manufacturing scalability and quality control, the enucleated cell platform, itself, described herein possess certain advantages over existing cell-based therapeutic platforms that make it uniquely suitable for large scale use as therapeutic compositions. Additional disclosure of the enucleated cells described herein may be found in U.S. Pat. No. 10,927,349, which is hereby incorporated by reference in its entirety. In addition, additional utility and advantages of the enucleated cells disclosed herein are provided in International Application No. PCT/US2022/018007, filed Feb. 25, 2022, and published as WO/20221/83057 A1; and U.S. patent application Ser. No. 17/885,867, filed Aug. 11, 2022, and published in WO/20211/63222 A1, each of which is hereby incorporated by reference in its entirety.

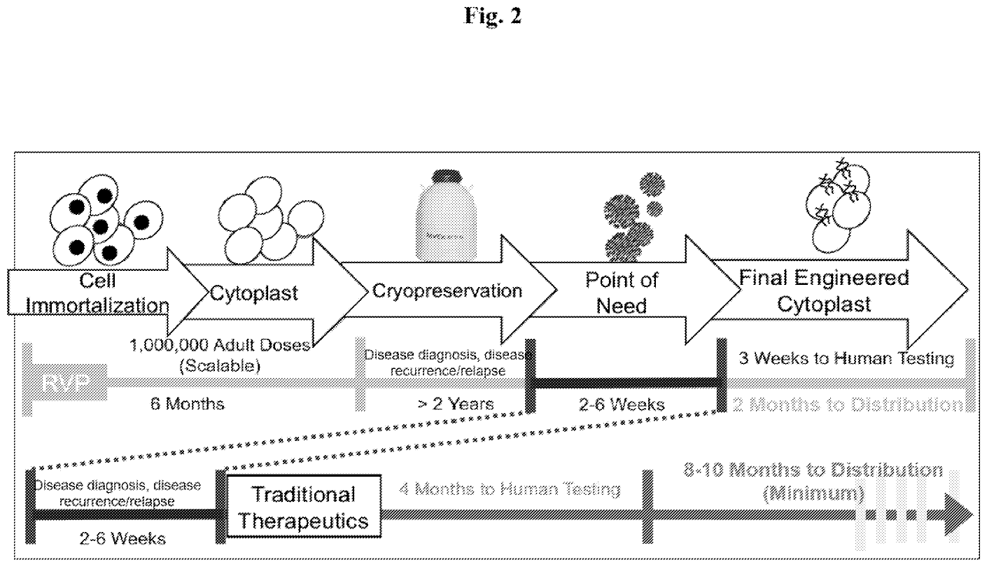

For example, there are certain therapeutic applications of cellular delivery platforms, such as in response to a disease reoccurrence, for which existing manufacturing timelines can limit scalability and speed necessary to address a disease reoccurrence in an individual. Existing therapeutic cellular therapies requiring extensive engineering take on the order of 12 months to develop at minimum. Whereas the enucleated cells disclosed herein can be extensively engineered before and after enucleation (e.g., with targeting moieties specific to target tissue, immune-system evading moieties to reduce phagocytosis in vivo, etc.), and then stored by suitable means disclosed here (e.g., cryopreservation) for extended periods of time without sacrificing viability once revived. When a new pathogen or new strain of a known pathogen is identified, the biological activity of the enucleated cells (already engineered to express the appropriate targeting moieties, immune-system evading moieties, immune activators, etc.) can be restored (e.g., rehydration, thawing, etc.) and further engineered to express or carry a therapeutic agent for the prophylaxis or treatment of a recurring disease or condition. These benefits can be seen in FIG. 2, which illustrates that the process of manufacturing the enucleated cells of the present disclosure is roughly 2 months, as compared with suitable timelines, which is 12 months or longer.

Existing red blood cell or platelet therapeutic platforms are enucleated by erythropoiesis in which the blood cell is terminally differenced and intracellular organelles and ribosomes are eliminated, some of which are responsible for protein synthesis and secretion. Thus, the resulting red blood cell or platelet loses the cell-like functionality (e.g., protein expression, secretion, cell motility, chemokine sensing, homing capabilities, etc.) after enucleation by erythropoiesis that may be important for therapeutic applications, such as producing, delivering or secreting a therapeutic agent in vivo. By contrast, the enucleated cells described herein retain one or more intracellular organelles after enucleation that are endogenous to the parent cell. In some embodiments, all of the one or more intracellular organelles are retained. In some embodiments, fewer than all of the one or more intracellular organelles are retained. In some embodiments, the Golgi apparatus and/or the endoplasmic reticulum are retained, which are involved in protein synthesis and secretion. Retention of the one or more intracellular organelles at least partially enables the enucleated cells to synthesize or release the biomolecule disclosed herein (e.g., single-domain antibody, or portion thereof, targeting moiety, immune-evading moiety, etc.) in the absence of the nucleus.

The enucleated cells disclosed herein may be derived from virtually any nucleated cell (referred to herein as “parent” cell). In some embodiments, the parent cell is an immune cell. In some embodiments, the immune cell is a neutrophil, eosinophil, basophil, mast cell, monocyte, macrophage, dendritic cell, natural killer cell, or lymphocyte (B cells and T cells). In some embodiments, the parent cell is a stem cell. In some embodiments, the parent cell is an adult stem cell. In some embodiments, the parent cell is a mesenchymal stromal cell (MSC). In some embodiments, the enucleated cell is derived from an inducible pluripotent stem cell (iPSC). In some embodiments, the parent cell is not an erythrocyte. In some embodiments, the parent cell is not an erythroid precursor cell. In some embodiments, the parent cell is not an endothelial cell. In some embodiments, the parent cell is not an endothelial precursor cell.

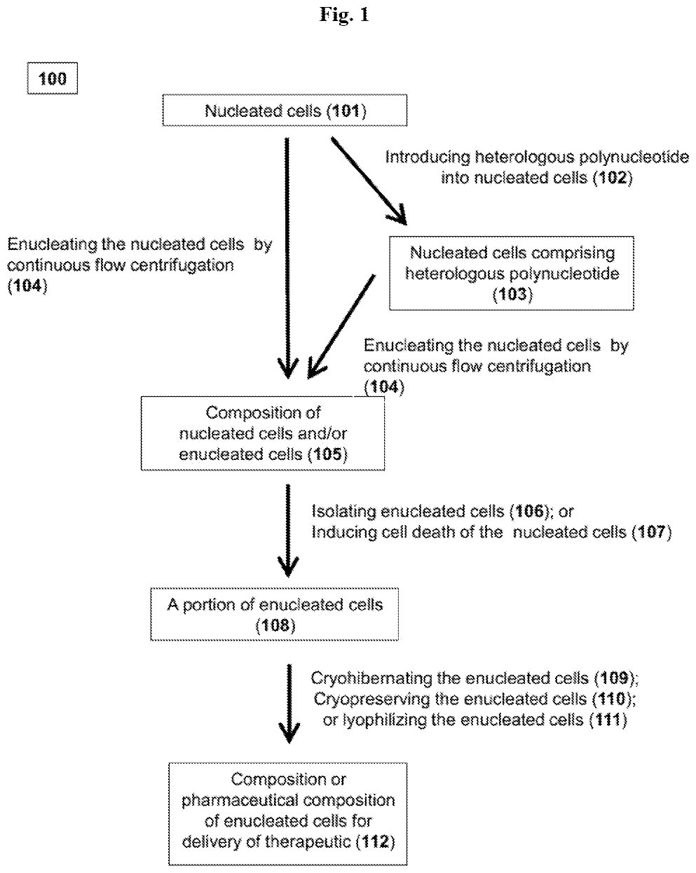

Described herein are methods for manufacturing enucleated cells in an increased quantity and purity, where the manufactured enucleated cells can be formulated into a composition or a pharmaceutical composition for treating a disease or condition in a subject in need thereof. FIG. 1 illustrates a non-limiting example of the manufacturing of the enucleated cells described herein (100). Nucleated cells (101) can be isolated from the subject and cultured in vitro for clonal expansion. In some embodiments, the nucleated cells (101) can also be immortalized or derived from a cell line. In some embodiments, the nucleated cells can be engineered (103) to comprise a heterologous polynucleotide (102). The nucleated cells can then be enucleated by continuous flow centrifugation (104). The use of continuous flow centrifugation for enucleating cells presents an improvement over the currently available methods for enucleation, where the enucleation conducted via continuous flow centrifugation increases the quantity (e.g., yield) or purity of the enucleated cells obtained from the nucleated cells. After obtaining the composition (105) of the enucleated cells (which may have residual nucleated cells), the composition can be further purified for the enucleated cells by selecting for markers of the enucleated cells (106) or by inducing cell death of the remaining residual nucleated cells (107) to obtain a portion of enucleated cells (108). The portion of enucleated cells can be cryohibernated (109), cryopreserved (110), lyophilized (111), or a combination thereof and be formulated into a composition or a pharmaceutical composition for delivery of therapeutic for treating the disease or condition in the subject.

Described herein, in some aspects, is a method of delivering a therapeutic agent to a target cell of a subject. In some embodiments, the target cell of the subject comprises a lung cell. In some embodiments, the target cell of the subject comprises a liver cell. In some embodiments, the method comprises introducing a plurality of enucleated cells comprising the therapeutic agent to the subject or a sample of the subject in vivo or ex vivo under conditions sufficient to deliver the therapeutic agent to the target cell of the subject. In some embodiments, the plurality of enucleated cells is obtained from a cryopreserved composition or a cryohibernated composition. In some embodiments, the therapeutic agent is delivered to the target cell in an amount that is greater than or equal to about an amount of the therapeutic agent delivered to an otherwise comparable target cell of the subject by otherwise comparable enucleated cells that were not cryopreserved or not cryohibernated. In some embodiments, the method comprises preparing a fluid formulation comprising the plurality of enucleated cells from the cryopreserved composition or the cryohibernated composition. In some embodiments, the cryopreserved composition is cryopreserved for at least about 24 hours, at least about 48 hours, at least about 72 hours, at least 96 about hours, at least about 5 days, at least about 6 days, at least about 7 days, at least about 10 days, at least about 15 days, at least about one month, at least about one month, or at least about one year, or for an indefinite period of time. In some embodiments, the cryopreservation comprises storing the plurality of enucleated cells at a temperature at about −80° C. In some embodiments, the cryopreserved composition is stored at most at about −80° C. In some embodiments, the cryopreserved composition is stored at most at about −80° C. for at least 24 hours. In some embodiments, the cryopreservation comprises storing the plurality of enucleated cells at a temperature at about −20° C. In some embodiments, the cryopreservation comprises storing the plurality of enucleated cells in liquid nitrogen. In some embodiments, the cryopreservation comprises contacting and storing the plurality of enucleated cells with a freezing media described herein. In some embodiments, the freezing media comprises an xeno-free media. In some embodiments, the plurality of enucleated cells from the cryopreserved composition are suspended in Xeno-free media. In some embodiments, the plurality of enucleated cells from the cryopreserved composition are suspended in a freezing media. In some embodiments, the freezing media comprises about 2% DMSO. In some embodiments, the freezing media comprises about 5% DMSO. In some embodiments, the freezing media comprises about 10% DMSO. In some embodiments, the freezing media comprises at least 5% or at least 10% DMSO. In some embodiments, the freezing media comprises CryoStor® media. In some embodiments, the CryoStor® media is CryoStor® CS5. In some embodiments, the CryoStor® media is CryoStor® CS10. In some embodiments, the method comprises thawing the plurality of enucleated cells, where the plurality of enucleated cells exhibit comparable cellular function or vitality compared to the plurality of enucleated cells without being cryopreserved. In some embodiments, the thawing comprises contacting the plurality of enucleated cells in a water bath. In some embodiments, the thawing comprises contacting the plurality of enucleated cells at room temperature. In some embodiments, the thawing the cryopreserved composition is performed at room temperature. In some embodiments, the thawing comprises contacting the plurality of enucleated cells in at 37° C. In some embodiments, the thawing the cryopreserved composition is performed at 37° C.

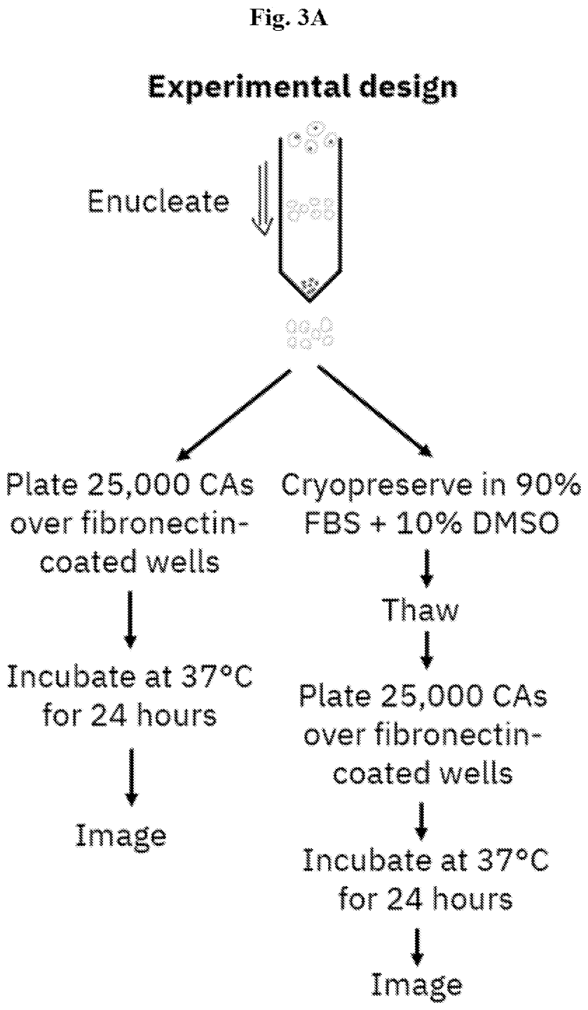

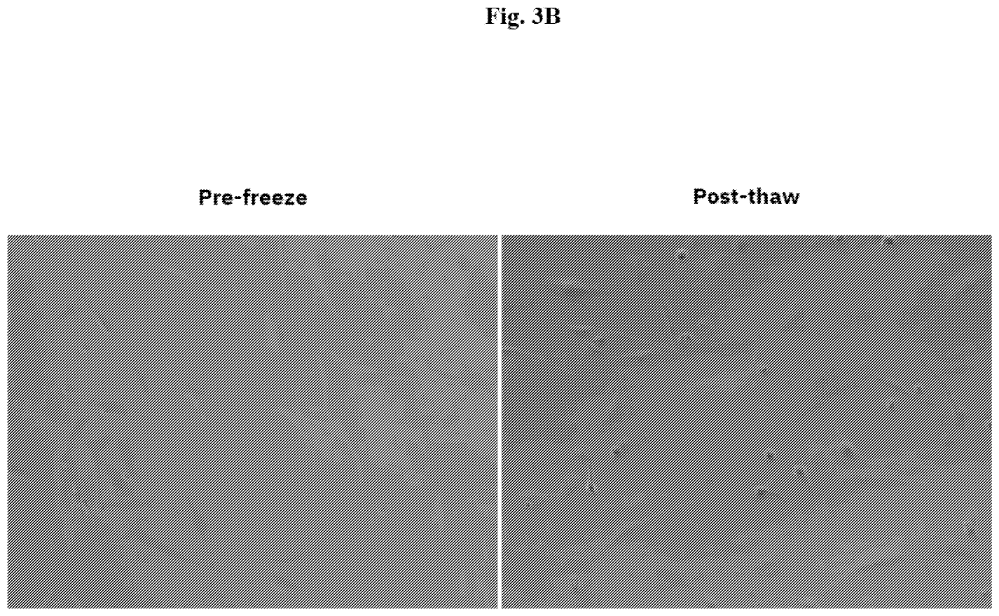

As a nonlimiting example, FIG. 3A shows a workflow starting with thawing the cryopreserved vials of enucleated cells in a 37° C. water bath and ending with imagine the cells to identify enucleation efficiency. The left branch of FIG. 3A is shown as an image prior to cryopreservation as shown in FIG. 3B (left). The right branch of FIG. 3A is shown as an image after cryopreservation as shown in FIG. 3B (right).

In some embodiments, the plurality of enucleated cells comprises a therapeutic agent. In some embodiments, the therapeutic agent comprises a virus, an exogenous DNA molecule, an exogenous RNA molecule, an exogenous protein, an exogenous peptide, or any combination thereof. In some embodiments, the therapeutic agent comprises the virus. In some embodiments, the virus is an adeno-associated virus (AAV), an adenovirus, a reovirus, a coxsackie virus, a retrovirus, a poxvirus, a baculovirus, or a herpes virus. In some embodiments, the virus comprises an oncolytic virus. In some embodiments, the oncolytic virus is an adenovirus, a human immunodeficiency virus, a Maraba virus, a Measles virus, a Newcastle disease virus, a poliovirus, a Seneca Valley virus, a parvovirus, a Semliki Forest virus, a Vesicular Stomatitis virus, a Sindbis virus, or any combination thereof. In some embodiments, the amount of the virus delivered to the subject is measured in viral titers in the target cell. In some embodiments, the viral titers measured in the target cell are greater than the viral titers measured in the otherwise comparable target cell. In some embodiments, the viral titers measured in the target cell are equal to about the viral titers measured in the otherwise comparable target cell. In some embodiments, the exogenous protein comprises a cytokine or a cytokine receptor-binding fragment thereof. In some embodiments, the amount of the cytokine or the cytokine receptor-binding fragment thereof delivered to the subject is measured by the secretion of the cytokine or the cytokine receptor-binding fragment thereof from the plurality of enucleated cells. In some embodiments, the secretion of the cytokine or the cytokine receptor-binding fragment thereof measured is greater than or equal to about the secretion of the cytokine or the cytokine receptor-binding fragment thereof by an otherwise comparable enucleated cell that was not cryopreserved. In some embodiments, the secretion of the cytokine or the cytokine receptor-binding fragment thereof measured is greater than or equal to about the secretion of the cytokine or the cytokine receptor-binding fragment thereof by an otherwise comparable nucleated cell that was cryopreserved. In some embodiments, the exogenous protein comprises an immune checkpoint inhibitor. In some embodiments, the exogenous protein comprises an antigen. In some embodiments, the exogenous protein comprises an immunomodulatory protein. In some embodiments, the immune checkpoint inhibitor comprises an inhibitor specific to PD-L1, PD-1, or a combination thereof. In some embodiments, the therapeutic agent comprises the exogenous RNA molecule. In some embodiments, the exogenous RNA molecule encodes a cytokine or the cytokine receptor-binding fragment thereof, a chemokine, or any combination thereof. In some embodiments, the exogenous RNA molecule encodes the cytokine or the cytokine receptor-binding fragment thereof. In some embodiments, the cytokine or the cytokine receptor-binding fragment thereof comprises interleukin-12 (IL-12), interferon-α (IFN-α), interferon-β (IFN-β), interferon-γ (IFN-γ), interleukin-7 (IL-7), interleukin-21 (IL-21), tumor necrosis factor α (TNF-α), granulocyte-macrophage colony-stimulating factor (GM-CSF), interleukin-15 (IL-15), or any combination thereof. In some embodiments, the exogenous RNA molecule encodes the chemokine. In some embodiments, the chemokine comprises stromal cell-derived factor-1α (SDF1α), C-C motif chemokine ligand 2 (CCL2), C-C motif chemokine ligand 3 (CCL3), C-C motif chemokine ligand 5 (CCL5), C-C motif chemokine ligand 8 (CCL8), C-C motif chemokine ligand 1 (CCL1), CXC motif chemokine ligand 9 (CXCL9), CXC motif chemokine ligand 10 (CXCL10), C-C motif chemokine ligand 11 (CCL11), CXC motif chemokine ligand 12) CXCL12, or any combination thereof. In some embodiments, the exogenous RNA molecule encodes an antigen. In some embodiments, the exogenous RNA molecule encodes an immunomodulatory protein. In some embodiments, the exogenous RNA molecule encodes an immune checkpoint inhibitor. In some embodiments, the immune checkpoint inhibitor comprises an inhibitor specific to PD-L1, PD-1, or a combination thereof. In some embodiments, the therapeutic agent comprises a cytokine or cytokine receptor-binding fragment thereof. In some embodiments, the amount of the cytokine or the cytokine receptor-binding fragment released in vivo or ex vivo is a measurement of the secretion of the cytokine or the cytokine receptor-binding fragment thereof from the plurality of enucleated cells. In some embodiments, the amount of the cytokine or the cytokine receptor-binding fragment thereof measured is greater than or equal to about the secretion of the cytokine or the cytokine receptor-binding fragment thereof by the otherwise comparable enucleated cells that were not cryopreserved. In some embodiments, the amount of the cytokine or the cytokine receptor-binding fragment thereof measured is greater than or equal to about the secretion of the cytokine or the cytokine receptor-binding fragment thereof by the otherwise comparable nucleated cells that were cryopreserved. In some embodiments, the exogenous RNA molecule encodes an antigen. In some embodiments, the exogenous RNA molecule encodes an immunomodulatory protein.

In some embodiments, the plurality of the nucleated cells encoding the therapeutic agent can treat a disease or condition described herein. In some embodiments, the method further comprises treating a disease or a condition in the subject. In some embodiments, the disease is cancer. In some embodiments, the cancer comprises a solid tumor. In some embodiments, the cancer is lung cancer, cancer metastases in lung tissue, liver cancer, or cancer metastases in liver tissue. In some embodiments, the liver cancer is a hepatocellular carcinoma or a cholangiocarcinoma. In some embodiments, the cancer is the lung cancer. In some embodiments, the lung cancer is a small cell lung cancer, a non-small lung cancer, or a bronchial carcinoids. In some embodiments, the lung cancer is a small cell lung cancer. In some embodiments, the lung cancer is bronchial carcinoids. In some embodiments, the lung cancer is a non-small cell lung cancer. In some embodiments, the non-small cell lung cancer is an adenocarcinomas, squamous cell carcinomas, or large cell carcinomas.

In some embodiments, the methods of enucleation disclosed herein result in a composition comprising the enucleated cells (also referred to herein as “enucleated cell fraction” of the composition). In some embodiments, the composition further comprises less than or equal to about one (1) percent (%) residual nucleated cells (also referred to herein as “nucleated cell fraction” of the composition) by volume that were not enucleated. In some embodiments, the nucleated cell fraction comprises less than or equal to about 0.1%, 0.2%, 0.3%, 0.4%, 0.5%, 0.6%, 0.7%, 0.8%, or 0.9% of the composition by volume. In some embodiments, the nucleated cell fraction comprises 0.1% to about 0.2%, about 0.1% to about 0.3%, about 0.1% to about 0.4%, about 0.1% to about 0.5%, about 0.1% to about 0.6%, about 0.1% to about 0.7%, about 0.1% to about 0.8%, about 0.1% to about 0.9%, or about 0.1% to about 1.0% of the composition by volume. In some embodiments, the nucleated cell fraction comprises about 0.2% to about 0.3%, about 0.2% to about 0.4%, about 0.2% to about 0.5%, about 0.2% to about 0.6%, about 0.2% to about 0.7%, about 0.2% to about 0.8%, about 0.2% to about 0.9%, or about 0.2% to about 1.0% of the composition by volume. In some embodiments, the nucleated cell fraction comprises about 0.3% to about 0.4%, about 0.3% to about 0.5%, about 0.3% to about 0.6%, about 0.3% to about 0.7%, about 0.3% to about 0.8%, about 0.3% to about 0.9%, or about 0.3% to about 1.0% of the composition by volume. In some embodiments, the nucleated cell fraction comprises about 0.4% to about 0.5%, about 0.4% to about 0.6%, about 0.4% to about 0.7%, about 0.4% to about 0.8%, about 0.4% to about 0.9%, or about 0.4% to about 1.0% of the composition by volume. In some embodiments, the nucleated cell fraction comprises about 0.5% to about 0.6%, about 0.5% to about 0.7%, about 0.5% to about 0.8%, about 0.5% to about 0.9%, or about 0.5% to about 1.0% of the composition by volume. In some embodiments, the nucleated cell fraction comprises about 0.6% to about 0.7%, about 0.6% to about 0.8%, about 0.6% to about 0.9%, or about 0.6% to about 1.0% of the composition by volume. In some embodiments, the nucleated cell fraction comprises about 0.7% to about 0.8%, about 0.7% to about 0.9%, or about 0.7% to about 1.0% of the composition by volume. In some embodiments, the nucleated cell fraction comprises about 0.8% to about 0.9%, or about 0.8% to about 1.0% of the composition by volume. In some embodiments, the nucleated cell fraction comprises about 0.9% to about 1.0% of the composition by volume. In some embodiments, the nucleated cell fraction is eliminated by induced cell death following the enucleation. In some embodiments, the induced cell death is employed using biomolecular suicide switches that are expressed in response to an external stimulus, such as for example, exposure to a small molecule drug (e.g., rimiducid), a prodrug (e.g., ganciclovir), or the like.

Also described herein are pharmaceutical compositions and formulations comprising the compositions described herein, and a pharmaceutically acceptable: carrier, excipient, diluent, or nebulized inhalant. The pharmaceutical compositions are provided in pharmaceutical formulations. In some embodiments, the pharmaceutical formulations are formulated for administration to a subject as a combination therapy (e.g., prodrug, adjuvant, additional therapeutic agent, or other therapy) or monotherapy. In some embodiments, the pharmaceutical formulations are formulated for systemic administration or at the site of action, such as intratumoral administration.

Disclosed herein are kits comprising the composition disclosed herein and packaging material configured to deliver the composition to an individual. The kits disclosed herein may comprise a composition comprising a enucleated cell fraction and less than 0.1% nucleated cell fraction. In some embodiments, the kits further comprise instructions for further engineering the enucleated cells in the enucleated cell fraction, such as for example, to produce or secrete a therapeutic agent disclosed herein. In some embodiments, the kits further comprise a stimulus used to trigger expression or activity of biomolecular suicide switch in the nucleated cell fraction of the composition. In either case, the instructions may further comprise instructions for how to formulate the resulting composition into a pharmaceutical formulation for administration to a subject disclosed herein.

### Compositions

Disclosed herein are compositions thereof comprising enucleated cells capable of being extensively engineered to express an active agent, or portion thereof, in the absence of a nucleus. Such enucleated cells are viable cell-like entities capable of synthesizing, releasing (e.g., secreting), or delivering the active agent to a target cell or tissue in the absence of the nucleus. The compositions disclosed herein can be stored in a suspended biological stage by means such as cryohibernation, cryopreservation, or lyophilization for any period of time without impacting the viability of the enucleated cell once the biological activity is revived. In some embodiments, the compositions disclosed here are cryopreserved. Moreover, the compositions disclosed herein comprise less than or equal to about 0.1% of nucleated cells (e.g., parent cells that were not enucleated during the enucleation process), rendering the compositions disclosed herein optimal for therapeutic applications. The enucleated cells (as referred to here as “cytoplasts”) may further comprise naturally occurring cell-surface molecules retained from the parent cell. In some embodiments, the enucleated cells further comprise exogenous molecules, such as a targeting moiety, a transmembrane moiety, an additional therapeutic agent (e.g., other than the active agent) such as those disclosed herein.

### (a) Enucleated Cell

The enucleated cells of the present disclosure are obtained or derived from a corresponding nucleated cell (referred to herein as a “parent cell”). The parent cell may be derived from a variety of different cell types, including eukaryotic cells. For example, an enucleated cell may be derived from an adult stem cell, a mesenchymal stromal cell (MSC), a natural killer (NK) cell, a macrophage, a myoblast, a neutrophil, endothelial cell, endothelial precursor cell, and/or a fibroblast. In some embodiments, an enucleated cell is derived from a mesenchymal stromal cell. In some embodiments, the enucleated cell is derived from an inducible pluripotent stem cell (iPSC). In some embodiments, the parent cell is derived from a cell is immortalized using suitable methods. In some embodiments, the enucleated cell comprises or retains one or more structural features of the parent cell, including intracellular organelles, one or more tunneling nanotubes, or a combination thereof. In some embodiments, the enucleated cell comprises one or more intracellular organelles for synthesis or secretion of an exogenous polypeptide (e.g., therapeutic agent) in absence of the nucleus. In some embodiments, the one or more intracellular organelles comprise a Golgi apparatus, an endoplasmic reticulum, or a combination thereof. In some embodiments, the enucleated cell comprises or expresses any one of the therapeutic agents described herein.

In some embodiments, the cell can originate from any organism having one or more cells. Non-limiting examples of cells include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell). In some embodiments, the cell is a somatic cell. In some embodiments, the cell is a stem cell or a progenitor cell. In some embodiments, the cell is a mesenchymal stem or progenitor cell. In some embodiments, the cell is a hematopoietic stem or progenitor cell. In some embodiments, the cell is a muscle cell, a skin cell, a blood cell, or an immune cell. Other non-limiting example of cells includes lymphoid cells, such as B cell, T cell (Cytotoxic T cell, Natural Killer T cell, Regulatory T cell, T helper cell), Natural killer cell, cytokine induced killer (CIK) cells; myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

In some embodiments, the cell is a eukaryotic cell. Non-limiting examples of eukaryotic cells include mammalian (e.g., rodent, non-human primate, or human), non-mammalian animal (e.g., fish, bird, reptile, or amphibian), invertebrate, insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. In some embodiments, the nucleated cell is a primary cell. In some embodiments, the nucleated cell is an immune cell (e.g., a lymphocyte (e.g., a T cell, a B cell), a macrophage, a natural killer cell, a neutrophil, a mast cell, a basophil, a dendritic cell, a monocyte, a myeloid-derived suppressor cell, an eosinophil). In some embodiments, the nucleated cell is a phagocyte or a leukocyte. In some embodiments, the nucleated cell is a stem cell (e.g., an adult stem cell (e.g., a hematopoietic stem cell, a mammary stem cell, an intestinal stem cell, mesenchymal stem cell, an endothelial stem cell, a neural stem cell, an olfactory adult stem cell, a neural crest stem cell, a testicular cell), an embryonic stem cell, an inducible pluripotent stem cell (iPS)). In some embodiments, the nucleated cell is a progenitor cell. In some embodiments, the nucleated cell is from a cell line. In some embodiments, the nucleated cell is a suspension cell. In some embodiments, the nucleated cell is an adherent cell. In some embodiments, the nucleated cell is a cell that has been immortalized by expression of an oncogene. In some embodiments, the nucleated cell is immortalized by the expression of human telomerase reverse transcriptase (hTERT) or any oncogene. In some embodiments, the nucleated cell is a patient or subject derived cell (e.g., an autologous patient-derived cell, or an allogenic patient-derived cell). In some embodiments, the nucleated cell is transfected with a vector (e.g., a viral vector (e.g., a retrovirus vector (e.g., a lentivirus vector), an adeno-associated virus (AAV) vector, a vesicular virus vector (e.g., vesicular stomatitis virus (VSV) vector), or a hybrid virus vector), a plasmid) before the nucleated cell is enucleated using any of the enucleation techniques described herein and known in the art.

In some embodiments, the cytoplast is derived from a cell autologous to the subject. In some embodiments, the cytoplast is derived from a cell allogenic to the subject.

In some embodiments, the cytoplast is derived from an immune cell. In some embodiments, the cytoplast is derived from a natural killer (NK) cell, a neutrophil, a macrophage, a lymphocyte, a fibroblast, an adult stem cell (e.g., hematopoietic stem cell, a mammary stem cell, an intestinal stem cell, a mesenchymal stem cell, a mesenchymal stromal cell, an endothelial stem cell, a neural stem cell, an olfactory adult stem cell, a neural crest stem cell, a skin stem cell, or a testicular cell), a mast cell, a basophil, an eosinophil, an endothelial cell, an endothelial cell precursor cell, or an inducible pluripotent stem cell.

In some embodiments, the parent cell may be enucleated and engineered for therapeutic use. In some embodiments, a parent cell may be treated with cytochalasin to soften the cortical actin cytoskeleton. In some embodiments, the nucleus is then physically extracted from the cell body by high-speed centrifugation in gradients of polysaccharide to generate an enucleated cell. In some embodiments, the polysaccharide is Ficoll for generating Ficoll gradients to generate an enucleated cell. Because enucleate cells and intact nucleated cells sediment to different layers in the Ficoll gradient, enucleated cells may be isolated and prepared for therapeutic purposes or fusion to other cells (nucleated or enucleated). The enucleation process can be clinically scalable to process tens of millions of cells by utilizing the methods described herein. In some embodiments, enucleated cells may be used as a disease-homing vehicle to deliver clinically relevant cargos or payloads to treat various diseases or conditions described herein.

In some embodiments, the enucleated cell comprises at least one therapeutic agent. In some embodiments, the enucleated cells disclosed herein express the therapeutic agent with one or more intracellular organelles in the absence of the nucleus. In some embodiments, the therapeutic agent is exogenous to the enucleated cell or parent (nucleated) cell thereof. In some embodiments, the enucleated cell expresses the therapeutic agent at the surface of the enucleated cell. In some embodiments, the therapeutic agent is secreted by the enucleated cell into extracellular space at a target tissue (e.g., a microenvironment). In some embodiments, the therapeutic agent is cargo (e.g., encapsulated by the enucleated cell) of the enucleated cell.

In some embodiments, the enucleated cell is obtained from a first subset of a plurality of nucleated cells. In some embodiments, the enucleated cells are in a composition, which further comprises a second subset of the plurality of the nucleated cells. In some embodiments, the second subset of the nucleated cells comprises less than about 0.1% by volume of the composition. In some embodiments, the second subset of the nucleated cells comprises less than about 0.5% by volume of the composition. In some embodiments, the second subset of the nucleated cells comprises less than about 1% by volume of the composition. In some embodiments, the second subset of the nucleated cells comprises less than about 5% by volume of the composition. In some embodiments, the second subset of the nucleated cells comprises less than about 10% by volume of the composition. In some embodiments, the second subset of the nucleated cells comprises less than about 15% by volume of the composition. In some embodiments, the second subset of the nucleated cells comprises less than about 20% by volume of the composition. In some embodiments, the second subset of the nucleated cells comprises less than about 25% by volume of the composition. In some embodiments, the second subset of the nucleated cells comprises less than about 30% by volume of the composition. In some embodiments, the second subset of the nucleated cells comprises less than about 40% by volume of the composition. In some embodiments, the second subset of the nucleated cells comprises less than about 50% by volume of the composition.

In one aspect, the nucleated cell (e.g., the parent cell prior to enucleation to yield the enucleated cell described herein) comprises a heterologous polynucleotide encoding a heterologous gene product configured to induce cell death of the nucleated cell. In some embodiments, the heterologous polynucleotide comprises a promoter. In some embodiments, the promoter is configured to activate transcription of the heterologous polynucleotide under conditions sufficient to express the heterologous gene product. In some embodiments, the promoter comprises an inducible promoter. In some embodiments, an inducible promoter is configured to activate transcription of the heterologous polynucleotide under conditions sufficient to express the heterologous gene product when induced.

In some embodiments, the enucleated cell described herein can be cryopreserved, cryohibernated, lyophilized, or a combination thereof. In some embodiments, the cryopreserved enucleated cell, following thawing, the enucleated cell is as viable as an otherwise comparable enucleated cell that is not cryopreserved. In some embodiments, the lyophilized enucleated cell is as viable as an otherwise comparable enucleated cell that is not lyophilized. In some embodiments, the cryohibernated enucleated cell is as viable as an otherwise comparable enucleated cell that is not cryohibernated.

In some embodiments, the enucleated cell or the composition comprising the enucleated cell may be cryopreserved (e.g., storing the enucleated cell or the composition comprising the enucleated cell at freezing temperature) or cryohibernated (e.g., storing the enucleated cell or the composition comprising the enucleated cell at a temperature that is between the ambient temperature and freezing temperature). The duration of cryopreservation or cryohibernation may be greater than or equal to about one hour, two hours, six hours, 12 hours, one day, two days, three days, four days, five days, six days, one week, two weeks, three weeks, four weeks, one month, two months, three months, or longer period of time. In some embodiments, the enucleated cell exhibits a viability after cryopreservation or cryohibernation that is greater than or equal to about 50%, 55%, 60%, 65%, 70%, 75%, 80%, 85%, 90%, 95%, or 99% similar to a comparable cell (e.g., a parent cell or an enucleated cell described herein that has not been cryopreserved or cryo-hibernated) after same the period of time of cryopreservation or cryohibernation. In some embodiments, the enucleated cell exhibits the viability following the cryohibernation as measured at 24 hours following the cryohibernation that is equal to or greater than the viability of a comparable enucleated cell that is not cryohibernated. In some embodiments, the enucleated cell exhibits the viability following the cryopreservation as measured at 24 hours following the cryopreservation that is equal to or greater than the viability of a comparable enucleated cell that is not cryopreserved. Viability in this context may be measured by Trypan blue dye exclusion as described herein. In some embodiments, the Trypan blue dye exclusion is performed by: (a) centrifuging an aliquot of a plurality of the cell without the nucleus in a suspension to create a cell pellet; (b) resuspending the cell pellet in serum-free medium to produce a serum-free cell suspension; (c) mixing 1 part Trypan blue dye and 1 part of the serum-free cell suspension; (d) counting the plurality of the cells without the nucleus within 3-5 minutes of (c), wherein at least some of the plurality of cells without the nucleus are unstained with the Trypan blue dye, which is indicative of viability. In some embodiments, the viability is measured using Annexin-V cell surface staining. In some embodiments, the viability is measured by expression of the exogenous polypeptide. For example, the viability of the enucleated cell can be determined by the expression of the exogenous antibody or single-domain antibody expressed by the enucleated cell. In some embodiments, the viability is measured by expression of cell surface markers of any one of the cell surface markers described herein such as CD105, CD90, CD45, CXCR4, PSGL-1, or CCR2. In some embodiments, the viability is measured by the cell activity of the enucleated cell. In some embodiments, the viability is measured by the homing capability of the enucleated cell as determined by the chemosensing or chemokine homing activity described herein.

In one aspect, the enucleated cells of the present disclosure are cryopreserved. In some embodiments, the enucleated cells are cryopreserved for between about 24 hours to about 5 years. In some embodiments, the enucleated cells are cryopreserved for between about 12 hours to about 24 hours, between about 24 hours to about 48 hours, between about 48 hours to about 72 hours, between about 72 hours to about 96 hours, between about 96 hours to about 120 hours, between about 120 hours to about 144 hours, between about 144 hours to about 168 hours, between seven days to about 14 days, between about 14 days to about 21 days, between about 21 days to about 28 days, between about 28 days to about 30 days, between about 30 days to about 31 days, between about one month to about two months, between about two months to about three months, between about three months to about four months, between about four months to about five months, between about five months to about six months between about six months to about seven months, between about seven months to about eight months, between about eight months to about nine months, between about nine months to about ten months, between about ten months to about 11 months, between about 11 months to about 12 months, between about one year to about 1.5 years, between about 1.5 years to about 2 years, between about 2 years to about 2.5 years, between about 2.5 years to about 4 years, between about 4 years to about 4.5 years, between about 4.5 years to about 5 years, or more.

In some embodiments, the enucleated cells are cryopreserved for at least about 12 hours, at least about 24 hours, at least about 36 hours, at least about 48 hours, at least about 60 hours, at least about 72 hours, at least about 84 hours, at least about 96 hours, at least about 108 hours, at least about 120 hours, at least about 132 hours, at least about 144 hours, at least about 156 hours, at least about 168 hours, at least about 14 days, at least about 21 days, at least about 28 hours, at least about 30 days, at least about 31 days, at least about two months, at least about three months, at least about four months, at least about five months, at least about six months, at least about seven months, at least about eight months, at least about nine months, at least about ten months, at least about 11 months, at least about 12 months, at least about 1.5 years, at least about 2 years, at least about 2.5 years, at least about 3 years, at least about 3.5 years, at least about 4 years, at least about 4.5 years, at least about 5 years, or more. In some embodiments, the enucleated cells are cryopreserved for at least about 24 hours. In some embodiments, the enucleated cells are cryopreserved for at least about 7 days. In some embodiments, the enucleated cells are cryopreserved for at least about one month. In some embodiments, the enucleated cells are cryopreserved for at least about one year.

In some embodiments, the enucleated cells are cryopreserved for about 12 hours, about 24 hours, about 36 hours, about 48 hours, about 60 hours, about 72 hours, about 84 hours, about 96 hours, about 108 hours, about 120 hours, about 132 hours, about 144 hours, about 156 hours, about 168 hours, about 14 days, about 21 days, about 28 hours, about 30 days, about 31 days, about two months, about three months, about four months, about five months, about six months, about seven months, about eight months, about nine months, about ten months, about 11 months, about 12 months, about 1.5 years, about 2 years, about 2.5 years, about 3 years, about 3.5 years, about 4 years, about 4.5 years or about 5 years.

In some embodiments, the enucleated cells are cryopreserved at temperature below at least about −70° C. In some embodiments, the enucleated cells are cryopreserved between about −70° C. to about −80° C., between about −80° C. to about −90° C., between about −90° C. to about −100° C., between about −100° C. to about −110° C., between about −110° C. to about −120° C., between about −120° C. to about −130° C., between about −130° C. to about −140° C., between about −140° C. to about −150° C., between about −150 to about −160° C., between about −160° C. to about −170° C., between about −170° C. to about −180° C., between about −180° C. to about −190° C., or between −190° C. to about −200° C.

In some embodiments, the enucleated cells are cryopreserved at a temperature of at least about −70° C., at least about −80° C., at least about −90° C., at least about −100° C., at least about −110° C., at least about −120° C., at least about −130° C., at least about −140° C., at least about −150° C., at least about −160° C., at least about −170° C., at least about −180° C., at least about −190° C., at least about −200° C., or more. In some embodiments, the enucleated cells are cryopreserved at a temperature of about −70° C., about −80° C., about −90° C., about −100° C., about −110° C., about −120° C., about −130° C., about −140° C., about −150° C., about −160° C., about −170° C., about −180° C., about −190° C., about −200° C., or more.

In some embodiments, the enucleated cells are cryopreserved in liquid nitrogen. In some embodiments, the cryopreserved composition is cryopreserved in liquid nitrogen. In some embodiments, the enucleated cells are cryopreserved in dry ice. In some embodiments, the enucleated cells are cryopreserved in a freezer. In some embodiments, the freezer is set at any temperature in the present disclosure.

In some embodiments, the cryopreserved composition further comprises a freezing media. In some embodiments, the freezing media is serum freezing media. In some embodiments, the serum freezing media has fetal bovine serum. In some embodiments, the freezing media is serum-free media. In some embodiments, the freezing media comprises dimethyl sulfoxide (DMSO). In some embodiments, the freezing media comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% DMSO. In some embodiments, the freezing media comprises glycerol. In some embodiments, the freezing media is a Xeno-free media. Non-limiting examples of Xeno-free media includes Xeno-free X-VIVO media, Xeno-free mesenchymal stem cell media, and StemPro™ MSC SFM Xeno-free media. In some embodiments, the Xeno-free media is supplemented with human blood serum, platelet lysates, holo-transferrin, or insulin. In some embodiments, the Xeno-free media is supplemented with human blood serum. In some embodiments, the Xeno-free media is supplemented with platelet lysates. In some embodiments, the Xeno-free media is supplemented with holo-transferrin. In some embodiments, the Xeno-free media is supplemented with insulin. In some embodiments, the freezing media is a CryoStor® media. Non-limiting examples of CryoStor® media include CS10, CS5, CS2, and CSB. In some embodiments, the CryoStor® is CryoStor® CS10 media.

In some embodiments, the cryopreserved composition further comprises a nonpyrogenic solution. Non-limiting examples of nonpyrogenic solution include lactated ringer's solution, cupric chloride solution, mannitol solution, phosphate buffer solution (PBS), sodium chloride solution, and sodium lactate solution. In some embodiments, the nonpyrogenic solution is PBS. In some embodiments, the nonpyrogenic solution is sodium lactate solution. In some embodiments, the nonpyrogenic solution is sterile.

In some embodiments, the cryopreserved enucleated cells further comprise one or more therapeutic agents.

In some embodiments, the cryopreserved enucleated cells release one or more therapeutic agent. In some embodiments, the cryopreserved enucleated cell releases one or more therapeutic agent in an amount that is greater than or equal to about an amount of the therapeutic agent released by an otherwise identical target cell by otherwise identical enucleated cells that were not cryopreserved. In some embodiments, the cryopreserved enucleated cell releases between about 1-fold greater to about 50-fold greater than the amount of the one or more therapeutic agents compared to the otherwise comparable enucleated cell. In some embodiments, the cryopreserved enucleated cell releases between about 1-fold greater to about 2 fold greater, between about 2-fold greater to about 3-fold greater, between about 3-fold greater to about 4-fold greater, between about 4-fold greater to about 5-fold greater, between about 5-fold between to about 10-fold greater, between about 10-fold greater to about 15-fold greater, between about 15-fold greater to about 20 fold greater, between about 20-fold greater to about 25-fold greater, between about 25-fold greater to about 30-fold greater, between about 30-fold greater to about 35-fold greater, between about 35-fold greater to about 40-fold greater, between about 40-fold greater to about 45-fold greater, or between about 45-fold greater to about 50-fold greater than the amount of the one or more therapeutic agents that is released by the otherwise identical enucleated cell. In some embodiments, the cryopreserved enucleated cell releases a therapeutic agent in an amount that is about one-fold greater, about two-fold greater, about three-fold greater, about four-fold greater, about five-fold greater, about 10-fold greater, about 15-fold greater, about 20-fold greater, about 25-fold greater, about 30-fold greater, about 35-fold greater, about 40-fold greater, about 45-fold greater, about 50-fold greater, or more compared to the amount of the one or more therapeutic agents that is released by the otherwise identical enucleated cell. In some embodiments, the amount of the one or more therapeutic agents that is released by the cryopreserved enucleated cell is equal to the amount of the one or more therapeutic agents that is released by the otherwise identical enucleated cell.

In some embodiments, the amount of the one or more therapeutic agents released is measured in vitro. In some embodiments, the amount of the one or more therapeutic agents released is measured in vivo. In some embodiments, the amount of the one or more therapeutic agents released is measured ex vivo.

In some embodiments, the therapeutic agent is a virus. In some embodiments, the amount of the therapeutic agent released is a measurement of viral titers in a target cell. In some embodiments, the viral titer is measured in plaque-forming units (PFU). In some embodiments, the PFU is measured as PFU per milliliter (mL). In some embodiments, the PFU is measured as PFU is measured as PFU per gram. In some embodiments, the target cell is a biological sample. Non-limiting examples of biological samples include isolated cells, cellular supernatant, tissue biopsy, tumor biopsy, cell lines, cell cultures, and biological fluids (e.g., saliva, blood, plasma, serum, urine, feces, lymphatic fluid, cerebrospinal fluid). In some embodiments, the target cell is a lung cell. In some embodiments, the target cell is a liver cell. In some embodiments, the target cell is one or more cells from a subject. In some embodiments, the subject has a disease. In some embodiments, the target cell is a diseased cell. In some embodiments, the target cell is a cancer cell. In some embodiments, the target cell is a solid tumor cell.

In some embodiments, the cryopreserved enucleated cell releases the virus to a target cell in an amount that is greater than or equal to about an amount of the virus released to an otherwise identical target cell by otherwise identical enucleated cells that were not cryopreserved. In some embodiments, the cryopreserved enucleated cell releases the virus to a target cell in an amount that is greater than about an amount of the virus released to an otherwise identical target cell by otherwise identical enucleated cells that were not cryopreserved. In some embodiments, the cryopreserved enucleated cell releases to the target cell between about 1-fold greater to about 50-fold greater than the amount of the virus released to the otherwise identical target cell by the otherwise comparable enucleated cell. In some embodiments, the cryopreserved enucleated cell releases to the target cell between about 1-fold greater to about 2 fold greater, between about 2-fold greater to about 3-fold greater, between about 3-fold greater to about 4-fold greater, between about 4-fold greater to about 5-fold greater, between about 5-fold between to about 10-fold greater, between about 10-fold greater to about 15-fold greater, between about 15-fold greater to about 20 fold greater, between about 20-fold greater to about 25-fold greater, between about 25-fold greater to about 30-fold greater, between about 30-fold greater to about 35-fold greater, between about 35-fold greater to about 40-fold greater, between about 40-fold greater to about 45-fold greater, or between about 45-fold greater to about 50-fold greater than the amount of the virus that is released to the otherwise identical target cell by the otherwise identical enucleated cell. In some embodiments, the cryopreserved enucleated cell releases the virus to the target cell in an amount that is about one-fold greater, about two-fold greater, about three-fold greater, about four-fold greater, about five-fold greater, about 10-fold greater, about 15-fold greater, about 20-fold greater, about 25-fold greater, about 30-fold greater, about 35-fold greater, about 40-fold greater, about 45-fold greater, about 50-fold greater, or more compared to the amount of the virus that is released to the otherwise identical target cell by the otherwise identical enucleated cell. In some embodiments, the amount of the virus that is released by the cryopreserved enucleated cell to the target cell is equal to the amount of the virus that is released to the otherwise identical target cell by the otherwise identical enucleated cell.

In some embodiments, the therapeutic agent is a cytokine or a cytokine receptor-binding fragment thereof. In some embodiments, the amount of the cytokine or a cytokine receptor-binding fragment thereof released is measured by the amount of soluble cytokine or a cytokine receptor-binding fragment thereof present. In some embodiments, the amount of the cytokine or a cytokine receptor-binding fragment thereof released is measured in micrograms (μg) per mL. In some embodiments, the amount of the cytokine or a cytokine receptor-binding fragment thereof released is measured in nanograms (ng) per mL. In some embodiments, the amount of the cytokine or a cytokine receptor-binding fragment thereof released is measured in picograms (pg) per mL. In some embodiments, the amount of the cytokine or a cytokine receptor-binding fragment thereof released is measured in the supernatant of the cryopreserved enucleated cells. In some embodiments, the amount of the cytokine or a cytokine receptor-binding fragment thereof released is measured in the supernatant of the otherwise identical enucleated cells. In some embodiments, the amount of the cytokine or a cytokine receptor-binding fragment thereof released is measured in the supernatant of the cryopreserved enucleated cells and the supernatant of the otherwise identical enucleated cells.

In some embodiments, the cryopreserved nucleated cell releases a greater amount of the cytokine or cytokine receptor-binding fragment thereof compared to the otherwise identical enucleated cells. In some embodiments, the cryopreserved enucleated cell releases between about 1-fold greater to about 50-fold greater than the amount of the cytokine or a cytokine receptor-binding fragment thereof compared to the otherwise comparable enucleated cell. In some embodiments, the cryopreserved enucleated cell releases between about 1-fold greater to about 2 fold greater, between about 2-fold greater to about 3-fold greater, between about 3-fold greater to about 4-fold greater, between about 4-fold greater to about 5-fold greater, between about 5-fold between to about 10-fold greater, between about 10-fold greater to about 15-fold greater, between about 15-fold greater to about 20 fold greater, between about 20-fold greater to about 25-fold greater, between about 25-fold greater to about 30-fold greater, between about 30-fold greater to about 35-fold greater, between about 35-fold greater to about 40-fold greater, between about 40-fold greater to about 45-fold greater, or between about 45-fold greater to about 50-fold greater than the amount of the cytokine or a cytokine receptor-binding fragment thereof that is released by the otherwise identical enucleated cell. In some embodiments, the cryopreserved enucleated cell releases a cytokine or a cytokine receptor-binding fragment thereof in an amount that is about one-fold greater, about two-fold greater, about three-fold greater, about four-fold greater, about five-fold greater, about 10-fold greater, about 15-fold greater, about 20-fold greater, about 25-fold greater, about 30-fold greater, about 35-fold greater, about 40-fold greater, about 45-fold greater, about 50-fold greater, or more compared to the amount of the cytokine or a cytokine receptor-binding fragment thereof that is released by the otherwise identical enucleated cell. In some embodiments, the amount of cytokine or a cytokine receptor-binding fragment thereof that is released by the cryopreserved enucleated cell is equal to the amount of the cytokine or a cytokine receptor-binding fragment thereof that is released by the otherwise identical enucleated cell.

In some embodiments, the amount of the cytokine or a cytokine receptor-binding fragment thereof released is measured in a target cell introduced to the cryopreserved enucleated cells. In some embodiments, the amount of the cytokine or a cytokine receptor-binding fragment thereof released is measured in an otherwise identical target cell introduced to the otherwise identical enucleated cells. In some embodiments, the amount of the cytokine or a cytokine receptor-binding fragment thereof released is measured in target cells introduced to the cryopreserved nucleated cells and in otherwise identical target cells introduced to the otherwise identical enucleated cells. In some embodiments, the amount of the cytokine or a cytokine receptor-binding fragment thereof is measured as the amount of cytokine or a cytokine receptor-binding fragment thereof mRNA. In some embodiments, the amount of the cytokine or a cytokine receptor-binding fragment thereof released is measured by the amount of the cytokine or a cytokine receptor-binding fragment thereof mRNA present in a target cell introduced to cryopreserved enucleated cells. In some embodiments, the amount of the cytokine or a cytokine receptor-binding fragment thereof released is measured by the amount of the cytokine or a cytokine receptor-binding fragment thereof mRNA present in an otherwise identical target cell introduced to the otherwise identical enucleated cells. In some embodiments, the amount of the cytokine or a cytokine receptor-binding fragment thereof released is measure by the amount of the cytokine or a cytokine receptor-binding fragment thereof mRNA present in a target cell introduced to cryopreserved enucleated cells and the amount of the cytokine or a cytokine receptor-binding fragment thereof mRNA present in an otherwise identical target cell introduced to the otherwise identical enucleated cells.

In some embodiments, the target cell introduced to the cryopreserved enucleated cells has a greater amount of the cytokine or cytokine receptor-binding fragment thereof mRNA compared to an otherwise identical target cell introduced to the otherwise identical enucleated cells. In some embodiments, the target cell introduced to the cryopreserved enucleated cell has between about 1-fold greater to about 50-fold greater than the amount of the cytokine or a cytokine receptor-binding fragment thereof mRNA compared to the otherwise identical target cells introduced to the otherwise comparable enucleated cell. In some embodiments, the target cell introduced to the cryopreserved enucleated cell has between about 1-fold greater to about 2 fold greater, between about 2-fold greater to about 3-fold greater, between about 3-fold greater to about 4-fold greater, between about 4-fold greater to about 5-fold greater, between about 5-fold between to about 10-fold greater, between about 10-fold greater to about 15-fold greater, between about 15-fold greater to about 20 fold greater, between about 20-fold greater to about 25-fold greater, between about 25-fold greater to about 30-fold greater, between about 30-fold greater to about 35-fold greater, between about 35-fold greater to about 40-fold greater, between about 40-fold greater to about 45-fold greater, or between about 45-fold greater to about 50-fold greater than the amount of the cytokine or a cytokine receptor-binding fragment thereof mRNA that is present in an otherwise identical target cell introduced to the otherwise identical enucleated cell. In some embodiments, the target cell introduced to the cryopreserved enucleated cell about one-fold greater, about two-fold greater, about three-fold greater, about four-fold greater, about five-fold greater, about 10-fold greater, about 15-fold greater, about 20-fold greater, about 25-fold greater, about 30-fold greater, about 35-fold greater, about 40-fold greater, about 45-fold greater, about 50-fold greater, or more the amount of the cytokine or cytokine receptor-binding fragment thereof mRNA compared to the otherwise identical target cell introduced to otherwise identical enucleated cell. In some embodiments, the amount of cytokine or a cytokine receptor-binding fragment thereof mRNA present in the target cell introduced to the cryopreserved enucleated cell is equal to the amount of the cytokine or a cytokine receptor-binding fragment thereof mRNA present in the otherwise identical target cell introduced to the otherwise identical enucleated cell.

In some embodiments, the amount of the cytokine or cytokine receptor-binding fragment thereof released is measured through release of an another cytokine or cytokine receptor-binding fragment thereof by a target cell introduced to the cryopreserved enucleated cells. In some embodiments, the another cytokine or cytokine receptor-binding fragment thereof is a soluble cytokine or cytokine receptor-binding fragment thereof. In some embodiments, the another cytokine or cytokine receptor-binding fragment thereof is any cytokine or cytokine receptor-binding fragment thereof of the present disclosure. In some embodiments, the another cytokine or cytokine receptor-binding fragment thereof is IFN-γ. In some embodiments, the another cytokine or cytokine receptor-binding fragment thereof is TNF-α. In some embodiments, the another cytokine or cytokine receptor-binding fragment thereof is IL-6.

In some embodiments, the amount of the cytokine or a cytokine receptor-binding fragment thereof released is measured in an otherwise identical target cell introduced to the otherwise identical enucleated cells. In some embodiments, the amount of the cytokine or a cytokine receptor-binding fragment thereof released is measured in target cells introduced to the cryopreserved nucleated cells and in otherwise identical target cells introduced to the otherwise identical enucleated cells. In some embodiments, the amount of the cytokine or a cytokine receptor-binding fragment thereof is measured as the amount of cytokine or a cytokine receptor-binding fragment thereof mRNA. In some embodiments, the amount of the cytokine or a cytokine receptor-binding fragment thereof released is measured by the amount of the cytokine or a cytokine receptor-binding fragment thereof mRNA present in a target cell introduced to cryopreserved enucleated cells. In some embodiments, the amount of the cytokine or a cytokine receptor-binding fragment thereof released is measured by the amount of the cytokine or a cytokine receptor-binding fragment thereof mRNA present in an otherwise identical target cell introduced to the otherwise identical enucleated cells. In some embodiments, the amount of the cytokine or a cytokine receptor-binding fragment thereof released is measure by the amount of the cytokine or a cytokine receptor-binding fragment thereof mRNA present in a target cell introduced to cryopreserved enucleated cells and the amount of the cytokine or a cytokine receptor-binding fragment thereof mRNA present in an otherwise identical target cell introduced to the otherwise identical enucleated cells.

In some embodiments, the target cell introduced to the cryopreserved enucleated cells releases a greater amount of the another cytokine or cytokine receptor-binding fragment thereof compared to an otherwise identical target cell introduced to the otherwise identical enucleated cells. In some embodiments, the target cell introduced to the cryopreserved enucleated cell releases between about 1-fold greater to about 50-fold greater than the amount of the another cytokine or a cytokine receptor-binding fragment thereof compared to the otherwise identical target cells introduced to the otherwise comparable enucleated cell. In some embodiments, the target cell introduced to the cryopreserved enucleated cell releases between about 1-fold greater to about 2 fold greater, between about 2-fold greater to about 3-fold greater, between about 3-fold greater to about 4-fold greater, between about 4-fold greater to about 5-fold greater, between about 5-fold between to about 10-fold greater, between about 10-fold greater to about 15-fold greater, between about 15-fold greater to about 20 fold greater, between about 20-fold greater to about 25-fold greater, between about 25-fold greater to about 30-fold greater, between about 30-fold greater to about 35-fold greater, between about 35-fold greater to about 40-fold greater, between about 40-fold greater to about 45-fold greater, or between about 45-fold greater to about 50-fold greater than the amount of the another cytokine or a cytokine receptor-binding fragment thereof that is released in an otherwise identical target cell introduced to the otherwise identical enucleated cell. In some embodiments, the target cell introduced to the cryopreserved enucleated cell releases about one-fold greater, about two-fold greater, about three-fold greater, about four-fold greater, about five-fold greater, about 10-fold greater, about 15-fold greater, about 20-fold greater, about 25-fold greater, about 30-fold greater, about 35-fold greater, about 40-fold greater, about 45-fold greater, about 50-fold greater, or more the amount of the another cytokine or cytokine receptor-binding fragment thereof compared to the otherwise identical target cell introduced to otherwise identical enucleated cell. In some embodiments, the amount of the another cytokine or a cytokine receptor-binding fragment thereof released in the target cell introduced to the cryopreserved enucleated cell is equal to the amount of the another cytokine or a cytokine receptor-binding fragment thereof released by the otherwise identical target cell introduced to the otherwise identical enucleated cell.

In some embodiments, the amount of the cytokine or a cytokine receptor-binding fragment thereof is measured as the amount of the another cytokine or a cytokine receptor-binding fragment thereof mRNA present in the target cell introduced to the cryopreserved enucleated cells. In some embodiments, the amount of the cytokine or cytokine receptor-binding fragment thereof is measured as the amount of the another cytokine or a cytokine receptor-binding fragment thereof mRNA present in the otherwise identical target cell introduced to the otherwise identical enucleated cells. In some embodiments, the amount of the cytokine or a cytokine receptor-binding fragment thereof mRNA is measured as the amount of the another cytokine or cytokine receptor-binding fragment thereof mRNA present in the target introduced to the cryopreserved enucleated cells and the amount of the another cytokine or a cytokine receptor-binding fragment thereof mRNA present in the otherwise identical target cell introduced to the otherwise identical enucleated cells.

In some embodiments, the target cell introduced to the cryopreserved enucleated cells has a great amount of the another cytokine or cytokine receptor-binding fragment thereof mRNA compared to an otherwise identical target cell introduced to the otherwise identical enucleated cells. In some embodiments, the target cell introduced to the cryopreserved enucleated cell has between about 1-fold greater to about 50-fold greater than the amount of the another cytokine or a cytokine receptor-binding fragment thereof mRNA compared to the otherwise identical target cells introduced to the otherwise comparable enucleated cell. In some embodiments, the target cell introduced to the cryopreserved enucleated cell has between about 1-fold greater to about 2 fold greater, between about 2-fold greater to about 3-fold greater, between about 3-fold greater to about 4-fold greater, between about 4-fold greater to about 5-fold greater, between about 5-fold between to about 10-fold greater, between about 10-fold greater to about 15-fold greater, between about 15-fold greater to about 20 fold greater, between about 20-fold greater to about 25-fold greater, between about 25-fold greater to about 30-fold greater, between about 30-fold greater to about 35-fold greater, between about 35-fold greater to about 40-fold greater, between about 40-fold greater to about 45-fold greater, or between about 45-fold greater to about 50-fold greater than the amount of the another cytokine or a cytokine receptor-binding fragment thereof mRNA that is present in an otherwise identical target cell introduced to the otherwise identical enucleated cell. In some embodiments, the target cell introduced to the cryopreserved enucleated cell about one-fold greater, about two-fold greater, about three-fold greater, about four-fold greater, about five-fold greater, about 10-fold greater, about 15-fold greater, about 20-fold greater, about 25-fold greater, about 30-fold greater, about 35-fold greater, about 40-fold greater, about 45-fold greater, about 50-fold greater, or more the amount of the another cytokine or cytokine receptor-binding fragment thereof mRNA compared to the otherwise identical target cell introduced to otherwise identical enucleated cell. In some embodiments, the amount of another cytokine or a cytokine receptor-binding fragment thereof mRNA present in the target cell introduced to the cryopreserved enucleated cell is equal to the amount of the another cytokine or a cytokine receptor-binding fragment thereof mRNA present in the otherwise identical target cell introduced to the otherwise identical enucleated cell. In some embodiments, the cryopreserved composition further comprises intracellular organelles. In some embodiments, the intracellular organelles for synthesis or secretion of a therapeutic agent. In some embodiments, the intracellular organelles synthesize or secrete the therapeutic agent in absence of a nucleus. In some embodiments, the intracellular organelle is a Golgi apparatus, an endoplasmic reticulum, or a combination thereof.

In some embodiments, the enucleated cell or the composition comprising the enucleated cell is stable at 4° C. for greater than or equal to about one hour, two hours, six hours, 12 hours, one day, two days, three days, four days, five days, six days, one week, two weeks, three weeks, four weeks, one month, two months, three months, or longer period of time. In some embodiments, the composition is stable at room temperature for greater than or equal to about one hour, two hours, six hours, 12 hours, one day, two days, three days, four days, five days, six days, one week, two weeks, three weeks, four weeks, one month, two months, three months, or longer period of time. In some embodiments, the composition is stable at 37° C. for greater than or equal to about one hour, two hours, six hours, 12 hours, one day, two days, three days, four days, five days, six days, one week, two weeks, three weeks, four weeks, one month, two months, three months, or longer period of time. In some embodiments, the enucleated cell or the composition comprising the enucleated cell may remain viable after being administered to a subject in need thereof for treating the disease or condition described herein. In some embodiments, the enucleated cell or the composition comprising the enucleated cell may remain viable after being administered to the subject for greater than or equal to about one hour, two hours, six hours, 12 hours, one day, two days, three days, four days, five days, six days, one week, two weeks, three weeks, four weeks, one month, two months, three months, or longer period of time.

In some embodiments, the enucleated cell may be obtained from a parent cell that is autologous to the subject, who is in need of the treatment by the enucleated cell described herein. In some embodiments, the enucleated cell may be obtained from a parent cell that is allogenic to the subject, who is in need of the treatment by the enucleated cell described herein.

Enucleated cells may be smaller than their nucleated counterparts (e.g. the nucleated parent cells), and for this reason may migrate better through small openings in the vasculature and tissue parenchyma. In addition, removing the large dense nucleus alleviates a major physical barrier allowing the cell to move freely through small openings in the vessels and tissue parenchyma. Therefore, enucleated cells have improved bio-distribution in the body and movement into target tissues. In some embodiments, an enucleated cell comprises at least 1 micrometer (μm) in diameter. In some embodiments, an enucleated cell is greater than 1 μm in diameter. In some embodiments, an enucleated cell is 1-100 μm in diameter (e.g., 1-90 μm, 1-80 μm, 1-70 μm, 1-60 μm, 1-50 μm, 1-40 μm, 1-30 μm, 1-20 μm, 1-10 μm, 1-5 μm, 5-90 μm, 5-80 μm, 5-70 μm, 5-60 μm, 5-50 μm, 5-40 μm, 5-30 μm, 5-20 μm, 5-10 μm, 10-90 μm, 10-80 μm, 10-70 μm, 10-60 μm, 10-50 μm, 10-40 μm, 10-30 μm, 10-20 μm, 10-15 μm 15-90 μm, 15-80 μm, 15-70 μm, 15-60 μm, 15-50 μm, 15-40 μm, 15-30 μm, 15-20 μm). In some embodiments, an enucleated cell is 10-30 μm in diameter. In some embodiments, the diameter of an enucleated cell is between 5-25 μm (e.g., 5-20 μm, 5-15 μm, 5-10 μm, 10-25 μm, 10-20 μm, 10-15 μm, 15-25 μm, 15-20 μm, or 20-25 μm). In some embodiments, the enucleated cell has a diameter that is about 8 μm. In some embodiments, some enucleated cells may advantageously be small enough to allow for better homing or delivery to a target site. For examples, the enucleated cells described herein may pass through passages in narrow lung tissues or lung structures such as alveolar duct or microcapillary that most cells such as the parent cells may not pass through.

In some embodiments, the enucleated cell of the enucleated cell fraction has a diameter comprising less than or equal to about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of an average diameter of a nucleated parent cell. In some embodiments, the enucleated cell of the enucleated cell fraction has a diameter comprising less than or equal to about 10%, about 20%, about 30%, about 40%, about 50%, about 60%, about 70%, about 80%, about 90%, about 95%, or about 99% of an average diameter of the nucleated cells. In some embodiments, the enucleated cell of the enucleated cell fraction has a diameter comprising less than or equal to about 50% of an average diameter of the nucleated cells. In some embodiments, the enucleated cell of the enucleated cell fraction has a diameter comprising less than or equal to about 60% of an average diameter of the nucleated cells. In some embodiments, the enucleated cell of the enucleated cell fraction has a diameter comprising less than or equal to about 70% of an average diameter of the nucleated cells. In some embodiments, the enucleated cell of the enucleated cell fraction has a diameter comprising less than or equal to about 80% of an average diameter of the nucleated cells. In some embodiments, the enucleated cell of the enucleated cell fraction has a diameter comprising less than or equal to about 90% of an average diameter of the nucleated cells.

In some embodiments, enucleated cells possess significant therapeutic value, because they remain viable, do not differentiate into other cell types, secrete bioactive molecules, and may physically migrate/home for fewer than or equal to about 5 days, may be extensively enucleated ex vivo to perform specific therapeutic functions, and may be fused to the same or other cell types to transfer desirable production, natural or enucleated. Therefore, enucleated cells have wide utility as a cellular vehicle to deliver therapeutically important biomolecules and disease-targeting cargos including genes, viruses, bacteria, mRNAs, shRNAs, siRNA, polypeptides (including antibodies and antigen binding fragments), plasmids, gene-editing machinery, or nanoparticles. The present disclosure enables the generation of safe (e.g., no unwanted DNA is transferred to the subject), and controllable (e.g., cell death occurs in approximately 5 days) cell-based carrier that may be genetically enucleated to deliver specific disease-fighting and health promoting cargos to humans. In some embodiments, the enucleated cell remains viable and retain the function to migrate or home for greater than or equal to about 12 hours, 24 hours, 36 hours, 48 hours, 60 hours, 72 hours, 84 hours, 96 hours, 108 hours, 5 days, 6 days, 7 days, 8 days, 9 days, or longer after being administered to the subject in need thereof.

In some embodiments, the enucleated cell is engineered to express at least one of an exogenous DNA molecule, an exogenous RNA molecule, an exogenous protein, or an exogenous protein, gene-editing machinery or combinations thereof. In some embodiments, the exogenous DNA molecule is a single-stranded DNA, a double-stranded DNA, an oligonucleotide, a plasmid, a bacterial DNA molecule, a DNA virus, or combinations thereof. In some embodiments, the exogenous RNA molecule is messenger RNA (mRNA), small interfering RNA (siRNA), microRNA (miRNA), short hairpin RNA (shRNA), an RNA virus, or combinations thereof. In some embodiments, the exogenous protein is a cytokine, a growth factor, a hormone, an antibody or the antigen-binding fragment thereof, an enzyme, an antigen, an immunomodulatory protein, or combinations thereof. In some embodiments, the exogenous protein comprises a functional fragment of a full length protein. In some embodiments, the antibody is a single-domain antibody or antigen-binding fragment thereof. In some embodiments, parental cells (e.g., nucleated cells) are genetically enucleated before enucleation (e.g., pre-enucleation). In some embodiments, the parent cell is genetically enucleated after enucleation (e.g., post-enucleation). For example, the enucleated cell disclosed herein may be engineered to express interleukin-12 (IL-12). In another example, the enucleated cell disclosed herein may be engineered to contain a virus, such as Vesicular Stomatitis Virus (VSV). In some embodiments, the VSV may encode an active agent, such as Interferon-β (IFN-β).

### (b) Transmembrane Moiety

Described herein, in some embodiments, are enucleated cells or compositions comprising the enucleated cell comprising at least one transmembrane moiety. In some embodiments, the enucleated cell comprises an exogenous polypeptide. The exogenous polypeptide may be covalently fused to a transmembrane moiety. In some embodiments, the exogenous polypeptide is complexed to the transmembrane moiety. In some embodiments, the transmembrane moiety comprises a full-length protein or a variation thereof or a fragment thereof. In some embodiments, the transmembrane moiety is endogenous to the parent cell that is being enucleated for obtaining the enucleated cell. In some embodiments, the transmembrane moiety may be an exogenous transmembrane moiety to the parent cell or to the enucleated cell.

In some embodiments, the transmembrane moiety is selected from a transmembrane protein comprising a single transmembrane α-helix (bitopic membrane protein). The transmembrane moiety comprises a polytopic transmembrane α-helical protein. In some embodiments, the transmembrane moiety comprises a polytopic transmembrane β-sheet protein. In some embodiments, the transmembrane moiety comprises a Type I, II, III, or IV transmembrane protein. Non-limiting examples of transmembrane protein may include CD4, CD14, glycophorin a (GPA), or any combination of integrins.

In some embodiments, the transmembrane moiety is added to the exogenous polypeptide by way of a modification. For example, a transmembrane moiety may be added to the N or C-terminus of the exogenous polypeptide to insert the exogenous polypeptide into the cell membrane of the enucleated cell described herein. Non-limiting examples of modifications that are made to the exogenous polypeptide to add the transmembrane moiety may include adding glycosylphosphatidylinositol, farnesyl, palmitate, myristate, or a combination thereof to the exogenous polypeptide.

In some embodiments, the transmembrane moiety is genetically modified to be fused or complexed with the at least one exogenous therapeutic agent described herein. In some embodiments, the transmembrane moiety is genetically modified to fuse to the at least one exogenous therapeutic agent described herein. In some embodiments, the enucleated cell comprises an immune-evading moiety. In some embodiments, the immune-evading comprises a “don't eat me” signaling peptide, such as CD47 (e.g., NCBI Gene ID 961), programmed cell death 1 ligand 1 (PD-L1, e.g., NCBI Gene ID 29126), major histocompatibility complex, class I, E (HLA-E, e.g., NCBI Gene ID 3133), major histocompatibility complex, class I, G (HLA-I, e.g., NCBI Gene ID 3135), a fragment thereof, or a combination thereof.

### (c) Targeting Moiety

Described herein, in some embodiments, are enucleated cells comprising a targeting moiety. The targeting moiety described herein is designed to guide the enucleated cell to a target cell or target environment (e.g., tissue) in a subject following delivery (e.g., systemic delivery) of the enucleated cell to the subject. In some embodiments, the targeting moiety is expressed on the surface of the enucleated cell. In some embodiments, the targeting moiety is complexed with a transmembrane moiety described herein. In some embodiments, the targeting moiety is secreted by the enucleated cell. In some embodiments, the enucleated cells comprising the targeting moiety localizes at the target cell or target environment with a 2-fold, 5-fold, 10-fold, 50-fold, 100-fold, 200-fold, 500-fold, 1,000-fold, 5,000-fold, or 10,000-fold increase as compared to localization of a comparable enucleated cell lacking the targeting moiety. In some embodiments, the enucleated cell comprising the targeting moiety localizes at the target cell or target environment with an increase of 50%, 55%, 60%, 65%, 70%, 75%, 80%, 85%, 90%, 95%, or 99% as compared with a comparable enucleated cell lacking the targeting moiety. In some embodiments, the target cell or target environment is in vivo. In some embodiments, the target cell or target environment is ex vivo.

In some embodiments, the targeting moiety comprises an exogenous antibody or an exogenous antigen-binding fragment for targeting a biomarker described herein. In some embodiments, the targeting moiety comprises an exogenous antibody or an exogenous antigen-binding fragment for targeting a chemokine receptor or a chemokine ligand, or portion thereof, involved in chemokine signaling. In some embodiments, the exogenous antibody is an exogenous single-domain antibody or fragment thereof.

In some embodiments, the targeting moiety targets the biomarker expressed by, or associated with, a target cell or with a microenvironment. In some embodiments, the biomarker may be released by the target cell. The biomarker may indicate the presence of the disease or the condition. In some embodiments, the biomarker is expressed by immune cells responding to the target cell or the microenvironment associated with the disease or the condition. In some embodiments, the biomarker may be an epitope or antigen. In some embodiments, the biomarker comprising the epitope may be bound by an antibody that is different from the antibody or the antigen-binding fragment thereof that confers therapeutic property (e.g., the therapeutic agent).

In some embodiments, the targeting moiety targets a biomarker expressed or released by a lung cell or a lung cancer cell. Non-limiting example of cancer cell biomarkers includes carbonic anhydrase 9 (CA9, e.g., NCBI Gene ID 768), carbonic anhydrase 12 (CA12, e.g., NCBI Gene ID 771), cancer/testis antigen 83 (CXorf61; e.g., NCBI Gene ID203413), desmoglein 3 (DSG3 (e.g., NCBI Gene ID 1830), FAT atypical cadherin 2 (FAT2 (e.g., NCBI Gene ID 2196), G protein-coupled receptor 87 (GPR87, e.g., NCBI Gene ID 53836), KISS1 receptor (KISS1R, e.g., NCBI Gene ID 84634), LY6/PLAUR domain containing 3 (LYPD3. e.g., NCBI Gene ID 27076), solute carrier family 7 member 11 (SLC7A11, e.g., NCBI Gene ID 23657), TMPRSS4 (e.g., NCBI Gene ID 56649), transmembrane serine protease 4 (TFPI, e.g., NCBI Gene ID 7035), midkine (MDK, e.g., NCBI Gene ID 4192), secreted phosphoprotein 1 (OPN, e.g., NCBI Gene ID 6696), matrix metallopeptidase 2 (MMP2, e.g., NCBI Gene ID 4313), TIMP metallopeptidase inhibitor 1 (TIMP1, e.g., NCBI Gene ID 7076), cell adhesion molecule 5 (CEA, e.g., NCBI Gene ID 1048), cytokeratin 19 fragment (CYFRA 21-1, e.g., NCBI Gene ID 3880), serpin family B member 3 (SCC, e.g., NCBI Gene ID 6317), advanced glycosylation end-product specific receptor (AGER, e.g., NCBI Gene ID 177), adipogenesis regulatory factor (C10orf116, e.g., NCBI Gene ID 10974), adducin 2 (ADD2, e.g., NCBI Gene ID 119), periaxin (PRX, e.g., NCBI Gene ID 57716), laminin subunit beta 3 (LAMB3, e.g., NCBI Gene ID 3914), synemin (SYNM, e.g., NCBI Gene ID 23336), spectrin alpha, erythrocytic 1 (SPTA1, e.g., NCBI Gene ID 6708), ankyrin 1 (ANK1, e.g., NCBI Gene ID 286), hemoglobin subunit epsilon 1 (HBE1, e.g., NCBI Gene ID 3046), hemoglobin subunit gamma 1 (HBG1, e.g., NCBI Gene ID 3047), carbonic anhydrase 1 (CA1, e.g., NCBI Gene ID 759), tenascin XB (TNXB, e.g., NCBI Gene ID 7148), multimerin 2 (MMRN2, e.g., NCBI Gene ID 79812), hemoglobin subunit alpha 1 (HBA1, e.g., NCBI Gene ID 3039), caveolin 1 (CAVI, e.g., NCBI Gene ID 857), hemoglobin subunit beta (HBB, e.g., NCBI Gene ID 3043), collagen type VI alpha 6 chain (COL6A6, e.g., NCBI Gene ID 131873), chromosome 1 open reading frame 198 (C1orf198, e.g., NCBI Gene ID 84886), chloride intracellular channel 2 (CLIC2, e.g., NCBI Gene ID 1193), transcriptional regulator of SdpC synthesis operon (ArsR family) (SDPR, e.g., NCBI Gene ID 8436), EH domain containing 2 (EHD2, e.g., NCBI Gene ID 30846), apolipoprotein A2 (APOA2, e.g., NCBI Gene ID 336), NADH: ubiquinone oxidoreductase subunit B7 (NDUFB7, e.g., NCBI Gene ID 4713), protein kinase C delta binding protein (PRKCDBP, e.g., NCBI Gene ID 112464), laminin subunit alpha 3 (LAMA3, e.g., NCBI Gene ID), EvC ciliary complex subunit 2 (LBN, e.g., NCBI Gene ID 132884), serpin family A member 3 (ACT, e.g., NCBI Gene ID 12), insulin like growth factor binding protein 3 (3 IGFBP3, e.g., NCBI Gene ID 3486), prostaglandin D2 synthase (L-PGDS, e.g., NCBI Gene ID 5730), retinoic acid receptor beta (HAP, e.g., NCBI Gene ID 5915), hepatocyte growth factor (HGF, e.g., NCBI Gene ID 3082), eukaryotic translation initiation factor 4 gamma 2 (AAG1/2, e.g., NCBI Gene ID 1982), clusterin (CLU, e.g., NCBI Gene ID 1191), streptococcal superantigen SSA (SSA, e.g., NCBI Gene ID 6737), tetanic (TTA, e.g., NCBI Gene ID 100189453), apolipoprotein A4 (APOA4, e.g., NCBI Gene ID 337), fibrinogen-like protein A (FIBA, e.g., NCBI Gene ID 105209070), serum amyloid A cluster (SAA, e.g., NCBI Gene ID 6288), ceruloplasmin (CP, e.g., NCBI Gene ID 1356), haptoglobin (HP, e.g., NCBI Gene ID 3240), transthyretin (TTR, e.g., NCBI Gene ID 7276), keratin 2 (KRT2A, e.g., NCBI Gene ID 3849), glutamate transporter (GLT1B, e.g., NCBI Gene ID 6506), casein kinase 1 (CK1, e.g., NCBI Gene ID 1452), AKT serine/threonine kinase 1 (AKT, e.g., NCBI Gene ID 207), mannose binding lectin 2 (MBL2, e.g., NCBI Gene ID 4153), fibrinogen alpha chain (FGA, e.g., NCBI Gene ID 2243), gelsolin (GSN, e.g., NCBI Gene ID 2934), haptoglobin (HP, e.g., NCBI Gene ID 3240), ficolin 3 (FCN3, e.g., NCBI Gene ID 8547), carnosine dipeptidase 1 (CNDP1, e.g., NCBI Gene ID 84735), calcitonin related polypeptide alpha (CALCA, e.g., NCBI Gene ID 796), carbamoyl-phosphate synthase 1 (CPS1, e.g., NCBI Gene ID 1373), chromogranin B (CHGB, e.g., NCBI Gene ID 1114), involucrin (IVL, e.g., NCBI Gene ID 3713), anterior gradient 2 (AGR2, e.g., NCBI Gene ID 10551), nuclear autoantigenic sperm protein (NASP, e.g., NCBI Gene ID 4678), phosphofructokinase, platelet (PFKP, e.g., NCBI Gene ID 5214), thrombospondin 2 (THBS2, e.g., NCBI Gene ID 7058), thioredoxin domain containing 17 (TXNDC17, e.g., NCBI Gene ID 84817), proprotein convertase subtilisin/kexin type 1 (PCSK1, e.g., NCBI Gene ID 5122), cellular retinoic acid binding protein 2 (CRABP2, e.g., NCBI Gene ID 1382), acyl-CoA binding domain containing 3 (ACBD3, e.g., NCBI Gene ID 64746), desmoglein 2 (DSG2, e.g., NCBI Gene ID 1829), LPS responsive beige-like anchor protein (LRBA, e.g., NCBI Gene ID 987), serine/threonine kinase receptor associated protein (STRAP, e.g., NCBI Gene ID 11171), VGF nerve growth factor inducible (VGF, e.g., NCBI Gene ID 7425), NOP2 nucleolar protein (NOP2, e.g., NCBI Gene ID 4839), lipocalin 2 (LCN2, e.g., NCBI Gene ID 3934), creatine kinase, mitochondrial 1B (CKMT1B, e.g., NCBI Gene ID 1159), aldo-keto reductase family 1 member B10 (AKR1B10, e.g., NCBI Gene ID 57016), carboxypeptidase D (CPD, e.g., NCBI Gene ID 1362), proteasome activator subunit 3 (PSME3, e.g., NCBI Gene ID 10197), villin 1 (VILI, e.g., NCBI Gene ID 7429), serpin family B member 5 (SERPINB5, e.g., NCBI Gene ID 5268), ribosomal protein L5 (RPL5, e.g., NCBI Gene ID 6125), plakophilin 1 (PKP1, e.g., NCBI Gene ID 5317), ribosomal protein L10 (RPL10, e.g., NCBI Gene ID 6134), aldo-keto reductase family 1 member B10 (AKR1B10, e.g., NCBI Gene ID 57016), aldo-keto reductase family 1 member C1 (AKR1C1, e.g., NCBI Gene ID 1645), proliferating cell nuclear antigen (PCNA, e.g., NCBI Gene ID 5111), ribosomal protein S2 (RPS2, e.g., NCBI Gene ID 6187), aldo-keto reductase family 1 member C3 (AKR1C3, e.g., NCBI Gene ID 8644), acyl-CoA binding domain containing 3 (ACBD3, e.g., NCBI Gene ID 64746), visinin like 1 (VSNL1, e.g., NCBI Gene ID 7447), adenosylhomocysteinase (AHCY, e.g., NCBI Gene ID 191), IMMP10, activated kinase 2 (PAK2, e.g., NCBI Gene ID 5062), involucrin (IVL, e.g., NCBI Gene ID 3713), isoleucine-tRNA synthetase (IARS, e.g., NCBI Gene ID 3376), proteasome 26S subunit ubiquitin receptor, non-ATPase 2 (PSMD2, e.g., NCBI Gene ID 5708), guanylate binding protein 5 (GBP5, e.g., NCBI Gene ID 115362), minichromosome maintenance complex component 6 (MCM6, e.g., NCBI Gene ID 4175), N-myc downstream regulated 1 (NDRG1, e.g., NCBI Gene ID 10397), NOP58 ribonucleoprotein (NOP58, e.g., NCBI Gene ID 51602), S100 calcium binding protein A2 (S100A2, e.g., NCBI Gene ID 6273), neuregulin 1 (NRG1, e.g., NCBI Gene ID 3084), neuregulin 2 (NRG2, e.g., NCBI Gene ID 9542), carnosine dipeptidase 1 (CNDP1, e.g., NCBI Gene ID 84735), ubiquitin cross-reactive protein (UCRP, e.g., NCBI Gene ID 9636), crammer (CER, e.g., NCBI Gene ID 8110), plasminogen activator (UPA, e.g., NCBI Gene ID 5328), matrix metallopeptidase 14 (MT1-MMP, e.g., NCBI Gene ID 4323), stratifin (SFN, e.g., NCBI Gene ID 2810), transferrin (TF, e.g., NCBI Gene ID 7018), albumin (ALB, e.g., NCBI Gene ID 213), S100 calcium binding protein A9 (S100A9, e.g., NCBI Gene ID 6280), stathmin 1 (STMN, e.g., NCBI Gene ID 3925), Enolase (ENO), plasminogen activator (PLAU, e.g., NCBI Gene ID 5328), insulin like growth factor binding protein 7 (IGFBP7, e.g., NCBI Gene ID 3490), matrix metallopeptidase 14

(MMP14, e.g., NCBI Gene ID 4323), thrombospondin 1 (THBS1, e.g., NCBI Gene ID 7057), or thrombospondin 2 (THBS2, e.g., NCBI Gene ID 7058).

In some embodiments, the targeting moiety targets a biomarker expressed or released by a cancer cell that has metastasized. For example, the cancer cell may arise from one tissue and subsequently metastasizes to a different location. In some embodiments, the metastasized cancer cell expresses the non-limiting example of cancer biomarker described herein. In some embodiments, the metastasized cancer cell expresses cancer biomarker includes Melanoma Associated Antigen (MAGE family member A3 (MAGE-A3, e.g., NCBI Gene ID 4102)), Membrane associated glycoprotein (MUC-1, e.g., NCBI Gene ID 4582), glycoprotein-epithelial cell adhesion molecule (EpCAM, e.g., NCBI Gene ID 4072), KRAS Proto-Oncogene (KRAS, e.g., NCBI Gene ID 3845), Anaplastic lymphoma kinase (ALK, e.g., NCBI Gene ID 238), Cytotoxic T-Lymphocyte Associated Protein 4 (CTLA-4, e.g., NCBI Gene ID 1493), Programmed cell death protein 1 (PD-1, e.g., NCBI Gene ID 5133), Epidermal growth factor (EGF, e.g., NCBI Gene ID 1950), Serine protease ester (EA, e.g., NCBI Gene ID 5328), Telomerase reverse transcriptaseh (TERT, e.g., NCBI Gene ID 7015), PRAME Nuclear Receptor Transcriptional Regulator (PRAME, e.g., NCBI Gene ID 23532), Receptor tyrosine-protein kinase erbB-2 (HER, e.g., NCBI Gene ID 2064), or Vascular endothelial growth factor (VEGF, e.g., NCBI Gene ID 7422), Carcinoembryonic antigen (CEA, e.g., NCBI Gene ID 1048), MAGE family member A1 (MAGE-A1, e.g., NCBI Gene ID 4100), MAGE family member A1 MAGE-A4, e.g., NCBI Gene ID 4103), Survivin, Six Transmembrane Epithelial Antigene of the Prostate 1 (STEAP1, e.g., NCBI Gene ID 26872), SRY (sex determining region Y)-box 2 (SOX2, e.g., NCBI Gene ID 6657), or Cancer/testis antigen 1 (CTAG1B, e.g., NCBI Gene ID 1485).

In some embodiments, the targeting moiety targets a biomarker expressed or released by an endothelial cell. In some embodiments, the endothelial cell is a blood vessel cell. In some embodiments, the endothelial cell is a lymphatic vessel cell. In some embodiments, the biomarker is expressed or released by a blood vessel cell. In some embodiments, the biomarker is expressed or released by a lymphatic vessel cell. Non-limiting examples of the endothelial cell biomarker include angiotensin I converting enzyme (ACE/CD143, e.g., NCBI Gene ID 1636), CD93 molecule (C1qR1/CD93, e.g., NCBI Gene ID 22918), cadherin 5 (VE-Cadherin, e.g., NCBI Gene ID 1003), D6 protein (CC Chemokine Receptor D6, e.g., NCBI Gene ID 1238), platelet and endothelial cell adhesion molecule 1 (CD31/PECAM-1, e.g., NCBI Gene ID 5175), CD34 molecule (CD34, e.g., NCBI Gene ID 947), CD36 molecule (CD36/SR-B3, e.g., NCBI Gene ID 948), CD151 molecule (CD151, e.g., NCBI Gene ID 977), CD160 molecule (CD160, e.g., NCBI Gene ID 11126), CD300 molecule like family member g (CD300g/Nepmucin, e.g., NCBI Gene ID 146894), CDC like kinase 1 (CL-K1/COLEC11, e.g., NCBI Gene ID 78989), cleavage factor polyribonucleotide kinase subunit 1 (CL-P1/COLEC12, e.g., NCBI Gene ID 81035), Coagulation Factor III/Tissue Factor (e.g., NCBI Gene ID 2152), C-type lectin domain family 4 member M (DC-SIGNR/CD299, e.g., NCBI Gene ID 10332), discoidin, CUB and LCCL domain containing 2 (DCBLD2/ESDN, e.g., NCBI Gene ID 131566), endothelial cell surface expressed chemotaxis and apoptosis regulator (ECSCR, e.g., NCBI Gene ID 641700), basigin (Ok blood group) (EMMPRIN/CD147, e.g., NCBI Gene ID 682), Endoglin/CD105 (e.g., NCBI Gene ID 5077), Endomucin (e.g., NCBI Gene ID 2022), Endosialin/CD248 (e.g., NCBI Gene ID 57124), protein C receptor (EPCR, e.g., NCBI Gene ID 10544), Erythropoietin R (e.g., NCBI Gene ID 2056), endothelial cell adhesion molecule (ESAM, e.g., NCBI Gene ID 90952), fatty acid binding protein 5 (FABP5/E-FABP, e.g., NCBI Gene ID 2171), fatty acid binding protein 6 (FABP6, e.g., NCBI Gene ID 2172), intercellular adhesion molecule 1 (ICAM-1/CD54, e.g., NCBI Gene ID 3383), intercellular adhesion molecule 2 (ICAM-2/CD102, e.g., NCBI Gene ID 3384), interleukin 1 receptor (IL-1 RI, e.g., NCBI Gene ID 3553), Interleukin 13 receptor, alpha 1 (IL-13 R alpha 1, e.g., NCBI Gene ID 3597), Integrin alpha 4/CD49d (e.g., NCBI Gene ID 3676), Integrin alpha 4 beta 1 (e.g., NCBI Gene ID 3688), Integrin alpha 4 beta 7/LPAM-1 (e.g., NCBI Gene ID 3676), Integrin beta 2/CD18 (e.g., NCBI Gene ID 3689), KLF transcription factor 4 (KLF4, e.g., NCBI Gene ID 9314), lymphatic vessel endothelial hyaluronan receptor 1 (LYVE-1, e.g., NCBI Gene ID 10894), melanoma cell adhesion molecule (MCAM/CD146, e.g., NCBI Gene ID 4162), nectin cell adhesion molecule 2 (Nectin-2/CD112, e.g., NCBI Gene ID 5819), PD-ECGF/Thymidine Phosphorylase (e.g., NCBI Gene ID 1890), Podocalyxin (e.g., NCBI Gene ID 5420), Podoplanin (e.g., NCBI Gene ID 10630), sphingosine-1-phosphate receptor 1 (S1P1/EDG-1, e.g., NCBI Gene ID 1901), sphingosine-1-phosphate receptor 2 (S1P2/EDG-5, e.g., NCBI Gene ID 9294), sphingosine-1-phosphate receptor 3 (S1P3/EDG-3, e.g., NCBI Gene ID 1903), sphingosine-1-phosphate receptor 4 (S1P4/EDG-6, e.g., NCBI Gene ID 8698), sphingosine-1-phosphate receptor 5 (S1P5/EDG-8, e.g., NCBI Gene ID 53637), E-Selectin/CD62E (e.g., NCBI Gene ID 6401), P-Selectin/CD62P (e.g., NCBI Gene ID 6403), slow as molasses (SLAM/CD150, e.g., NCBI Gene ID 6504), Stabilin-1 (e.g., NCBI Gene ID 23166), Stabilin-2 (e.g., NCBI Gene ID 55576), plexin domain containing 1 (TEM7/PLXDC1, e.g., NCBI Gene ID 57125), ANTXR cell adhesion molecule 1 (TEM8/ANTXR1, e.g., NCBI Gene ID 84168), Thrombomodulin/BDCA-3 (e.g., NCBI Gene ID Thrombomodulin), thrombospondin type 1 domain containing 1 (THSD1, e.g., NCBI Gene ID 55901), thrombospondin type 1 domain containing 7A (THSD7A, e.g., NCBI Gene ID 221981), TEK receptor tyrosine kinase (Tie-2, e.g., NCBI Gene ID 7010), TNF receptor superfamily member 1A (TNF RI/TNFRSF1A, e.g., NCBI Gene ID 7132), TNF receptor superfamily member 1B (TNF RII/TNFRSF1B, e.g., NCBI Gene ID 7133), basigin (Ok blood group) (TRA-1-85/CD147, e.g., NCBI Gene ID 682), TNF receptor superfamily member 10b (TRAIL R2/TNFRSF10B, e.g., NCBI Gene ID 8795), TNF receptor superfamily member 10a (TRAILR1/TNFRSF10A, e.g., NCBI Gene ID 8797), vascular cell adhesion molecule 1 (VCAM-1/CD106, e.g., NCBI Gene ID 7412), EGF like domain multiple 7 (VE-Statin, e.g., NCBI Gene ID: 51162), fms related receptor tyrosine kinase 1 (VEGFR1/Flt-1, e.g., NCBI Gene ID 2321), kinase insert domain receptor (VEGFR2/KDR/Flk-1, e.g., NCBI Gene ID 3791), fms related receptor tyrosine kinase 4 (VEGFR3/Flt-4, e.g., NCBI Gene ID 2324), angiogenic factor with G-patch and FHA domains 1 (VG5Q, e.g., NCBI Gene ID 55109), or von Willebrand Factor domain 2 (vWF-A2, e.g., NCBI Gene ID 7450).

In some embodiments, the targeting moiety comprises a chemokine receptor or a chemokine ligand, or portion thereof, involved in chemokine signaling, such as for example, SDF-1α/CXCR4, CCL2/CCR2, or adhesion molecules, such as for example, PSGL-1. As shown herein, the enucleated cell may be enucleated to express functional CXCR4, CCR2 as well as glycosylated PSGL-1, which may greatly promote the specific targeting of the enucleated cell. In some embodiments, the targeting moiety, such as CXCR4, CCR2 or PSGL-1 may be expressed on the surface of the enucleated cell. Non-limiting examples of cell surface proteins that may be expressed on the cell surface of the enucleated cell as the targeting moiety include chemokines such as CXCR4, CCR2, CCR1, CCR5, CXCR7, CXCR2, and CXCR1. In some embodiments, the enucleated cell may be enucleated to secrete the targeting moiety or is tethered to the extracellular matrix, e.g., SDF1α or CCL2. Non-limiting examples of targeting moiety that may be secreted by the enucleated cell include SDF1α, CCL2, CCL3, CCL5, CCL8, CCL1, CXCL9, CXCL10, CCL11 and CXCL12. In some embodiments, the enucleated cell comprises cell-matrix receptors and cell-cell adhesion molecules include integrins, cadherins, glycoproteins, and heparin sulfate proteoglycans.

In some embodiments, the enucleated cells may further include (e.g. by engineering or from the cell from which they were obtained) a surface marker that aids in their evasion of the subject immune system. For example, in some embodiments, the enucleated cells may include a CD47, PD-L1, HLA-E, HLA-G, a fragment thereof, or a combination thereof. Without being bound by any particular theory, it is believed that a CD47, PD-L1, HLA-E, HLA-G, a fragment thereof, or a combination thereof helps to prevent the enucleated cells from being phagocytosed by macrophages. Non-limiting examples of cell-matrix receptors and cell-cell adhesion molecules include integrins, cadherins, glycoproteins, or heparin sulfate proteoglycans. In some embodiments, the cell-matrix receptors or cell-cell adhesion molecules include PD-L1, HLA-E, or HLA-G. Non-limiting examples of therapeutic molecules include tumor antigens and immunomodulatory peptides, polyamines, and ATP. In some embodiments, the therapeutic molecules can be recognized by immune cells and can induce immune response. For example, the therapeutic molecules can be 4-1BB or any one of the cytokines described herein to induce immune response.

### (d) Therapeutic Agent

In some embodiments, the enucleated cell of the present disclosure comprises at least one therapeutic agent. In some embodiments, the enucleated cell of the present disclosure comprises at least two, three, four, five, six, seven, eight, nine, ten, or more therapeutic agents. In some embodiments, the therapeutic agent comprises an active agent. In some embodiments, the therapeutic agent is exogenous to the enucleated cell or parent cell thereof. An active agent comprises at least one of a DNA molecule, a RNA molecule, a protein (e.g., an enzyme, an antibody, an antigen, a toxin, cytokine, a protein hormone, a growth factor, a cell surface receptor, or a vaccine), a peptide (e.g., a peptide hormone or an antigen), a small molecule (e.g., a steroid, a polyketide, an alkaloid, a toxin, an antibiotic, an antiviral, a colchicine, a taxol, a mitomycin, or emtansine), an exogenous gene editing system, a nanoparticle, or another active agent (e.g., bacteria, bacterial spores, bacteriophages, bacterial components, viruses, exosomes, lipids, or ions). In some embodiments, an enucleated cell is engineered to produce (e.g., express, and in some cases, release or secrete) the therapeutic agent. In some embodiments, the parent may be engineered to produce the therapeutic agent prior to enucleation to produce the enucleated cell.

In some embodiments, the therapeutic agent is a virus. In some embodiments, the virus is a replication component virus. In some embodiments, the virus is a replication deficient virus. In some embodiments, the virus is an adeno-associated virus (AAV). Non-limiting examples of oncolytic viruses include AAV1, AAV2, AAV3B, AAV5, AAV6, AAV8, and AAV9. In some embodiments, the virus is a retrovirus. In some embodiments, the retrovirus is human T-lymphotropic virus (HTLV). In some embodiments, the retrovirus is human foamy virus. In some embodiments, the retrovirus is a γ-retrovirus. In some embodiments, the γ-retrovirus is murine leukemia virus (MLV). In some embodiments, the γ-retrovirus is Moloney murine sarcoma virus. In some embodiments, the retrovirus is an endogenous retrovirus. In some embodiments, the virus is a lentivirus. In some embodiments, the lentivirus is human immunodeficiency virus 1 (HIV-1). In some embodiments, the lentivirus is HIV-2. In some embodiments, the virus is a poxvirus. In some embodiments, the poxvirus is a vaccinia virus. In some embodiments, the poxvirus is a monkey pox virus. In some embodiments, the poxvirus is a variola virus. In some embodiments, the virus is a parvovirus. In some embodiments, the parvovirus is parvovirus B19. In some embodiments, the virus is a baculovirus. In some embodiments, the baculovirus is a nuclear polyhedrosis virus (NPV). In some embodiments, the virus is a herpes virus. Non-limiting examples of herpes viruses include varicella zoster virus, Epstein-Barr virus, cytomegalovirus, Kaposi's sarcoma-associated herpes virus, B virus, herpes simplex virus 1 (HSV-1), HSV-2, and varicellovirus.

In some embodiments, the virus is an oncolytic virus. Non-limiting examples of oncolytic viruses include Talimogene laherparepvec, Onyx-015, GL-ONC1, CV706, Voyager-V1, HSV-1716, Vaccinia virus, Adenovirus, HSV-1, HSV-2, B19PV, H1PV, Monkey pox virus, Sindbis virus, Zika virus, Measles virus, Newcastle disease virus, coxsackievirus A21, poliovirus, Seneca valley virus, reovirus, Maraba virus, Vesicular stomatitis virus, Poliovirus, Reovirus, Senecavirus, ECHO-7, and Semliki Forest virus. In some embodiments, oncolytic virus is a Coxsackie virus. In some embodiments, the oncolytic virus is a Vesicular Stomatitis Virus (VSV). In some embodiments, the oncolytic virus is an adenovirus. In some embodiments, the oncolytic virus is a retrovirus.

In some embodiments, the at least one therapeutic agent comprises an engineered oncolytic moiety. In some embodiments, the engineered oncolytic moiety comprises an oncolytic virus. In some embodiments, the oncolytic virus comprises an adenovirus, a vaccinia virus (e.g., Copenhagen strain, Western Reserve strain, or Wyeth strain), a reovirus, a herpes simplex virus, a Newcastle disease virus, a poxvirus, a myxoma virus, a picornavirus, an influenza virus, a coxsackievirus, a parvovirus, or a rhabdovirus (e.g., vesicular stomatitis virus), or a variant thereof. In some embodiments, the engineered oncolytic moiety comprises an adenoviral moiety or an adenovirus (e.g., Delta-24 strain or ONYX-015 strain). In some embodiments, the engineered oncolytic moiety comprises a modification to an oncolytic viral genome encoding an engineered oncolytic virus. In some embodiments, the engineered oncolytic virus is engineered for increasing selectivity for infecting cancer cells. In some embodiments, the engineered onco-lytic virus comprises an oncolytic viral genomic mutation, deletion, truncation, substitution, or a combination thereof. In some embodiments, the viral genome of the engineered oncolytic virus comprises an expression cassette. For example, an expression cassette may comprise an exogenous gene encoding an exogenous protein, and a promoter for driving expression of the exogenous protein. In some embodiments, the oncolytic viral genome of the engineered oncolytic virus comprises a substitution of one or more of genes in the oncolytic viral genome with the expression cassette disclosed herein. In some embodiments, the exogenous protein encoded by the expression cassette may exert additive or synergistic effect to the therapeutic effect of the oncolytic virus (e.g., targeting and killing cancer cells). For example, the expression cassette may encode an exogenous cytokine or immune checkpoint inhibitor.

In some embodiments, the oncolytic moiety comprises one or more polynucleotides encoding one or more components of an oncolytic viruses or an engineered oncolytic virus described herein. In some embodiments, the one or more polynucleotides is engineered to produce the engineered oncolytic virus or engineered oncolytic virus described herein. Adenovirus

In some embodiments, an oncolytic virus described herein is or comprises an adenovirus or an engineered adenovirus. In some embodiments, the engineered adenovirus comprises viral genomic regions of an adenovirus being deleted (complete or partial deletions), made non-functional, modified to attenuate function, or substituted by other sequences, or any combination thereof. In some embodiments, the engineered adenovirus can enhance tumor selectivity, for example, by attenuating the ability of the virus to replicate within normal quiescent cells without affecting the ability of the virus to replicate in tumor cells. In some embodiments, the engineered adenovirus has improved properties for use in treatment of cancer, for example, selective replication in cancer cells, attenuation of viral pathogenesis, enhancing lytic activity, modification of the antiviral immune response that can lead to rapid clearance of adenovirus, or modification of viral-induced systemic anti-tumor immunity, or any combination thereof.

In general, the infectious cycle of an adenovirus takes place in two steps: the early phase pre-cedes initiation of the replication of the adenoviral genome and permits production of the regulatory proteins and proteins involved in the replication and transcription of the viral DNA; and the late phase leads to the synthesis of the structural proteins. The early phase genes are dis-tributed in four regions-E1, E2, E3, and E4 (E denotes “early”). Three regions, E1, E2, and E4 are essential to replication of the virus. E1 is located at the 5′ end of the adenoviral genome and contains two viral transcription units, E1A and E1B. In particular, the E1A transcription unit codes for a protein that transactivates the transcription of the other viral genes, inducing transcription from the promoters of the EIB, E2A, E2B, E3, and E4 regions and the late genes.

In some embodiments, the adenovirus comprises a Delta-24 strain, a Delta-24-RGD strain, an ICOVIR-5 strain, an ICOVIR-7 strain, an ONYX-015 strain, a ColoAd1 strain, an H101 strain, or an AD5/3-D24-GMCSF strain, or a variant thereof. In some embodiments, the Delta-24 strain comprises a deletion of 24 nucleotides (SEQ ID NO: 3) within the CR2 portion of the ELA gene (SEQ ID NO: 2) that includes the area responsible for binding retinoblastoma (Rb) protein (nucleotides 923-946) corresponding to amino acids 122-129 in the encoded E1A protein. In some embodiments, the deletion within the CR2 portion of the EIA (SEQ ID NO: 1) comprises residues 122-129 of the E1A amino acid sequence (SEQ ID NO: 4). E1A binds Rb protein, releasing E2F, which serves as a transcription factor for genes that promote progression into the S-phase of the cell cycle, which enhances viral replication. In some embodiments, the E1A deletion increases the selectivity of the virus for cancer cells. The Delta-24 adenovirus cannot replicate in normal cells since mE1A cannot bind Rb protein to release E2F. However, in some embodiments, in cancer cells, the Rb deficiency indicates that there is E2F protein in the free state that drives cells into the S-state; thus, viral replication is enhanced. In some embodiments, this approach is the backbone of the Delta-24 adenoviral-based oncolytic virus therapy and mediates selectivity of a Delta-24 adenovirus for cancer cells. In some embodiments, the E1A region comprises a nucleic acid sequence that shares at least 80%, 85%, 90%, 95%, or 99% homology to SEQ ID NO: 2. In some embodiments, the E1A region comprises a nucleic acid sequence that is at least 80%, 85%, 90%, 95%, or 99% identical SEQ ID NO: 2. In some embodiments, the E1A region comprises a nucleic acid sequence comprising SEQ ID NO: 2. In some embodiments, the Delta-24 adenovirus is derived from any adenovirus serotype. In some embodiments, the Delta-24 adenovirus is derived from adenovirus serotype 5 (Ad-5, SEQ ID NO: 6).

In some embodiments, the Delta-24 strain comprises one or more nucleotide substitutions at the 24 nucleotides. For example, the 24 nucleotides can be substituted with nucleotides encoding RGD, yielding a modified adenoviral strain of Delta-24-RGD. In some embodiments, the Delta-24-RGD comprises substitution of a peptide in the adenovirus genome. In some embodiments, the peptide makes the Deta-24-RGD virus capable of attaching and infecting the cell through integrins, which are a membrane receptor very abundant in tumor cells, such as glioblastoma cells. In some embodiments, Delta-24-RGD is an adenovirus containing a deletion of 24 bases (bases 923-946) in the ELA gene and an insertion of an amino acid sequence (RGD-4C peptide) that is at least 80% identical to SEQ ID NO: 5 or shares at least 80% homology to SEQ ID NO: 5. In some embodiments, the Delta-24-RGD is an adenovirus containing a deletion of 24 bases (bases 923-946) in the ELA gene and an insertion of an amino acid sequence of SEQ ID NO: 5, an integrin-binding motif, which binds strongly to ανβ3 and ανβ5 integrins, into the HI loop of the fiber knob protein. In some embodiments, the E1A deletion increases the selectivity of the virus for cancer cells, and the RGD-4C sequence increases the infectivity of the virus for tumors, such as gliomas, which express low level of adenovirus receptors. In some embodiments, Delta-24-RGD has a potent anti-tumor mechanism of action by replicating in human tumors; eliciting tumor necrosis; and triggering an immune response.

In some embodiments, the ONYX-015 strain is a hybrid of virus Ad2 serotype and Ad5 serotype with deletions in the E1B-55K and E3B regions to enhance cancer selectivity. In some embodiments, H101 is a modified version of ONYX-015. In some embodiments, ICOVIR-5 and ICOVIR-7 comprise a retinoblastoma (Rb)-binding site deletion of E1A and a replacement of the E1A promoter by an E2F promoter. In some embodiments, ColoAd1 is a chimeric Ad-dllp/Ad3 serotype. In some embodiments, AD5/3-D24-GMCSF (CGTG-102) is an Ad5 serotype and Ad3 serotype capsid-modified adenovirus encoding GM-CSF (the Ad5 serotype capsid protein knob is replaced with a knob domain from Ad3 serotype).

In some embodiments, the enucleated cells described herein comprises one or more polynucleotides encoding one or more components of the Delta-24 virus or variant thereof disclosed herein. In some embodiments, the one or more polynucleotides comprise a nucleic acid sequence that shares at least 80%, 85%, 90%, 95%, or 99% homology to SEQ ID NO: 6 (human adenovirus 5; NCBI Reference Sequence: AC_000008.1). In some embodiments, the one or more polynucleotides comprise a nucleic acid sequence that is at least 80%, 85%, 90%, 95%, or 99% identical to SEQ ID NO: 6.

The therapeutic agent may be, or include, a targeting moiety described herein. Non-limiting example of the targeting moieties that may be produced by or contained in an enucleated cell includes chemokine receptors, adhesion molecules, and antigens. In some embodiments, the therapeutic agent may be, or include, a transmembrane moiety described herein.

In some embodiments, the therapeutic agent is recombinantly expressed by the enucleated cell or parent cell thereof. In some embodiments, the parent cell from which the enucleated cell is derived or obtained is engineered to produce or express the therapeutic agent. In some embodiments, expression of the therapeutic agent is stable (e.g., permanent). In some embodiments, the expression of the therapeutic agent by the parent cell is transient (e.g., non-permanent). In some embodiments, the parent cell is enucleated prior to engineering the enucleated cell to recombinantly express the therapeutic agent.

In some embodiments, the therapeutic agent is not naturally expressed (e.g., in the absence of engineering) in the cell from which the enucleated cell was derived or obtained (e.g., the therapeutic agent is exogenous to the parent cell). In some embodiments, the therapeutic agent is not naturally expressed in the subject (e.g., the therapeutic agent is exogenous to the subject). In some embodiments, the therapeutic agent is not naturally expressed in the subject at the intended site of therapy (e.g., a tumor, or a particular tissue, such as the brain, the intestine, the lungs, the heart, the liver, the spleen, the pancreas, muscles, eyes, and the like) (e.g., the therapeutic agent is exogenous to the intended site of therapy). In some embodiments, the level of the therapeutic agent is not naturally occurring in the enucleated cell of the parent cell.

In some embodiments, the therapeutic agent is naturally expressed (e.g., in the absence of engineering) in the cell from which the enucleated cell was derived or obtained (e.g., the therapeutic agent is endogenous to the enucleated cell). In some embodiments, the therapeutic agent is naturally expressed in the subject (e.g., the therapeutic agent is endogenous to the subject). In some embodiments, therapeutic agent is naturally expressed in the subject at the intended site of therapy (e.g., a tumor, or a particular tissue, such as the brain, the intestine, the lungs, the heart, the liver, the spleen, the pancreas, muscles, eyes, and the like) (e.g., the therapeutic agent is endogenous to the intended site of therapy).

In some embodiments, the therapeutic agent is derived from a synthetic cell and loaded into the enucleated cell. For example, the therapeutic agent may be endocytosed into the cell. Alternatively, the therapeutic agent may be synthesized by the cell and subsequently delivered to a target cell.

In some embodiments, the therapeutic agent comprises a corrected, a truncated, or a non-mutated version and/or copy of the DNA molecule, the RNA molecule, the protein, the peptide, the small molecule active agent, and/or the gene-editing factor as compared to the cell from which the enucleated cell was derived or obtained. For example, the therapeutic agent can correct a mutated p53 or EGFR in the target cell as part of the treatment for lung cancer.

In some embodiments, therapeutic agent comprises at least 2 (e.g., at least 2, 3, 4, 5, or more) different therapeutic DNA molecules, therapeutic RNA molecules, therapeutic proteins, therapeutic peptides, small molecule active agents, or therapeutic gene-editing factors, in any combination. For example, in some embodiments, a therapeutic agent comprises a therapeutic DNA molecule and a small molecule active agent. For example, in some embodiments, the therapeutic agent comprises two different small molecule active agents. For example, in some embodiments, the therapeutic agent comprises a chemokine receptor (e.g., for targeting) and a small molecule active agent.

In some embodiments, the therapeutic agent comprises an RNA molecule comprising messenger RNA (mRNA), short hairpin RNA (shRNA), small interfering RNA (siRNA), microRNA, long non-coding RNA (lncRNA) or an RNA virus. In some embodiments, the therapeutic agent comprises a DNA molecule that is single-stranded DNA, double-stranded DNA, an oligonucleotide, a plasmid, a bacterial DNA molecule or a DNA virus. In some embodiments, the therapeutic agent comprises a protein, or a portion thereof. In some embodiments, the protein is a cytokine, a growth factor, a hormone, an antibody or an antigen-binding fragment thereof, a small-peptide based drug, or an enzyme. In some embodiments, the enucleated cell transiently expresses the therapeutic agent. In some embodiments, the expression of the therapeutic agent is inducible. In some embodiments, the expression of the therapeutic agent permanent.

In some embodiments, the therapeutic agent comprises an exogenous agent. In some embodiments, the exogenous agent is an exogenous polypeptide. In some embodiments, the exogenous polypeptide is encoded by an exogenous polynucleotide delivered into the parent cell or the enucleated cell. In some embodiments, the exogenous polypeptide is synthesized or released by at least one intracellular organelle of the enucleated cell. In some embodiments, the exogenous polypeptide is released by the enucleated cell. In some embodiments, the exogenous polypeptide is expressed on the cell surface or the enucleated cell. In some embodiments, the enucleated cell delivers the exogenous polypeptide to a target cell. In some embodiments, the target cell is a cancer cell expressing the cancer biomarker of any cancer described herein. In some embodiments, the target cell is an endothelial cell expressing an endothelial biomarker described herein. In some embodiments, the endothelial cell is a blood vessel cell. In some embodiments, the endothelial cell is a lymphatic vessel cell.

In some embodiments, the exogenous polypeptide comprises a cytokine of any one of the cytokine or cytokine receptor binding fragment thereof. described herein. Non-limiting examples of cytokines include interleukin (IL) 1, IL-2, IL-3, IL-4, IL-5, IL-6, IL-7, IL-9, IL-10, IL-11, IL-13, IL-14, IL-15, IFN-α, IFN-β, IFN-γ, IL-17, and granulocyte-macrophage colony-stimulating factor (GM-CSF). In some embodiments, the exogenous polypeptide comprises a soluble cytokine. For example, the exogenous polypeptide can comprise an extracellular domain or fragment of the cytokine. In some embodiments, the exogenous polypeptide comprises a solubility as determined by turbidimetric solubility assay or thermodynamic solubility assay by dissolving the exogenous polypeptide in solvent such as organic solvent, including dimethyl sulfoxide (DMSO), dimethylformamide (DMF), acetonitrile, etc., or inorganic solvent, including water or phosphate-buffered saline (PBS). In some embodiments, the exogenous polypeptide comprises a solubility that is at least 0.0001 mg/ml, 0.0005 mg/ml, 0.001 mg/ml, 0.005 mg/ml, 0.01 mg/ml, 0.05 mg/ml, 0.1 mg/ml, 0.5 mg/ml, 1.0 mg/ml, 5.0 mg/ml, 10 mg/ml, 50 mg/ml, 100 mg/ml, 500 mg/ml 1,000 mg/ml 5,000 mg/ml, 10,000 mg/ml, 50,000 mg/ml, or 100,000 mg/ml.

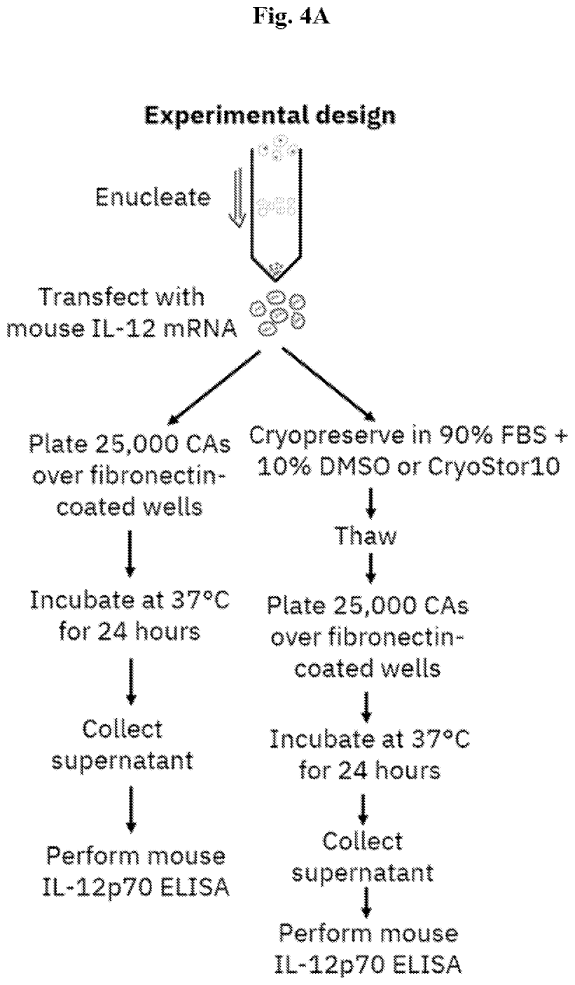

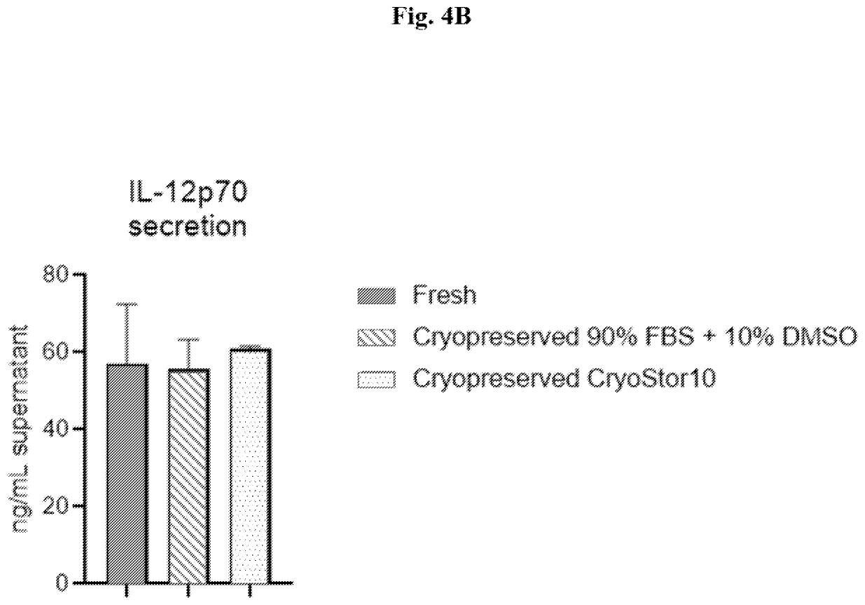

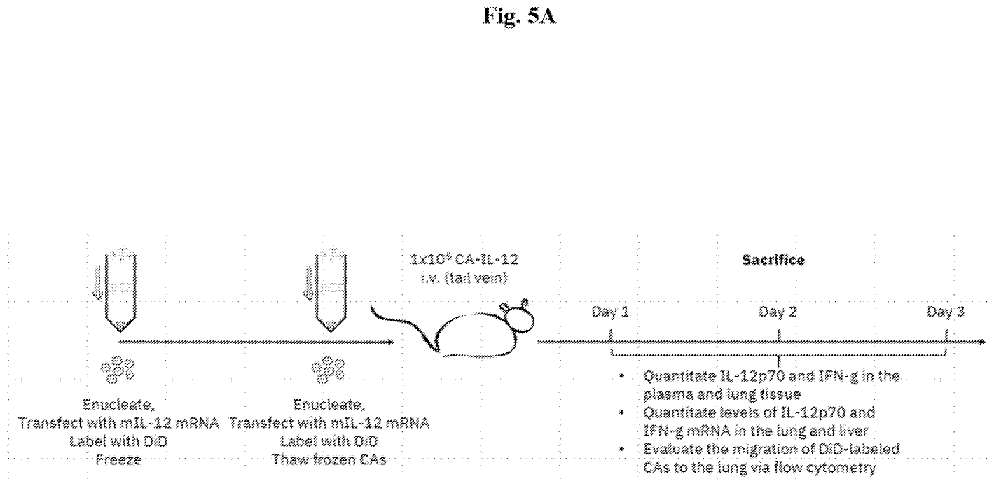

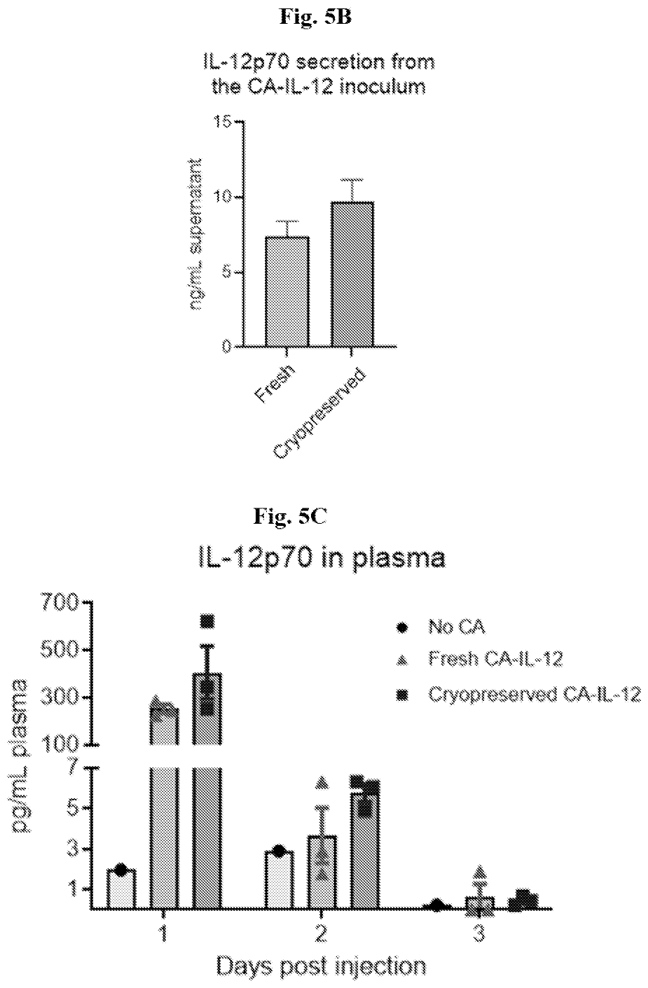

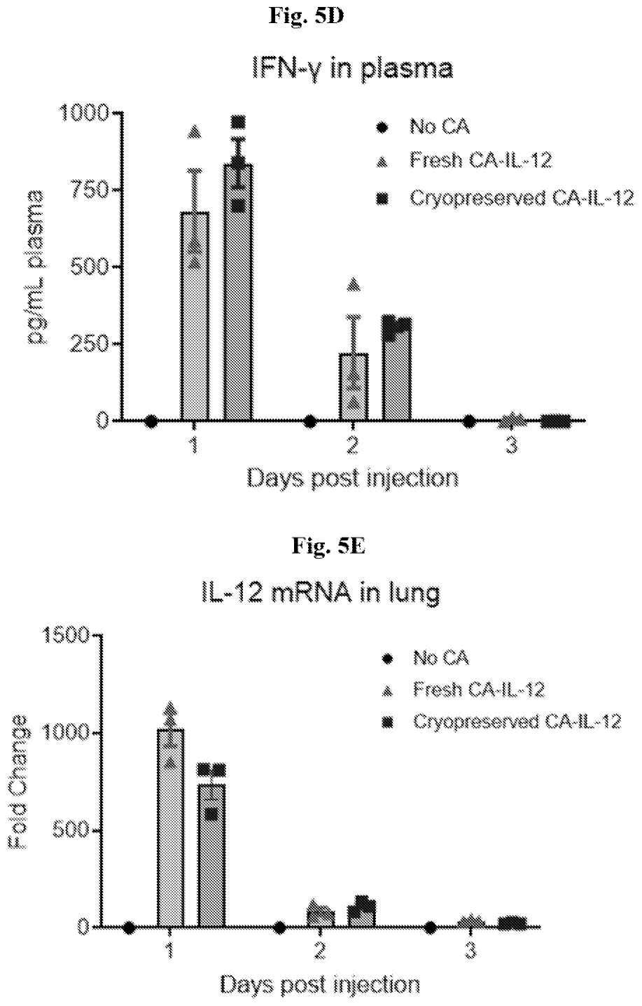

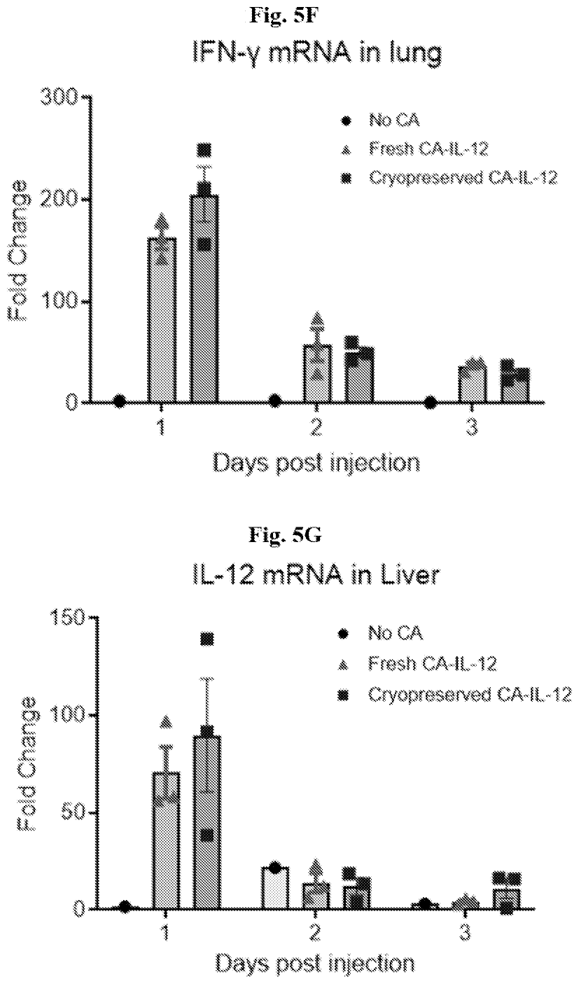

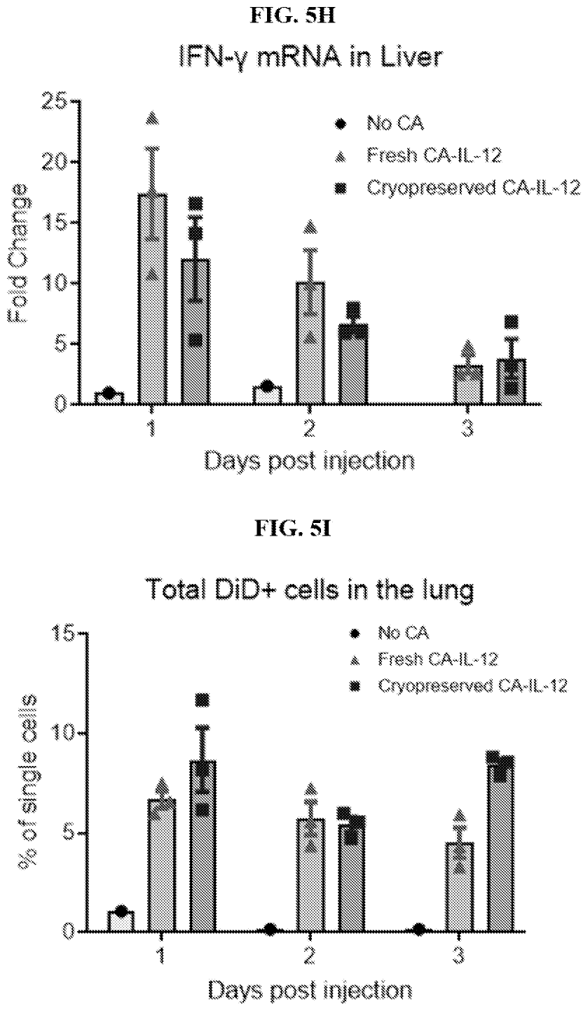

FIG. 4A shows the work flow for incorporating a targeting agent into the, in this case IL-12p70. FIG. 4B shows the results of following the workflow in FIG. 4A wherein there is similar supernatant values for secretion of IL-12p70 between fresh and cells cryopreserved two ways. FIG. 5A has a similar starting protocol but a different sample type. In FIG. 4A the sample type assessed was supernatant and in FIG. 5A different tissue type and markers were analyzed. and detailed in the right side of FIG. 5A under sacrifice. Following the protocol of FIG. 5A, the graph in FIG. 5B shows a comparison between the supernatant measurements of IL-12 in fresh and cryopreserved cells (similar to FIG. 4B). FIG. 5C analyzes the amount of IL-12p70 in plasma of the mice 1 day, 2 days, and 3 days post injection for mice containing no enucleated cells, fresh enucleated cells with IL-12, and cryopreserved enucleated cells with IL 12. FIG. 5D analyzes the amount of IFN-γ in plasma of the mice 1 day, 2 days, and 3 days post injection for mice containing no enucleated cells, fresh enucleated cells with IL-12, and cryopreserved enucleated cells with IL12. FIG. 5E analyzes the amount of IL-12 mRNA in lung tissue of the mice 1 day, 2 days, and 3 days post injection for mice containing no enucleated cells, fresh enucleated cells with IL-12, and cryopreserved enucleated cells with IL12. FIG. 5F analyzes the amount of IFN-γ mRNA in lung tissue of the mice 1 day, 2 days, and 3 days post injection for mice containing no enucleated cells, fresh enucleated cells with IL-12, and cryopreserved enucleated cells with IL12. FIG. 5G analyzes the amount of IL-12 mRNA in liver tissue of the mice 1 day, 2 days, and 3 days post injection for mice containing no enucleated cells, fresh enucleated cells with IL-12, and cryopreserved enucleated cells with IL12. FIG. 5H analyzes the amount of IFN-γ mRNA in liver tissue of the mice 1 day, 2 days, and 3 days post injection for mice containing no enucleated cells, fresh enucleated cells with IL-12, and cryopreserved enucleated cells with IL12. FIG. 5I analyzes total DiD+ cells in lung tissue of the mice 1 day, 2 days, and 3 days post injection for mice containing no enucleated cells, fresh enucleated cells with IL-12, and cryopreserved enucleated cells with IL 12.

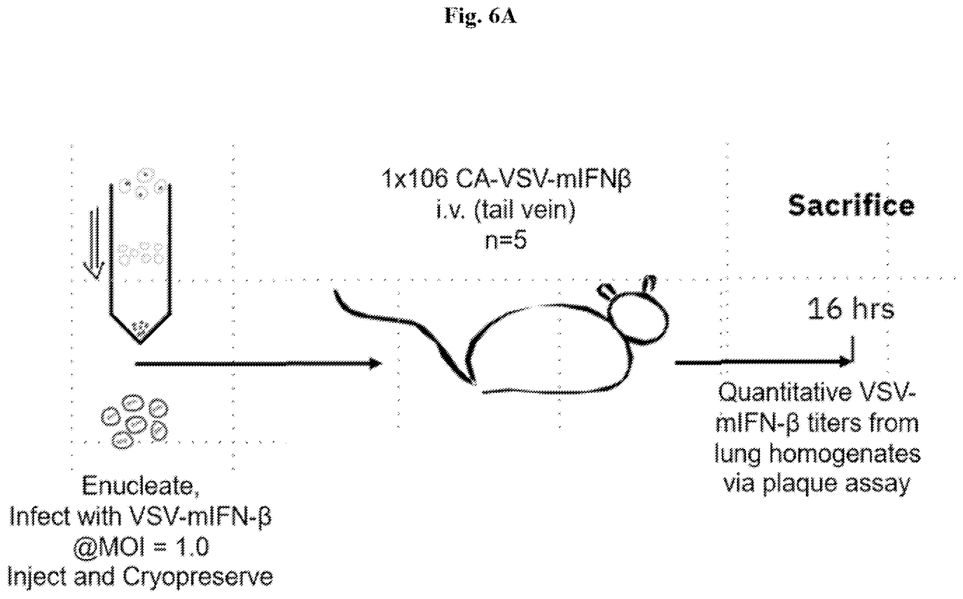

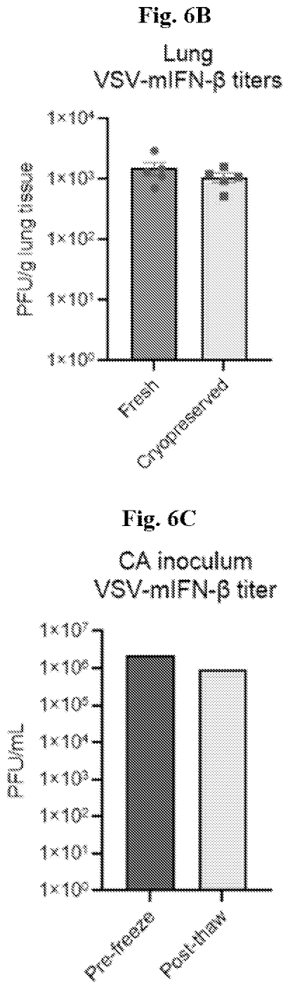

As shown in FIG. 6A enucleated cells with an oncolytic virus containing mlIFN-β were injected through the tail vein of mice and quantitated the virus titers after 16 hours as shown in FIG. 6B. Additional data were generated for the PFU/mL for the enucleated cell inoculum comparing pre-freeze and post-freeze enucleated cells (FIG. 6C).

In some embodiments, the exogenous polypeptide comprises a tumor necrosis factor (TNF) superfamily member or a catalytically active fragment thereof. Non-limiting examples of the TNF superfamily member include Lymphotoxin alpha (TNFβ), Tumor necrosis factor (TNFα), Lymphotoxin beta (TNFγ), OX40 ligand (CD252, Gp34, or CD134L), CD40 ligand (CD154, TRAP, Gp39, or T-BAM), Fas ligand (CD178, APTL, or CD95L), CD27 ligand (CD70), CD30 ligand (CD153), CD137 ligand (4-1 BBL), TNF-related apoptosis-inducing ligand (CD253 or APO-2L), Receptor activator of nuclear factor kappa-B ligand (CD254, OPGL, TRANCE, or ODF), TNF-related weak inducer of apoptosis (APO-3L or DR3L), a proliferation-inducing ligand (CD256, TALL-2, or TRDL1), B-cell activating factor (CD257, BLyS, TALL-1, or TNFSF20), LIGHT (CD258 or HVEML), Vascular endothelial growth inhibitor (TL1 or TL-1A), TNF superfamily member 18 (GITRL, AITRL, or TL-6), or Ectodysplasin A (ED1-A1 or ED1-A2).

In some embodiments, the therapeutic agent comprises any one of the immune checkpoint proteins described herein or an immune checkpoint inhibitor for inhibiting any one of the immune checkpoint protein described herein. Non-limiting examples of the immune checkpoint protein include PD-1, PD-L1, CTLA-4, VISTA, B7-H3 (also called CD276), A2AR, CD27, LAG3, TIM-3, T cell immunoreceptor with Ig and ITIM domains (TIGIT), CD73, NKG2A, PVRIG, PVRL2, CEACAM1, CEACAM5, CEACAM6, FAK, CCR-2, CCL-2, LIF, CD47, SIRPα, M-CSF, CSF-1R, IL-3, IL-1RAP, IL-8, SEMA4D, Angiopoietin-2, CLEVER-1, Axl, phosphatidylserine or a fragment thereof.

In some embodiments, the therapeutic agent is an exogenous gene editing system. In some embodiments, the exogenous gene editing system comprises at least one nucleic acid contacting moiety. In some embodiments, the exogenous gene editing system modulates expression of a target gene in a target cell. For example, an enucleated cell can deliver the exogenous gene editing system to the target cell, where the exogenous gene editing system modulate the expression of the target gene in the target cell. In some embodiments, the exogenous gene editing system comprises at least one guide nucleic acid. In some embodiments, the at least one guide nucleic acid is encoded by the enucleated cell. In some embodiments, the at least one guide nucleic acid is encoded from an exogenous nucleic acid as part of the exogenous gene editing system. In some embodiments, the at least one guide nucleic acid can be complexed with the at least one nucleic acid contacting moiety in the nanoparticle. For example, the exogenous gene editing system can comprise a nucleic acid contacting moiety comprising Cas RNP, where the nucleic acid contacting moiety is Cas9 or fragment thereof, and where the Cas9 is complexed with the guide nucleic acid, forming the Cas9 RNP.

In some embodiments, the nucleic acid contacting moiety can be complexed with the at least one guide nucleic acid and recruited to the target gene for modulating the expression of the target gene. In some embodiments, the nucleic acid contacting moiety can modulate the expression of the target gene by directly contacting and cleaving the target gene or transcript of the target gene. For example, the nucleic acid contacting moiety can comprise Cas9 or Cas12, where the nucleic acid contacting moiety can be complexed and recruited by the guide nucleic acid that is at least partially complementary to a nucleic acid sequence of the target gene. Upon contacting with the nucleic acid sequence of the target gene, the nucleic acid contacting moiety can then cleave the target gene. In some embodiments, the nucleic acid contacting moiety can modulate the expression of the target gene by cleaving transcript of the target gene. For example, the nucleic acid contacting moiety can comprise Cas13, where the nucleic acid contacting moiety can be complexed and recruited by the guide nucleic acid that is at least partially complementary to transcript of the target gene. Upon contacting with the transcript of the target gene, the nucleic acid contacting moiety can then cleave the transcript, decreasing expressing of the target gene.

In some embodiments, the nucleic acid contacting moiety can be deactivated for enzymatic cleavage activity (e.g., a deactivated Cas9 or dCas9). In such scenario, the nucleic acid contacting moiety can be operatively coupled to a transcriptional regulator such as a transcriptional activator or transcriptional repressor. For example, the nucleic acid contacting moiety can be covalently connected to a transcriptional activator or transcriptional repressor, where the transcriptional activator or transcriptional repressor can be recruited to a target gene by the nucleic acid contacting moiety complexing with the at least one guide nucleic acid and a nucleic acid sequence of the target gene. The recruitment of the transcriptional activator or transcriptional repressor to the target gene can the exert transcriptional activation or transcriptional repression of the target gene.

In some embodiments, the nucleic acid contacting moiety comprises a nucleic acid of ribonucleic acid interference (RNAi). For example, the nucleic acid contacting moiety can be a siRNA or anti-sense oligonucleotide for binding to a target gene or transcript of the target gene In some embodiments, the nucleic acid contacting moiety comprises a nuclease, such as an endonuclease (e.g., a heterologous endonuclease). In some embodiments, the nuclease can be a restriction enzyme. Suitable nucleases include, but are not limited to, CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), and eukaryotic Argonaute (eAgo)); any derivative thereof; any variant thereof and any fragment thereof.

In some embodiments, the nucleic acid contacting moiety comprises a DNA nuclease such as an engineered (e.g., programmable or targetable) DNA nuclease that is nuclease-deficient. In some embodiments, the nucleic acid contacting moiety comprises a nuclease-null DNA binding protein derived from a DNA nuclease that does not induce transcriptional activation or repression of a target DNA sequence unless it is present in a complex with one or more heterologous gene effectors of the disclosure. In some embodiments, the nucleic acid contacting moiety comprises a nuclease-null DNA binding protein derived from a DNA nuclease that can induce transcriptional activation or repression of a target DNA sequence (e.g., which can be altered or augmented by the presence of a heterologous gene effector of the disclosure).

In some embodiments, the nucleic acid contacting moiety comprises an RNA nuclease such as an engineered (e.g., programmable or targetable) RNA nuclease. In some embodiments, the nucleic acid contacting moiety comprises a nuclease-null RNA binding protein derived from an RNA nuclease that does not induce transcriptional activation or repression of a target RNA sequence unless it is present in a complex with one or more heterologous gene effectors of the disclosure. In some embodiments, the nucleic acid contacting moiety comprises a nuclease-null RNA binding protein derived from a RNA nuclease that can induce transcriptional activation or repression of a target RNA sequence (e.g., which can be altered or augmented by the presence of a heterologous gene effector of the disclosure).

In some embodiments, the nucleic acid contacting moiety comprises a nucleic acid-guided targeting system. In some embodiments, the nucleic acid contacting moiety comprises a DNA-guided targeting system. In some embodiments, the nucleic acid contacting moiety comprises an RNA-guided targeting system. The nucleic acid-guided targeting system can comprise and utilize, for example, at least one guide nucleic acid described herein that facilitates specific binding of a CRISPR-Cas system (e.g., a nuclease deficient form thereof, such as dCas9 or dCas14) to a target gene (e.g., target endogenous gene) or target gene regulatory sequence. Binding specificity can be determined by use of a guide nucleic acid, such as a single guide RNA (sgRNA) or a part thereof. In some embodiments, the use of different sgRNAs allows the compositions and methods of the disclosure to be used with (e.g., targeted to) different target genes (e.g., target endogenous genes) or target gene regulatory sequences.

Prokaryotic CRISPR-Cas (Clustered regularly interspaced short palindromic repeats-CRISPR associated) systems, for example, Class II CRISPR-Cas systems such as Cas9 and Cpfl, can be repurposed as a tool for regulation of gene expression, epigenome editing, and chromatin looping in compositions and methods of the disclosure. Nuclease-deactivated Cas (dCas) proteins complexed with heterologous gene effectors can allow for regulation of expression of target genes (e.g., target endogenous genes) adjacent to a site bound by the dCas.

In some embodiments, the nucleic acid contacting moiety comprises a CRISPR-associated (Cas) protein or a Cas nuclease that functions in a non-naturally occurring CRISPR (Clustered Regularly Interspaced Short Palindromic Repeats)/Cas (CRISPR-associated) system. In bacteria, this system can provide adaptive immunity against foreign DNA.

In a wide variety of organisms including diverse mammals, animals, plants, microbes, and yeast, a CRISPR/Cas system (e.g., modified and/or unmodified) can be utilized as a genome engineering tool, or can be modified to direct specific binding of engineered proteins to target loci as disclosed herein. A CRISPR/Cas system can comprise a guide nucleic acid described herein such as a guide RNA (gRNA) complexed with a Cas protein for targeted regulation of gene expression and/or activity or nucleic acid binding. An RNA-guided Cas protein (e.g., a Cas nuclease such as a Cas9 nuclease) can specifically bind a target polynucleotide (e.g., DNA) in a sequence-dependent manner. The Cas protein, if possessing nuclease activity, can cleave the DNA.

In some cases, the Cas protein is mutated and/or modified to yield a nuclease deficient protein or a protein with decreased nuclease activity relative to a wild-type Cas protein. A nuclease deficient protein can retain the ability to bind DNA, but may lack or have reduced nucleic acid cleavage activity.

In some embodiments, the nucleic acid contacting moiety comprises a Cas protein that forms a complex with a guide nucleic acid, such as a guide RNA or a part thereof. In some embodiments, the nucleic acid contacting moiety comprises a Cas protein that forms a complex with a single guide nucleic acid, such as a single guide RNA (sgRNA). In some embodiments, the nucleic acid contacting moiety comprises a RNA-binding protein (RBP) optionally complexed with a guide nucleic acid, such as a guide RNA (e.g., sgRNA), which is able to form a complex with a Cas protein. In some embodiments, the nucleic acid contacting moiety comprises a nuclease-null DNA binding protein derived from a DNA nuclease that can induce transcriptional activation or repression of a target DNA sequence. In some embodiments, the nucleic acid contacting moiety comprises a nuclease-null RNA binding protein derived from a RNA.

Any suitable CRISPR/Cas system can be used herein. A CRISPR/Cas system can be referred to using a variety of naming systems. A CRISPR/Cas system can be a type I, a type II, a type III, a type IV, a type V, a type VI system, or any other suitable CRISPR/Cas system. A CRISPR/Cas system as used herein can be a Class 1, Class 2, or any other suitably classified CRISPR/Cas system. Class 1 or Class 2 determination can be based upon the genes encoding the effector module. Class 1 systems generally have a multi-subunit crRNA-effector complex, whereas Class 2 systems generally have a single protein, such as Cas9, Cpfl, C2c1, C2c2, C2c3 or a crRNA-effector complex. A Class 1 CRISPR/Cas system can use a complex of multiple Cas proteins to effect regulation. A Class 1 CRISPR/Cas system can comprise, for example, type I (e.g., I, IA, IB, IC, ID, IE, IF, IU), type III (e.g., III, IIIA, IIIB, IIIC, IIID), and type IV (e.g., IV, IVA, IVB) CRISPR/Cas type. A Class 2 CRISPR/Cas system can use a single large Cas protein to effect regulation. A Class 2 CRISPR/Cas systems can comprise, for example, type II (e.g., II, IIA, IIB) and type V CRISPR/Cas type. CRISPR systems can be complementary to each other, and/or can lend functional units in trans to facilitate CRISPR locus targeting.

When a nucleic acid contacting moiety can comprise a Cas protein or derivative thereof, the Cas protein or derivative thereof can be a Class 1 or a Class 2 Cas protein. A Cas protein can be a type I, type II, type III, type IV, type V Cas protein, or type VI Cas protein. A Cas protein can comprise one or more domains. Non-limiting examples of domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. A guide nucleic acid recognition and/or binding domain can interact with a guide nucleic acid. A nuclease domain can comprise catalytic activity for nucleic acid cleavage. A nuclease domain can lack catalytic activity to prevent nucleic acid cleavage. A Cas protein can be a chimeric Cas protein or fragment thereof that is fused to other proteins or polypeptides. A Cas protein can be a chimera of various Cas proteins, for example, comprising domains from different Cas proteins.

Non-limiting examples of Cas proteins include c2c1, C2c2, c2c3, Casl, Cas1B, Cas2, Cas3, Cas4, Cas5, Cas5e (CasD), Cash, Cashe, Cas6f, Cas7, Cas8a, Cas8a1, Cas8a2, Cas8b, Cas8c, Cas9 (Csnl or Csx12), Cas10, CaslOd, Cas10, CaslOd, CasF, CasG, CasH, Cpfl, Csyl, Csy2, Csy3, Csel (CasA), Cse2 (CasB), Cse3 (CasE), Cse4 (CasC), Cscl, Csc2, Csa5, Csn2, Csm2, Csm3, Csm4, Csm5, Csm6, Cmr1, Cmr3, Cmr4, Cmr5, Cmr6, Csbl, Csb2, Csb3, Csx17, Csx14, Csx10, Csx16, CsaX, Csx3, Csx1, Csx15, Csfl, Csf2, Csf3, Csf4, Cul966, Cas13a, Cas13b, Cas13c, Cas13d, Cas13X, Cas13Y, Cas14 (e.g., Cas14 variants, such as Cas14a, Cas14b, Cas14c, etc.) and homologs or modified versions thereof.

A Cas protein or fragment or derivative thereof can be from any suitable organism. Non-limiting examples include Streptococcus pyogenes, Streptococcus thermophilus, Streptococcus sp., Staphylococcus aureus, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some aspects, the organism is Streptococcus pyogenes (S. pyogenes). In some aspects, the organism is Staphylococcus aureus (S. aureus). In some aspects, the organism is Streptococcus thermophilus (S. thermophilus).

A Cas protein can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractorsalsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinellasuccinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

A Cas protein as used herein can be a wildtype or a modified form of a Cas protein. A Cas protein can be an active variant, inactive variant, or fragment of a wild type or modified Cas protein. A Cas protein can comprise an amino acid change such as a deletion, insertion, substitution, variant, mutation, fusion, chimera, or any combination thereof relative to a wild-type version of the Cas protein (e.g., a wild-type version of Cas14). A Cas protein can be a polypeptide with at least about 5%, at least about 10%, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or 100% sequence identity or sequence similarity to a wild type Cas protein. A Cas protein can be a polypeptide with at most about 5%, at most about 10%, at most about 20%, at most about 30%, at most about 40%, at most about 50%, at most about 60%, at most about 70%, at most about 80%, at most about 90%, or at most about 100% sequence identity and/or sequence similarity to a wild type exemplary Cas protein. Variants or fragments can comprise at least about 5%, at least about 10%, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or 100% sequence identity or sequence similarity to a wild type or modified Cas protein or a portion thereof. Variants or fragments can be targeted to a nucleic acid locus in complex with a guide nucleic acid while lacking nucleic acid cleavage activity.

A Cas protein can comprise one or more nuclease domains, such as DNase domains. For example, a Cas9 protein can comprise a RuvC-like nuclease domain and/or an HNH-like 20 nuclease domain. The in a nuclease active form of Cas9, RuvC and HNH domains can each cut a different strand of double-stranded DNA to make a double-stranded break in the DNA. A Cas protein can comprise only one nuclease domain (e.g., Cpfl comprises RuvC domain but lacks HNH domain). In some embodiments, nuclease domains are absent. In some embodiments, nuclease domains are present but inactive or have reduced or minimal activity. In some embodiments, nuclease domains are present and active.

One or a plurality of the nuclease domains (e.g., RuvC, HNH) of a Cas protein can be deleted or mutated so that they are no longer functional or comprise reduced nuclease activity. For example, in a Cas protein comprising at least two nuclease domains (e.g., Cas9), if one of the nuclease domains is deleted or mutated, the resulting Cas protein, known as a nickase, can generate a single-strand break at a CRISPR RNA (crRNA) recognition sequence within a double-stranded DNA but not a double-strand break. Such a nickase can cleave the complementary strand or the non-complementary strand, but may not cleave both. If all of the nuclease domains of a Cas protein (e.g., both RuvC and HNH nuclease domains in a Cas9 protein; RuvC nuclease domain in a Cpfl protein) are deleted or mutated, the resulting Cas protein can have a reduced or no ability to cleave both strands of a double-stranded DNA. An example of a mutation that can convert a Cas9 protein into a nickase is a D10A (aspartate to alanine at position 10 of Cas9) mutation in the RuvC domain of Cas9 from S. pyogenes. H939A (histidine to alanine at amino acid position 839) or H840A (histidine to alanine at amino acid position 840) in the HNH domain of Cas9 from S. pyogenes can convert the Cas9 into a nickase. An example of a mutation that can convert a Cas9 protein into a dead Cas9 is a D10A (aspartate to alanine at position 10 of Cas9) mutation in the RuvC domain and H939A (histidine to alanine at amino acid position 839) or H840A (histidine to alanine at amino acid position 840) in the HNH domain of Cas9 from S. pyogenes.

A nuclease dead Cas protein can comprise one or more mutations relative to a wild-type version of the protein. The mutation can result in no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity in one or more of the plurality of nucleic acid-cleaving domains of the wild-type Cas protein. The mutation can result in one or more of the plurality of nucleic acid-cleaving domains retaining the ability to cleave the complementary strand of the target nucleic acid but reducing its ability to cleave the non-complementary strand of the target nucleic acid. The mutation can result in one or more of the plurality of nucleic acid-cleaving domains retaining the ability to cleave the non-complementary strand of the target nucleic acid but reducing its ability to cleave the complementary strand of the target nucleic acid. The mutation can result in one or more of the plurality of nucleic acid-cleaving domains lacking the ability to cleave the complementary strand and the non-complementary strand of the target nucleic acid. The residues to be mutated in a nuclease domain can correspond to one or more catalytic residues of the nuclease. For example, residues in the wild type exemplary S. pyogenes Cas9 polypeptide such as Asp10, His840, Asn854 and Asn856 can be mutated to inactivate one or more of the plurality of nucleic acid-cleaving domains (e.g., nuclease domains). The residues to be mutated in a nuclease domain of a Cas protein can correspond to residues Asp10, His840, Asn854 and Asn856 in the wild type S. pyogenes Cas9 polypeptide, for example, as determined by sequence and/or structural alignment.

A Cas protein can comprise an amino acid sequence having at least about 5%, at least about 10%, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or 100% sequence identity or sequence similarity to a nuclease domain (e.g., RuvC domain, HNH domain) of a wild-type Cas protein.

A Cas protein, variant or derivative thereof can be modified to enhance regulation of gene expression by compositions and methods of the disclosure, e.g., as part of a complex disclosed herein. A Cas protein can be modified to increase or decrease nucleic acid binding affinity, nucleic acid binding specificity, enzymatic activity, and/or binding to other factors, such as heterodimerization or oligomerization domains and induce ligands. Cas proteins can also be modified to change any other activity or property of the protein, such as stability. For example, one or more nuclease domains of the Cas protein can be modified, deleted, or inactivated, or a Cas protein can be truncated to remove domains that are not essential for the desired function of the protein or complex. A Cas protein can be modified to modulate (e.g., enhance or reduce) the activity of the Cas protein for regulating gene expression by a complex of the disclosure that comprises a heterologous gene effector.

For example, a Cas protein can be coupled (e.g., fused, covalently coupled, or non-covalently coupled) to a heterologous gene effector (e.g., an epigenetic modification domain, a transcriptional activation domain, and/or a transcriptional repressor domain). A Cas protein can be coupled (e.g., fused, covalently coupled, or non-covalently coupled) to an oligomerization or dimerization domain as disclosed herein (e.g., a heterodimerization domain). A Cas protein can be coupled (e.g., fused, covalently coupled, or non-covalently coupled) to a heterologous polypeptide that provides increased or decreased stability. A Cas protein can be coupled (e.g., fused, covalently coupled, or non-covalently coupled) to a sequence that can facilitate degradation of the Cas protein or a complex containing the Cas protein, for example, a degron, such as an inducible degron (e.g., auxin inducible).

A Cas protein can be a fusion protein, e.g., a fusion comprising the Cas protein and one or more of the partners as disclosed herein. The fused domain or heterologous polypeptide can be located at the N-terminus, the C-terminus, or internally within the Cas protein.

A partner of the Cas protein (e.g., covalently or non-covalently coupled to a dCas protein as disclosed herein) can be a transcriptional effector (e.g., a transcriptional activator or a transcriptional repressor). The transcriptional effector can be heterologous to the cell as provided herein.

In some embodiments, the transcriptional effector can be a histone epigenetic modifier (or a histone modifier). In some cases, the histone epigenetic modifier can modulate histones through methylation (e.g., a histone methylation modifier, such as an amino acid methyltransferase, e.g., KRAB). In some cases, the histone epigenetic modifier can modulate histones through acetylation. In some cases, the histone epigenetic modifier can modulate histones through phosphorylation. In some cases, the histone epigenetic modifier can modulate histones through ADP-ribosylation. In some cases, the histone epigenetic modifier can modulate histones through glycosylation. In some cases, the histone epigenetic modifier can modulate histones through SUMOylation. In some cases, the histone epigenetic modifier can modulate histones through ubiquitination. In some cases, the histone epigenetic modifier can modulate histones by remodeling histone structure, e.g., via an ATP hydrolysis-dependent process.

In some embodiments, the transcriptional effector can be a gene epigenetic modifier (or a gene modifier). In some cases, a gene modifier can modulate genes through methylation (e.g., a gene methylation modifier, such as a DNA methyltransferase or DNMT). In some cases, a gene modifier can modulate genes through acetylation.

In some embodiments, the transcriptional effector is from a family of related histone acetyltransferases. Non-limiting examples of histone acetyltransferases include GNAT subfamily, MYST subfamily, p300/CBP subfamily, HAT1 subfamily, GCN5, PCAF, Tip60, MOZ, MORF, MOF, HBO1, p300, CBP, HAT1, ATF-2, SRC1, and TAFII250.

In some embodiments, the transcriptional effector is from a histone lysine methyltransferase. Non-limiting examples of histone lysine methyltransferases include EZH subfamily, Non-SET subfamily, Other SET subfamily, PRDM subfamily, SET1 subfamily, SET2 subfamily, SUV39 subfamily, SYMD subfamily, ASH1L, EHMT1, EHMT2, EZH1, EZH2, MLL, MLL2, MLL3, MLL4, MLL5, NSD1, NSD2, NSD3, PRDM1, PRDM10, PRDM11, PRDM12, PRDM13, PRDM14, PRDM15, PRDM16, PRDM2, PRDM4, PRDM5, PRDM6, PRDM7, PRDM8, PRDM9, SET1, SET1L, SET2L, SETD2, SETD3, SETD4, SETD5, SETD6, SETD7, SETD8, SETDB1, SETDB2, SETMAR, SUV39H1, SUV39H2, SUV420H1, SUV420H2, SYMD1, SYMD2, SYMD3, SYMD4, and SYMD5.

Examples of proteins (or fragments thereof) that can be used as a fusion partner to increase transcription include but are not limited to: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1, and the like; histone lysine demethylases such as JHDM2a/b, UTX, JMJD3, and the like; histone acetyltransferases such as GCN5, PCAF, CBP, p300, TAF1, TIP60/PLIP, MOZMYST3, MORFMYST4, SRC1, ACTR, PI 60, CLOCK, and the like; and DNA demethylases such as Ten-Eleven Translocation (TET) dioxygenase 1 (TET1CD), TET1, DME, DML1, DML2, ROSI, or a combination thereof.

Examples of proteins (or fragments thereof) that can be used as a fusion partner to decrease transcription include but are not limited to: transcriptional repressors such as the Kruppel associated box (KRAB or SKD); KOX1 repression domain; the Mad mSIN3 interaction domain (SID); the ERF repressor domain (ERD), the SRDX repression domain (e.g., for repression in plants), and the like; histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARJD 1 A/RBP2, JARID1B/PLU-1, JARID 1C/SMCX, JARIDID/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; DNA methylases such as Hhal DNA m5c-methyltransferase (M.Hhal), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like; and periphery recruitment elements such as Lamin A, Lamin B, or a combination thereof.

A Cas protein can be provided in any form. For example, a Cas protein can be provided in the form of a protein, such as a Cas protein alone or complexed with a guide nucleic acid as a ribonucleoprotein. A Cas protein can be provided in a complex, for example, complexed with a guide nucleic acid and/or one or more heterologous gene effectors of the disclosure. A Cas protein can be provided in the form of a nucleic acid encoding the Cas protein, such as an RNA (e.g., messenger RNA (mRNA)), or DNA. The nucleic acid encoding the Cas protein can be codon optimized for efficient translation into protein in a particular cell or organism.

In some embodiments, a Cas protein, variant or derivative thereof is a nuclease dead Cas (dCas) protein. A dead Cas protein can be a protein that lacks nucleic acid cleavage activity.

A Cas protein can comprise a modified form of a wild type Cas protein. The modified form of the wild type Cas protein can comprise an amino acid change (e.g., deletion, insertion, or substitution) that reduces the nucleic acid-cleaving activity of the Cas protein. For example, the modified form of the Cas protein can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the wild-type Cas protein (e.g., Cas9 from S. pyogenes). The modified form of Cas protein can have no substantial nucleic acid-cleaving activity. When a Cas protein is a modified form that has no substantial nucleic acid-cleaving activity, it can be referred to as enzymatically inactive, “deactivated” and/or “dead” (abbreviated by “d”). A dead Cas protein (e.g., dCas, dCas9, dCas14) can bind to a target polynucleotide but may not cleave or minimally cleaves the target polynucleotide. In some aspects, a dead Cas protein is a dead Cas14 protein.

A dCas polypeptide (e.g., dCas14 polypeptide) can associate with a single guide RNA (sgRNA) to activate or repress transcription of a target gene (e.g., target endogenous gene), for example, in combination with heterologous gene effector(s) disclosed herein. sgRNAs can be introduced into cells expressing the Cas or variant thereof, as provided herein. In some cases, such cells can contain one or more different sgRNAs that target the same target gene (e.g., target endogenous gene) or target gene regulatory sequence. In other cases, the sgRNAs target different nucleic acids in the cell (e.g., different target genes, different target gene regulatory sequences, or different sequences within the same target gene or target gene regulatory sequence).

Enzymatically inactive can refer to a nuclease that can bind to a nucleic acid sequence in a polynucleotide in a sequence-specific manner, but will not cleave a target polynucleotide or will cleave it at a substantially reduced frequency. An enzymatically inactive guide moiety can comprise an enzymatically inactive domain (e.g. nuclease domain). Enzymatically inactive can refer to no activity. Enzymatically inactive can refer to substantially no activity. Enzymatically inactive can refer to essentially no activity. Enzymatically inactive can refer to an activity no more than 1%, no more than 2%, no more than 3%, no more than 4%, no more than 5%, no more than 6%, no more than 7%, no more than 8%, no more than 9%, or no more than 10% activity compared to a comparable wild-type activity.

In some embodiments, the nucleic acid contacting moiety as disclosed herein does not contain a nucleic acid-guided targeting system. For example, the nucleic acid contacting moiety can include proteins that bind to a target gene (e.g., target endogenous gene) or target gene regulatory sequence based on protein structural features, such as certain nucleases disclosed herein.

In some embodiments, the enucleated cells comprise an additional therapeutic agent, such as those disclosed herein. In some embodiments, the composition comprising the enucleated cells is formulated for administration to a subject disclosed herein with an additional therapeutic agent. In some embodiments, the additional therapeutic agent is administered to the subject sequentially, simultaneously, substantially sequentially, or substantially simultaneously.

### (e) Pharmaceutical Formulations

Disclosed herein, in some embodiments, are pharmaceutical formulations and/or compositions comprising the compositions disclosed herein and a pharmaceutically acceptable: carrier, excipient, diluent, or nebulized inhalant. In some embodiments, the pharmaceutical formulation is in a unit dose form. In some embodiments, the compositions disclosed herein comprise one or more active agents or therapeutic agents.

In some embodiments, the compositions comprise two or more active agents, or two or more therapeutic agents as disclosed herein. In some embodiments, the two or more active agents are contained in a single dosage unit, such as for example, when the enucleated cell comprises two or more therapeutic agents. In embodiments, the two or more active agents are contained in separate dosage units, such as when the enucleated cell is administered separately from an additional therapeutic agent or adjuvant. In some embodiments, the pharmaceutical composition described herein includes at least one additional active agent other than the enucleated cell described herein. In some embodiments, the at least one additional active agent is a chemotherapeutic agent, cytotoxic agent, cytokine, growth-inhibitory agent, anti-hormonal agent, anti-angiogenic agent, cardio protectant, and/or checkpoint inhibitor. Non-limiting checkpoint inhibitor includes IMP321/Eftilagimod alpha (Immutep), Relatlimab BMS-986016, Ipilimumab (Yervoy), Pembrolizumab (Keytruda), Nivolumab (Opdivo), Cemiplimab (Libtayo), Atezolizumab (Tecentriq), Avelumab (Bavencio), Durvalumab (Imfinzi), Ipilimumab (Yervoy), LAG525, MK-4280, Irinotecan, Oxaliplatin, REGN3767, TSR-033, BI754111, Sym022, FS118 (a bi-specific anti-LAG3/PD-L1 antagonistic mAb), MGD013 (a bi-specific anti-LAG3/PD-1 antagonistic mAb), TSR-022, Niraparib, Bevacizumab, MBG453, Decitabine, Spartalizumab, Sym023, INCAGN2390, LY3321367, Ramucirumab, Abemaciclib, Merestinib, BMS-986258, SHR-1702, Camrelizumab, MK-7684, Etigilimab/OMP-313 M32, Tiragolumab/MTIG7192A/RG-6058, BMS-986207, AB-154, ASP-8374, JNJ-61610588, CA-170d, Enoblituzumab/MGA271, MGD009, I-8H9/omburtamab, Trastuzumab, MGD013 (Anti-PD-1, anti-LAG-3 dual checkpoint inhibitor), BGB-A1217, CM-24 (MK-6018), BMS 986178, MEDI6469, PF-04518600, GSK3174998, MOXR0916, Utomilimab (PF-05082566), Urelumab (BMS-663513) ES101, BMS-986156, TRX-518, AMG 228, JTX-2011, GSK3359609, BMS-986226, MEDI-570, or Varlilumab (CDX-1127). Such compounds or drugs may be present in combination in amounts that are effective for the purpose intended. Additional non-limiting examples of the additional therapeutic agent include CPI-006 (for inhibiting CD73 and allowing T cell and APC activation); Monalizumab (for inhibiting NKG2A); COM701 (for inhibiting PVRIG/PVRL2 and activating T cell); CM24 (for inhibiting CEACAM1 and allowing T and NK cells activation); NEO-201 (for inhibiting CEACAM5 and CEACAM6 which allows T cell activation while interfering with tumor cell growth); Defactinib (for inhibiting FAK and interfering with tumor growth); PF-04136309 (for inhibiting CCR-2 and CCL-2 and allowing T cell recruitment and activation); MSC-1 (for inhibiting LIF and allowing T cell and APC activation while interfering with cancer growth); Hu5F9-G4 (5F9), ALX148, TTI-662, and RRx-001 (for inhibiting CD47 or SIRPα and allowing T cell and APC activation); Lacnotuzumab (MCS-110), LY3022855, SNDX-6352, Emactuzumab (RG7155), and Pexidartinib (PLX3397) (for inhibiting M-CSF or CSF-1R and allowing APC activation); CAN04 and Canakinumab (ACZ885) (for inhibiting IL-3 or IL-1RAP and allowing T cell and APC activation); BMS-986253 (for inhibiting IL-8 and decreasing immunosuppressive tumor microenvironment while interfering with tumor growth); Pepinemab (VX15/2503) (for inhibiting SEMA4D and decreasing immunosuppressive tumor microenvironment while interfering with tumor growth); Trebananib (for inhibiting Angiopoietin-2 and allowing APC activation while interfering with cancer growth); FP-1305 (for inhibiting CLEVER-1 and allowing APC activation); Enapotamab vedotin (EnaV) (for inhibiting Axl and allowing APC activation while interfering with cancer growth); or Bavituximab (for inhibiting phosphatidylserine and allowing T cell and APC activation while interfering with cancer growth).

The compositions may include at least an exogenous therapeutic agent as an active ingredient in free-acid or free-base form, or in a pharmaceutically acceptable salt form. In addition, the methods and compositions described herein include the use of N-oxides (if appropriate), crystalline forms, amorphous phases, as well as active metabolites of these compounds having the same type of activity. In some embodiments, therapeutic agents exist in unsolvated form or in solvated forms with pharmaceutically acceptable solvents such as water, ethanol, and the like. The solvated forms of the therapeutic agents are also considered to be disclosed herein.

In certain embodiments, compositions provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, compositions described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, I about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

In some embodiments, the pharmaceutical formulation is formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesicaly, intravaginally, intrarectally, orally, sublingually, transdermally, by inhalation, by inhaled nebulized form, by intraluminal-GI route, or any combination thereof, to a subject. In some embodiments, the pharmaceutical formulation is formulated for administering intravenously. In some embodiments, the pharmaceutical formulation further comprises at least one additional active agent. In some embodiments, the at least one additional active agent comprises a cytokine, a growth factor, a hormone, an enzyme, a small molecule, a compound, or any combination thereof.

### (f) Formulations for Administration

Disclosed herein are formulations. In some embodiments, the formulation comprises a plurality of enucleated cells formulated from a cryopreserved composition, wherein the cryopreserved composition comprises the plurality of enucleated cells that are cryopreserved, wherein at least a subset of the plurality of enucleated cells comprises (i) a therapeutic agent, and (ii) intracellular organelles sufficient to release the therapeutic agent in vivo or ex vivo in an amount that is greater than or equal to about an amount of the therapeutic agent released by otherwise identical enucleated cells that were not cryopreserved.

In some embodiments, the plurality of enucleated cells comprises a diameter comprising less than or equal to about 70% of an average diameter of a nucleated parent cell. In some embodiments, the plurality of enucleated cells comprises a diameter comprising between about 1 micrometer (μm) to about 100 μm. In some embodiments, the plurality of enucleated cells comprises a diameter comprising between about 5 μm to about 25 μm. In some embodiments, the plurality of enucleated cells comprises a diameter comprising about 8 μm.

In some embodiments, the one or more structural features comprise one or more tunneling nanotubes. In some embodiments, the intracellular organelles comprise a Golgi apparatus, an endoplasmic reticulum, or any combination thereof.

The compositions and/or formulations described herein are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for rehydration into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms may be ensured by various antibacterial and antifungal agents, such as parabens, chlorobutanol, phenol, sorbic acid, and the like. In some cases, it is desirable to include isotonic agents, such as sugars, sodium chloride, and the like. Prolonged absorption of the injectable pharmaceutical form may be brought about by the use of agents delaying absorption, such as aluminum monostearate and gelatin.

For intravenous injections or drips or infusions, a composition described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion.

Compositions for injection may be presented in unit dosage form, e.g., in ampoules or in multi dose containers, with an added preservative. The composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a therapeutic agent is formulated for use as an aerosol, a mist or a powder. Pharmaceutical compositions described herein are conveniently delivered in the form of an aerosol spray presentation from pressurized packs or nebulizers, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the therapeutic agent described herein and a suitable powder base such as lactose or starch. Formulations that include a composition are prepared as solutions in saline, employing benzyl alcohol or other suitable preservatives, fluorocarbons, and/or other solubilizing or dispersing agents known in the art. Preferably these compositions and formulations are prepared with suitable nontoxic pharmaceutically acceptable ingredients. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the compositions described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active therapeutic agent doses.

In some embodiments, the compositions of the exogenous therapeutic agents are in the form of a capsules, including push fit capsules made of gelatin, as well as soft, sealed capsules made of gelatin and a plasticizer, such as glycerol or sorbitol. The push fit capsules contain the active ingredients in admixture with filler such as lactose, binders such as starches, and/or lubricants such as talc or magnesium stearate and, optionally, stabilizers. In soft capsules, the active therapeutic agent is dissolved or suspended in suitable liquids, such as fatty oils, liquid paraffin, or liquid polyethylene glycols. In some embodiments, stabilizers are added. A capsule may be prepared, for example, by placing the bulk blend of the formulation of the therapeutic agent inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

Compositions for oral administration are in dosages suitable for such administration. In one aspect, solid oral dosage forms are prepared by mixing a composition with one or more of the following: antioxidants, flavoring agents, and carrier materials such as binders, suspending agents, disintegration agents, filling agents, surfactants, solubilizers, stabilizers, lubricants, wetting agents, and diluents. In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the composition is in the form of a powder. Compressed tablets are solid dosage forms prepared by compacting the bulk blend of the formulations described above. In various embodiments, tablets will include one or more flavoring agents. In other embodiments, the tablets will include a film surrounding the final compressed tablet. In some embodiments, the film coating may provide a delayed release of a therapeutic agent from the formulation. In other embodiments, the film coating aids in patient compliance. Film coatings may range from about 1% to about 3% of the tablet weight. In some embodiments, solid dosage forms, e.g., tablets, effervescent tablets, and capsules, are prepared by mixing particles of a therapeutic agent with one or more pharmaceutical excipients to form a bulk blend composition. The bulk blend is readily subdivided into equally effective unit dosage forms, such as tablets, pills, and capsules. In some embodiments, the individual unit dosages include film coatings.

In another aspect, dosage forms include microencapsulated formulations. In some embodiments, one or more other compatible materials are present in the microencapsulation material. Non-limiting example of materials includes pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Liquid formulation dosage forms for oral administration are optionally aqueous suspensions selected from the group including, but not limited to, pharmaceutically acceptable aqueous oral dispersions, emulsions, solutions, elixirs, gels, and syrups. In addition to therapeutic agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further include a crystal-forming inhibitor.

In some embodiments, the compositions described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients.

The compositions (e.g., pharmaceutical compositions) described herein may be formulated for administration to a subject by administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The composition described herein may include, but not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended-release formulations, pulsatile release formulations, multiparticulate formulations, and mixed immediate and controlled release formulations.

Buccal formulations are administered using a variety of formulations known in the art. In addition, the buccal dosage forms described herein may further include a bioerodible (hydrolysable) polymeric carrier that also serves to adhere the dosage form to the buccal mucosa. For buccal or sublingual administration, the compositions may take the form of tablets, lozenges, or gels formulated in a suitable manner.

For intravenous injections, a composition is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a composition described herein is in a form suitable for parenteral injection as a sterile suspensions, solutions or emulsions in oily or aqueous vehicles, and contain formulatory agents such as suspending, stabilizing and/or dispersing agents. The compositions for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Suitable formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

In some embodiments, the compositions are provided that include particles of a therapeutic agent and at least one dispersing agent or suspending agent for oral administration to a subject. The formulations may be a powder and/or granule for suspension, and upon admixture with water, a substantially uniform suspension is obtained.

Furthermore, the compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, the compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other the compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state for at least 4 hours. In one embodiment, an aqueous suspension is resuspended into a homogenous suspension by physical agitation lasting less than 1 minute. In still another embodiment, no agitation is necessary to maintain a homogeneous aqueous dispersion.

An aerosol formulation for nasal administration is generally an aqueous solution designed to be administered to the nasal passages in drops or sprays. Nasal solutions may be similar to nasal secretions in that they are generally isotonic and slightly buffered to maintain a pH of about 5.5 to about 6.5, although pH values outside of this range may additionally be used. Antimicrobial agents or preservatives may also be included in the formulation.

An aerosol formulation for inhalations and inhalants may be designed so that the agent or combination of agents is carried into the respiratory tree of the subject when administered by the nasal or oral respiratory route. Inhalation solutions may be administered, for example, by a nebulizer. Inhalations or insufflations, comprising finely powdered or liquid drugs, may be delivered to the respiratory system as a pharmaceutical aerosol of a solution or suspension of the agent or combination of agents in a propellant, e.g., to aid in disbursement. Propellants may be liquefied gases, including halocarbons, for example, fluorocarbons such as fluorinated chlorinated hydrocarbons, hydrochlorofluorocarbons, and hydrochlorocarbons, as well as hydrocarbons and hydrocarbon ethers.

Aerosol formulations may also include other components, for example, ethanol, isopropanol, propylene glycol, as well as surfactants or other components such as oils and detergents. These components may serve to stabilize the formulation and/or lubricate valve components.

The aerosol formulation may be packaged under pressure and may be formulated as an aerosol using solutions, suspensions, emulsions, powders and semisolid preparations. For example, a solution aerosol formulation comprises a solution of an agent such as a transporter, carrier, or ion channel inhibitor in (substantially) pure propellant or as a mixture of propellant and solvent. The solvent may be used to dissolve the agent and/or retard the evaporation of the propellant. Solvents may include, for example, water, ethanol and glycols. Any combination of suitable solvents may be use, optionally combined with preservatives, antioxidants, and/or other aerosol components.

An aerosol formulation may be a dispersion or suspension. A suspension aerosol formulation comprises a suspension of an agent or combination of agents, e.g., a transporter, carrier, or ion channel inhibitor, and a dispersing agent. Dispersing agents may include, for example, sorbitan trioleate, oleyl alcohol, oleic acid, lecithin and corn oil. A suspension aerosol formulation may also include lubricants, preservatives, antioxidant, and/or other aerosol components.

An aerosol formulation may similarly be formulated as an emulsion. An emulsion aerosol formulation may include, for example, an alcohol such as ethanol, a surfactant, water and a propellant, as well as an agent or combination of agents, e.g., a transporter, carrier, or ion channel. The surfactant used may be nonionic, anionic or cationic. One example of an emulsion aerosol formulation comprises, for example, ethanol, surfactant, water and propellant. Another example of an emulsion aerosol formulation comprises, for example, vegetable oil, glyceryl monostearate and propane.

### Methods

Disclosed herein, in some embodiments, are methods of producing or using the compositions disclosed herein. In some embodiments, methods comprise high throughput techniques for enucleated cells to produce compositions comprising enucleated cells for biomedical applications with minimal residual nucleated parent cells. In some embodiments, are methods for cryopreserving enucleated cells. In some embodiments, the cryopreserved enucleated cells release greater than or equal to amounts of a therapeutic agent compared to otherwise identical enucleated cells that were not cryopreserved.

In some embodiments, methods comprising inducing expression of a suicide gene under conditions suitable to kill the residual nucleated parent cells in the composition. The methods disclosed herein also provide methods of using the enucleated cells as fusion partners (e.g., fusion to another cell in vivo or ex vivo), or a therapeutic agent delivery vehicle, or a combination thereof.

Disclosed herein, in some aspects, are methods of producing an enucleated cell described herein comprising enucleating a nucleated parent cell. In some embodiments, the parent cell may be treated with an exogenous molecule to soften cytoskeleton of the parent cell. For example, the parent cell can be treated with cytochalasin to soften the cortical actin cytoskeleton. In some embodiments, the nucleus is physically extracted from the cell body by centrifugation to generate an enucleated cell. In some embodiments, the centrifugation comprises use of density gradients, where the enucleated cells are isolated at least because the enucleate cells and intact nucleated cells sediment to different layers in the density gradient. In some embodiments, the centrifugation comprises continuous-flow centrifugation. Example 3 illustrates an exemplary continuous flow centrifugation experiment for obtaining enucleated cells from nucleated cells. In some embodiments, the continuous-flow centrifugation is fixed angle centrifugation. In some embodiments, the use of continuous-flow centrifugation increases the volume that can be centrifuged. For example, the use of continuous-flow centrifugation increases a volume that can be centrifuged compared to swinging-bucket centrifugation (for generating a comparable density gradient). In some embodiments, the centrifugation comprises zonal centrifugation, where the enucleated cell is separated from the nucleated cell based on a difference in size, difference in mass, or a combination thereof. Example 2 illustrates generating enucleated cells by zonal centrifugation. In some embodiments, the method comprises inducing cell death of the nucleated cells after the centrifugation or enucleation. For example, the nucleated cells can be engineered to possess a heterologous polynucleotide encoding a heterologous gene product described herein, where expression of the heterologous gene product induces cell death of at least one nucleated cell.

In some embodiments, methods disclosed herein result in a composition comprising tens of millions of enucleated cells (“enucleated cell fraction”). In some embodiments, the composition also includes residual nucleated cells (“nucleated cell fraction”). In some embodiments, the composition is further processed to purify the enucleated cell fraction from the nucleated cell fraction. In some embodiments, the enucleated cell fraction is formulated in a pharmaceutical composition comprising a pharmaceutically acceptable: carrier, excipient, or diluent.

In some embodiments, methods of producing the enucleated cell do not consist or comprise of differentiation of the parent cell. For example, the enucleated cell is not obtained by differentiating a nucleated erythroid progenitor cell into a differentiated and enucleated red blood cell. In some embodiments, the enucleated cell is not a terminally differentiated cell. In some embodiments, the enucleated cell is not a platelet. In some embodiments, the enucleated cell is not obtained from a platelet lineage cell. In some embodiments, the enucleated cell is not a red blood cell. In some embodiments, the enucleated cell is not obtained from a red blood cell lineage cell.

In some embodiments, the parent cell containing a nucleus is engineered to express at least one of therapeutic agent, transmembrane moiety, immune-evading moiety, or targeting moiety described herein; and subsequently, the nucleus of the parent cell is removed. In some embodiments, the parent cell containing the nucleus is enucleated, and the enucleated cell is engineered to express therapeutic agent, transmembrane moiety, immune-evading moiety, or targeting moiety described herein. In some embodiments, the parent cell is engineered to express one or more of the biomolecules above (e.g., immune-evading moiety and/or targeting moiety), and the resulting enucleated cell (e.g., already expressing the immune-evading moiety and/or targeting moiety) is further engineered to express a second of the biomolecules above (e.g., a therapeutic agent). In this manner, the enucleated cells of the present disclosure can be extensively engineered prior to enucleation, stored for long periods of time as needed (through for e.g., lyophilization, cryohibernation, cryopreservation), and quickly engineered to express a therapeutic agent closer to the time of need.

In some embodiments, the composition has the volume comprising more than or equal to about 10 milliliters (mL) to about 10,000 mL. In some embodiments, the composition has the volume comprising more than or equal to about 10 mL to about 100 mL, about 10 mL to about 1,000 mL, about 10 mL to about 2,000 mL, about 10 mL to about 3,000 mL, about 10 mL to about 4,000 mL, about 10 mL to about 5,000 mL, about 10 mL to about 6,000 mL, about 10 mL to about 7,000 mL, about 10 mL to about 8,000 mL, about 10 mL to about 9,000 mL, about 10 mL to about 10,000 mL, about 100 mL to about 1,000 mL, about 100 mL to about 2,000 mL, about 100 mL to about 3,000 mL, about 100 mL to about 4,000 mL, about 100 mL to about 5,000 mL, about 100 mL to about 6,000 mL, about 100 mL to about 7,000 mL, about 100 mL to about 8,000 mL, about 100 mL to about 9,000 mL, about 100 mL to about 10,000 mL, about 1,000 mL to about 2,000 mL, about 1,000 mL to about 3,000 mL, about 1,000 mL to about 4,000 mL, about 1,000 mL to about 5,000 mL, about 1,000 mL to about 6,000 mL, about 1,000 mL to about 7,000 mL, about 1,000 mL to about 8,000 mL, about 1,000 mL to about 9,000 mL, about 1,000 mL to about 10,000 mL, about 2,000 mL to about 3,000 mL, about 2,000 mL to about 4,000 mL, about 2,000 mL to about 5,000 mL, about 2,000 mL to about 6,000 mL, about 2,000 mL to about 7,000 mL, about 2,000 mL to about 8,000 mL, about 2,000 mL to about 9,000 mL, about 2,000 mL to about 10,000 mL, about 3,000 mL to about 4,000 mL, about 3,000 mL to about 5,000 mL, about 3,000 mL to about 6,000 mL, about 3,000 mL to about 7,000 mL, about 3,000 mL to about 8,000 mL, about 3,000 mL to about 9,000 mL, about 3,000 mL to about 10,000 mL, about 4,000 mL to about 5,000 mL, about 4,000 mL to about 6,000 mL, about 4,000 mL to about 7,000 mL, about 4,000 mL to about 8,000 mL, about 4,000 mL to about 9,000 mL, about 4,000 mL to about 10,000 mL, about 5,000 mL to about 6,000 mL, about 5,000 mL to about 7,000 mL, about 5,000 mL to about 8,000 mL, about 5,000 mL to about 9,000 mL, about 5,000 mL to about 10,000 mL, about 6,000 mL to about 7,000 mL, about 6,000 mL to about 8,000 mL, about 6,000 mL to about 9,000 mL, about 6,000 mL to about 10,000 mL, about 7,000 mL to about 8,000 mL, about 7,000 mL to about 9,000 mL, about 7,000 mL to about 10,000 mL, about 8,000 mL to about 9,000 mL, about 8,000 mL to about 10,000 mL, or about 9,000 mL to about 10,000 mL. In some embodiments, the composition has the volume comprising more than or equal to about 10 mL, about 100 mL, about 1,000 mL, about 2,000 mL, about 3,000 mL, about 4,000 mL, about 5,000 mL, about 6,000 mL, about 7,000 mL, about 8,000 mL, about 9,000 mL, or about 10,000 mL. In some embodiments, the composition has the volume comprising more than or equal to at least about 10 mL, about 100 mL, about 1,000 mL, about 2,000 mL, about 3,000 mL, about 4,000 mL, about 5,000 mL, about 6,000 mL, about 7,000 mL, about 8,000 mL, or about 9,000 mL. In some embodiments, the composition has the volume comprising more than or equal to at most about 100 mL, about 1,000 mL, about 2,000 mL, about 3,000 mL, about 4,000 mL, about 5,000 mL, about 6,000 mL, about 7,000 mL, about 8,000 mL, about 9,000 mL, or about 10,000 mL.

In some embodiments, the composition has a volume comprising between about 10 mL to about 10,000 mL. In some embodiments, the composition has a volume comprising between about 10 mL to about 100 mL, about 10 mL to about 1,000 mL, about 10 mL to about 2,000 mL, about 10 mL to about 3,000 mL, about 10 mL to about 4,000 mL, about 10 mL to about 5,000 mL, about 10 mL to about 6,000 mL, about 10 mL to about 7,000 mL, about 10 mL to about 8,000 mL, about 10 mL to about 9,000 mL, about 10 mL to about 10,000 mL, about 100 mL to about 1,000 mL, about 100 mL to about 2,000 mL, about 100 mL to about 3,000 mL, about 100 mL to about 4,000 mL, about 100 mL to about 5,000 mL, about 100 mL to about 6,000 mL, about 100 mL to about 7,000 mL, about 100 mL to about 8,000 mL, about 100 mL to about 9,000 mL, about 100 mL to about 10,000 mL, about 1,000 mL to about 2,000 mL, about 1,000 mL to about 3,000 mL, about 1,000 mL to about 4,000 mL, about 1,000 mL to about 5,000 mL, about 1,000 mL to about 6,000 mL, about 1,000 mL to about 7,000 mL, about 1,000 mL to about 8,000 mL, about 1,000 mL to about 9,000 mL, about 1,000 mL to about 10,000 mL, about 2,000 mL to about 3,000 mL, about 2,000 mL to about 4,000 mL, about 2,000 mL to about 5,000 mL, about 2,000 mL to about 6,000 mL, about 2,000 mL to about 7,000 mL, about 2,000 mL to about 8,000 mL, about 2,000 mL to about 9,000 mL, about 2,000 mL to about 10,000 mL, about 3,000 mL to about 4,000 mL, about 3,000 mL to about 5,000 mL, about 3,000 mL to about 6,000 mL, about 3,000 mL to about 7,000 mL, about 3,000 mL to about 8,000 mL, about 3,000 mL to about 9,000 mL, about 3,000 mL to about 10,000 mL, about 4,000 mL to about 5,000 mL, about 4,000 mL to about 6,000 mL, about 4,000 mL to about 7,000 mL, about 4,000 mL to about 8,000 mL, about 4,000 mL to about 9,000 mL, about 4,000 mL to about 10,000 mL, about 5,000 mL to about 6,000 mL, about 5,000 mL to about 7,000 mL, about 5,000 mL to about 8,000 mL, about 5,000 mL to about 9,000 mL, about 5,000 mL to about 10,000 mL, about 6,000 mL to about 7,000 mL, about 6,000 mL to about 8,000 mL, about 6,000 mL to about 9,000 mL, about 6,000 mL to about 10,000 mL, about 7,000 mL to about 8,000 mL, about 7,000 mL to about 9,000 mL, about 7,000 mL to about 10,000 mL, about 8,000 mL to about 9,000 mL, about 8,000 mL to about 10,000 mL, or about 9,000 mL to about 10,000 mL. In some embodiments, the composition has a volume comprising between about 10 mL, about 100 mL, about 1,000 mL, about 2,000 mL, about 3,000 mL, about 4,000 mL, about 5,000 mL, about 6,000 mL, about 7,000 mL, about 8,000 mL, about 9,000 mL, or about 10,000 mL. In some embodiments, the composition has a volume comprising between at least about 10 mL, about 100 mL, about 1,000 mL, about 2,000 mL, about 3,000 mL, about 4,000 mL, about 5,000 mL, about 6,000 mL, about 7,000 mL, about 8,000 mL, or about 9,000 mL. In some embodiments, the composition has a volume comprising between at most about 100 mL, about 1,000 mL, about 2,000 mL, about 3,000 mL, about 4,000 mL, about 5,000 mL, about 6,000 mL, about 7,000 mL, about 8,000 mL, about 9,000 mL, or about 10,000 mL.

In some embodiments, described herein are methods for cell processing by enucleating a portion of the nucleated cells (parent cells) to produce an enucleated cell fraction using continuous flow centrifugation, where the continuous flow centrifugation is fixed angle centrifugation. In some embodiments, the continuous flow centrifugation is swinging bucket centrifugation.

In some embodiments, the resulting composition comprises an enucleated cell fraction, which may be 100% of the composition. In other embodiments, there may be a nucleated cell fraction of the composition comprised of nucleated parent cells that were not enucleated. In some embodiments, the enucleated cell fraction is greater than or equal and about 30% composition. In some embodiments, the enucleated cell fraction is greater than or equal to about 35% of the composition. In some embodiments, the enucleated cell fraction is greater than or equal to about 40% of the composition. In some embodiments, the enucleated cell fraction is greater than or equal to about 45% of the composition. In some embodiments, the enucleated cell fraction is greater than or equal to about 50% of the composition. In some embodiments, the enucleated cell fraction is greater than or equal to about 55% of the composition. In some embodiments, the enucleated cell fraction is greater than or equal to about 60% of the composition. In some embodiments, the enucleated cell fraction is greater than or equal to about 65% of the composition. In some embodiments, the enucleated cell fraction is greater than or equal to about 70% of the composition. In some embodiments, the enucleated cell fraction is greater than or equal to about 75% of the composition. In some embodiments, the enucleated cell fraction is greater than or equal to about 80% of the composition. In some embodiments, the enucleated cell fraction is greater than or equal to about 85% of the composition. In some embodiments, the enucleated cell fraction is greater than or equal to about 90% of the composition. In some embodiments, the enucleated cell fraction is greater than or equal to about 95% of the composition. In some embodiments, the enucleated cell fraction is greater than or equal to about 96% of the composition. In some embodiments, the enucleated cell fraction is greater than or equal to about 97% of the composition. In some embodiments, the enucleated cell fraction is greater than or equal to about 98% of the composition. In some embodiments, the enucleated cell fraction is greater than or equal to about 99% of the composition.

In some embodiments, cell separation, cell isolation, or cell sorting, is a process to isolate one or more specific cell populations from a heterogeneous mixture of cells. In some embodiments, the methods of enucleating cells disclosed herein is performed on an isolated population of homogenous cells. In some embodiments, the methods of enucleated cells disclosed herein is performed on a heterogeneous mixture of cells. In some embodiments, methods disclosed herein comprise isolating a population of homogenous cells from a mixture of heterogenous cells using a suitable cell separation technique including, but not limited to, immunomagnetic cell separation, fluorescence-activated cell sorting, density gradient centrifugation, immunedensity cell isolation, microfluidic cell sorting, buoyancy-activated cell sorting, aptamer-based cell isolation, complement depletion, or any combination thereof.

In centrifugation, more dense particles can move to the outer edges of the mixture while less dense objects groups together further in as the sample is spun. A biological sample can be centrifuged until the cell types are isolated into layers. During centrifugation, each cell type can sediment to its isopycnic point, which is the place in the medium gradient, where the density of the cells and medium are equal. Examples of density gradient media include Lymphoprep™, Lympholyte®, Ficoll-Paque®, Percoll®, OptiPrep™, Cell Separation with Accuspin™ Aystem-Histopaque® Media, Histopaque® Media, Histopaque® Iodinated Gradient Media, inorganic salts, nonionic iodinated density gradient media, polyhydric alcohols, polysaccharides, etc. For instance, Lymphoprep™, Lympholyte®, and Ficoll-Paque® consists of saccharides and sodium diatrizoate and may be used to isolate mononuclear cells from peripheral blood, cord blood, and bone marrow. Percoll® consists of colloidal silica particles coated with polyvinylpyrrolidone and is widely used to separate cells, organelles, viruses, and other subcellular particles. OptiPrep™ is a medium consisting of iodixanol in water and used to isolate viruses, organelles, macromolecules, and cells.

In some embodiments, disclosed herein are methods for cell processing by enucleating a portion of the nucleated cells to produce an enucleated cell fraction using continuous flow centrifugation. In some embodiments, disclosed herein are methods for cell processing by enucleating a portion of the nucleated cells to produce an enucleated cell fraction using zonal centrifugation. In some embodiments, the continuous flow centrifugation is fixed angle centrifugation. In some embodiments, the continuous flow centrifugation is swinging bucket centrifugation. In some embodiments, the continuous flow centrifugation generates a density gradient. In some embodiments, the density gradient separates the enucleated cell fraction from the nucleated cells in the composition. In some embodiments, the density gradient comprises a polysaccharide density gradient. In some embodiments, the polysaccharide density gradient comprises a Ficoll density gradient. In some embodiments, methods further comprise producing the Ficoll gradient by polymerizing sucrose molecules with epichlorohydrin to give a polysaccharide that is osmotically inert.

In some embodiments, the gradient comprises between 2 ranges to 20 ranges of the density gradient. In some embodiments, the gradient comprises between 2 ranges to 3 ranges, 2 ranges to 4 ranges, 2 ranges to 5 ranges, 2 ranges to 6 ranges, 2 ranges to 8 ranges, 2 ranges to 10 ranges, 2 ranges to 12 ranges, 2 ranges to 14 ranges, 2 ranges to 16 ranges, 2 ranges to 18 ranges, 2 ranges to 20 ranges, 3 ranges to 4 ranges, 3 ranges to 5 ranges, 3 ranges to 6 ranges, 3 ranges to 8 ranges, 3 ranges to 10 ranges, 3 ranges to 12 ranges, 3 ranges to 14 ranges, 3 ranges to 16 ranges, 3 ranges to 18 ranges, 3 ranges to 20 ranges, 4 ranges to 5 ranges, 4 ranges to 6 ranges, 4 ranges to 8 ranges, 4 ranges to 10 ranges, 4 ranges to 12 ranges, 4 ranges to 14 ranges, 4 ranges to 16 ranges, 4 ranges to 18 ranges, 4 ranges to 20 ranges, 5 ranges to 6 ranges, 5 ranges to 8 ranges, 5 ranges to 10 ranges, 5 ranges to 12 ranges, 5 ranges to 14 ranges, 5 ranges to 16 ranges, 5 ranges to 18 ranges, 5 ranges to 20 ranges, 6 ranges to 8 ranges, 6 ranges to 10 ranges, 6 ranges to 12 ranges, 6 ranges to 14 ranges, 6 ranges to 16 ranges, 6 ranges to 18 ranges, 6 ranges to 20 ranges, 8 ranges to 10 ranges, 8 ranges to 12 ranges, 8 ranges to 14 ranges, 8 ranges to 16 ranges, 8 ranges to 18 ranges, 8 ranges to 20 ranges, 10 ranges to 12 ranges, 10 ranges to 14 ranges, 10 ranges to 16 ranges, 10 ranges to 18 ranges, 10 ranges to 20 ranges, 12 ranges to 14 ranges, 12 ranges to 16 ranges, 12 ranges to 18 ranges, 12 ranges to 20 ranges, 14 ranges to 16 ranges, 14 ranges to 18 ranges, 14 ranges to 20 ranges, 16 ranges to 18 ranges, 16 ranges to 20 ranges, or 18 ranges to 20 ranges of the density gradient. In some embodiments, the gradient comprises between 2 ranges, 3 ranges, 4 ranges, 5 ranges, 6 ranges, 8 ranges, 10 ranges, 12 ranges, 14 ranges, 16 ranges, 18 ranges, or 20 ranges. In some embodiments, the gradient comprises between at least 2 ranges, 3 ranges, 4 ranges, 5 ranges, 6 ranges, 8 ranges, 10 ranges, 12 ranges, 14 ranges, 16 ranges, or 18 ranges of the density gradient. In some embodiments, the gradient comprises between at most 3 ranges, 4 ranges, 5 ranges, 6 ranges, 8 ranges, 10 ranges, 12 ranges, 14 ranges, 16 ranges, 18 ranges, or 20 ranges of the density gradient. In some embodiments, the gradient comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty ranges of the density gradient. In some embodiments, the gradient comprises at least seven ranges of the density gradient. In some embodiments, the gradient comprises at least five ranges of the density gradient. In some embodiments, the gradient comprises at least three ranges of the density gradient. In some embodiments, the gradient comprises seven ranges of the density gradient. In some embodiments, the gradient comprises five ranges of the density gradient. In some embodiments, the gradient comprises three ranges of the density gradient.

In some embodiments, the gradient comprises about 7.5% density gradient media to about 30% density gradient media. 7.5% density gradient media to about 10% density gradient media, about 7.5% density gradient media to about 12.5% density gradient media, about 7.5% density gradient media to about 15% density gradient media, about 7.5% density gradient media to about 16% density gradient media, about 7.5% density gradient media to about 17% density gradient media, about 7.5% density gradient media to about 18% density gradient media, about 7.5% density gradient media to about 19% density gradient media, about 7.5% density gradient media to about 20% density gradient media, about 7.5% density gradient media to about 25% density gradient media, about 7.5% density gradient media to about 27.5% density gradient media, about 7.5% density gradient media to about 30% density gradient media, about 10% density gradient media to about 12.5% density gradient media, about 10% density gradient media to about 15% density gradient media, about 10% density gradient media to about 16% density gradient media, about 10% density gradient media to about 17% density gradient media, about 10% density gradient media to about 18% density gradient media, about 10% density gradient media to about 19% density gradient media, about 10% density gradient media to about 20% density gradient media, about 10% density gradient media to about 25% density gradient media, about 10% density gradient media to about 27.5% density gradient media, about 10% density gradient media to about 30% density gradient media, about 12.5% density gradient media to about 15% density gradient media, about 12.5% density gradient media to about 16% density gradient media, about 12.5% density gradient media to about 17% density gradient media, about 12.5% density gradient media to about 18% density gradient media, about 12.5% density gradient media to about 19% density gradient media, about 12.5% density gradient media to about 20% density gradient media, about 12.5% density gradient media to about 25% density gradient media, about 12.5% density gradient media to about 27.5% density gradient media, about 12.5% density gradient media to about 30% density gradient media, about 15% density gradient media to about 16% density gradient media, about 15% density gradient media to about 17% density gradient media, about 15% density gradient media to about 18% density gradient media, about 15% density gradient media to about 19% density gradient media, about 15% density gradient media to about 20% density gradient media, about 15% density gradient media to about 25% density gradient media, about 15% density gradient media to about 27.5% density gradient media, about 15% density gradient media to about 30% density gradient media, about 16% density gradient media to about 17% density gradient media, about 16% density gradient media to about 18% density gradient media, about 16% density gradient media to about 19% density gradient media, about 16% density gradient media to about 20% density gradient media, about 16% density gradient media to about 25% density gradient media, about 16% density gradient media to about 27.5% density gradient media, about 16% density gradient media to about 30% density gradient media, about 17% density gradient media to about 18% density gradient media, about 17% density gradient media to about 19% density gradient media, about 17% density gradient media to about 20% density gradient media, about 17% density gradient media to about 25% density gradient media, about 17% density gradient media to about 27.5% density gradient media, about 17% density gradient media to about 30% density gradient media, about 18% density gradient media to about 19% density gradient media, about 18% density gradient media to about 20% density gradient media, about 18% density gradient media to about 25% density gradient media, about 18% density gradient media to about 27.5% density gradient media, about 18% density gradient media to about 30% density gradient media, about 19% density gradient media to about 20% density gradient media, about 19% density gradient media to about 25% density gradient media, about 19% density gradient media to about 27.5% density gradient media, about 19% density gradient media to about 30% density gradient media, about 20% density gradient media to about 25% density gradient media, about 20% density gradient media to about 27.5% density gradient media, about 20% density gradient media to about 30% density gradient media, about 25% density gradient media to about 27.5% density gradient media, about 25% density gradient media to about 30% density gradient media, or about 27.5% density gradient media to about 30% density gradient media.

In some embodiments, the gradient is a Ficoll gradient. In some embodiments, the Ficoll gradient comprises between 2 ranges to 20 ranges of the density Ficoll gradient. In some embodiments, the Ficoll gradient comprises between 2 ranges to 3 ranges, 2 ranges to 4 ranges, 2 ranges to 5 ranges, 2 ranges to 6 ranges, 2 ranges to 8 ranges, 2 ranges to 10 ranges, 2 ranges to 12 ranges, 2 ranges to 14 ranges, 2 ranges to 16 ranges, 2 ranges to 18 ranges, 2 ranges to 20 ranges, 3 ranges to 4 ranges, 3 ranges to 5 ranges, 3 ranges to 6 ranges, 3 ranges to 8 ranges, 3 ranges to 10 ranges, 3 ranges to 12 ranges, 3 ranges to 14 ranges, 3 ranges to 16 ranges, 3 ranges to 18 ranges, 3 ranges to 20 ranges, 4 ranges to 5 ranges, 4 ranges to 6 ranges, 4 ranges to 8 ranges, 4 ranges to 10 ranges, 4 ranges to 12 ranges, 4 ranges to 14 ranges, 4 ranges to 16 ranges, 4 ranges to 18 ranges, 4 ranges to 20 ranges, 5 ranges to 6 ranges, 5 ranges to 8 ranges, 5 ranges to 10 ranges, 5 ranges to 12 ranges, 5 ranges to 14 ranges, 5 ranges to 16 ranges, 5 ranges to 18 ranges, 5 ranges to 20 ranges, 6 ranges to 8 ranges, 6 ranges to 10 ranges, 6 ranges to 12 ranges, 6 ranges to 14 ranges, 6 ranges to 16 ranges, 6 ranges to 18 ranges, 6 ranges to 20 ranges, 8 ranges to 10 ranges, 8 ranges to 12 ranges, 8 ranges to 14 ranges, 8 ranges to 16 ranges, 8 ranges to 18 ranges, 8 ranges to 20 ranges, 10 ranges to 12 ranges, 10 ranges to 14 ranges, 10 ranges to 16 ranges, 10 ranges to 18 ranges, 10 ranges to 20 ranges, 12 ranges to 14 ranges, 12 ranges to 16 ranges, 12 ranges to 18 ranges, 12 ranges to 20 ranges, 14 ranges to 16 ranges, 14 ranges to 18 ranges, 14 ranges to 20 ranges, 16 ranges to 18 ranges, 16 ranges to 20 ranges, or 18 ranges to 20 ranges of the density Ficoll gradient. In some embodiments, the Ficoll gradient comprises between 2 ranges, 3 ranges, 4 ranges, 5 ranges, 6 ranges, 8 ranges, 10 ranges, 12 ranges, 14 ranges, 16 ranges, 18 ranges, or 20 ranges. In some embodiments, the Ficoll gradient comprises between at least 2 ranges, 3 ranges, 4 ranges, 5 ranges, 6 ranges, 8 ranges, 10 ranges, 12 ranges, 14 ranges, 16 ranges, or 18 ranges of the density Ficoll gradient. In some embodiments, the Ficoll gradient comprises between at most 3 ranges, 4 ranges, 5 ranges, 6 ranges, 8 ranges, 10 ranges, 12 ranges, 14 ranges, 16 ranges, 18 ranges, or 20 ranges of the density Ficoll gradient. In some embodiments, the Ficoll gradient comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty ranges of the density Ficoll gradient. In some embodiments, the Ficoll gradient comprises at least seven ranges of the density Ficoll gradient. In some embodiments, the Ficoll gradient comprises at least five ranges of the density Ficoll gradient. In some embodiments, the Ficoll gradient comprises at least three ranges of the density Ficoll gradient. In some embodiments, the Ficoll gradient comprises seven ranges of the density Ficoll gradient. In some embodiments, the Ficoll gradient comprises five ranges of the density Ficoll gradient. In some embodiments, the Ficoll gradient comprises three ranges of the density Ficoll gradient.

In some embodiments, the Ficoll density gradient comprises about 7.5% Ficoll to about 10% Ficoll, about 7.5% Ficoll to about 12.5% Ficoll, about 7.5% Ficoll to about 15% Ficoll, about 7.5% Ficoll to about 16% Ficoll, about 7.5% Ficoll to about 17% Ficoll, about 7.5% Ficoll to about 18% Ficoll, about 7.5% Ficoll to about 19% Ficoll, about 7.5% Ficoll to about 20% Ficoll, about 7.5% Ficoll to about 25% Ficoll, about 7.5% Ficoll to about 27.5% Ficoll, about 7.5% Ficoll to about 30% Ficoll, about 10% Ficoll to about 12.5% Ficoll, about 10% Ficoll to about 15% Ficoll, about 10% Ficoll to about 16% Ficoll, about 10% Ficoll to about 17% Ficoll, about 10% Ficoll to about 18% Ficoll, about 10% Ficoll to about 19% Ficoll, about 10% Ficoll to about 20% Ficoll, about 10% Ficoll to about 25% Ficoll, about 10% Ficoll to about 27.5% Ficoll, about 10% Ficoll to about 30% Ficoll, about 12.5% Ficoll to about 15% Ficoll, about 12.5% Ficoll to about 16% Ficoll, about 12.5% Ficoll to about 17% Ficoll, about 12.5% Ficoll to about 18% Ficoll, about 12.5% Ficoll to about 19% Ficoll, about 12.5% Ficoll to about 20% Ficoll, about 12.5% Ficoll to about 25% Ficoll, about 12.5% Ficoll to about 27.5% Ficoll, about 12.5% Ficoll to about 30% Ficoll, about 15% Ficoll to about 16% Ficoll, about 15% Ficoll to about 17% Ficoll, about 15% Ficoll to about 18% Ficoll, about 15% Ficoll to about 19% Ficoll, about 15% Ficoll to about 20% Ficoll, about 15% Ficoll to about 25% Ficoll, about 15% Ficoll to about 27.5% Ficoll, about 15% Ficoll to about 30% Ficoll, about 16% Ficoll to about 17% Ficoll, about 16% Ficoll to about 18% Ficoll, about 16% Ficoll to about 19% Ficoll, about 16% Ficoll to about 20% Ficoll, about 16% Ficoll to about 25% Ficoll, about 16% Ficoll to about 27.5% Ficoll, about 16% Ficoll to about 30% Ficoll, about 17% Ficoll to about 18% Ficoll, about 17% Ficoll to about 19% Ficoll, about 17% Ficoll to about 20% Ficoll, about 17% Ficoll to about 25% Ficoll, about 17% Ficoll to about 27.5% Ficoll, about 17% Ficoll to about 30% Ficoll, about 18% Ficoll to about 19% Ficoll, about 18% Ficoll to about 20% Ficoll, about 18% Ficoll to about 25% Ficoll, about 18% Ficoll to about 27.5% Ficoll, about 18% Ficoll to about 30% Ficoll, about 19% Ficoll to about 20% Ficoll, about 19% Ficoll to about 25% Ficoll, about 19% Ficoll to about 27.5% Ficoll, about 19% Ficoll to about 30% Ficoll, about 20% Ficoll to about 25% Ficoll, about 20% Ficoll to about 27.5% Ficoll, about 20% Ficoll to about 30% Ficoll, about 25% Ficoll to about 27.5% Ficoll, about 25% Ficoll to about 30% Ficoll, or about 27.5% Ficoll to about 30% Ficoll.

In some embodiments, the Ficoll density gradient comprises about 7.5% Ficoll, about 10% Ficoll, about 12.5% Ficoll, about 15% Ficoll, about 16% Ficoll, about 17% Ficoll, about 18% Ficoll, about 19% Ficoll, about 20% Ficoll, about 25% Ficoll, about 27.5% Ficoll, or about 30% Ficoll. In some embodiments, the Ficoll density gradient comprises at least about 7.5% Ficoll, about 10% Ficoll, about 12.5% Ficoll, about 15% Ficoll, about 16% Ficoll, about 17% Ficoll, about 18% Ficoll, about 19% Ficoll, about 20% Ficoll, about 25% Ficoll, or about 27.5% Ficoll. In some embodiments, the Ficoll density gradient comprises at most about 10% Ficoll, about 12.5% Ficoll, about 15% Ficoll, about 16% Ficoll, about 17% Ficoll, about 18% Ficoll, about 19% Ficoll, about 20% Ficoll, about 25% Ficoll, about 27.5% Ficoll, or about 30% Ficoll. In some embodiments, the Ficoll density gradient comprises about 25% Ficoll, about 17% Ficoll, about 16% Ficoll, about 15% Ficoll, or about 12.5% Ficoll. In some embodiments, the Ficoll density gradient comprises about 25% Ficoll. In some embodiments, the Ficoll density gradient comprises about 17% Ficoll. In some embodiments, the Ficoll density gradient comprises about 16% Ficoll. In some embodiments, the Ficoll density gradient comprises about 15% Ficoll. In some embodiments, the Ficoll density gradient comprises about 12.5% Ficoll.

In some embodiments, the methods for cell processing disclosed herein include enucleating a portion of the nucleated cells to produce an enucleated cell fraction using continuous flow centrifugation, wherein the portion of the nucleated cells that is enucleated is greater than or equal to about 10% of the nucleated cells. In some embodiment, the portion of the nucleated cells is greater than or equal to about 20% of the nucleated cells. In some embodiment, the portion of the nucleated cells is greater than or equal to about 25% of the nucleated cells. In some embodiment, the portion of the nucleated cells is greater than or equal to about 30% of the nucleated cells. In some embodiment, the portion of the nucleated cells is greater than or equal to about 35% of the nucleated cells. In some embodiment, the portion of the nucleated cells is greater than or equal to about 40% of the nucleated cells. In some embodiment, the portion of the nucleated cells is greater than or equal to about 45% of the nucleated cells. In some embodiment, the portion of the nucleated cells is greater than or equal to about 50% of the nucleated cells. In some embodiment, the portion of the nucleated cells is greater than or equal to about 55% of the nucleated cells. In some embodiment, the portion of the nucleated cells is greater than or equal to about 60% of the nucleated cells. In some embodiment, the portion of the nucleated cells is greater than or equal to about 65% of the nucleated cells. In some embodiment, the portion of the nucleated cells is greater than or equal to about 70% of the nucleated cells. In some embodiment, the portion of the nucleated cells is greater than or equal to about 75% of the nucleated cells. In some embodiment, the portion of the nucleated cells is greater than or equal to about 80% of the nucleated cells. In some embodiment, the portion of the nucleated cells is greater than or equal to about 85% of the nucleated cells. In some embodiment, the portion of the nucleated cells is greater than or equal to about 90% of the nucleated cells. In some embodiment, the portion of the nucleated cells is greater than or equal to about 95% of the nucleated cells.

In some embodiments, the enucleated cell fraction produced by methods disclosed herein comprises more than or equal to about 1×10⁵ enucleated cells, 1×10⁶ enucleated cells, about 1×10⁷ enucleated cells, 3×10⁵ enucleated cells, 5×10⁵ enucleated cells, 7×10⁷ of enucleated cells, 8×10⁷ of enucleated cells, 9×10⁷ of enucleated cells, 10×10⁷ of enucleated cells, 15×10⁷ of enucleated cells, 20×10⁷ of enucleated cells, 50×10⁷ of enucleated cells, 70×10⁷ enucleated cells, 90×10⁷ enucleated cells, 100×10⁷ of enucleated cells, 150×10⁷ of enucleated cells, 200×10⁷ of enucleated cells, 250×10⁷ enucleated cells, 300×10⁷ enucleated cells, or 500×10⁷ of enucleated cells.

In some embodiments, the enucleated cell of the enucleated cell fraction has a diameter comprising less than or equal to about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of an average diameter of the nucleated cells. In some embodiments, the enucleated cell of the enucleated cell fraction has a diameter comprising less than or equal to about 10%, about 20%, about 30%, about 40%, about 50%, about 60%, about 70%, about 80%, about 90%, about 95%, or about 99% of an average diameter of the nucleated cells. In some embodiments, the enucleated cell of the enucleated cell fraction has a diameter comprising less than or equal to about 50% of an average diameter of the nucleated cells. In some embodiments, the enucleated cell of the enucleated cell fraction has a diameter comprising less than or equal to about 60% of an average diameter of the nucleated cells. In some embodiments, the enucleated cell of the enucleated cell fraction has a diameter comprising less than or equal to about 70% of an average diameter of the nucleated cells. In some embodiments, the enucleated cell of the enucleated cell fraction has a diameter comprising less than or equal to about 80% of an average diameter of the nucleated cells. In some embodiments, the enucleated cell of the enucleated cell fraction has a diameter comprising less than or equal to about 90% of an average diameter of the nucleated cells.

In some embodiments, the enucleated cell of the enucleated cell fraction has a diameter comprising more than or equal to about 5 μm, about 10 μm, about 20 μm, about 30 μm, about 40 μm, about 50 μm, about 60 μm, about 70 μm, about 80 μm, or about 90 μm. In some embodiments, the enucleated cell of the enucleated cell fraction has a diameter ranging from about 1 μm to about 10 μm. In some embodiments, the enucleated cell of the enucleated cell fraction has a diameter ranging from about 1 μm to about 2 μm, about 1 μm to about 3 μm, about 1 μm to about 4 μm, about 1 μm to about 5 μm, about 1 μm to about 6 μm, about 1 μm to about 7 μm, about 1 μm to about 8 μm, about 1 μm to about 9 μm, about 1 μm to about 10 μm, about 2 μm to about 3 μm, about 2 μm to about 4 μm, about 2 μm to about 5 μm, about 2 μm to about 6 μm, about 2 μm to about 7 μm, about 2 μm to about 8 μm, about 2 μm to about 9 μm, about 2 μm to about 10 μm, about 3 μm to about 4 μm, about 3 μm to about 5 μm, about 3 μm to about 6 μm, about 3 μm to about 7 μm, about 3 μm to about 8 μm, about 3 μm to about 9 μm, about 3 μm to about 10 μm, about 4 μm to about 5 μm, about 4 μm to about 6 μm, about 4 μm to about 7 μm, about 4 μm to about 8 μm, about 4 μm to about 9 μm, about 4 μm to about 10 μm, about 5 μm to about 6 μm, about 5 μm to about 7 μm, about 5 μm to about 8 μm, about 5 μm to about 9 μm, about 5 μm to about 10 μm, about 6 μm to about 7 μm, about 6 μm to about 8 μm, about 6 μm to about 9 μm, about 6 μm to about 10 μm, about 7 μm to about 8 μm, about 7 μm to about 9 μm, about 7 μm to about 10 μm, about 8 μm to about 9 μm, about 8 μm to about 10 μm, or about 9 μm to about 10 μm. In some embodiments, the enucleated cell of the enucleated cell fraction has a diameter ranging from about 1 μm, about 2 μm, about 3 μm, about 4 μm, about 5 μm, about 6 μm, about 7 μm, about 8 μm, about 9 μm, or about 10 μm. In some embodiments, the enucleated cell of the enucleated cell fraction has a diameter ranging from at least about 1 μm, about 2 μm, about 3 μm, about 4 μm, about 5 μm, about 6 μm, about 7 μm, about 8 μm, or about 9 μm. In some embodiments, the enucleated cell of the enucleated cell fraction has a diameter ranging from at most about 2 μm, about 3 μm, about 4 μm, about 5 μm, about 6 μm, about 7 μm, about 8 μm, about 9 μm, or about 10 μm. In some embodiments, the enucleated cell of the enucleated cell fraction has a diameter of about 8 μm.

In some embodiments, the methods for cell processing further include generating the density gradient by centrifuging a density gradient media with acceleration spanning over at least about 1 minute (min), at least about 5 min, at least about 10 min, at least about 15 min, at least about 20 min, at least about 25 min, at least about 30 min, at least about 35 min, at least about 40 min, at least about 45 min, at least about 50 min, at least about 55 min, at least about 60 min, at least about 90 min, or at least about 120 min. In some embodiments, the methods for cell processing further include generating the density gradient by centrifuging a polysaccharide with acceleration spanning over at least about 1 min, at least about 5 min, at least about 10 min, at least about 15 min, at least about 20 min, at least about 25 min, at least about 30 min, at least about 35 min, at least about 40 min, at least about 45 min, at least about 50 min, at least about 55 min, at least about 60 min, at least about 90 min, or at least about 120 min. In some embodiments, the methods for cell processing further include generating the density gradient by centrifuging a polysaccharide with acceleration spanning over at least about 10 min, at least about 20 min, at least about 30 min, at least about 40 min, or at least about 50 min. In some embodiments, the methods for cell processing further include generating the density gradient by centrifuging a polysaccharide with acceleration spanning over at least about 30 min.

In some embodiments, the methods of enucleating a portion of the nucleated cells to produce an enucleated cell fraction using continuous flow centrifugation for cell processing further include generating the density gradient by centrifuging a polysaccharide with minimal deceleration. In some embodiments, the density gradient comprising centrifuging a polysaccharide at a maximum centrifugal force of between about 20,000 relative centrifugal force (RCF) to about 250,000 RCF. In some embodiments, the density gradient comprising centrifuging a polysaccharide at a maximum centrifugal force of between about 20,000 RCF to about 30,000 RCF, about 20,000 RCF to about 40,000 RCF, about 20,000 RCF to about 50,000 RCF, about 20,000 RCF to about 60,000 RCF, about 20,000 RCF to about 70,000 RCF, about 20,000 RCF to about 80,000 RCF, about 20,000 RCF to about 100,000 RCF, about 20,000 RCF to about 120,000 RCF, about 20,000 RCF to about 150,000 RCF, about 20,000 RCF to about 200,000 RCF, about 20,000 RCF to about 250,000 RCF, about 30,000 RCF to about 40,000 RCF, about 30,000 RCF to about 50,000 RCF, about 30,000 RCF to about 60,000 RCF, about 30,000 RCF to about 70,000 RCF, about 30,000 RCF to about 80,000 RCF, about 30,000 RCF to about 100,000 RCF, about 30,000 RCF to about 120,000 RCF, about 30,000 RCF to about 150,000 RCF, about 30,000 RCF to about 200,000 RCF, about 30,000 RCF to about 250,000 RCF, about 40,000 RCF to about 50,000 RCF, about 40,000 RCF to about 60,000 RCF, about 40,000 RCF to about 70,000 RCF, about 40,000 RCF to about 80,000 RCF, about 40,000 RCF to about 100,000 RCF, about 40,000 RCF to about 120,000 RCF, about 40,000 RCF to about 150,000 RCF, about 40,000 RCF to about 200,000 RCF, about 40,000 RCF to about 250,000 RCF, about 50,000 RCF to about 60,000 RCF, about 50,000 RCF to about 70,000 RCF, about 50,000 RCF to about 80,000 RCF, about 50,000 RCF to about 100,000 RCF, about 50,000 RCF to about 120,000 RCF, about 50,000 RCF to about 150,000 RCF, about 50,000 RCF to about 200,000 RCF, about 50,000 RCF to about 250,000 RCF, about 60,000 RCF to about 70,000 RCF, about 60,000 RCF to about 80,000 RCF, about 60,000 RCF to about 100,000 RCF, about 60,000 RCF to about 120,000 RCF, about 60,000 RCF to about 150,000 RCF, about 60,000 RCF to about 200,000 RCF, about 60,000 RCF to about 250,000 RCF, about 70,000 RCF to about 80,000 RCF, about 70,000 RCF to about 100,000 RCF, about 70,000 RCF to about 120,000 RCF, about 70,000 RCF to about 150,000 RCF, about 70,000 RCF to about 200,000 RCF, about 70,000 RCF to about 250,000 RCF, about 80,000 RCF to about 100,000 RCF, about 80,000 RCF to about 120,000 RCF, about 80,000 RCF to about 150,000 RCF, about 80,000 RCF to about 200,000 RCF, about 80,000 RCF to about 250,000 RCF, about 100,000 RCF to about 120,000 RCF, about 100,000 RCF to about 150,000 RCF, about 100,000 RCF to about 200,000 RCF, about 100,000 RCF to about 250,000 RCF, about 120,000 RCF to about 150,000 RCF, about 120,000 RCF to about 200,000 RCF, about 120,000 RCF to about 250,000 RCF, about 150,000 RCF to about 200,000 RCF, about 150,000 RCF to about 250,000 RCF, or about 200,000 RCF to about 250,000 RCF. In some embodiments, the density gradient comprising centrifuging a polysaccharide at a maximum centrifugal force of between about 20,000 RCF, about 30,000 RCF, about 40,000 RCF, about 50,000 RCF, about 60,000 RCF, about 70,000 RCF, about 80,000 RCF, about 100,000 RCF, about 120,000 RCF, about 150,000 RCF, about 200,000 RCF, or about 250,000 RCF. In some embodiments, the density gradient comprising centrifuging a polysaccharide at a maximum centrifugal force of between about 20,000 RCF, about 30,000 RCF, about 40,000 RCF, about 50,000 RCF, about 60,000 RCF, about 70,000 RCF, about 80,000 RCF, about 100,000 RCF, about 120,000 RCF, about 150,000 RCF, or about 200,000 RCF. In some embodiments, the density gradient comprising centrifuging a polysaccharide at a maximum centrifugal force at most about 30,000 RCF, about 40,000 RCF, about 50,000 RCF, about 60,000 RCF, about 70,000 RCF, about 80,000 RCF, about 100,000 RCF, about 120,000 RCF, about 150,000 RCF, about 200,000 RCF, or about 250,000 RCF.

(a) Modifying and/or Storing Cells of the Disclosure

A nucleated (“parent”) cell may be engineered prior to enucleation to express one or more exogenous agents, or after enucleation, or a combination thereof. In some embodiments, the one or more exogenous biomolecule comprises a targeting moiety, a transmembrane moiety, a biomolecular suicide switch, or a therapeutic agent, or a combination thereof. In some embodiments, the targeting moiety comprises an adhesion molecule, chemokine or retention receptors or both. In some embodiments, the targeting moiety is engineered to target a target tissue, cell or environment disclosed herein (e.g., the lymph tissue in a subject). In addition, or alternatively, the resulting enucleated cell is engineered to express and, in some cases, to secrete the therapeutic agent. In some embodiments, the therapeutic agent comprises an antibody or an antigen-binding fragment thereof (e.g., single-domain antibody). In some embodiments, the enucleated cell may be administered to a subject in need thereof to treat a disease or a condition in the subject.

Various methods may be used to introduce a biomolecule (e.g., the therapeutic agent, transmembrane moiety, immune-evading moiety, and/or targeting moiety described herein) into the parent cell or the enucleated cell described herein. Non-limiting examples of methods that may be used to introduce a biomolecule into the parent cell or the enucleated cell include: liposome mediated transfer, an adenovirus, an adeno-associated virus, a herpes virus, a retroviral based vector, a lentiviral vector, electroporation, microinjection, lipofection, transfection, calcium phosphate transfection, dendrimer-based transfection, cationic polymer transfection, cell squeezing, sonoporation, optical transfection, impalection, hydrodynamic delivery, magnetofection, nanoparticle transfection, or combinations thereof. In some aspects of any of the compositions and methods provided herein, a therapeutic agent, a virus, an antibody, or a nanoparticle may be introduced into the enucleated cells.

In some embodiments, the biomolecule is introduced into the parent cell or the enucleated cell via mRNA transfection. In mRNA transduction, the mRNA can be incubated with a transfection reagent (e.g., lipofectamine). The mRNA-transfection reagent mixture can subsequently be added to parent cell or enucleated cell suspensions or cell cultures. The mRNA-transfection reagent and cell suspensions or cell cultures can then be incubated to allow transfection of the mRNA into the patent cell or the enucleated cell. In some embodiments, the biomolecule is introduced into the parent cell or the enucleated cell via siRNA transfection. In siRNA transduction, the siRNA can be incubated with a transfection reagent (e.g., lipofectamine). The siRNA-transfection reagent mixture can subsequently be added to parent cell or enucleated cell suspensions or cell cultures. The siRNA-transfection reagent and cell suspensions or cell cultures can then be incubated to allow transfection of the siRNA into the patent cell or the enucleated cell. In some embodiments, the biomolecule is introduced into the parent cell via oncolytic virus mediated infection. In oncolytic virus mediated infection, parent cells can be incubated with an oncolytic virus (e.g., adenovirus) to a multiplicity of infection (MOI) to allow for infection of the parent cells. In some embodiments, the oncolytic virus is carrying a therapeutic agent. Subsequent to incubation, the supernatant of the parent cells may be removed to eliminate any free virus remaining in the parent cell culture. In some embodiments, the biomolecule is introduced into the parent cell or the enucleated cell via lentivirus overexpression. In lentivirus overexpression, the parent cells or the enucleated cells can be incubated with a lentivirus (e.g., HIV) at a MOI to allow for infection of the parent cells or the enucleated cells. In some embodiments, the lentivirus is carrying a selection gene (e.g., an antibiotic-resistant gene). Following incubation, the supernatant of the parent cells or the enucleated cells may be removed to eliminate any free virus remaining in the parent cell culture or the enucleated cell culture. The infected parent cell culture or the infected enucleated cell culture can subsequently be cultured with a selection agent (e.g., an antibiotic). The surviving infected parent cells or infected enucleated cells can be isolated for further culturing and expansion. In some embodiments, the biomolecule is introduced into the parent cell or the enucleated cell via peptide loading. In peptide loading, the parent cells or the enucleated cells can be plated on a surface (e.g., a glass slide). In some embodiments, the parent cells or the enucleated cells attached to the surface. The parent cells or the enucleated cells can be incubated with the biomolecule (e.g., peptide) and Arg9. Arg9 can be a cell-permeable peptide that can traverse the membrane of the parent cells or the enucleated cells.

### (i) Cryopreservation Cryohibernation

In some embodiments, the enucleated cell is preserved via cryopreservation. Cryopreservation comprises freezing the enucleated cell, while cryohibernation comprises storing the enucleated cell at a temperature that is below room temperature but without freezing the enucleated cell. In some embodiments, the enucleated cell is preserved via cryohibernation. In some embodiments, the enucleated cell is preserved via cryopreservation, cryohibernation, or lyophilization. Cryopreservation comprises freezing the enucleated cell, while cryohibernation comprises storing the enucleated cell at a temperature that is below room temperature but without freezing the enucleated cell. In some embodiments, the cryohibernation comprises storing the enucleated cells at about 4.C. In some embodiments, the cryohibernation comprises storing the enucleated cells at at most about 4° C. In some embodiments, the cryohibernation comprises storing the enucleated cells for at least about 24 hours, for at least about 48 hours, at least about 72 hours, at least 96 about hours, at least about 5 days, at least about 6 days, at least about 7 days, at least about 10 days, at least about 15 days, at least about one month, at least about one month, or at least about one year. In some embodiments, the cryohibernation comprises contacting or storing the enucleated cells in a media such as a cell culture media. In some embodiments, the cell culture media comprises xeno-free media. In some embodiments, the cell culture media comprises about 5% to about 20% serum.

In some embodiments, the enucleated cell described herein can be cryopreserved. In some embodiments, the cryopreserved enucleated cell, following thawing, the enucleated cell is as viable as an otherwise comparable enucleated cell that is not cryopreserved.

The present disclosure also provides methods for delivering one or more therapeutic agents. In some embodiments, the one or more therapeutic agents is a virus. In some embodiments, the one or more therapeutic agents is a cytokine or a cytokine receptor-binding fragment thereof. In some embodiments, the one or more therapeutic agents is a virus or a cytokine receptor-binding fragment thereof.

In some embodiments, the one or more therapeutic agents are present in a plurality of enucleated cells of the present disclosure. In some embodiments, the plurality of enucleated cells is cryopreserved. In some embodiments, at least a subset of the plurality of cryopreserved enucleated cells comprises the one or more therapeutic agents.

In some embodiments, the method further comprises preparing a fluid formation. In some embodiments, the fluid formulation comprises cryopreserved enucleated cells of the present disclosure. In some embodiments, the fluid formulation further comprises a nonpyrogenic solution.

In some embodiments, the at least the subset of the plurality of cryopreserved enucleated cells with the one or more therapeutic agents is between about 5% to about 100% of the plurality of cryopreserved cells. In some embodiments, the at least the subset of the plurality of cryopreserved enucleated cells with the one or more therapeutic agents is between about 5% to about 10%, between about 10% to about 15%, between about 15% to about 20%, between about 20% to about 25%, between about 25% to about 30%, between about 30% to about 35%, between about 35% to about 40%, between about 40% to about 45%, between about 45% to about 50%, between about 50% to about 55%, between about 55% to about 60%, between about 60% to about 65%, between about 65% to about 70%, between about 70% to about 75%, between about 75% to about 80%, between about 80% to about 85%, between about 85% to about 90%, between about 90% to about 95%, or between about 95% to about 100% of the plurality of cryopreserved enucleated cells.

In some embodiments, the at least the subset of the plurality of cryopreserved enucleated cells with the one or more therapeutic agents is at least about 10%, at least about 15%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100% of the plurality of cryopreserved enucleated cells.

In some embodiments, the at least the subset of the plurality of cryopreserved enucleated cells with the one or more therapeutic agents is about 10%, about 15%, about 15%, about 20%, about 25%, about 30%, about 35%, about 40%, about 45%, about 50%, about 55%, about 60%, about 65%, about 70%, about 75%, about 80%, about 85%, about 90%, about 95%, about 98%, about 99%, or about 100% of the plurality of cryopreserved enucleated cells.

In some embodiments, the method further comprises introducing the fluid formulation to a sample. In some embodiments, the sample is a biopsy, a tissue sample, a blood sample, a serum sample, a cell line, or isolated cells. In some embodiments, the sample is a sample of a subject. In some embodiments, the sample is a subject. In some embodiments, the subject has a disease. Non-limiting examples of disease include cancer, autoimmune diseases, fibrotic diseases, and inflammatory diseases.

In some embodiments, the introducing the fluid formation is performed under conditions sufficient to deliver the one or more therapeutics agents. In some embodiments, the conditions sufficient to deliver the one or more therapeutics agents is incubating the fluid formation with a sample. In some embodiments, the fluid formation is incubated with the sample for between about one hour to about 14 days. In some embodiments, the fluid formation is incubated with the sample for between about one hour to about two hours, between about two hours to about three hours, between about three hours to about four hours, between about four hours to about five hours, between about five hours to about six hours, between about six hours to about seven hours, between about seven hours to about eight hours, between about eight hours to about nine hours, between about nine hours to about ten hours, between about ten hours to about 11 hours, between about 11 hours to about 12 hours, between about 12 hours to about 15 hours, between about 15 hours to about 18 hours, between about 18 hours to about 21 hours, between about 21 hours to about 24 hours, between about 24 hours to about 36 hours, between about 36 hours to about 48 hours, between about 48 hours to about 60 hours, between about 60 hours to about 72 hours, between about 72 hours to about 84 hours, between about 84 hours to about 96 hours, between about 96 hours to between about 108 hours, between about 108 hours to about 120 hours, between about 120 hours to about 132 hours, between about 132 hours to about 144 hours, between about 144 hours to about 156 hours, between about 156 hours to about 168 hours, between about 7 days to about 8 days, between about 8 days to about 9 days, between about 9 days to about 10 days, between about 10 days to about 11 days, between about 11 days to about 12 days, between about 12 days to about 13 days, or between about 13 days to about 14 days.

In some embodiments, the fluid formation is incubated with the sample for at least about one hour, at least about two hours, at least about three hours, at least about four hours, at least about five hours, at least about six hours, at least about seven hours, at least about eight hours, at least about nine hours, at least about ten hours, at least about 11 hours, at least about 12 hours, at least about 15 hours, at least about 18 hours, at least about 21 hours, at least about 24 hours, at least about 36 hours, at least about 48 hours, at least about 60 hours, at least about 72 hours, at least about 84 hours, at least about 96 hours, at least about 108 hours, at least about 120 hours, at least about 132 hours, at least about 144 hours, at least about 156 hours, at least about 168 hours, at least about 8 days, at least about 9 days, at least about 10 days, at least about 11 days, at least about 12 days, at least about 13 days, at least about 14 days, or more.

In some embodiments, the fluid formation is incubated with the sample for at most about 14 days, at most about 13 days, at most about 12 days, at most about 11 days, at most about 10 days, at most about 9 days, at most about 8 days, at most about 168 hours, at most about 156 hours, at most about 144 hours, at most about 132 hours, at most about 120 hours, at most about 108 hours, at most about 96 hours, at most about 84 hours, at most about 72 hours, at most about 60 hours, at most about 48 hours, at most about 36 hours, at most about 24 hours, at most about 21 hours, at most about 18 hours, at most about 15 hours, at most about 12 hours, at most about 11 hours, at most about 10 hours, at most about 9 hours, at most about 8 hours, at most about 7 hours, at most about 6 hours, at most about 5 hours, at most about 4 hours, at most about 3 hours, at most about 2 hours, at most about one hour, or less.

In some embodiments, the fluid formation is incubated with the sample for about one hour, about two hours, about three hours, about four hours, about five hours, about six hours, about seven hours, about eight hours, about nine hours, about ten hours, about 11 hours, about 12 hours, about 15 hours, about 18 hours, about 21 hours, about 24 hours, about 36 hours, about 48 hours, about 60 hours, about 72 hours, about 84 hours, about 96 hours, about 108 hours, about 120 hours, about 132 hours, about 144 hours, about 156 hours, about 168 hours, about 8 days, about 9 days, about 10 days, about 11 days, about 12 days, about 13 days, or about 14 days.

In some embodiments, the conditions sufficient to deliver the one or more therapeutics agents is administering the fluid formation to a patient.

In some embodiments, the one or more therapeutic agents are delivered to the sample in vitro. In some embodiments, the one or more therapeutic agents are delivered to the sample in vivo. In some embodiments, the one or more therapeutics agents are delivered to the sample ex vivo.

In some embodiments, the cryopreserved enucleated cells deliver one or more therapeutic agent to a sample. In some embodiments, the sample is a target cell. In some embodiments, the sample is a subject.

In some embodiments, the cryopreserved enucleated cell delivers one or more therapeutic agents to a sample in an amount that is greater than or equal to about an amount of the one or more therapeutic agents delivered to an otherwise identical sample by otherwise identical enucleated cells that were not cryopreserved. In some embodiments, the cryopreserved enucleated cell delivers between about 1-fold greater to about 50-fold greater than the amount of the one or more therapeutic agents compared to the otherwise comparable enucleated cell. In some embodiments, the cryopreserved enucleated cell delivers between about 1-fold greater to about 2 fold greater, between about 2-fold greater to about 3-fold greater, between about 3-fold greater to about 4-fold greater, between about 4-fold greater to about 5-fold greater, between about 5-fold between to about 10-fold greater, between about 10-fold greater to about 15-fold greater, between about 15-fold greater to about 20 fold greater, between about 20-fold greater to about 25-fold greater, between about 25-fold greater to about 30-fold greater, between about 30-fold greater to about 35-fold greater, between about 35-fold greater to about 40-fold greater, between about 40-fold greater to about 45-fold greater, or between about 45-fold greater to about 50-fold greater than the amount of the one or more therapeutic agents that is delivered by the otherwise identical enucleated cell. In some embodiments, the cryopreserved enucleated cell delivers one or more therapeutic agents in an amount that is about one-fold greater, about two-fold greater, about three-fold greater, about four-fold greater, about five-fold greater, about 10-fold greater, about 15-fold greater, about 20-fold greater, about 25-fold greater, about 30-fold greater, about 35-fold greater, about 40-fold greater, about 45-fold greater, about 50-fold greater, or more compared to the amount of the one or more therapeutic agents that is delivered by the otherwise identical enucleated cell. In some embodiments, the amount of the one or more therapeutic agents that is delivered by the cryopreserved enucleated cell is equal to the amount of the one or more therapeutic agents that is delivered by the otherwise identical enucleated cell.

In some embodiments, the amount of the one or more therapeutic agents delivered is measured in vitro. In some embodiments, the amount of the one or more therapeutic agents delivered is measured in vivo. In some embodiments, the amount of the one or more therapeutic agents delivered is measured ex vivo.

In some embodiments, the one or more therapeutic agents is a virus. In some embodiments, the amount of the one or more therapeutic agents delivered is a measurement of viral titers in a target cell. In some embodiments, the viral titer is measured in plaque-forming units (PFU). In some embodiments, the PFU is measured as PFU per milliliter (mL). In some embodiments, the PFU is measured as PFU is measured as PFU per gram.

In some embodiments, the target cell is a biological sample. Non-limiting examples of biological samples include isolated cells, cellular supernatant, tissue biopsy, tumor biopsy, cell lines, cell cultures, and biological fluids (e.g., saliva, blood, plasma, serum, urine, feces, lymphatic fluid, cerebrospinal fluid).

In some embodiments, the cryopreserved enucleated cell delivers the virus to a target cell in an amount that is greater than or equal to about an amount of the virus delivered to an otherwise identical target cell by otherwise identical enucleated cells that were not cryopreserved. In some embodiments, the cryopreserved enucleated cell delivers the virus to a target cell in an amount that is greater than about an amount of the virus delivered to an otherwise identical target cell by otherwise identical enucleated cells that were not cryopreserved. In some embodiments, the cryopreserved enucleated cell delivers to the target cell between about 1-fold greater to about 50-fold greater than the amount of the virus delivered to the otherwise identical target cell by the otherwise comparable enucleated cell. In some embodiments, the cryopreserved enucleated cell delivers to the target cell between about 1-fold greater to about 2 fold greater, between about 2-fold greater to about 3-fold greater, between about 3-fold greater to about 4-fold greater, between about 4-fold greater to about 5-fold greater, between about 5-fold between to about 10-fold greater, between about 10-fold greater to about 15-fold greater, between about 15-fold greater to about 20 fold greater, between about 20-fold greater to about 25-fold greater, between about 25-fold greater to about 30-fold greater, between about 30-fold greater to about 35-fold greater, between about 35-fold greater to about 40-fold greater, between about 40-fold greater to about 45-fold greater, or between about 45-fold greater to about 50-fold greater than the amount of the virus that is delivered to the otherwise identical target cell by the otherwise identical enucleated cell. In some embodiments, the cryopreserved enucleated cell delivers the virus to the target cell in an amount that is about one-fold greater, about two-fold greater, about three-fold greater, about four-fold greater, about five-fold greater, about 10-fold greater, about 15-fold greater, about 20-fold greater, about 25-fold greater, about 30-fold greater, about 35-fold greater, about 40-fold greater, about 45-fold greater, about 50-fold greater, or more compared to the amount of the virus that is delivered to the otherwise identical target cell by the otherwise identical enucleated cell. In some embodiments, the amount of the virus that is delivered by the cryopreserved enucleated cell to the target cell is equal to the amount of the virus that is delivered to the otherwise identical target cell by the otherwise identical enucleated cell.

In some embodiments, the one or more therapeutic agents is a cytokine or a cytokine receptor-binding fragment thereof. In some embodiments, the amount of the cytokine or a cytokine receptor-binding fragment thereof delivered is measured by the amount of soluble cytokine or a cytokine receptor-binding fragment thereof present. In some embodiments, the amount of the cytokine or a cytokine receptor-binding fragment thereof delivered is measured in micrograms (μg) per mL. In some embodiments, the amount of the cytokine or a cytokine receptor-binding fragment thereof delivered is measured in nanograms (ng) per mL. In some embodiments, the amount of the cytokine or a cytokine receptor-binding fragment thereof delivered is measured in picograms (pg) per mL. In some embodiments, the amount of the cytokine or a cytokine receptor-binding fragment thereof delivered is measured in the supernatant of the cryopreserved enucleated cells. In some embodiments, the amount of the cytokine or a cytokine receptor-binding fragment thereof delivered is measured in the supernatant of the otherwise identical enucleated cells. In some embodiments, the amount of the cytokine or a cytokine receptor-binding fragment thereof delivered is measured in the supernatant of the cryopreserved enucleated cells and the supernatant of the otherwise identical enucleated cells.

In some embodiments, the cryopreserved nucleated cell delivers a greater amount of the cytokine or cytokine receptor-binding fragment thereof compared to the otherwise identical enucleated cells. In some embodiments, the cryopreserved enucleated cell delivers between about 1-fold greater to about 50-fold greater than the amount of the cytokine or a cytokine receptor-binding fragment thereof delivered by the otherwise comparable enucleated cell. In some embodiments, the cryopreserved enucleated cell delivers between about 1-fold greater to about 2 fold greater, between about 2-fold greater to about 3-fold greater, between about 3-fold greater to about 4-fold greater, between about 4-fold greater to about 5-fold greater, between about 5-fold between to about 10-fold greater, between about 10-fold greater to about 15-fold greater, between about 15-fold greater to about 20 fold greater, between about 20-fold greater to about 25-fold greater, between about 25-fold greater to about 30-fold greater, between about 30-fold greater to about 35-fold greater, between about 35-fold greater to about 40-fold greater, between about 40-fold greater to about 45-fold greater, or between about 45-fold greater to about 50-fold greater than the amount of the cytokine or a cytokine receptor-binding fragment thereof that is delivered by the otherwise identical enucleated cell. In some embodiments, the cryopreserved enucleated cell delivers a cytokine or a cytokine receptor-binding fragment thereof in an amount that is about one-fold greater, about two-fold greater, about three-fold greater, about four-fold greater, about five-fold greater, about 10-fold greater, about 15-fold greater, about 20-fold greater, about 25-fold greater, about 30-fold greater, about 35-fold greater, about 40-fold greater, about 45-fold greater, about 50-fold greater, or more compared to the amount of the cytokine or a cytokine receptor-binding fragment thereof that is delivered by the otherwise identical enucleated cell. In some embodiments, the amount of cytokine or a cytokine receptor-binding fragment thereof that is delivered by the cryopreserved enucleated cell is equal to the amount of the cytokine or a cytokine receptor-binding fragment thereof that is delivered by the otherwise identical enucleated cell.

In some embodiments, the amount of the cytokine or a cytokine receptor-binding fragment thereof delivered is measured in a target cell introduced to the cryopreserved enucleated cells. In some embodiments, the amount of the cytokine or a cytokine receptor-binding fragment thereof delivered is measured in an otherwise identical target cell introduced to the otherwise identical enucleated cells. In some embodiments, the amount of the cytokine or a cytokine receptor-binding fragment thereof delivered is measured in target cells introduced to the cryopreserved nucleated cells and in otherwise identical target cells introduced to the otherwise identical enucleated cells. In some embodiments, the amount of the cytokine or a cytokine receptor-binding fragment thereof is measured as the amount of cytokine or a cytokine receptor-binding fragment thereof mRNA. In some embodiments, the amount of the cytokine or a cytokine receptor-binding fragment thereof delivered is measured by the amount of the cytokine or a cytokine receptor-binding fragment thereof mRNA present in a target cell introduced to cryopreserved enucleated cells. In some embodiments, the amount of the cytokine or a cytokine receptor-binding fragment thereof delivered is measured by the amount of the cytokine or a cytokine receptor-binding fragment thereof mRNA present in an otherwise identical target cell introduced to the otherwise identical enucleated cells. In some embodiments, the amount of the cytokine or a cytokine receptor-binding fragment thereof delivered is measured by the amount of the cytokine or a cytokine receptor-binding fragment thereof mRNA present in a target cell introduced to cryopreserved enucleated cells and the amount of the cytokine or a cytokine receptor-binding fragment thereof mRNA present in an otherwise identical target cell introduced to the otherwise identical enucleated cells.

In some embodiments, the target cell introduced to the cryopreserved enucleated cells has a greater amount of the cytokine or cytokine receptor-binding fragment thereof mRNA compared to an otherwise identical target cell introduced to the otherwise identical enucleated cells. In some embodiments, the target cell introduced to the cryopreserved enucleated cell has between about 1-fold greater to about 50-fold greater than the amount of the cytokine or a cytokine receptor-binding fragment thereof mRNA compared to the otherwise identical target cells introduced to the otherwise comparable enucleated cell. In some embodiments, the target cell introduced to the cryopreserved enucleated cell has between about 1-fold greater to about 2 fold greater, between about 2-fold greater to about 3-fold greater, between about 3-fold greater to about 4-fold greater, between about 4-fold greater to about 5-fold greater, between about 5-fold between to about 10-fold greater, between about 10-fold greater to about 15-fold greater, between about 15-fold greater to about 20 fold greater, between about 20-fold greater to about 25-fold greater, between about 25-fold greater to about 30-fold greater, between about 30-fold greater to about 35-fold greater, between about 35-fold greater to about 40-fold greater, between about 40-fold greater to about 45-fold greater, or between about 45-fold greater to about 50-fold greater than the amount of the cytokine or a cytokine receptor-binding fragment thereof mRNA that is present in an otherwise identical target cell introduced to the otherwise identical enucleated cell. In some embodiments, the target cell introduced to the cryopreserved enucleated cell about one-fold greater, about two-fold greater, about three-fold greater, about four-fold greater, about five-fold greater, about 10-fold greater, about 15-fold greater, about 20-fold greater, about 25-fold greater, about 30-fold greater, about 35-fold greater, about 40-fold greater, about 45-fold greater, about 50-fold greater, or more the amount of the cytokine or cytokine receptor-binding fragment thereof mRNA compared to the otherwise identical target cell introduced to otherwise identical enucleated cell. In some embodiments, the amount of cytokine or a cytokine receptor-binding fragment thereof mRNA present in the target cell introduced to the cryopreserved enucleated cell is equal to the amount of the cytokine or a cytokine receptor-binding fragment thereof mRNA present in the otherwise identical target cell introduced to the otherwise identical enucleated cell.

In some embodiments, the amount of the cytokine or cytokine receptor-binding fragment thereof delivered is measured through release of an another cytokine or cytokine receptor-binding fragment thereof by a target cell introduced to the cryopreserved enucleated cells. In some embodiments, the another cytokine or cytokine receptor-binding fragment thereof is a soluble cytokine or cytokine receptor-binding fragment thereof. In some embodiments, the another cytokine or cytokine receptor-binding fragment thereof is any cytokine or cytokine receptor-binding fragment thereof of the present disclosure. In some embodiments, the another cytokine or cytokine receptor-binding fragment thereof is IFN-γ. In some embodiments, the another cytokine or cytokine receptor-binding fragment thereof is TNF-α. In some embodiments, the another cytokine or cytokine receptor-binding fragment thereof is IL-6.

In some embodiments, the amount of the cytokine or a cytokine receptor-binding fragment thereof delivered is measured in an otherwise identical target cell introduced to the otherwise identical enucleated cells. In some embodiments, the amount of the cytokine or a cytokine receptor-binding fragment thereof delivered is measured in target cells introduced to the cryopreserved nucleated cells and in otherwise identical target cells introduced to the otherwise identical enucleated cells. In some embodiments, the amount of the cytokine or a cytokine receptor-binding fragment thereof is measured as the amount of cytokine or a cytokine receptor-binding fragment thereof mRNA. In some embodiments, the amount of the cytokine or a cytokine receptor-binding fragment thereof delivered is measured by the amount of the cytokine or a cytokine receptor-binding fragment thereof mRNA present in a target cell introduced to cryopreserved enucleated cells. In some embodiments, the amount of the cytokine or a cytokine receptor-binding fragment thereof delivered is measured by the amount of the cytokine or a cytokine receptor-binding fragment thereof mRNA present in an otherwise identical target cell introduced to the otherwise identical enucleated cells. In some embodiments, the amount of the cytokine or a cytokine receptor-binding fragment thereof delivered is measured by the amount of the cytokine or a cytokine receptor-binding fragment thereof mRNA present in a target cell introduced to cryopreserved enucleated cells and the amount of the cytokine or a cytokine receptor-binding fragment thereof mRNA present in an otherwise identical target cell introduced to the otherwise identical enucleated cells.

In some embodiments, the target cell introduced to the cryopreserved enucleated cells secretes a greater amount of the another cytokine or cytokine receptor-binding fragment thereof compared to an otherwise identical target cell introduced to the otherwise identical enucleated cells. In some embodiments, the target cell introduced to the cryopreserved enucleated cell secretes between about 1-fold greater to about 50-fold greater than the amount of the another cytokine or a cytokine receptor-binding fragment thereof compared to the otherwise identical target cells introduced to the otherwise comparable enucleated cell. In some embodiments, the target cell introduced to the cryopreserved enucleated cell secretes between about 1-fold greater to about 2 fold greater, between about 2-fold greater to about 3-fold greater, between about 3-fold greater to about 4-fold greater, between about 4-fold greater to about 5-fold greater, between about 5-fold between to about 10-fold greater, between about 10-fold greater to about 15-fold greater, between about 15-fold greater to about 20 fold greater, between about 20-fold greater to about 25-fold greater, between about 25-fold greater to about 30-fold greater, between about 30-fold greater to about 35-fold greater, between about 35-fold greater to about 40-fold greater, between about 40-fold greater to about 45-fold greater, or between about 45-fold greater to about 50-fold greater than the amount of the another cytokine or a cytokine receptor-binding fragment thereof that is secreted in an otherwise identical target cell introduced to the otherwise identical enucleated cell. In some embodiments, the target cell introduced to the cryopreserved enucleated cell secretes about one-fold greater, about two-fold greater, about three-fold greater, about four-fold greater, about five-fold greater, about 10-fold greater, about 15-fold greater, about 20-fold greater, about 25-fold greater, about 30-fold greater, about 35-fold greater, about 40-fold greater, about 45-fold greater, about 50-fold greater, or more of the amount of the another cytokine or cytokine receptor-binding fragment thereof compared to the otherwise identical target cell introduced to otherwise identical enucleated cell. In some embodiments, the amount of the another cytokine or a cytokine receptor-binding fragment thereof secreted in the target cell introduced to the cryopreserved enucleated cell is equal to the amount of the another cytokine or a cytokine receptor-binding fragment thereof secreted by the otherwise identical target cell introduced to the otherwise identical enucleated cell.

In some embodiments, the amount of the cytokine or a cytokine receptor-binding fragment thereof is measured as the amount of the another cytokine or a cytokine receptor-binding fragment thereof mRNA present in the target cell introduced to the cryopreserved enucleated cells. In some embodiments, the amount of the cytokine or cytokine receptor-binding fragment thereof is measured as the amount of the another cytokine or a cytokine receptor-binding fragment thereof mRNA present in the otherwise identical target cell introduced to the otherwise identical enucleated cells. In some embodiments, the amount of the cytokine or a cytokine receptor-binding fragment thereof mRNA is measured as the amount of the another cytokine or cytokine receptor-binding fragment thereof mRNA present in the target cell introduced to the cryopreserved enucleated cells and the amount of the another cytokine or a cytokine receptor-binding fragment thereof mRNA present in the otherwise identical target cell introduced to the otherwise identical enucleated cells.

In some embodiments, the target cell introduced to the cryopreserved enucleated cells has a great amount of the another cytokine or cytokine receptor-binding fragment thereof mRNA compared to an otherwise identical target cell introduced to the otherwise identical enucleated cells. In some embodiments, the target cell introduced to the cryopreserved enucleated cell has between about 1-fold greater to about 50-fold greater than the amount of the another cytokine or a cytokine receptor-binding fragment thereof mRNA compared to the otherwise identical target cells introduced to the otherwise comparable enucleated cell. In some embodiments, the target cell introduced to the cryopreserved enucleated cell has between about 1-fold greater to about 2 fold greater, between about 2-fold greater to about 3-fold greater, between about 3-fold greater to about 4-fold greater, between about 4-fold greater to about 5-fold greater, between about 5-fold between to about 10-fold greater, between about 10-fold greater to about 15-fold greater, between about 15-fold greater to about 20 fold greater, between about 20-fold greater to about 25-fold greater, between about 25-fold greater to about 30-fold greater, between about 30-fold greater to about 35-fold greater, between about 35-fold greater to about 40-fold greater, between about 40-fold greater to about 45-fold greater, or between about 45-fold greater to about 50-fold greater than the amount of the another cytokine or a cytokine receptor-binding fragment thereof mRNA that is present in an otherwise identical target cell introduced to the otherwise identical enucleated cell. In some embodiments, the target cell introduced to the cryopreserved enucleated cell about one-fold greater, about two-fold greater, about three-fold greater, about four-fold greater, about five-fold greater, about 10-fold greater, about 15-fold greater, about 20-fold greater, about 25-fold greater, about 30-fold greater, about 35-fold greater, about 40-fold greater, about 45-fold greater, about 50-fold greater, or more the amount of the another cytokine or cytokine receptor-binding fragment thereof mRNA compared to the otherwise identical target cell introduced to otherwise identical enucleated cell. In some embodiments, the amount of another cytokine or a cytokine receptor-binding fragment thereof mRNA present in the target cell introduced to the cryopreserved enucleated cell is equal to the amount of another cytokine or a cytokine receptor-binding fragment thereof mRNA present in the otherwise identical target cell introduced to the otherwise identical enucleated cell.

In some embodiments, prior to the preparing the fluid formation, storing the cryopreserved composition. In some embodiments, the cryopreserved composition is stored for between about 24 hours to about 5 years. In some embodiments, the cryopreserved composition is stored for between about 12 hours to about 24 hours, between about 24 hours to about 48 hours, between about 48 hours to about 72 hours, between about 72 hours to about 96 hours, between about 96 hours to about 120 hours, between about 120 hours to about 144 hours, between about 144 hours to about 168 hours, between seven days to about 14 days, between about 14 days to about 21 days, between about 21 days to about 28 days, between about 28 days to about 30 days, between about 30 days to about 31 days, between about one month to about two months, between about two months to about three months, between about three months to about four months, between about four months to about five months, between about five months to about six months between about six months to about seven months, between about seven months to about eight months, between about eight months to about nine months, between about nine months to about ten months, between about ten months to about 11 months, between about 11 months to about 12 months, between about one year to about 1.5 years, between about 1.5 years to about 2 years, between about 2 years to about 2.5 years, between about 2.5 years to about 4 years, between about 4 years to about 4.5 years, between about 4.5 years to about 5 years, or more.

In some embodiments, the cryopreserved composition is stored for at least about 12 hours, at least about 24 hours, at least about 36 hours, at least about 48 hours, at least about 60 hours, at least about 72 hours, at least about 84 hours, at least about 96 hours, at least about 108 hours, at least about 120 hours, at least about 132 hours, at least about 144 hours, at least about 156 hours, at least about 168 hours, at least about 14 days, at least about 21 days, at least about 28 hours, at least about 30 days, at least about 31 days, at least about two months, at least about three months, at least about four months, at least about five months, at least about six months, at least about seven months, at least about eight months, at least about nine months, at least about ten months, at least about 11 months, at least about 12 months, at least about 1.5 years, at least about 2 years, at least about 2.5 years, at least about 3 years, at least about 3.5 years, at least about 4 years, at least about 4.5 years, at least about 5 years, or more. In some embodiments, the enucleated cells are cryopreserved for at least about 24 hours. In some embodiments, the enucleated cells are cryopreserved for at least about 7 days. In some embodiments, the enucleated cells are cryopreserved for at least about one month. In some embodiments, the enucleated cells are cryopreserved for at least about one year.

In some embodiments, the cryopreserved composition is stored for about 12 hours, about 24 hours, about 36 hours, about 48 hours, about 60 hours, about 72 hours, about 84 hours, about 96 hours, about 108 hours, about 120 hours, about 132 hours, about 144 hours, about 156 hours, about 168 hours, about 14 days, about 21 days, about 28 hours, about 30 days, about 31 days, about two months, about three months, about four months, about five months, about six months, about seven months, about eight months, about nine months, about ten months, about 11 months, about 12 months, about 1.5 years, about 2 years, about 2.5 years, about 3 years, about 3.5 years, about 4 years, about 4.5 years or about 5 years.

In some embodiments, the cryopreserved composition is stored at temperature below at least about −70° C. In some embodiments, the cryopreserved composition is stored at a temperature between about −70° C. to about −80° C., between about −80° C. to about −90° C., between about −90° C. to about −100° C., between about −100° C. to about −110° C., between about −110° C. to about −120° C., between about −120° C. to about −130° C., between about −130° C. to about −140° C., between about −140° C. to about −150° C., between about −150 to about −160° C., between about −160° C. to about −170° C., between about −170° C. to about −180° C., between about −180° C. to about −190° C., or between −190° C. to about −200° C.

In some embodiments, the cryopreserved composition is stored at a temperature of at least about −70° C., at least about −80° C., at least about −90° C., at least about −100° C., at least about −110° C., at least about −120° C., at least about −130° C., at least about −140° C., at least about −150° C., at least about −160° C., at least about −170° C., at least about −180° C., at least about −190° C., at least about −200° C., or more. In some embodiments, the cryopreserved composition is stored at a temperature of about −70° C., about −80° C., about −90° C., about −100° C., about −110° C., about −120° C., about −130° C., about −140° C., about −150° C., about −160° C., about −170° C., about −180° C., about −190° C., about −200° C., or more.

In some embodiments, the cryopreserved composition is stored in liquid nitrogen. In some embodiments, the temperature of liquid nitrogen is between about −190° C. to about −220° C. In some embodiments, the temperature of liquid nitrogen is between about −190° C. to about −195° C., between about −195° C. to about −200° C., between about −200° C. to about −205° C., between about −205° C. to about −210° C., between about −210° C. to about −215° C., or between about −215° C. to about −220° C. In some embodiments, the temperature of the liquid nitrogen is at least about −190° C., at least about −195° C., at least about −200° C., at least about −205° C., at least about −210° C., at least about −215° C., at least about −220° C., or more. In some embodiments, the temperature of the liquid nitrogen is about −190° C., about −195° C., about −200° C., about −205° C., about −210° C., about −215° C., or about −220° C.

In some embodiments, the cryopreserved composition is stored in dry ice. In some embodiments, the temperature of the dry ice is between about −50° C. to about −90° C. In some embodiments, the temperature of the dry ice is between about −50° C. to about −55° C., between about −55° C. to about −60° C., between about −60° C. to about −65° C., between about −65° C. to about −70° C., between about −70° C. to about −75° C., between about −75° C. to about −80° C., between about −80° C. to about −85° C., or between about −85° C. to about −90° C. In some embodiments, the temperature of the dry ice is at least about −50° C., at least about −55° C., at least about −60° C., at least about −65° C., at least about −70° C., at least about −75° C., at least about −80° C., at least about −85° C., at least about −90° C., or more. In some embodiments, the temperature of the dry ice is about −50° C., about −55° C., about −60° C., about −65° C., about −70° C., about −75° C., about −80° C., about −85° C., or about −90° C.

In some embodiments, the cryopreserved composition is stored in a freezer. In some embodiments, the temperature of the freezer is between about 0° C. to about −90° C. . . . In some embodiments, the temperature of the freezer is between about 0° C. to about −10° C., between about −10° C. to about −20° C., between about −20° C. to about −30° C., between about −30° C. to about −40° C., between about −40° C. to about −50° C., between about −50° C. to about −60° C., between about −60° C. to about −70° C., between about −70° C. to about −80° C., or between about −80° C. to about −90° C. . . . In some embodiments, the temperature of the freezer is at least about 0° C., at least about −5° C., at least about −10° C., at least about −15° C., at least about −20° C., at least about −25° C., at least about −30° C., at least about −35° C., at least about −40° C., at least about −45° C., at least about −50° C., at least about −55° C., at least about −60° C., at least about −65° C., at least about −70° C., at least about −75° C., at least about −80° C., at least about −85° C., at least about −90° C., or more.

In some embodiments, the temperature of the freezer is at most about −90° C., at most about −85° C., at most about −80° C., at most about −75° C., at most about −70° C., at most about −65° C., at most about −60° C., at most about −55° C., at most about −50° C., at most about −45° C., at most about −40° C., at most about −35° C., at most about −30° C., at most about −25° C., at most about −20° C., at most about −15° C., at most about −10° C., at most about −5° C., at most about 0° C., or less.

In some embodiments, the temperature of the freezer is about 0° C., about −5° C., about −10° C., about −15° C., about −20° C., about −25° C., about −30° C., about −35° C., about −40° C., about −45° C., about −50° C., about −55° C., about −60° C., about −65° C., about −70° C., about −75° C., about −80° C., about −85° C., or about −90° C.

In some embodiments, the freezer is set at any temperature in the present disclosure.

In some embodiments, the storing comprises placing the cryopreserved composition at a first temperature. In some embodiments, the storing comprises placing the cryopreserved composition at a second temperature. In some embodiments, the storing comprises placing the cryopreserved composition at the first temperature and placing the cryopreserved composition at the second temperature. In some embodiments, the storing comprising placing the cryopreserved composition at the first temperature and subsequently placing the cryopreserved composition at the second temperature. In some embodiments, the storing comprising placing the cryopreserved composition at the second temperature and subsequently placing the cryopreserved composition at the first temperature.

In some embodiments, the first temperature is between about −20° C. to about −90° C. In some embodiments, the first temperature is between about −20° C. to about −25° C., between about −25° C. to about −30° C., between about −30° C. to about −35° C., between about −35° C. to about −40° C., between about −40° C. to about −45° C., between about −45° C. to about −50° C., between about −50° C. to about −55° C., between about −55° C. to about −60° C., between about −60° C. to about −65° C., between about −65° C. to about −70° C., between about −70° C. to about −75° C., between about −75° C. to about −80° C., between about −80° C. to about −85° C., or between about −85° C. to about −90° C.

In some embodiments, the first temperature is at least about −20° C., at least about −25° C., at least about −30° C., at least about −35° C., at least about −40° C., at least about −45° C., at least about −50° C., at least about −55° C., at least about −60° C., at least about −65° C., at least about −70° C., at least about −75° C., at least about −80° C., at least about −85° C., at least about −90° C., or more. In some embodiments, the first temperature is at most about −90° C., at most about −85° C., at most about −80° C., at most about −75° C., at most about −70° C., at most about −65° C., at most about −60° C., at most about −55° C., at most about −50° C., at most about −45° C., at most about −40° C., at most about −35° C., at most about −30° C., at most about −25° C., at most about −20° C., or less. In some embodiments, the first temperature is about −20° C., about −25° C., about −30° C., about −35° C., about −40° C., about −45° C., about −50° C., about −55° C., about −60° C., about −65° C., about −70° C., about −75° C., about −80° C., about −85° C., or about −90° C.

In some embodiments, the first temperature is about −80° C.

In some embodiments, the cryopreserved composition is placed at the first temperature for between about one hour to about 168 hours. In some embodiments, the cryopreserved composition is placed at the first temperature for between about one hour to about six hours, between about 6 hours to about 12 hours, between about 12 hours to about 18 hours, between about 18 hours to about 24 hours, between about 24 hours to about 36 hours, between about 36 hours to about 48 hours, between about 48 hours to about 60 hours, between about 60 hours to about 72 hours, between about 72 hours to about 84 hours, between about 84 hours to about 96 hours, between about 96 hours to about 108 hours, between about 108 hours to about 120 hours, between about 120 hours to about 132 hours, between about 132 hours to about 144 hours, between about 144 hours to about 156 hours, or between about 156 hours to about 168 hours.

In some embodiments, the cryopreserved composition is placed at the first temperature for at least about one hour, at least about six hours, at least about 12 hours, at least about 18 hours, at least about 24 hours, at least about 36 hours, at least about 48 hours, at least about 60 hours, at least about 72 hours, at least about 84 hours, at least about 96 hours, at least about 108 hours, at least about 120 hours, at least about 132 hours, at least about 144 hours, at least about 168 hours, or more. In some embodiments, the cryopreserved compositions are placed at the first temperature for at most about 168 hours, at most about 156 hours, at most about 144 hours, at most about 132 hours, at most about 120 hours, at most about 108 hours, at most about 96 hours, at most about 84 hours, at most about 72 hours, at most about 60 hours, at most about 48 hours, at most about 36 hours, at most about 24 hours, at most about 12 hours, at most about six hours, at most about one hour, or less. In some embodiments, the cryopreserved composition is placed at the first temperature for about one hour, about six hours, about 12 hours, about 24 hours, about 36 hours, about 48 hours, about 60 hours, about 72 hours, about 84 hours, about 96 hours, about 108 hours, about 120 hours, about 132 hours, about 144 hours, about 156 hours, or about 168 hours. In some embodiments, the cryopreserved composition is placed at the first temperature for about 24 hours. In some embodiments, the cryopreserved composition is placed at the first temperature for about 48 hours. In some embodiments, the cryopreserved composition is placed at the first temperature for about 72 hours.

In some embodiments, the second temperature is between about −90° C. to about −100° C., between about −100° C. to about −120° C., between about −120° C. to about −130° C., between about −130° C. to about −140° C., between about −140° C. to about −150° C., between about −150° C. to about −160° C., between about −160° C. to about −170° C., between about −170° C. to about −180° C., between about −180° C. to about −190° C., between about −190° C. to about −200° C., between about −200° C. to about −210° C., or between about −210° C. to about −220° C.

In some embodiments, the second temperature is at least about −90° C., at least about −100° C., at least about −110° C., at least about −120° C., at least about −130° C., at least about −140° C., at least about −150° C., at least about −160° C., at least about −170° C., at least about −180° C., at least about −190° C., at least about −200° C., at least about −200° C., at least about −210° C., at least about −220° C., or more. In some embodiments, the second temperature is at most about at most about −220° C., at most about −210° C., at most about −200° C., at most about −190° C., at most about −180° C., at most about −170° C., at most about −160° C., at most about −150° C., at most about −140° C., at most about −130° C., at most about −120° C., at most about −110° C., at most about −100° C., at most about −90° C., or less. In some embodiments, the second temperature is about −90° C., about −100° C., about −110° C., about −120° C., about −130° C., about −140° C., about −150° C., about −160° C., about −170° C., about −180° C., about −190° C., about −200° C., about −210° C., or about −220° C. In some embodiments, the second temperature is at least about −220° C.

In some embodiments, the cryopreserved composition is placed at the second temperature for between about 24 hours to about 5 years. In some embodiments, the cryopreserved composition is placed at the second temperature for between about 12 hours to about 24 hours, between about 24 hours to about 48 hours, between about 48 hours to about 72 hours, between about 72 hours to about 96 hours, between about 96 hours to about 120 hours, between about 120 hours to about 144 hours, between about 144 hours to about 168 hours, between seven days to about 14 days, between about 14 days to about 21 days, between about 21 days to about 28 days, between about 28 days to about 30 days, between about 30 days to about 31 days, between about one month to about two months, between about two months to about three months, between about three months to about four months, between about four months to about five months, between about five months to about six months between about six months to about seven months, between about seven months to about eight months, between about eight months to about nine months, between about nine months to about ten months, between about ten months to about 11 months, between about 11 months to about 12 months, between about one year to about 1.5 years, between about 1.5 years to about 2 years, between about 2 years to about 2.5 years, between about 2.5 years to about 4 years, between about 4 years to about 4.5 years, between about 4.5 years to about 5 years, or more.

In some embodiments, the cryopreserved composition is placed at the second temperature for at least about 12 hours, at least about 24 hours, at least about 36 hours, at least about 48 hours, at least about 60 hours, at least about 72 hours, at least about 84 hours, at least about 96 hours, at least about 108 hours, at least about 120 hours, at least about 132 hours, at least about 144 hours, at least about 156 hours, at least about 168 hours, at least about 14 days, at least about 21 days, at least about 28 hours, at least about 30 days, at least about 31 days, at least about two months, at least about three months, at least about four months, at least about five months, at least about six months, at least about seven months, at least about eight months, at least about nine months, at least about ten months, at least about 11 months, at least about 12 months, at least about 1.5 years, at least about 2 years, at least about 2.5 years, at least about 3 years, at least about 3.5 years, at least about 4 years, at least about 4.5 years, at least about 5 years, or more. In some embodiments, the cryopreserved composition is placed at the second temperature for at least about 24 hours. In some embodiments, the cryopreserved composition is placed at the second temperature for at least about 7 days. In some embodiments, the cryopreserved composition is placed at the second temperature for at least about one month. In some embodiments, the cryopreserved composition is placed at the second temperature for at least about one year.

In some embodiments, the cryopreserved composition is placed at the second temperature for about 12 hours, about 24 hours, about 36 hours, about 48 hours, about 60 hours, about 72 hours, about 84 hours, about 96 hours, about 108 hours, about 120 hours, about 132 hours, about 144 hours, about 156 hours, about 168 hours, about 14 days, about 21 days, about 28 hours, about 30 days, about 31 days, about two months, about three months, about four months, about five months, about six months, about seven months, about eight months, about nine months, about ten months, about 11 months, about 12 months, about 1.5 years, about 2 years, about 2.5 years, about 3 years, about 3.5 years, about 4 years, about 4.5 years or about 5 years.

In some embodiments, the cryopreserved composition further comprises a freezing media. In some embodiments, the freezing media is serum freezing media. In some embodiments, the serum freezing media has fetal bovine serum. In some embodiments, the freezing media is serum-free media. In some embodiments, the freezing media comprises dimethyl sulfoxide (DMSO). In some embodiments, the freezing media comprises glycerol. In some embodiments, the freezing media is a Xeno-free media. Non-limiting examples of Xeno-free media includes Xeno-free X-VIVO media, Xeno-free mesenchymal stem cell media, and StemPro™ MSC SFM Xeno-free media. In some embodiments, the Xeno-free media is supplemented with human blood serum, platelet lysates, holo-transferrin, or insulin. In some embodiments, the Xeno-free media is supplemented with human blood serum. In some embodiments, the Xeno-free media is supplemented with platelet lysates. In some embodiments, the Xeno-free media is supplemented with holo-transferrin. In some embodiments, the Xeno-free media is supplemented with insulin. In some embodiments, the freezing media is a CryoStor® media. Non-limiting examples of CryoStor® media include CS10, CS5, CS2, and CSB. In some embodiments, the CryoStor® is CryoStor® CS10 media. In some embodiments, the freezing media comprises DMSO, sucrose, sodium hydroxide, potassium hydroxide, or a combination thereof. In some embodiments, the freezing media comprises about 2% to about 15% DMSO. In some embodiments, the freezing media comprises about 0.5% to about 2% sucrose. In some embodiments, the freezing media comprises about 1% sucrose. In some embodiments, the freezing media comprises about 0.5% to about 1% sodium hydroxide. In some embodiments, the freezing media comprises about 0.6% sodium hydroxide. In some embodiments, the freezing media comprises about 0.05% to about 0.5% potassium hydroxide. In some embodiments, the freezing media comprises about 0.1% potassium hydroxide.

In some embodiments, the cryopreserved composition further comprises a nonpyrogenic solution. Non-limiting examples of nonpyrogenic solution include lactated ringer's solution, cupric chloride solution, mannitol solution, phosphate buffer solution (PBS), sodium chloride solution, and sodium lactate solution. In some embodiments, the nonpyrogenic solution is PBS. In some embodiments, the nonpyrogenic solution is sodium lactate solution. In some embodiments, the nonpyrogenic solution is sterile.

In some embodiments, the preparing the fluid composition comprises thawing the cryopreserved composition. In some embodiments, the thawing is performed at room temperature. In some embodiments, the thawing is performed at a temperature between about 20° C. to about 50° C. In some embodiments, the thawing is performed at a temperature between about 20° C. to about 25° C., between about 25° C. to about 28° C., between about 28° C. to about 30° C., between about 30° C. to about 35° C., between about 35° C. to about 40° C., between about 40° C. to about 45° C., or between 45° C. to about 50° C. In some embodiments, the thawing is performed at a temperature of at least about 20%, at least about 25%, at least about 28° C., at least about 30° C., at least about 35° C., at least about 40° C., at least about 45° C., at least about 50° C., or more. In some embodiments, the thawing is performed at a temperature of at most about 50° C., at most about 45° C., at most about 40° C., at most about 35° C., at most about 30° C., at most about 28° C., at most about 25° C., at most about 20° C., or less. In some embodiments, the thawing is performed at a temperature of about 20° C., about 25° C., about 28° C., about 30° C., about 35° C., about 40° C., about 45° C., or about 50° C.

In some embodiments, the thawing is performed for between about five minutes to about two hours. In some embodiments, the thawing is performed for between about five minutes to about ten minutes, between about ten minutes to about 15 minutes, between about 15 minutes to about 30 minutes, between about 30 minutes to about 35 minutes, between about 35 minutes to about 40 minutes, between about 40 minutes to about 45 minutes, between about 45 minutes to about 50 minutes, between about 50 minutes to about 60 minutes, between about 60 minutes to about 70 minutes, between about 70 minutes to about 80 minutes, between about 80 minutes to about 90 minutes, between about 90 minutes to about 100 minutes, between about 100 minutes to about 110 minutes, or between about 110 minutes to about 120 minutes.

In some embodiments, the thawing is performed for at least about five minutes, at least about ten minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, at least about 60 minutes, at least about 70 minutes, at least about 80 minutes, at least about 90 minutes, at least about 100 minutes, at least about 110 minutes, at least about 120 minutes, or more. In some embodiments, the thawing is performed for at most about 120 minutes, at most about 110 minutes, at most about 100 minutes, at most about 90 minutes, at most about 80 minutes, at most about 70 minutes, at most about 60 minutes, at most about 55 minutes, at most about 50 minutes, at most about 45 minutes, at most about 40 minutes, at most about 35 minutes, at most about 30 minutes, at most about 25 minutes, at most about 20 minutes, at most about 15 minutes, at most about 10 minutes, at most about five minutes, or less. In some embodiments, the thawing is performed for about five minutes, about ten minutes, about 15 minutes, about 20 minutes, about 25 minutes, about 30 minutes, about 35 minutes, about 40 minutes, about 45 minutes, about 50 minutes, about 55 minutes, about 60 minutes, about 70 minutes, about 80 minutes, about 90 minutes, about 100 minutes, about 110 minutes, or about 120 minutes.

In some embodiments, the thawing is performed in an incubator. In some embodiments, the thawing is performed in a water bath. In some embodiments, the thawing is performed in a heat block. In some embodiments, the thawing is performed on a surface.

In some embodiments, the plurality of enucleated cells from the cryopreserved composition are suspended in a freezing media. In some embodiments, the plurality of enucleated cells from the cryopreserved composition are suspended in a Xeno-free media.

In some embodiments, preparing the fluid composition comprises thawing the cryopreserved composition. In some embodiments, the thawing the cryopreserved composition is performed at room temperature. In some embodiments, the thawing the cryopreserved composition is performed at 37° C. In some embodiments, the preparing the fluid formulation further comprises reconstituting the plurality of cryopreserved enucleated cells from the cryopreserved composition subsequent to the thawing. In some embodiments, the reconstituting uses Xeno-free media. Non-limiting examples of Xeno-free media includes Xeno-free X-VIVO media, Xeno-free mesenchymal stem cell media, and StemPro™ MSC SFM Xeno-free media. In some embodiments, the Xeno-free media is supplemented with human blood serum, platelet lysates, holo-transferrin, or insulin. In some embodiments, the Xeno-free media is supplemented with human blood serum. In some embodiments, the Xeno-free media is supplemented with platelet lysates. In some embodiments, the Xeno-free media is supplemented with holo-transferrin. In some embodiments, the Xeno-free media is supplemented with insulin. In some embodiments, the reconstituting uses a nonpyrogenic solution. Non-limiting examples of nonpyrogenic solution include lactated ringer's solution, cupric chloride solution, mannitol solution, phosphate buffer solution (PBS), sodium chloride solution, and sodium lactate solution. In some embodiments, the nonpyrogenic solution is PBS. In some embodiments, the nonpyrogenic solution is sodium lactate solution. In some embodiments, the nonpyrogenic solution is sterile. In some embodiments, the reconstituting the plurality of enucleated cells from the cryopreserved composition uses PBS. In some embodiments, the reconstituting the plurality of enucleated cells from the cryopreserved composition uses sodium lactate solution. In some embodiments, the reconstituting the plurality of enucleated cells from the cryopreserved composition uses saline solution.

Alternatively, in some embodiments, the plurality of cryopreserved enucleated cells in the cryopreserved composition are not reconstituted subsequent to the thawing. In some embodiments, the plurality of cryopreserved cells in the cryopreserved composition are not reconstituted prior to administering. In some embodiments, the plurality of cryopreserved cells in the cryopreserved compositions are not reconstituted subsequent to thawing and prior to administering.

### (ii) Lyophilization

In some embodiments, the enucleated cell is lyophilized. In some embodiments, the lyophilized enucleated cell can be reconstituted, and the reconstituted enucleated cell exhibits comparable viability to the enucleated cell that has not been lyophilized. In some embodiments, the lyophilization comprises components: freezing the cell; subjecting the cell to drying under a very low pressure (e.g., <3000 mTorr) using vacuum. The drying component can lead to sublimation and dehydrate the cell while maintaining cellular viability and biologic function. In some embodiments, the freezing phase comprises balancing the duration and temperature of the freezing to for maintaining cell viability and stability, appropriate crystal formation, and the speed of reconstitution. The triple point of a substance is the temperature and the pressure at which the sublimation curve, fusion curve, and vaporization curve meet. Achievement of the triple point which varies for different substances ensures that sublimation rather than melting will occur in the following drying steps. To facilitate faster and more efficient freeze-drying, larger ice crystals are preferred, because they form a network within the product that promotes faster removal of water vapor during sublimation. To produce larger crystals, the product should be frozen slowly or the temperature can be cycled up and down in a process called annealing. Fresh or frozen living tissue or cells do not have a single homogeneous melting point (eutectic point) and consequently the freezing stage of the material (cells or tissue) is cooled below its triple point which represents the temperature and pressure at which the solid, liquid and gas phases of the material can coexist. Living cells do have a critical point on a phase diagram at which both the liquid and the gas phase of an object or substance have the same density and are therefore indistinguishable. The product critical point temperature must be maintained to prevent melt-back or cake collapse occurring during primary and secondary drying which reflects incomplete sublimation. In the case of substances where preservation of structure is required like living cells, large ice crystals maybe detrimental and may break the cell walls which can result in increasingly poor texture and loss of nutritive content. In this case, the freezing should be done rapidly, in order to lower the material to below its critical point quickly, thus avoiding the formation of large ice crystals. The freezing temperatures for cells or tissue can vary but ranges in general between −50° C. (−58° F.) and −80° C. (−112° F.).

During the drying phase, the ambient pressure is lowered to the range of a few millibars, and then heat is supplied by conduction or radiation to the material for the ice to sublime. The amount of heat necessary can be calculated using the sublimating molecules' latent heat of sublimation. In this initial drying phase, about 95% of the water in the material or substance is sublimated. This phase is often slow and can even last for several days depending on the substance and technology employed but if too much heat is added to quickly the material's structure could be altered. In this phase, pressure is controlled through the application of a partial vacuum. The vacuum speeds up the sublimation, making it useful as a deliberate drying process. A cold condenser chamber and/or condenser plates are used as a surface(s) for the water vapor to re-liquify and solidify on. It is important to note that in this range of pressure, the heat cannot be provided by a convection effect because of the low air density. The drying phase also aims to remove remaining unfrozen water molecules since the ice induced with freezing should be removed during the primary drying phase. This part of the freeze-drying process is governed by the material's adsorption isotherms. In this phase, the temperature is raised higher than in the primary drying phase and can even be above 0° C. (32° F.), to break any physico-chemical interactions that have formed between the water molecules and the frozen material. Usually, during this phase the pressure is also lowered in this stage to encourage desorption. However, there are products that benefit from increased pressure as well. After the freeze-drying process is complete, the vacuum is usually broken with an inert gas, such as nitrogen, before the material is sealed. At the end of the operation, the residual water content in the product is extremely low and should range from <1% to 4% of the original concentration.

In some embodiments, the lyophilization of the enucleated cell comprises the use of lyoprotectants for retaining cell viability and biologic function. Lyoprotectant comprises addition of reagents, salts, or additives that protects cell during the desiccation process. Common lyoprotectants include trehalose, DMSO, methylcellulose, sucrose, antioxidants, human or animal serum proteins, and cellular stress proteins. Additionally, methods for increasing the transport of lyoprotectants inside the cells in suspension can be utilized as a way of improving the viability and function of cells after lyophilization. These methods include electroporation, addition of reagents that enhance intracellular transport, genetic modification of cells to upregulate the expression of pores on cell membranes, and mechanical microfluidic devices that partially disrupt cell membrane integrity and potentially promote intracellular transport of lyoprotectants.

In some embodiments, the nucleated cell described herein can be modified to express a targeting moiety (e.g., an antibody or antigen binding fragment thereof), a therapeutic agent, a transmembrane moiety, a heterologous gene product, or a combination thereof. In some embodiments, the nucleated cell can be modified to express at least one heterologous polynucleotide, where the at least one heterologous polynucleotide encodes a targeting moiety, a therapeutic agent, a transmembrane moiety, heterologous gene product, or a combination thereof.

In one aspect, disclosed herein are methods for modifying a cell by introducing at least one heterologous polynucleotide into the cell. In some embodiments, the heterologous polynucleotide encodes promoter, a heterologous gene product, or a combination thereof. In some embodiments, the methods comprise providing a composition comprising a first subset of nucleated cells and enucleated cells derived from a second subset of the nucleated cells. In some embodiments, the first subset of the nucleated cells comprises a heterologous polynucleotide encoding a heterologous gene product. In some embodiments, the methods comprise expressing the heterologous gene product thereby inducing cell death of at least one nucleated cell of the first subset of the nucleated cells. The heterologous polynucleotide can be introduced into any types of cells that could be enucleated, for instance, but not limited to, hTERT-immobilized Mesenchymal stem cells, by transfection of plasmids, transposons, zinc finger nucleases (ZFNs), transcription activator-like effector nucleases (TALENs), CRISPR-Cas technologies, viral transduction, etc.

In some embodiments, the heterologous polynucleotide comprises a promoter. In some embodiments, the promoter comprises an inducible promoter. In some embodiments, the promoter should be compatible with mammalian gene expression, provide rapid, strong gene expression, only in the presence of its induction stimulus. Associated suicide genes may include, but are not limited to, caspases, “eat me” signals, DNA crosslinkers, death inducing synthetic NOTCH receptors, toxins, and apoptosis/autophagy/entosis/necrosis/necroptosis/ferroptosis induction agents. In some embodiments, a promoter has its own activation protocol in terms of temperature, incubation time, light wavelength, inducer concentrations, and so forth.

In some embodiments, the inducible promoter is hypothermic. In some embodiments, the inducible promoter is induced by contacting the nucleated cells with a temperature that is below about 40 degrees Celsius (° C.). In some embodiments, the inducible promoter is induced by contacting the nucleated cells with a temperature that is below about 39° C. In some embodiments, the inducible promoter is induced by contacting the nucleated cells with a temperature that is below about 38° C. In some embodiments, the inducible promoter is induced by contacting the nucleated cells with a temperature that is below about 37° C. In some embodiments, the inducible promoter is induced by contacting the nucleated cells with a temperature that is below about 36° C. In some embodiments, the inducible promoter is induced by contacting the nucleated cells with a temperature that is below about 35° C. In some embodiments, examples of the inducible promoter include, but not limited to, comprises dsrA or CIRP.

In some embodiments, the inducible promoter is hyperthermic. In some embodiments, the inducible promoter is induced by contacting the nucleated cells with a temperature that is above 35° C. In some embodiments, the inducible promoter is induced by contacting the nucleated cells with a temperature that is above 36° C. In some embodiments, the inducible promoter is induced by contacting the nucleated cells with a temperature that is above 37° C. In some embodiments, the inducible promoter is induced by contacting the nucleated cells with a temperature that is above 38° C. In some embodiments, the inducible promoter is induced by contacting the nucleated cells with a temperature that is above 39° C. In some embodiments, the inducible promoter is induced by contacting the nucleated cells with a temperature that is above 40° C. In some embodiments, examples of the inducible promoter include, but not limited to, heat shock protein 70 (HSP70), heat shock protein 90 (HSP90), growth arrest- and DNA damage-inducible gene 153 (GADD153), multidrug resistance mutation 1 (MDR1), or cytomegalovirus (HSE-CMV).

In some embodiments, the inducible promoter is induced by contacting the nucleated cells with a molecule. In some embodiments, examples of the molecule include, but not limited to, rtTA, TRE, TetR, Cumate, Rapamycin, Abscisic acid, IPTG, or Methallothionein.

In some embodiments, the inducible promoter is induced by contacting the nucleated cells with light. In some embodiments, examples of the inducible promoter include, but not limited to, CIB1-CRY2 or GAL4-VVD.

In some embodiments, the inducible promoter is induced by contacting with the nucleated cells with a hormone. In some embodiments, examples of the inducible promoter include, but not limited to, Estradiol-Gal4.

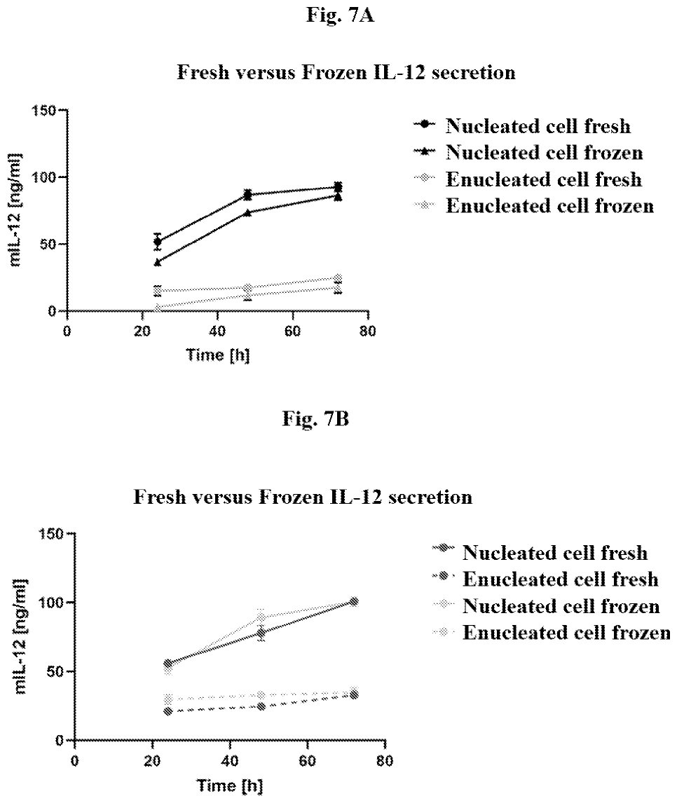

In some embodiments, the promoter comprises a constitutively active promoter. The promoter will be continually active, but suicide will be induced under certain circumstances. In some embodiments, the constitutively active promoter is configured to activate transcription of the heterologous polynucleotide under conditions sufficient to express the heterologous gene product. In some embodiments, examples of the heterologous gene product include, but not limited to, herpes simplex virus-thymidine kinase (HSV-TK), cytosine deaminase (CD), Varicalla-zoster-TK (VZV-TK), Nitroreductase, Carboxypeptidase G2 (CPG2), Cytochrome P450, or purine nucleoside phosphorylase. In some embodiments, examples of the heterologous gene product include, but not limited to, FKBP or a caspase. In some embodiments, an example of heterologous gene product includes, but not limited to, an antigen. In some embodiments, the heterologous polynucleotide is integrated into chromosome of the nucleated cells. In some embodiments, an example of the heterologous polynucleotide includes, but not limited to, a vector. Data examining enucleated and nucleated cells that were fresh or frozen (e.g., cryopreserved) are shown in FIG. 7 (FIG. 7A: enucleated human mesenchymal stem cells and FIG. 7B: enucleated human mesenchymal stem cells).

### (b) Method of Treatment

Disclosed herein, in some embodiments, are methods of using the enucleated cell, the composition, or the pharmaceutical composition described herein for treating a disease or condition. In some embodiments, the methods include treating the disease or condition of the subject by administering a composition described herein (e.g., a pharmaceutical composition containing enucleated cells engineered to express a therapeutic agent) to the subject. In some embodiments, the enucleated cells disclosed herein may be loaded, transfected or transduced with a therapeutic agent disclosed herein or any existing therapeutic agent, formulated in a pharmaceutical formulation, and the pharmaceutical formulation may be delivered to a subject according to various embodiments herein. The pharmaceutical formulations disclosed herein increases biodistribution and/or homing of the therapeutic agent to target cells or tissues in vivo, as compared to administering the therapeutic agent that is not encapsulated by or expressed in the enucleated cells disclosed herein.

The present disclosure also provides methods for the use of enucleated cells (natural or enucleated) as fusion partners to other cells (therapeutic or natural) to enhance and/or transfer biomolecules described herein, such as for example, a therapeutic agent. In some embodiments, the biomolecules include, DNA/genes, RNA (mRNA, shRNA, siRNA, miRNA), nanoparticles, peptides, proteins, and plasmids, bacteria, viruses, small molecule drugs, ions, cytokines, growth factors, and hormones. In some embodiments, the enucleated cell is engineered to express a fusogenic moiety. The fusogenic moiety can be any biomolecule (e.g., sugar, lipid, or protein) that promotes fusion of the membrane. In some embodiments, the fusogenic moiety is a fusogenic protein. A fusogenic protein allows the enucleated cell expressing the fusogenic protein to fuse with a target cell. In some embodiments, the fusogenic protein facilitates the merging of an enucleated cell expressing the fusogenic protein with a target cell, allowing the contents of the enucleated cell to enter into the target cell. In some embodiments, the fusogenic protein is heterotypic such as viral classes I-III or HAP2/GCS1 or SNARE. In some embodiments, the fusogenic protein is homoleptic such as EFF-1/AFF-1. Other non-limiting examples of the fusogenic protein is Izumol or Syncytin. In some embodiments, the fusogenic protein is a viral protein. In some embodiments, the fusogenic protein from a virus is VSV-g, hERV-W-ENV (Syncytin), or MV-Ed-F+MV-Ed-H (Hemagglutinin). Unlike nucleated cells, the fusion of enucleated cells to the same or another cell type of similar or different origin generates a unique cell hybrid that lacks problematic nuclear transfer, while maintaining desirable therapeutic attributes including, but not limited to, cell surface proteins, signal transduction molecules, secreted proteins, and epigenetic changes.

### (i) Subject

The methods disclosed herein, in some embodiments, comprise administering or delivering a composition (e.g., pharmaceutical composition) to a subject. In some embodiments, the subject has a disease, disorder, or condition, (e.g., cancer, fibrotic disease, autoimmune diseases, inflammatory diseases). In some embodiments, the subject is a mammal. In some embodiments, the mammal is a mouse. In some embodiments, the mammal is a rat. In some embodiments, the mammal is a human. In some embodiments, the subject is an adolescent, an adult, or an elderly subject. In some embodiments, the human subject is at least 18 years of age. In some embodiments, the human subject is age 18 to about 55 years. In some embodiments, the human subject is more than 55 years old. In some embodiments, the subject is age 18 to about 65 years. In some embodiments, the subject is more than 65 years old. In some embodiments, the subject is a female. In some embodiments, the subject is a male. In some embodiments, the subject is immunocompromised, or at increased risk for being immunocompromised.

### (ii) Disease or Condition

Provided herein are methods of treating a disease or a condition in a subject by administering a composition described herein to the subject. In some embodiments, administration is by any suitable mode of administration, including systemic administration (e.g., intravenous, inhalation, etc.). In some embodiments, the subject is human. In some embodiments, the disease or the condition comprises an infection (e.g., human immunodeficiency virus (HIV)-infection, Chagas disease, tuberculosis), a neurological disease (e.g., Parkinson's Disease, Huntington's Disease, Alzheimer's Disease) an autoimmune disease (e.g., diabetes, Crohn's disease, multiple sclerosis, sickle cell anemia), a cardiovascular disease (e.g., acute myocardial infarction, heart failure, refractory angina), a ophthalmologic disease, a skeletal disease, a metabolic disease (e.g., phenylketonuria, glycogen storage deficiency type 1A, Gaucher disease), an inflammatory disease (e.g., cancer, inflammatory bowel disease), or a disease caused by external pathogen or toxin in a subject. In some embodiments, the disease or the condition comprises idiopathic pulmonary fibrosis. In some embodiments, the subject is in need of, or has been determined to be in need of, such an enucleated cell treatment.

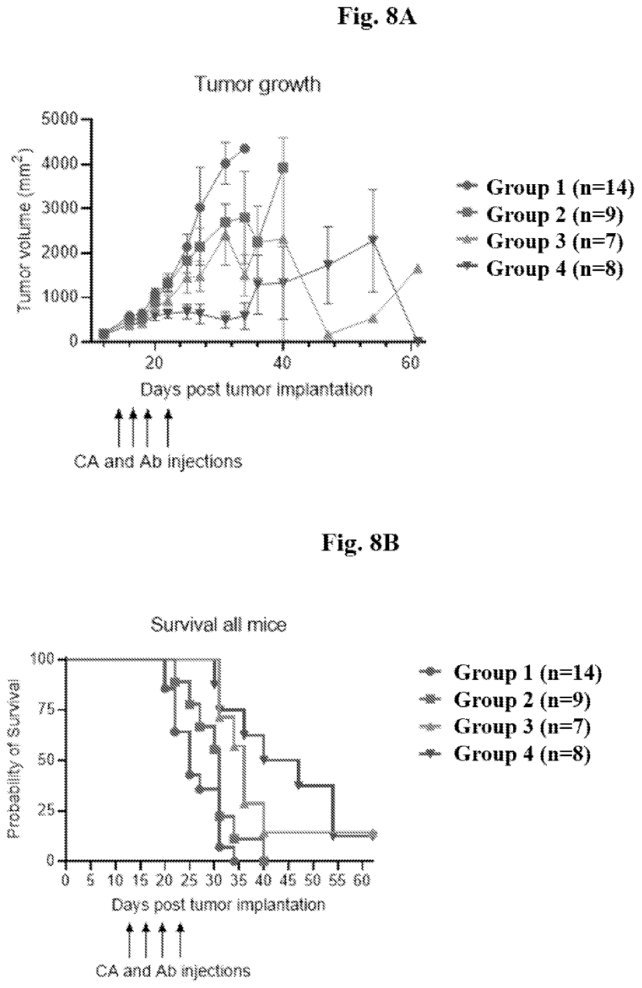

In some embodiments, the cancer may be lung cancer, including non-small cell lung cancer (NSCLC), small cell lung cancer (SCLC), or any other lung cancer type. For example, the lung cancer may include adenocarcinoma, squamous cell carcinoma, large cell (undifferentiated) carcinoma, large cell neuroendocrine carcinoma, adenosquamous carcinoma, sarcomatoid carcinoma, lung carcinoid tumor, bronchial carcinoids, or adenoid cystic carcinoma. Other non-limiting example of lung cancer includes lymphoma, sarcoma, benign lung tumor, or hamartoma. In some embodiments, the cancer is metastatic cancer. In some embodiments, the cancer metastasized to the lung from a different tissue or source. For example, the metastatic cancer that may be found in the lung may include breast cancer, colon cancer, prostate cancer, sarcoma, bladder cancer, neuroblastoma, and Wilm's tumor. In some embodiments, the cancer may be liver cancer. Non-limiting examples of lung cancers include hepatocellular carcinoma (HCC), fibrolamellar HCC, cholangiocarcinoma, angiosarcoma of the liver, metastatic cancer to the liver, and hepatoblastoma. Exemplary cancers that may be treated with a composition, pharmaceutical formulation, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g., triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g., non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer. FIG. 8 illustrates in vivo therapeutic assessment of the cryopreserved enucleated cells. FIG. 8A shows tumor growth over 60 days in four groups with various injections. FIG. 8B shows the probability of survival of the four groups investigated.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound, pharmaceutical composition, or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound, pharmaceutical composition, or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewings sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocyte sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound, pharmaceutical composition, or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

In some embodiments, the cancer cells express PD-1. In some embodiments, the cancer cells express PD-L1. In some embodiments, the cancer cells express PD-1 and PD-L1. Non-limiting examples of cancers associated with PD-1 expression include melanoma, lung cancer, kidney cancer, Hodgkin lymphoma, classical Hodgkin lymphoma, gastrointestinal cancers, cervical cancer, primary mediastinal large B cell lymphoma (PMBCL), hepatocellular carcinoma (HCC), merkel cell carcinoma, renal cell carcinoma, esophageal squamous cell cancer, endometrial carcinoma, non-muscle-invasive bladder cancer, melanoma, non-small cell lung cancer, lung cancer, squamous cell non-small cell lung cancer, urothelial carcinoma, head and neck squamous cell carcinomas, small cell lung cancer, colorectal cancer, pancreatic cancer, glioma, lymphoma, multiple myeloma, prostate cancer, ovarian cancer, cervical cancer, and cutaneous squamous cell carcinoma.

Non-limiting examples of cancer associated with PD-L1 expression include non-small cell lung cancer, small cell lung cancer, diffuse large-B cell lymphoma, cutaneous melanoma, solid tumors, bladder cancer, colorectal cancer, urothelial carcinoma, non-squamous non-small lung cell cancer, triple negative breast cancer, extensive-stage small cell lung cancer, Hodgkin lymphoma, hepatocellular carcinoma, colorectal cancer, urothelial cancer, bladder cancer, merkel cell carcinoma, renal cell carcinoma, head and neck squamous cell carcinoma, esophageal cancer, and bronchial cancer.

In some embodiments, the disease or condition comprises lung disease, such as lung cancer or cancer metastasized in the lung tissue of a subject. In some embodiments, the disease or condition is fibrosis, such as idiopathic pulmonary fibrosis. In some embodiments, methods of delivering the enucleated cells described herein comprises administering the enucleated cells or a pharmaceutical formulation containing enucleated cells to the subject. In some embodiments, the method utilizes an enucleated cell described herein for modulating expression of a target gene for treating a disease or condition in the subject. In some embodiments, the enucleated cell delivers an engineered oncolytic moiety or a polynucleotide encoding the engineered oncolytic moiety to a target cell, thereby treating the disease or condition. In some embodiments, the oncolytic moiety induces killing of the target cell. In some embodiments, the oncolytic moiety induces endogenous immune response for killing the cells in proximity of the target cell. In some embodiments, the target cell is a cancer cell. In some embodiments, the target cell is a tumor cell. In some embodiments, the enucleated cell delivers the engineered oncolytic moiety or the polynucleotide encoding the engineered oncolytic moiety to a microenvironment. In some embodiments, the microenvironment is a tumor microenvironment. In some embodiments, the microenvironment is a cancer microenvironment.

In some embodiments, the diseases or condition is an inflammatory disease or condition such as fibrosis. In some embodiments, the fibrosis can be fibrosis of any organ in a body (e.g., lung fibrosis, liver fibrosis, kidney fibrosis, brain fibrosis, or heart fibrosis). In some embodiments, the fibrosis is lung fibrosis including, but not limited to, fibrothorax, pulmonary fibrosis (e.g., idiopathic pulmonary fibrosis), or radiation-induced lung injury

In some embodiments, the disease is a transforming growth factor (TGF) associated disease. In some embodiments, the TGF is TGF-β. Non-limiting TGF-associated diseases include pulmonary fibrosis, liver fibrosis, glomerulosclerosis, renal interstitial fibrosis, cirrhosis, Crohn's disease, cardiomyopathy, scleroderma, and chronic graft-vs-host disease.

In some embodiments, the disease is a fibrotic disease. In some embodiments, the fibrotic disease is eye fibrosis. In some embodiments, the fibrotic disease is heart fibrosis. Non-limiting examples of heart fibrosis include reactive interstitial fibrosis, replacement fibrosis, infiltrative interstitial fibrosis and endomyocardial fibrosis. In some embodiments, the fibrotic disease is hepatic fibrosis. Non-limiting examples of hepatic fibrosis include non-alcoholic steatohepatitis (NASH), congenital hepatic fibrosis, and cystic fibrosis. In some embodiments, the fibrotic disease is intestinal fibrosis. In some embodiments, the fibrotic disease is lung fibrosis. Non-limiting examples of lung fibrotic diseases include idiopathic pulmonary fibrosis, systemic sclerosis (SSc), and pulmonary fibrosis. In some embodiments, the lung fibrotic disease is idiopathic pulmonary fibrosis. In some embodiments, the lung fibrotic disease is Ssc. In some embodiments, the fibrotic disease is pancreas fibrosis. In some embodiments, the fibrotic disease is renal fibrosis. In some embodiments, the renal fibrosis is tubulointerstitial fibrosis. In some embodiments, the renal fibrosis is glomerulosclerosis. In some embodiments, the fibrotic disease is skin fibrosis. Non-limiting examples of skin fibrotic diseases include systemic sclerosis (e.g., scleroderma), localized scleroderma, nephrogenic fibrosing dermopathy, mixed connective tissue disease, scleromyxederma, eosinophilic fasciitis, and calcinosis.

In some embodiments, the disease is an inflammatory disease. An inflammatory disease may refer to a disease, disorder, or other abnormal condition of a subject that is characterized by inflammation. In some embodiments, the inflammatory disease is an autoimmune disease. Non-limiting examples of inflammatory diseases or autoimmune diseases include Acanthosis Nigricans, Achalasia, Achlorhydria/Hypochlorhydria, Acquired Immunodeficiency Syndrome, Addison's disease, Adrenal Fatigue, Adrenal Insufficiency, Adult Still's disease, Agammaglobulinemia, Albinism, Amyloidosis, Amyotrophic Lateral Sclerosis/Lou Gehrig's Disease, Androgen Deficiency, Anemia, Aneurysm, Angina, Ankylosing Spondylitis, Anorexia, Anti-GBM/Anti-TBM nephritis, Antiphospholipid syndrome, Ataxia, Atherosclerosis, Atopic dermatitis, Atrial Fibrillation, Axial Spondyloarthritis, Axonal & neuronal neuropathy, Baló disease, Barrett's Esophagus, Basal Cell Cancer, Behcet's disease, Benign mucosal pemphigoid, Benign Prostatic Hyperplasia/Enlarged Prostate, Benign Tumor/Non-Cancerous Polyps, Biliary Insufficiency, Bipolar Disorder, Bladder Cancer, Blood Cell Disorder, Bone Cancer, Bone Spur, Bowel Cancer, Bradycardia, Brain Cancer, Branched-Chain Ketoaciduria, Breast Cancer, Brody Myopathy, Bronchitis, Bulimia, Bullous pemphigoid, Bursitis, Butterbur Allergy, Cancer/Malignant Tumor(s), Canker Sores/Aphthous Ulcers, Carcinoma, Cardiomyopathy, Cardiovascular Condition/Heart Disease, Castleman disease, Cervical Cancer, Chagas disease, Cholecystitis/Inflamed Gallbladder, Cholestasis/Impaired Bile Flow, Chronic Fatigue Syndrome, Chronic inflammatory demyelinating polyneuropathy, Chronic Laryngitis, Chronic Obstructive Pulmonary Disease, Chronic Pain, Chronic Pancreatitis, Chronic peptic ulcer, Chronic Pharyngitis, Chronic recurrent multifocal osteomyelitis, Churg-Strauss Syndrome/Eosinophilic Granulomatosis, Cicatricial pemphigoid, Cirrhosis/Liver Cirrhosis, Cobalamin Cobalt Hypersensitivity, Cogan's syndrome, Cognitive Dysfunction/Cognitive Impairment, Cold agglutinin disease, Colitis, Colon Polyps, Colorectal Cancer, Congenital heart block, Congestive Heart Failure, Connective Tissue Disease, Coronary Microvascular Disease, Coxsackie myocarditis, CREST syndrome, Crohn's Disease, Cushing's Syndrome, Cystic Acne, Cystic Fibrosis, Cytomegalovirus Infection, Degenerative Disc Disease, Degenerative Muscle Condition, Dermatitis/Skin rashes or irritation, Dermatitis herpetiformis, Dermatomyositis, Devic's disease/Neuromyelitis optica, Diabetes, Discoid lupus, Diverticular Condition, Diverticulitis, Diverticulosis, Down's Syndrome, Dressler's syndrome, Duchenne Muscular Dystrophy, Dysbiosis, Dysmotility, Eczema, Edema, Emphysema, Encephalitis, Endometrial Cancer, Endometriosis, Endothelial Dysfunction, Enterovirus, Eosinophilic Esophagitis, Eosinophilic fasciitis, Epilepsy, Epstein-Barr Virus Infection, Erectile Dysfunction, Erythema nodosum, Erythroplakia, Essential mixed cryoglobulinemia, Evans syndrome, Familial Adenomatous Polyposis, Fibromyalgia, Fibrosing alveolitis, Gallstones, Gastric Ulcers/Stomach Ulcers, Gastritis, Gastroenteritis, Gastroparesis, Genital Warts, Geographic Tongue, Gestational Diabetes, GI Inflammation/Gut Inflammation, Giant cell arteritis/Temporal arteritis, Giant cell myocarditis, Gilbert's Syndrome, Glaucoma, Glomerulonephritis, Goodpasture's syndrome, Gout, Granulomatosis with Polyangiitis, Graves' disease, Guillain-Barre Syndrome, Hand-Foot-and-Mouth Disease, Hashimoto's Disease/Autoimmune Thyroid Condition, Hashimoto's thyroiditis, Head and Neck Cancer, Helicobacter pylori Infection, Hemochromatosis, Hemolytic anemia, Hemorrhoids, Henoch-Schonlein purpura, Hepatitis, Hepatitis/Active hepatitis, Hepatitis A, Hepatitis B Virus, Hepatitis C Virus, Hernia, Herniated Disc, Herpangina, Herpes, Herpes gestationis or pemphigoid gestationis, Hiatal Hernia, Hidradenitis Suppurativa, Hirsutism/Hodgkin's Lymphoma, Hookworms, Hops Allergy, Hormone Sensitive Conditions, Human Immunodeficiency Virus, Human Papillomavirus Infection, Huntington's Disease, Hypercholesterolemia/High Cholesterol, Hyperlipidemia/High Lipids, Hyperthyroid/Graves' Disease, Hypothyroid/Hypothyroidism, IgA Nephropathy, IgG4-related sclerosing disease, Immune thrombocytopenia purpura, Immunodeficiency/Compromised Immunity, Impetigo, Inborn Errors of Metabolism, Inclusion Body Myositis, Inflammatory Bowel Disease, Inflammatory Disease, Influenza, Insect-Borne Infectious Disease, Insulin Resistance, Interstitial Cystitis, Irritable Bowel Syndrome, Irritable Bowel Syndrome-Constipation, Irritable Bowel Syndrome-Diarrhea, Irritable Bowel Syndrome-Mixed Type, Ischemic Colitis, Jaundice, Juvenile arthritis, Juvenile diabetes, Juvenile myositis, Kaposi's Sarcoma, Kawasaki disease, Kidney Cyst(s), Kidney Disease, Kidney Stones, Lambert-Eaton syndrome, Laryngitis, Latent Autoimmune Diabetes in Adults Leaky Gut/Intestinal Hyperpermeability, Leukemia, Leukocytoclastic vasculitis, Leukoplakia, Lichen Planus, Lichen Sclerosus, Licorice Allergy, Ligneous conjunctivitis, Limb-Girdle Muscular Dystrophy, Linear IgA disease, Liver Cancer, Liver Condition, Lung Cancer, Lupus, Lyme Disease, Lyme disease chronic, Lymphoma, Lynch Syndrome, Macular Degeneration Mast Cell Activation Disorder, Measles, Melanoma, Meniere's disease, Meningitis, Menopause, Metabolic Syndrome, Microscopic Colitis, Microscopic polyangiitis, Mixed Connective Tissue Disease, Miyoshi Myopathy, Mooren's ulcer, Mucha-Habermann disease, Multiple sclerosis, Multiple Sclerosis (MS), Mumps, Muscular Dystrophy, Myasthenia gravis, Myeloma, Myocarditis, Myopathy, Myositis, Neonatal Lupus, Neutropenia, Non-Hodgkin's Lymphoma, Non-STD Infectious Disease, Nonalcoholic Fatty Liver Disease/SteatoHepatitis, Norovirus, Ocular cicatricial pemphigoid, Optic neuritis, Oral Cancer, Oral Submucous Fibrosis, Orthostatic Hypotension, Osteoarthritis, Osteopenia, Osteoporosis, Ovarian Cancer, Ovarian Cyst, Palindromic rheumatism, Pancreatic Cancer, Pancreatic Insufficiency, Pancreatitis, PANDAS, Parasitic Intestinal Worms, Parkinson's Disease, Paroxysmal nocturnal hemoglobinuria, Parry Romberg syndrome, Pars planitis (peripheral uveitis, Parsonage-Turner syndrome, Pelvic inflammatory disease, Pemphigus, Peptic Ulcer Disease, Pericarditis, Periodontal disease/Periodontitis, Periodontal Gum Disease, Perivenous encephalomyelitis, Pernicious anemia, Peyronie's Disease, Phenylketonuria Plantar Fasciitis, Pleurisy, POEMS syndrome, Polyarteritis nodosa, Polycystic Ovarian Syndrome, Polycythemia Vera, Polyglandular syndromes type I/II/III, Polymyalgia Rheumatica, Polymyositis, Postmyocardial infarction syndrome, Postpericardiotomy syndrome, Preperfusion injury transplant rejection, Primary biliary cirrhosis, Primary sclerosing cholangitis, Proctitis/Inflammation of the rectum lining, Progesterone dermatitis, Prostate Cancer, Psoriasis, Psoriatic Arthritis, Pure red cell aplasia, Pyoderma gangrenosum, Raynaud's phenomenon, Raynaud's Phenomenon/Raynaud's Syndrome, Reactive Arthritis, Relapsing polychondritis, Renal Cancer/Kidney Cancer, Retinitis Pigmentosa, Retinopathy, Retroperitoneal fibrosis, Rheumatic disease, Rheumatic fever, Rheumatoid Arthritis, Roundworms, Rubella, Salivary Gland Infection or Sialadenitis, Sarcoidosis, Sarcoma, Schmidt syndrome, Sciatica/Sciatic Nerve Pain, Scleritis, Scleroderma, Sclerosing Cholangitis, Scoliosis, Short Bowel Syndrome, Sickle Cell Disease, Sinusitis, Sjögren's Syndrome, Skin Cancer, Sperm & testicular autoimmunity, Spinal Stenosis, Stiff person syndrome, Stomach Cancer, Subacute bacterial endocarditis, Supraventricular Tachycardia, Susac's syndrome, Sympathetic ophthalmia, Takayasu's arteritis, Temporal arteritis/Giant cell arteritis, Tendonitis, Testicular Cancer, Throat Cancer, Thrombocytopenia purpura, Thrombosis/Formation of blood clots, Thyroid Cancer, Thyroid Nodule(s), Tolosa-Hunt syndrome, Transient Ischemic Attack, Transverse myelitis, Tuberculosis, Type 1 Diabetes, Type 2 Diabetes, Ulcerative Colitis, Undifferentiated connective tissue disease, Urethritis, Uterine Cancer, Uterine Fibroids/Fibrosis, Uveitis, Vaginitis, Valvular Heart Disease, Varicella Zoster Infection/Chickenpox/Shingles, Vasculitis, Vertigo, Vitiligo, and Vogt-Koyanagi-Harada Disease.

In some embodiments, the enucleated cell described herein comprises a targeting moiety described herein for binding to an epitope expressed by a cancer cell or an epitope associated with a tumor microenvironment. In some embodiments, the targeting moiety comprises an antibody or antigen-binding fragment thereof described herein. In some embodiments, the antibody or antigen-binding fragment thereof comprises a single-domain antibody. In some embodiments, the antibody or antigen-binding fragment binds to an epitope expressed by a cancer cell or an epitope associated with a tumor microenvironment. In some embodiments, the binding of the targeting moiety (e.g., the antibody or the antigen-binding fragment thereof) to the epitope provides a therapeutic effect to treat cancer in a subject. In some embodiments, the binding of the targeting moiety (e.g., antibody or the antigen-binding fragment thereof) to the epitope recruits immune cells to activate immune response against the cancer.

In some embodiments, described herein are enucleated cells and methods of using these enucleated cells to treat a disease or condition associated with abnormal vasculature in a subject. Abnormal vasculature can be associated with disease or condition such as inflammation and cancer (e.g., any one of the cancers described herein). In some embodiments, the enucleated cells described herein, when contacted with the abnormal vasculature, increases the normalization of the abnormal vasculature, where the adhesion between endothelial cells is increased to prevent leakage of intravascular factors out of the vasculature. In some embodiments, the normalization of the abnormal vasculature includes decreasing of damages such as cell dead of the endothelial cells of the vasculature. In some embodiments, the normalization of the abnormal vasculature includes angiogenesis of immature or leaky vessels. In some embodiments, the normalization exerted by the enucleated cells can include normalization of blood vessel, lymphatic vessel, or a combination thereof.

In some embodiments, the disease or condition may be caused by a pathogen. In some embodiments, the enucleated cell described herein comprises an antibody or an antigen-binding fragment thereof or single-domain antibody that binds to an epitope expressed by the pathogen or an epitope associated with a microenvironment associated with the pathogen. In some cases, the binding of the antibody or the antigen-binding fragment thereof or single-domain antibody to the epitope confers therapeutic property against the pathogen. In some embodiments, the binding of the antibody or the antigen-binding fragment thereof or single-domain antibody to the epitope recruits immune cells to activate immune response to confer therapeutic property against the pathogen. For example, the disease or condition may be caused by virus, bacterium, fungus, parasite, or molecule resulted from detoxification. In some embodiments, the pathogens: may be disseminated or transmitted from person to person; result in high mortality rates and have the potential for major public health impact; may cause public panic and social disruption; and require special action for public health preparedness. Example of these pathogens may include Anthrax (Bacillus anthracis), Botulism (Clostridium botulinum toxin), Plague (Yersinia pestis), Smallpox (variola major), Tularemia (Francisella tularensis), or Viral hemorrhagic fevers, including Filoviruses (Ebola, Marburg) and Arenaviruses (Lassa, Machupo).

In some embodiments, the pathogen: may be disseminated; resulting in moderate morbidity rates and low mortality rates; and require specific enhancements of diagnostic capacity and enhanced disease surveillance. Example of these pathogens may include Brucellosis (Brucella species), Epsilon toxin of Clostridium perfringens, Food safety threats (e.g., Salmonella species, Escherichia coli 0157: H7, or Shigella), Glanders (Burkholderia mallei), Melioidosis (Burkholderia pseudomallei), Psittacosis (Chlamydia psittaci), Q fever (Coxiella burnetii), Ricin toxin from Ricinus communis (castor beans), Staphylococcal enterotoxin B, Typhus fever (Rickettsia prowazekii), Viral encephalitis (alphaviruses, such as eastern equine encephalitis, Venezuelan equine encephalitis, and western equine encephalitis), or Water safety threats (e.g., Vibrio cholerae and Cryptosporidium parvum).

In some embodiments, the pathogen may include emerging pathogen that has a high potential for mortality and morbidity, but the extend of which is not fully understood. Non-limiting examples of these pathogens may include Nipah virus and hantavirus.

The enucleated cells described herein, or the composition containing such enucleated cells (referred to in this section as “composition”) may be administered to a subject in a suitable dose, mod of administration, and frequency, which depends on the intended effect.

In some embodiments, the composition is administered at least once during a period of time (e.g., every 2 days, twice a week, once a week, every week, three times per month, two times per month, one time per month, every 2 months, every 3 months, every 4 months, every 5 months, every 6 months, every 7 months, every 8 months, every 9 months, every 10 months, every 11 months, once a year). In some embodiments, the composition is administered two or more times (e.g., 2, 3, 4, 5, 6, 7, 8, 9, 10, 11, 12, 13, 14, 15, 16, 17, 18, 19, 20, 30, 40, 50, 60,70, 80, 90, 100 times) during a period of time.

In some embodiments, the composition is administered in a therapeutically-effective amount by various forms and routes including, for example, oral, or topical administration. In some embodiments, a composition may be administered by parenteral, intravenous, subcutaneous, intramuscular, intradermal, intraperitoneal, intracerebral, subarachnoid, intraocular, intrasternal, ophthalmic, endothelial, local, intranasal, intrapulmonary, rectal, intraarterial, intrathecal, inhalation, intralesional, intradermal, epidural, intracapsular, subcapsular, intracardiac, transtracheal, subcuticular, subarachnoid, or intraspinal administration, e.g., injection or infusion. In some embodiments, a composition may be administered by absorption through epithelial or mucocutaneous linings (e.g., oral mucosa, rectal and intestinal mucosa administration). In some embodiments, the composition is delivered via multiple administration routes.

In some embodiments, the composition is administered by intravenous infusion. In some embodiments, the composition is administered by slow continuous infusion over a long period, such as more than 24 hours. In some embodiments, the composition is administered as an intravenous injection or a short infusion.

A composition may be administered in a local manner, for example, via injection of the agent directly into an organ, optionally in a depot or sustained release formulation or implant. A composition may be provided in the form of a rapid release formulation, in the form of an extended release formulation, or in the form of an intermediate release formulation. A rapid release form may provide an immediate release. An extended release formulation may provide a controlled release or a sustained delayed release. In some embodiments, a pump may be used for delivery of the composition. In some embodiments, a pen delivery device may be used, for example, for subcutaneous delivery of a composition of the disclosure. The composition provided herein may be administered in conjunction with other therapies, for example, an antiviral therapy, a chemotherapy, an antibiotic, a cell therapy, a cytokine therapy, or an anti-inflammatory agent.

The compositions (e.g., enucleated cells or pharmaceutical composition comprising the enucleated cell described herein) may be administered before, during, or after the occurrence of a disease or condition, and the timing of administering the composition containing a therapeutic agent may vary. In some cases, the composition may be used as a prophylactic and may be administered continuously to subjects (e.g., the subject for immunization or the subject for treatment) with a susceptibility to a coronavirus or a propensity to a condition or disease associated with a coronavirus. Prophylactic administration may lessen a likelihood of the occurrence of the infection, disease or condition, or may reduce the severity of the infection, disease or condition.

The composition may be administered to a subject before the onset of the symptoms. The composition may be administered to a subject (e.g., the subject for immunization or the subject for treatment) after (e.g., as soon as possible after) a test result, for example, a test result that provides a diagnosis, a test that shows the presence of a coronavirus in a subject (e.g., the subject for immunization or the subject for treatment), or a test showing progress of a condition, e.g., a decreased blood oxygen level. A composition may be administered after (e.g., as soon as is practicable after) the onset of a disease or condition is detected or suspected. A composition may be administered after (e.g., as soon as is practicable after) a potential exposure to a coronavirus, for example, after a subject (e.g., the subject for immunization or the subject for treatment) has contact with an infected subject or learns they had contact with an infected subject that may be contagious.

Actual dosage levels of an agent of the disclosure (e.g., antibody or antigen-binding fragment thereof, or therapeutic agent) may be varied so as to obtain an amount of the agent to achieve the desired therapeutic response for a particular subject, composition, and mode of administration, without being toxic to the subject (e.g., the subject for immunization or the subject for treatment). The selected dosage level may depend upon a variety of pharmacokinetic factors including the activity of the particular compositions employed herein, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

Dosage regimens may be adjusted to provide the optimum desired response (e.g., a therapeutic and/or prophylactic response). For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects (e.g., the subjects for immunization or the subjects for treatment); each unit contains a predetermined quantity of active agent calculated to produce the desired therapeutic effect in association with the required pharmaceutical carrier. The specification for the dosage unit forms of the disclosure may be determined by and directly dependent on (a) the unique characteristics of the active agent and the particular therapeutic effect to be achieved, and (b) the limitations inherent in the art of compounding such an active agent for the treatment of sensitivity in individuals. A dose may be determined by reference to a plasma concentration or a local concentration of the circular polyribonucleotide or antibody or antigen-binding fragment thereof. A dose may be determined by reference to a plasma concentration or a local concentration of the linear polyribonucleotide or antibody or antigen-binding fragment thereof.

A composition described herein may be in a unit dosage form suitable for a single administration of a precise dosage. In unit dosage form, the formulation may be divided into unit doses containing appropriate quantities of the compositions. In unit dosage form, the formulation may be divided into unit doses containing appropriate quantities of one or more linear polyribonucleotides, antibodies or the antigen-binding fragments thereof, and/or therapeutic agents. The unit dosage may be in the form of a package containing discrete quantities of the formulation. Non-limiting examples are packaged injectables, vials, and ampoules. An aqueous suspension composition disclosed herein may be packaged in a single-dose non-reclosable container. Multiple-dose reclosable containers may be used, for example, in combination with or without a preservative. A formulation for injection disclosed herein may be present in a unit dosage form, for example, in ampoules, or in multi dose containers with a preservative.

A dose may be based on the amount of the agent per kilogram of body weight of a subject (e.g., the subject for immunization or the subject for treatment). A dose of an agent (e.g., antibody) is in the range of 10-3000 mg/kg, e.g., 100-2000 mg/kg, e.g., 300-500 mg/kg/day for 1-10 or 1-5 days; e.g., 400 mg/kg/day for 3-6 days; e.g., 1 g/kg/d for 2-3 days In some embodiments, a dose may be based on the number of the enucleated cells per kilogram of body weight of a subject. In some embodiments, a dose may be is administered in a dosage amount of between about 1,000 cells/kg body weight to about 1,000,000,000,000 cells/kg body weight. In some embodiments, a dose may be is administered in a dosage amount of between about 1000 cells/kg body weight to about 1,000,000,000,000 cells/kg body weight. about 1,000 cells/kg body weight to about 10,000 cells/kg body weight, about 1,000 cells/kg body weight to about 100,000 cells/kg body weight, about 1,000 cells/kg body weight to about 1,000,000 cells/kg body weight, about 1,000 cells/kg body weight to about 10,000,000 cells/kg body weight, about 1,000 cells/kg body weight to about 100,000,000 cells/kg body weight, about 1,000 cells/kg body weight to about 1,000,000,000 cells/kg body weight, about 1,000 cells/kg body weight to about 10,000,000,000 cells/kg body weight, about 1,000 cells/kg body weight to about 100,000,000,000 cells/kg body weight, about 1,000 cells/kg body weight to about 1,000,000,000,000 cells/kg body weight, about 10,000 cells/kg body weight to about 100,000 cells/kg body weight, about 10,000 cells/kg body weight to about 1,000,000 cells/kg body weight, about 10,000 cells/kg body weight to about 10,000,000 cells/kg body weight, about 10,000 cells/kg body weight to about 100,000,000 cells/kg body weight, about 10,000 cells/kg body weight to about 1,000,000,000 cells/kg body weight, about 10,000 cells/kg body weight to about 10,000,000,000 cells/kg body weight, about 10,000 cells/kg body weight to about 100,000,000,000 cells/kg body weight, about 10,000 cells/kg body weight to about 1,000,000,000,000 cells/kg body weight, about 100,000 cells/kg body weight to about 1,000,000 cells/kg body weight, about 100,000 cells/kg body weight to about 10,000,000 cells/kg body weight, about 100,000 cells/kg body weight to about 100,000,000 cells/kg body weight, about 100,000 cells/kg body weight to about 1,000,000,000 cells/kg body weight, about 100,000 cells/kg body weight to about 10,000,000,000 cells/kg body weight, about 100,000 cells/kg body weight to about 100,000,000,000 cells/kg body weight, about 100,000 cells/kg body weight to about 1,000,000,000,000 cells/kg body weight, about 1,000,000 cells/kg body weight to about 10,000,000 cells/kg body weight, about 1,000,000 cells/kg body weight to about 100,000,000 cells/kg body weight, about 1,000,000 cells/kg body weight to about 1,000,000,000 cells/kg body weight, about 1,000,000 cells/kg body weight to about 10,000,000,000 cells/kg body weight, about 1,000,000 cells/kg body weight to about 100,000,000,000 cells/kg body weight, about 1,000,000 cells/kg body weight to about 1,000,000,000,000 cells/kg body weight, about 10,000,000 cells/kg body weight to about 100,000,000 cells/kg body weight, about 10,000,000 cells/kg body weight to about 1,000,000,000 cells/kg body weight, about 10,000,000 cells/kg body weight to about 10,000,000,000 cells/kg body weight, about 10,000,000 cells/kg body weight to about 100,000,000,000 cells/kg body weight, about 10,000,000 cells/kg body weight to about 1,000,000,000,000 cells/kg body weight, about 100,000,000 cells/kg body weight to about 1,000,000,000 cells/kg body weight, about 100,000,000 cells/kg body weight to about 10,000,000,000 cells/kg body weight, about 100,000,000 cells/kg body weight to about 100,000,000,000 cells/kg body weight, about 100,000,000 cells/kg body weight to about 1,000,000,000,000 cells/kg body weight, about 1,000,000,000 cells/kg body weight to about 10,000,000,000 cells/kg body weight, about 1,000,000,000 cells/kg body weight to about 100,000,000,000 cells/kg body weight, about 1,000,000,000 cells/kg body weight to about 1,000,000,000,000 cells/kg body weight, about 10,000,000,000 cells/kg body weight to about 100,000,000,000 cells/kg body weight, about 10,000,000,000 cells/kg body weight to about 1,000,000,000,000 cells/kg body weight, or about 100,000,000,000 cells/kg body weight to about 1,000,000,000,000 cells/kg body weight. In some embodiments, a dose may be is administered in a dosage amount of between about 1000 cells/kg body weight to about 1000000000000 cells/kg body weight. about 1,000 cells/kg body weight, about 10,000 cells/kg body weight, about 100,000 cells/kg body weight, about 1,000,000 cells/kg body weight, about 10,000,000 cells/kg body weight, about 100,000,000 cells/kg body weight, about 1,000,000,000 cells/kg body weight, about 10,000,000,000 cells/kg body weight, about 100,000,000,000 cells/kg body weight, or about 1,000,000,000,000 cells/kg body weight. In some embodiments, a dose may be is administered in a dosage amount of between about 1000 cells/kg body weight to about 1000000000000 cells/kg body weight. at least about 1,000 cells/kg body weight, about 10,000 cells/kg body weight, about 100,000 cells/kg body weight, about 1,000,000 cells/kg body weight, about 10,000,000 cells/kg body weight, about 100,000,000 cells/kg body weight, about 1,000,000,000 cells/kg body weight, about 10,000,000,000 cells/kg body weight, or about 100,000,000,000 cells/kg body weight. In some embodiments, a dose may be is administered in a dosage amount of between about 1000 cells/kg body weight to about 1000000000000 cells/kg body weight. at most about 10,000 cells/kg body weight, about 100,000 cells/kg body weight, about 1,000,000 cells/kg body weight, about 10,000,000 cells/kg body weight, about 100,000,000 cells/kg body weight, about 1,000,000,000 cells/kg body weight, about 10,000,000,000 cells/kg body weight, about 100,000,000,000 cells/kg body weight, or about 1,000,000,000,000 cells/kg body weight. In some embodiments, the cell without the nucleus is administered to the subject twice within at least an hour, 2 hours, 4 hours, 6 hours, 8 hours, 12 hours, 1 day, 2 days, a week, 2 weeks, 3 weeks, a month, 2 months, 3 months, 4 months, 5 months, 6 months, 7 months, 8 months, 9 months, 10 months, 11 months, a year, 2 years, 3 years, or 4 years.

Disclosed herein are methods of repeatedly administering the composition or the pharmaceutical composition to the subject in need thereof. In some embodiments, the first administrations of the composition or pharmaceutical composition comprising the enucleated cell normalizes blood vessel or lymphatic vessel. In some embodiments, the composition or pharmaceutical composition comprising the same enucleated cell can be subsequently administered to the subject for: maintaining the normalization of the blood vessel or lymphatic vessel; and delivering the exogenous agent for treating a disease or condition described herein.

### Kits

2. Disclosed herein, in some aspects, are kits for using the compositions described herein. The kit may contain one or more formulations, one or more pharmaceutical formulation, and one or more containers for storing the one or more formulations or the one or more pharmaceutical formulation, or any combination thereof. In some embodiments, the kit comprises the formulations disclosed herein. In some embodiments, the kit comprises pharmaceutical formulations disclosed herein. In some embodiments, the kit comprises a container storing the formulation or the pharmaceutical formulation. In some embodiments, the kits disclosed herein may be used to treat a disease or condition in a subject. In some embodiments, the kits comprise an assemblage of materials or components apart from the composition. In some embodiments, the kit comprises nucleated cell described herein (e.g., nucleated cell engineered to express a targeting moiety (e.g., antibody or antigen-binding fragment thereof), a therapeutic agent, a transmembrane moiety, an immune-evading moiety, a heterologous gene produce, or a combination thereof. In some embodiments, the kit can include enucleated cells obtained from the nucleated cells. In some embodiments, the kit can include a mixed population of nucleated cells and enucleated cells obtained from the nucleated cells. In some embodiments, the kit can include a substantially pure population of enucleated cells. In some embodiments, the kit comprises nucleated cells, enucleated cells, or a combination thereof suspended in at least one density gradient. In some embodiments, the kit comprises a resuspension buffer. In some embodiments, the resuspension buffer comprises PBS. In some embodiments, the resuspension buffer comprises saline solution. In some embodiments, the resuspension buffer comprises sodium lactate solution.

In some embodiments, the kit comprises the cryopreserved or the cryohibernated enucleated cells of the present disclosure. In some embodiments, the kit further comprises a container for storing the cryopreserved or the cryohibernated enucleated cells. In some embodiments, the kit further comprises a container for storing a pharmaceutical formulation of the present disclosure. In some embodiments, the container is a microcentrifuge tube. In some embodiments, the container is a cryovial. In some embodiments, the container is a centrifuge tube. In some embodiments, the container is a cell culture bag. Non-limiting examples of cell culture bags include Gibco cell culture bags, VueLife cell culture bags, CryoMACS® bags, and EVA bags.

In some embodiments, the container has a volume of between about 1 mL to about 5,000 mL. In some embodiments, the container has a volume between about 1 mL to about 2 mL, between about 2 mL to about 5 mL, between about 5 mL to about 10 mL, between about 15 mL to about 15 mL, between about 15 mL to about 20 mL, between about 20 mL to about 40 mL, between about 40 mL to about 60 mL, between about 60 mL to about 80 mL, between about 80 mL to about 100 mL, between 100 mL to about 150 mL, between about 150 mL to about 200 mL, between about 200 mL to about 250 mL, between 250 mL to about 300 mL, between about 300 mL to about 350 mL, between about 350 mL to about 400 mL, between about 400 mL to about 450 mL, between about 450 mL to about 500 mL, between about 500 mL to about 600 mL, between about 600 mL to about 700 mL, between about 700 mL to about 800 mL, between about 800 mL to about 900 mL, between about 900 mL to about 1,000 mL, between 1,000 mL to about 1,500 mL, between about 1,500 mL to about 2,000 mL, between about 2,000 mL to about 2,500 mL, between about 2,500 mL to about 3,000 mL, between about 3,000 mL to about 3,500 mL, between about 3,500 mL to about 4,000 mL, between about 4,000 mL to about 4,500 mL, or between about 4,500 mL to about 5,000 mL.

In some embodiments, the container has a volume of at least about 1 mL, at least about 2 mL, at least about 5 mL, at least about 10 mL, at least about 15 mL, at least about 20 mL, at least about 25 mL, at least about 50 mL, at least about 100 mL, at least about 150 mL, at least about 200 mL, at least about 250 mL, at least about 300 mL, at least about 350 mL, at least about 400 mL, at least about 450 mL, at least about 500 mL, at least about 550 mL, at least about 600 mL, at least about 650 mL, at least about 700 mL, at least about 750 mL, at least about 800 mL, at least about 850 mL, at least about 900 mL, at least about 950 mL, at least about 1,000 mL, at least about 1,500 mL, at least about 2,000 mL, at least about 2,500 mL, at least about 3,000 mL, at least about 3,500 mL, at least about 4,000 mL, at least about 4,500 mL, at least about 5,000 mL, or more.

In some embodiments, the container has a volume of about 1 mL, about 2 mL, about 5 mL, about 10 mL, about 15 mL, about 20 mL, about 25 mL, about 50 mL, about 100 mL, about 150 mL, about 200 mL, about 250 mL, about 300 mL, about 350 mL, about 400 mL, about 450 mL, about 500 mL, about 550 mL, about 600 mL, about 650 mL, about 700 mL, about 750 mL, about 800 mL, about 850 mL, about 900 mL, about 950 mL, about 1,000 mL, about 1,500 mL, about 2,000 mL, about 2,500 mL, about 3,000 mL, about 3,500 mL, about 4,000 mL, about 4,500 mL, or about 5,000 mL.

In some embodiments, the kit further comprises a resuspension buffer. In some embodiments, the resuspension buffer is PBS. In some embodiments, the resuspension buffer is sodium lactate solution. In some embodiments, the resuspension buffer is a nonpyrogenic solution. In some embodiments, the nonpyrogenic solution is PBS. In some embodiments, the nonpyrogenic solution is sodium lactate solution.

In some embodiments, the kit comprises a pharmaceutical formulation disclosed herein, comprising the enucleated cells engineered to express (and in some cases secrete) a targeting moiety (e.g., antibody or antigen-binding fragment thereof), a therapeutic agent, a transmembrane moiety, an immune-evading moiety, a heterologous gene produce, or a combination thereof. In some embodiments, the enucleated cell expresses or secretes the therapeutic agent such as an immune checkpoint molecule or an immune checkpoint inhibitor for treating a disease or condition in a subject. In some embodiments, the enucleated cells are further engineered to express a targeting moiety, such as a chemokine receptor, an integrin signaling molecule or an antibody or antigen-binding fragment thereof that enables the enucleated cells to efficiently migrate to the target tissue in a subject, once administered. In some embodiments, the kits further comprise an additional therapeutic agent, such as those disclosed herein. In some embodiments, the kit further comprises instructions for administering the pharmaceutical formulation and/or additional therapeutic agent to the subject to treat a disease or a condition in the subject such as cancer. In some embodiments, the cancer comprises cancer of the lung tissue. In some embodiments, the cancer is lung cancer.

In some embodiments, the kit comprises components for purifying enucleated cells from nucleated cells or other cellular debris. For example, the kit can include filter membranes with different pore sizes to isolate and purify the enucleated cells. In some embodiments, the kit comprises components for staining and selecting for enucleated cells. For example, the kit can include florescent dye for staining nucleus, where the nucleated cell can be stained and selectively removed, leaving a population of enucleated cells. In some embodiments, the kit comprises components for inducing cell death of the nucleated cell. For example, the kit can include molecules for inducing expression of the heterologous gene product described herein for inducing cell death of nucleated cell.

In some embodiments, the kit described herein comprises components for selecting for a homogenous population of the enucleated cells. In some embodiments, the kit described herein comprises components for selecting for a heterogenous population of the enucleated cells. In some embodiments, the kit comprises the components for assaying the number of units of a biomolecule (e.g., a therapeutic agent) synthesized, and/or released or expressed on the surface by the enucleated cell. In some embodiments, the kit comprises components for performing assays such as enzyme-linked immunosorbent assay (ELISA), single-molecular array (Simoa), PCR, and qPCR. The exact nature of the components configured in the kit depends on its intended purpose. For example, some embodiments are configured for the purpose of treating a disease or condition disclosed herein (e.g., cancer) in a subject. In some embodiments, the kit is configured particularly for the purpose of treating mammalian subjects. In some embodiments, the kit is configured particularly for the purpose of treating human subjects.

Instructions for use may be included in the kit. In some embodiments, the kit comprises instructions for administering the composition to a subject in need thereof. In some embodiments, the kit comprises instructions for further engineering the composition to express a biomolecule (e.g., a therapeutic agent). In some embodiments, the kit comprises instructions thawing the cryopreserved composition. In some embodiments, the kit comprises instructions for measure viability of the restored compositions, to ensure efficacy for its intended purpose (e.g., therapeutic efficacy if used for treating a subject). In some embodiments, the kit comprises instructions comprising a method for delivering the formulation or the pharmaceutical formulation to a target cell of a subject, wherein the method comprises: introducing the formulation or the pharmaceutical formulation to the target cell of a subject in vivo or ex vivo under conditions sufficient to deliver the therapeutic agent to the target cell. In some embodiments, the method of the instructions comprises treating a disease or a condition of the subject by administering the therapeutic agent to the target cell of the subject. In some embodiments, the disease or the condition comprises cancer. In some embodiments, the cancer is a lung cancer, a cancer metastases in lung tissue, a liver cancer, or a cancer metastases in liver tissue. In some embodiments, the introducing the formulation or the pharmaceutical formulation to the target cell of a subject comprises administering the formulation or the pharmaceutical formulation to the subject intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesicaly, intravaginally, intrarectally, orally, sublingually, transdermally, by inhalation, by inhaled nebulized form, by intraluminal-GI route, or any combination thereof. In some embodiments, the kit comprises at least one additional active agent, wherein the at least one additional active agent comprises a cytokine, a growth factor, a hormone, an enzyme, a small molecule, a compound, or any combination thereof.

Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit may be provided to the practitioner stored in any convenient and suitable ways that preserve their operability and utility. For example, the components may be in dissolved, dehydrated, or lyophilized form; they may be provided at room, refrigerated or frozen temperatures. The components may be contained in suitable packaging material(s).

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to +10% of a stated number or value.

The terms “increased”, “increasing”, or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease may be, for example, at least 10%, at least 20%, at least 30%, at least 40% or more, and is preferably down to a level accepted as within the range of normal for an individual without a given disease. Other examples of “decrease” include a decrease of at least 2-fold, at least 5-fold, at least 10-fold, at least 20-fold, at least 50-fold, at least 100-fold, at least 1000-fold or more as compared to a reference level.

The terms “individual” or “subject” are used interchangeably and encompass mammals. Non-limiting examples of mammal include any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The mammal may be a human. The term “animal” as used herein comprises human beings and non-human animals. In one embodiment, a “non-human animal” is a mammal, for example a rodent such as rat or a mouse. A “patient,” as used herein refers to a subject that has, or has been diagnosed with, a disease or a condition described herein.

The term “immune-evading moiety,” as used herein, refers to a signaling peptide, or portion thereof, that reduces cellular phagocytosis through its interaction with a signal receptor protein expressed by phagocytic cells, such as macrophages and dendritic cells. In some embodiments, the immune-evading moiety blocks immune cell recognition or immune cell activation.

The term “targeting moiety,” as used herein, refers to an entity that guides a cell, such as for e.g., an enucleated cell, to a target tissue or cell. The targeting moiety can be virtually any biomolecule, including a protein, polypeptide, a sugar, a nucleic acid, or a small molecule, or portions thereof.

The term “transmembrane moiety,” as used herein, refers to an entity that spans (at least partially) the cell membrane of a cell (e.g., enucleated cell).

The terms “expression” or “expressing” refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state.

As used herein, a “cell” generally refers to a biological cell.

As used herein, “enucleation” is the rendering of a cell to a non-replicative state, such as, for example, through removal of the nucleus.

As used herein, the term “cytoplast,” “cell without a nucleus,” or “enucleated cell” are used interchangeably to refer to a nucleus-free cell that was obtained from a previously nucleated cell (e.g., any cell described herein). In some embodiments, the nucleated cell comprises cell organelles and the cytoplast derived from the nucleated cell retains such organelles, which in some cases, enables cellular functions such as cell motility, protein synthesis, protein secretion, and the like. In some embodiments, “obtaining” does not involve differentiating the nucleated cell into an enucleated cell using natural processes or otherwise.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence. The term “gene” is to be interpreted broadly, and may encompass mRNA, cDNA, CRNA and genomic DNA forms of a gene.

In some uses, the term “gene” encompasses the transcribed sequences, including 5′ and 3′ untranslated regions (5′-UTR and 3′-UTR), exons and introns. In some genes, the transcribed region may contain “open reading frames” that encode polypeptides. In some uses of the term, a “gene” comprises only the coding sequences (e.g., an “open reading frame” or “coding region”) necessary for encoding a polypeptide. In some embodiments, genes do not encode a polypeptide, for example, ribosomal RNA genes (rRNA) and transfer RNA (tRNA) genes. In some embodiments, the term “gene” includes not only the transcribed sequences, but in addition, also includes non-transcribed regions including upstream and downstream regulatory regions, enhancers and promoters. The term “gene” may encompass mRNA, cDNA and genomic forms of a gene.

The term “packaging material” refers to one or more physical structures used to house the contents of the kit, such as compositions and the like. The packaging material is constructed by well-known methods, preferably to provide a sterile, contaminant-free environment. The packaging materials employed in the kit are those customarily utilized in gene expression assays and in the administration of treatments.

As used herein, the term “package” refers to a suitable solid matrix or material such as glass, plastic, paper, foil, and the like, capable of holding the individual kit components. For example, a package may be a glass vial or prefilled syringes used to contain suitable quantities of the pharmaceutical. The packaging material has an external label which indicates the contents and/or purpose of the kit and its components.

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure, and may perform any function, known or unknown. A polynucleotide comprises one or more analogs (e.g., altered backbone, sugar, or nucleobase). Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

As used herein, the terms “polypeptide,” “peptide” and “protein” may be used interchangeably herein in reference to a polymer of amino acid residues. A protein may refer to a full-length polypeptide as translated from a coding open reading frame, or as processed to its mature form, while a polypeptide or peptide may refer to a degradation fragment or a processing fragment of a protein that nonetheless uniquely or identifiably maps to a particular protein. A polypeptide may be a single linear polymer chain of amino acids bonded together by peptide bonds between the carboxyl and amino groups of adjacent amino acid residues. Polypeptides may be modified, for example, by the addition of carbohydrate, phosphorylation, etc.

As used herein, the terms “fragment,” or “portion,” or equivalent terms may refer to a portion of an entity that has less than the full length of the entity and optionally maintains the function of the entity. In some embodiments, the entity is a protein.

The terms “complement,” “complements,” “complementary,” and “complementarity,” as used herein, generally refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g., thermodynamically more stable under a given set of conditions, such as stringent conditions used in the relevant field) to hybridization with non-target sequences during a hybridization reaction. Typically, hybridizable sequences share a degree of sequence complementarity over all or a portion of their respective lengths, such as between 25%-100% complementarity, including greater than or equal to about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity, such as for the purpose of assessing percent complementarity, may be measured by any suitable alignment algorithm, including but not limited to the Needleman-Wunsch algorithm (see e.g., the EMBOSS Needle aligner available at www.ebi.ac.uk/Tools/psa/emboss_needle/nucleotide.html, optionally with default settings), the BLAST algorithm. Optimal alignment may be assessed using any suitable parameters of a chosen algorithm, including default parameters.

The term “percent (%) identity,” as used herein, generally refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (e.g., gaps may be introduced in one or both of the candidate and reference sequences for optimal alignment and non-homologous sequences may be disregarded for comparison purposes). Alignment, for purposes of determining percent identity, may be achieved in various ways that are known in the relevant field. Percent identity of two sequences may be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

As used herein, the term “in vivo” may be used to describe an event that takes place in an organism, such as a subject's body.

As used herein, the term “ex vivo” may be used to describe an event that takes place outside of an organism, such as subject's body. An “ex vivo” assay cannot be performed on a subject. Rather, it may be performed upon a sample separate from a subject. Ex vivo may be used to describe an event occurring in an intact cell outside a subject's body.

As used herein, the term “in vitro” may be used to describe an event that takes places contained in a container for holding laboratory reagent such that it is separated from the living biological source organism from which the material is obtained. In vitro assays may encompass cell-based assays in which cells alive or dead are employed. In vitro assays may also encompass a cell-free assay in which no intact cells are employed.

“Treat, “treating,” or “treatment,” as used herein, refers to alleviating or abrogating a disorder, disease, or condition; or one or more of the symptoms associated with the disorder, disease, or condition; or alleviating or eradicating a cause of the disorder, disease, or condition itself. Desirable effects of treatment may include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “effective amount” and “therapeutically effective amount,” as used interchangeably herein, generally refer to the quantity of a composition, for example a composition comprising immune cells such as lymphocytes (e.g., T lymphocytes and/or NK cells) comprising a system of the present disclosure, that is sufficient to result in a desired activity upon administration to a subject in need thereof. Within the context of the present disclosure, the term “therapeutically effective” refers to that quantity of a composition that is sufficient to delay the manifestation, arrest the progression, relieve or alleviate at least one symptom of a disorder treated by the methods of the present disclosure.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. A component may be “pharmaceutically acceptable” in the sense of being compatible with the other ingredients of a pharmaceutical formulation. It may also be suitable for use in contact with the tissue or organ of humans and animals without excessive toxicity, irritation, allergic response, immunogenicity, or other problems or complications, commensurate with a reasonable benefit/risk ratio.

As used herein, the term “administration,” “administering” and variants thereof means introducing a composition or agent into a subject and includes concurrent and sequential introduction of a composition or agent. The introduction of a composition or agent into a subject is by any suitable route, including orally, pulmonarily, intranasally, parenterally (intravenously, intramuscularly, intraperitoneally, or subcutaneously), rectally, intralymphatically, or topically. Administration includes self-administration and administration by another. In some embodiments, the method further comprises administering the plurality of enucleated cells to the subject intravenously. A suitable route of administration allows the composition or the agent to perform its intended function. For example, if a suitable route is intravenous, the composition is administered by introducing the composition or agent into a vein of the subject. Administration may be carried out by any suitable route. In some embodiments, the administering is intravenous administration. In some embodiments, the administering is pulmonary administration. In some embodiments, the administering is inhalation.

The term “pharmaceutical composition” refers to a mixture of a composition disclosed herein with other chemical components, such as diluents or carriers (e.g., pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition may facilitate administration of the composition to an organism. Multiple techniques of administering a compound exist in the art including, but not limited to, oral, injection, aerosol, parenteral, and topical administration.

The term “fusogenic protein”, as used herein, refers to a polypeptide that, when expressed on the surface of cell, such as the enucleated cell, facilitates fusion of cell-to-cell membranes of the cell expressing the fusogenic protein and a target cell.

While preferred embodiments of the present inventive concepts have been shown and described herein, it will be obvious to those skilled in the art that such embodiments are provided by way of example only. It is not intended that the inventive concepts be limited by the specific examples provided within the specification. While the inventive concepts have been described with reference to the aforementioned specification, the descriptions and illustrations of the embodiments herein are not meant to be construed in a limiting sense. Numerous variations, changes, and substitutions will now occur to those skilled in the art without departing from the inventive concepts. Furthermore, it shall be understood that all aspects of the inventive concepts are not limited to the specific depictions, configurations or relative proportions set forth herein which depend upon a variety of conditions and variables. It should be understood that various alternatives to the embodiments of the inventive concepts described herein may be employed in practicing the inventive concepts. It is therefore contemplated that the inventive concepts shall also cover any such alternatives, modifications, variations or equivalents. It is intended that the following claims define the scope of the inventive concepts and that methods and structures within the scope of these claims and their equivalents be covered thereby.

### EMBODIMENTS

Embodiment 1. A method of delivering a therapeutic agent to a target cell of a subject, the method comprising: preparing a fluid formulation comprising a plurality of enucleated cells from a cryopreserved composition, wherein the cryopreserved composition comprises the plurality of enucleated cells that are cryopreserved, wherein at least a subset of the plurality of enucleated cells comprises a therapeutic agent; and introducing the fluid formulation to the subject or a sample of the subject under conditions sufficient to deliver the therapeutic agent to the target cell of the subject in vivo or ex vivo in an amount that is greater than or equal to about an amount of the therapeutic agent delivered to an otherwise identical target cell of the subject by otherwise identical enucleated cells that were not cryopreserved.

Embodiment 2. The method of Embodiment 1, wherein the cryopreserved composition is cryopreserved in liquid nitrogen.

Embodiment 3. The method of Embodiment 2, wherein the cryopreserved composition is cryopreserved for at least about 24 hours.

Embodiment 4. The method of Embodiment 2, wherein the cryopreserved composition is cryopreserved for at least about 7 days.

Embodiment 5. The method of Embodiment 2, wherein the cryopreserved composition is cryopreserved for at least about one month.

Embodiment 6. The method of Embodiment 2, wherein the cryopreserved composition is cryopreserved for at least about one year.

Embodiment 7. The method of Embodiment 2, wherein the cryopreserved composition is placed at most about −80° C. prior to cryopreserving the cryopreserved composition.

Embodiment 8. The method of Embodiment 7, wherein the cryopreserved composition is placed at most about −80° C. for at least about 24 hours.

Embodiment 9. The method of Embodiment 2, wherein the plurality of enucleated cells from the cryopreserved composition are suspended in Xeno-free media.

Embodiment 10. The method of Embodiment 2, wherein the plurality of enucleated cells from the cryopreserved composition are suspended in CryoStor® media.

Embodiment 11. The method of Embodiment 10, wherein the CryoStor® media is CryoStor® CS10 media.

Embodiment 12. The method of Embodiment 1, wherein the preparing the fluid composition comprises thawing the cryopreserved composition.

Embodiment 13. The method of Embodiment 12, wherein the thawing of the cryopreserved composition is performed at room temperature.

Embodiment 14. The method of Embodiment 12, wherein the thawing the cryopreserved composition is performed at 37° C.

Embodiment 15. The method of Embodiment 14, wherein the thawing the cryopreserved composition is performed at 37° C. in a water bath.

Embodiment 16. The method of Embodiment 12, further comprising: reconstituting the plurality of enucleated cells from the cryopreserved composition subsequent to the thawing.

Embodiment 17. The method of Embodiment 16, wherein the reconstituting the plurality of enucleated cells from the cryopreserved composition uses phosphate buffer solution (PBS).

Embodiment 18. The method of Embodiment 16, wherein the reconstituting the plurality of enucleated cells from the cryopreserved composition uses sodium lactate solution.

Embodiment 19. The method of Embodiment 1, wherein the therapeutic agent comprises a virus, an exogenous DNA molecule, an exogenous RNA molecule, an exogenous protein, an exogenous peptide, or a combination thereof.

Embodiment 20. The method of Embodiment 19, wherein the therapeutic agent comprises a virus.

Embodiment 21. The method of Embodiment 19, wherein the virus is an adeno-associated virus (AAV), an adenovirus, a reovirus, a coxsackie virus, a retrovirus, a poxvirus, a baculovirus, or a herpes virus.

Embodiment 22. The method of Embodiment 20, wherein the virus comprises an oncolytic virus.

Embodiment 23. The method of Embodiment 22, wherein the oncolytic virus is an adenovirus, a human immunodeficiency virus, a Maraba virus, a Measles virus, a Newcastle disease virus, a poliovirus, a Seneca Valley virus, a parvovirus, a Semliki Forest virus, a Vesicular Stomatitis virus, a Sindbis virus, or a combination thereof.

Embodiment 24. The method of Embodiment 20, wherein the amount of the virus delivered to the subject in vivo or ex vivo is a measurement of viral titers in the target cell.

Embodiment 25. The method of Embodiment 24, wherein the viral titers measured in the target cell are greater than the viral titers measured in the otherwise identical target cell.

Embodiment 26. The method of Embodiment 24, wherein the viral titers measured in the target cell are equal to about the viral titers measured in the otherwise identical target cell.

Embodiment 27. The method of Embodiment 19, wherein the therapeutic agent comprises a cytokine or cytokine receptor-binding fragment thereof.

Embodiment 28. The method of Embodiment 27, wherein the amount of the cytokine or the cytokine receptor-binding fragment thereof delivered to the subject in vivo or ex vivo is a measurement of the secretion of the cytokine or the cytokine receptor-binding fragment thereof from the plurality of enucleated cells.

Embodiment 29. The method of Embodiment 28, wherein the secretion of the cytokine or the cytokine receptor-binding fragment thereof measured is greater than or equal to about the secretion of the cytokine or the cytokine receptor-binding fragment thereof by an otherwise identical enucleated cell that was not cryopreserved.

Embodiment 30. The method of Embodiment 29, wherein the secretion of the cytokine or the cytokine receptor-binding fragment thereof measured is greater than or equal to about the secretion of the cytokine or the cytokine receptor-binding fragment thereof by an otherwise identical nucleated cell that was cryopreserved. 33. The method of Embodiment 21, wherein the therapeutic agent comprises the exogenous RNA molecule.

Embodiment 31. The method of Embodiment 19, wherein the exogenous RNA molecule encodes a cytokine or the cytokine receptor-binding fragment thereof, a chemokine, or a combination thereof.

Embodiment 32. The method of Embodiment 19, wherein the exogenous RNA molecule encodes a cytokine or the cytokine receptor-binding fragment thereof.

Embodiment 33. The method of Embodiment 32, wherein the cytokine or cytokine receptor-binding fragment thereof comprises interleukin-12 (IL-12), interferon-α (IFN-α), interferon-β (IFN-β), interferon-γ (IFN-γ), interleukin-7 (IL-7), interleukin-21 (IL-21), tumor necrosis factor α (TNF-α), granulocyte-macrophage colony-stimulating factor (GM-CSF), interleukin-15 (IL-15), or a combination thereof.

Embodiment 34. The method of Embodiment 19, wherein the exogenous RNA molecule encodes a chemokine.

Embodiment 35. The method of Embodiment 34, wherein the chemokine comprises stromal cell-derived factor-1α (SDF1α), C-C motif chemokine ligand 2 (CCL2), C-C motif chemokine ligand 3 (CCL3), C-C motif chemokine ligand 5 (CCL5), C-C motif chemokine ligand 8 (CCL8), C-C motif chemokine ligand 1 (CCL1), CXC motif chemokine ligand 9 (CXCL9), CXC motif chemokine ligand 10 (CXCL10), C-C motif chemokine ligand 11 (CCL11), CXC motif chemokine ligand 12) CXCL12, or a combination thereof.

Embodiment 36. The method of Embodiment 1, further comprising treating a disease or a condition in the subject.

Embodiment 37. The method of Embodiment 36, wherein the disease is cancer.

Embodiment 38. The method of Embodiment 37, wherein the cancer is lung cancer, cancer metastases in lung tissue, liver cancer, or cancer metastases in liver tissue.

Embodiment 39. The method of Embodiment 37, wherein the liver cancer is hepatocellular carcinoma or cholangiocarcinoma.

Embodiment 40. The method of Embodiment 37, wherein the cancer is lung cancer.

Embodiment 41. The method of Embodiment 40, wherein the lung cancer is small cell lung cancer, non-small lung cancer, or bronchial carcinoids.

Embodiment 42. The method of Embodiment 41, wherein the lung cancer is small cell lung cancer.

Embodiment 43. The method of Embodiment 41, wherein the lung cancer is bronchial carcinoids.

Embodiment 44. The method of Embodiment 41, wherein the lung cancer is non-small cell lung cancer.

Embodiment 45. The method of Embodiment 44, wherein the non-small cell lung cancer is adenocarcinomas, squamous cell carcinomas, or large cell carcinomas.

Embodiment 46. The method of Embodiment 36, wherein the treating the disease or the condition in the subject comprises administering the fluid formulation to the subject intravenously.

Embodiment 47. The method of Embodiment 1, wherein the target cell of the subject comprises a lung cell.

Embodiment 48. The method of Embodiment 1, wherein the target cell of the subject comprises a liver cell.

Embodiment 49. A formulation, comprising: a plurality of enucleated cells formulated from a cryopreserved composition, wherein the cryopreserved composition comprises the plurality of enucleated cells that are cryopreserved, wherein at least a subset of the plurality of enucleated cells comprises a therapeutic agent and intracellular organelles sufficient to release the therapeutic agent in vivo or ex vivo in an amount that is greater than or equal to about an amount of the therapeutic agent released by otherwise identical enucleated cells that were not cryopreserved.

Embodiment 50. The formulation of Embodiment 49, wherein each enucleated cell of the plurality of enucleated cells comprises a diameter comprising less than or equal to about 70% of an average diameter of a nucleated parent cell.

Embodiment 51. The formulation of Embodiment 49, wherein each enucleated cell of the plurality of enucleated cells comprises a diameter comprising between about 1 micrometer (μm) to about 100 μm.

Embodiment 52. The formulation of Embodiment 51, wherein each enucleated cell of the plurality of enucleated cells comprises a diameter comprising between about 5 μm to about 25 μm.

Embodiment 53. The formulation of Embodiment 51, wherein each nucleated cell of the plurality of enucleated cells comprises a diameter comprising about 8 μm.

Embodiment 54. The formulation of Embodiment 49, wherein the therapeutic agent comprises a virus, an exogenous DNA molecule, an exogenous RNA molecule, an exogenous protein, or an exogenous peptide, or any combination thereof.

Embodiment 55. The formulation of Embodiment 54, wherein the therapeutic agent comprises a virus.

Embodiment 56. The formulation of Embodiment 55, wherein the virus is an adeno-associated virus (AAV), an adenovirus, a reovirus, a coxsackie virus, a retrovirus, a poxvirus, a baculovirus, or a herpes virus.

Embodiment 57. The formulation of Embodiment 55, wherein the virus comprises an oncolytic virus.

Embodiment 58. The formulation of Embodiment 57, wherein the oncolytic virus is an adenovirus delta 24, a human immunodeficiency disease, a Maraba virus, a Measles virus, a Newcastle disease virus, a poliovirus, a Seneca Valley virus, a parvovirus, a Semliki Forest virus, a Vesicular Stomatitis virus, a Sindbis virus, or a combination thereof.

Embodiment 59. The formulation of Embodiment 55, wherein the amount of the virus released in vivo or ex vivo is a measurement of viral titers in a target cell.

Embodiment 60. The formulation of Embodiment 59, wherein the viral titer measured in the target cell are greater than the viral titers measured in an otherwise identical target cell.

Embodiment 61. The formulation of Embodiment 59, wherein the viral titers measured in the target cell are equal to about the viral titers measured in an otherwise identical target cell.

Embodiment 62. The formulation of Embodiment 54, wherein the therapeutic agent comprises a cytokine or cytokine receptor-binding fragment thereof.

Embodiment 63. The formulation of Embodiment 62, wherein the amount of the cytokine or the cytokine receptor-binding fragment released in vivo or ex vivo is a measurement of the secretion of the cytokine or the cytokine receptor-binding fragment thereof from the plurality of enucleated cells.

Embodiment 64. The formulation of Embodiment 63, wherein the amount of the cytokine or the cytokine receptor-binding fragment thereof measured is greater than or equal to about the secretion of the cytokine or the cytokine receptor-binding fragment thereof by the otherwise identical enucleated cells that was not cryopreserved.

Embodiment 65. The formulation of Embodiment 64, wherein the amount of the cytokine or the cytokine receptor-binding fragment thereof measured is greater than or equal to about the secretion of the cytokine or the cytokine receptor-binding fragment thereof by the otherwise identical nucleated cells that was not cryopreserved.

Embodiment 66. The formulation of Embodiment 54, wherein the therapeutic agent comprises an exogenous RNA molecule.

Embodiment 67. The formulation of Embodiment 66, wherein the exogenous RNA molecule encodes a cytokine or the cytokine receptor-binding fragment thereof, a chemokine, or a combination thereof.

Embodiment 68. The formulation of Embodiment 66, wherein the exogenous RNA molecule encodes a cytokine or the cytokine receptor-binding fragment thereof.

Embodiment 69. The formulation of Embodiment 68, wherein the cytokine or the cytokine receptor-binding fragment thereof comprises IL-12, IFN-α, IFN-β, IFN-γ, IL-7, IL-21, TNF-α, GM-CSF, IL-15, or a combination thereof.

Embodiment 70. The formulation of Embodiment 66, wherein the exogenous RNA molecule encodes a chemokine.

Embodiment 71. The formulation of Embodiment 70, wherein the chemokine comprises SDF1α, CCL2, CCL3, CCL5, CCL8, CCL1, CXCL9, CXCL10, CCL11, CXCL12, or combination thereof.

Embodiment 72. The formulation of Embodiment 49, wherein each enucleated cell of the plurality of enucleated cells lacks a nucleus and comprise one or more structural features of a nucleated cell.

Embodiment 73. The formulation of Embodiment 72, wherein the one or more structural features comprise one or more tunneling nanotubes.

Embodiment 74. The formulation of Embodiment 49, wherein the intracellular organelles comprise a Golgi apparatus, an endoplasmic reticulum, or a combination thereof.

Embodiment 75. A pharmaceutical formulation, comprising: the formulation of Embodiments 49-74, and a pharmaceutically acceptable: excipient, diluent, or carrier.

Embodiment 76. The pharmaceutical formulation of Embodiment 75, wherein the pharmaceutical formulation is in an unit dose form.

Embodiment 77. The pharmaceutical formulation of Embodiment 75, wherein the pharmaceutical formulation is formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesicaly, intravaginally, intrarectally, orally, sublingually, transdermally, by inhalation, by inhaled nebulized form, by intraluminal-GI route, or a combination thereof, to a subject.

Embodiment 78. The pharmaceutical formulation of Embodiment 77, wherein the pharmaceutical formulation is formulated for administering intravenously.

Embodiment 79. The pharmaceutical formulation of Embodiment 75, further comprising at least one additional active agent.

Embodiment 80. The pharmaceutical formulation of Embodiment 79, wherein the at least one additional active agent comprises a cytokine, a growth factor, a hormone, an enzyme, a small molecule, a compound, or any combination thereof.

Embodiment 81. A kit, comprising: the formulation of Embodiments 49-74 or the pharmaceutical formulation of any one of Embodiment 75-80; and a container storing the formulation or the pharmaceutical formulation.

Embodiment 82. The kit of Embodiment 81, further comprising a resuspension buffer.

Embodiment 83. The kit of Embodiment 82, wherein the resuspension buffer comprises PBS.

Embodiment 84. The kit of Embodiment 82, wherein the resuspension buffer comprises sodium lactate solution.

Embodiment 85. The kit of Embodiment 81, further comprising instructions comprising a method for delivering the formulation or the pharmaceutical formulation to a target cell of a subject, wherein the method comprising: introducing the formulation or the pharmaceutical formulation to the target cell of a subject under conditions sufficient to deliver the therapeutic agent to the target cell in vivo or ex vivo.

Embodiment 86. The kit of Embodiment 85, wherein the method further comprising treating a disease or a condition of the subject by administering the therapeutic agent to the target cell of the subject in vivo.

Embodiment 87. The kit of Embodiment 86, wherein the disease or the condition comprises cancer.

Embodiment 88. The kit of Embodiment 87, wherein the cancer is lung cancer, cancer metastases in lung tissue, liver cancer, or cancer metastases in liver tissue.

Embodiment 89. The kit of any one of Embodiments 85-88, wherein the introducing the formulation or the pharmaceutical formulation to the target cell of a subject comprises administering the formulation or the pharmaceutical formulation to the subject intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesicaly, intravaginally, intrarectally, orally, sublingually, transdermally, by inhalation, by inhaled nebulized form, by intraluminal-GI route, or a combination thereof.

Embodiment 90. The kit of any one of Embodiment 81-89, further comprising at least one additional active agent, wherein the at least one additional active agent comprises a cytokine, a growth factor, a hormone, an enzyme, a small molecule, a compound, or any combination thereof.

### EXAMPLES

The following illustrative examples are representative of embodiments of the stimulation, systems, and methods described herein and are not meant to be limiting in any way.

### Example 1. Manufacturing of Enucleated Cells

### Preparation of 2×MEM

2×MEM was prepared by mixing 10 mL of 10×MEM (Gibco, 11430-030, 2.94 mL of sodium bicarbonate (7.5%, Gibco, 25080-094), 1 mL of 100× Pen-Strep (Gibco, 15140-122), and 36 mL of ultrapure water (Invitrogen, 10977-015) for each 50 mL quantity. Then, Cytochalasin B (Sigma Aldrich, C6762) was added to the 2×MEM to a final concentration of 20 μg/mL.

### Preparation of 1×MEM

1×MEM was prepared by mixing equal amount of 2×MEM with Cytochalasin B and ultrapure water.

### Preparation of 25% Ficoll

25% Ficoll was prepared by mixing equal amounts of 2×MEM with Cytochalasin B and 50% Ficoll which was prepared by mixing equal amounts of Ficoll (in grams) and ultrapure water (in milliliters) and autoclaving. Refraction measured was between 1.3790-1.3810.

### Preparation of Ficoll Gradient

The Ficoll gradient and Ficoll fraction were made according to Table 1 and Table 2 and incubated overnight at 37° C., 5% CO₂ with loosen caps. (For 200 mL: 240 mL of 50% Ficoll, 400 mL of 2×MEM, 300 mL of 1×MEM).

TABLE 1
Non-limiting example of Ficoll gradient and volume for
enucleating the cells with the method described herein

| | 25% | 17% | 16% | 15% | 12.5% | Total |
|---|---|---|---|---|---|---|
| 25% Ficoll | 150 | 102 | 64 | 60 | 90 | 466 |
| 1X MEM | 0 | 48 | 36 | 40 | 90 | 214 |
| Total | 150 | 150 | 100 | 100 | 180 | |

TABLE 2
Non-limiting example of Ficoll fraction for enucleating
the cells with the method described herein

| | | Fractionation volume | |
|---|---|---|---|
| Fractionation order | Layer | [tube#] | |
| 1 | 25% | 35.7 | [1-8] |
| 2 | 17% | 35.7 | [8-15] |
| 3 | 16% | 8.93 | [15-16] |
| 4 | 15% | 8.93 | [16-17] |
| 5 | 12.5% | 35.7 | [17-25] |
| 6 | 12.5% with cells | 57.14 | [25-37] |
| 7 | 1X MEM | 17.86 | [37-40] |

### Enucleation of Mesenchymal Stromal Cells (MSCs)

One day before enucleating, MSCs were seeded at density of 2.95×10² per T75 flask to reach ideal cell density (0.0168×10²/cm²). CC40NX ultracentrifuge was loaded with 200 mL of 1×MEM (without Cytochalasin B), vacuumed, and warmed to 31° C. overnight. On the day of enucleation, cells were washed with PBS and detached with trypsin. The cells were resuspended with PBS buffer and glucose until enucleation. Right before enucleation, the cells were centrifuged in 300 g for 5 min, resuspended in 65 mL of 12.5% Ficoll, mixed well, and transferred through a 40 μm strainer into a fresh 50 mL tube.

Loading of the ultracentrifuge tubes was done with a peristaltic (10 rpm) in the following order: (1) 17.86 mL of 1×MEM; (2) cells in 57.14 mL of 12.5% Ficoll; (3) 35.71 mL of 12.5% Ficoll; (4) 8.93 mL of 15% Ficoll; (5) 8.93 mL of 16% Ficoll; (6) 35.71 mL of 17% Ficoll; and 35.71 mL of 25% Ficoll.

Centrifugation was done with a 30 min acceleration to a maximum speed of 36,000 rpm for another 30 min and minimal deceleration. When centrifugation was done, 200 mL was fractioned into 15 ml tubes containing 5 mL each. The density could be measured based on both Ficoll density (g/cm³) or BRIX (% of 1 g of sugar such as Ficoll per 100 g of aqueous solution).

Fractions containing enucleated cells were merged into one 50 mL tube, span down at 300 g for 6 min, and washed with warm serum-free αMEM. 35 mL was removed, and cells were resuspended again with serum-free αMEM. Then, cell viability, size, and enucleation efficiency were measured.

### Measurement of Cell Size and Viability

10 μL of cell suspension wee diluted with 10 μL of Trypan Blue solution (0.4%) and mixed well. 10 μL of the mixture was loaded onto a cell counting slide. An automated cell counter was used to calculate cell size and viability.

### Measuring Enucleated Efficiency

50 μL of cell suspension was pipetted in a 96 well plate. It was stained with 1:2000 dilution of Hoechst 33342 (stains nuclei) and 1:500 dilution of Calcein AM and incubated at 37° C. for 5 min. Images of the cells were collected under a fluorescent microscope using the DAPI and FITC channels.

### Example 2. Generating Enucleated Cells by Zonal Centrifugation

### Preparation of 2×MEM

2× Modified Eagle Medium (MEM) is prepared by mixing 200 mL of 10×MEM (Gibco, 11430-030), 58.8 mL of sodium bicarbonate (7.5%, Gibco, 25080-094), 20 mL of 100×Pen-Strep (Gibco, 15140-122), and 720 mL of ultrapure water (Invitrogen, 10977-015) for each 1 L quantity. Then, 2×MEM is filtered through a 0.22 μm filter. 50 mL aliquots are made and stored at 4° C. for one month or until sedimentation is noticed.

### Preparation of Cytochalasin B Working Solution

One day before enucleation, 10 mg of Cytochalasin B (Sigma Aldrich, C6762) powder is dissolved in 1 mL of DMSO to generate a 10 mg/mL stock. 100 μL aliquots is prepared and stored at −20° C. until use. 100 μL of 10 mg/mL Cytochalasin B stock is added to 400 μL of DMSO to generate a 2 mg/mL working solution. Leftovers of the working solution can be stored at 4° C. for up to 2 weeks. 6 ml of 2 mg/mL of Cytochalasin B working solution is added to 600 mL of 2×MEM, creating a final solution containing 20 μL/mL of Cytochalasin B.

Preparation of 1×MEM without Cytochalasin B

1×MEM without Cytochalasin B is prepared in a 2 L bottle by adding 900 mL of 2×MEM without Cytochalasin B to 900 mL of ultrapure water.

Preparation of 1×MEM with Cytochalasin B

1×MEM with Cytochalasin B is prepared in a 1 L bottle by adding 270 mL of the final solution containing 20 μL/mL of Cytochalasin B to 270 mL of ultrapure water to generate a 1×MEM with Cytochalasin B working solution containing 10 μL/mL of Cytochalasin B.

### Preparation of 25% Ficoll

50% Ficoll is prepared by mixing equal amounts of Ficoll (in grams) and ultrapure water (in milliliters). 25% Ficoll is prepared in a 50 mL conical by mixing 520 mL of 2×MEM with Cytochalasin B and 520 mL of 50% Ficoll, creating a Ficoll working solution containing 10 μg/mL of Cytochalasin B. 25% Ficoll is vortexed for 1 min, then allow it to rest until bubbles dissipate. 70 μL of the 25% Ficoll is used to measure the refractive index. Refraction between 1.3790-1.3810 is measured. Optimal refractive index can be achieved by adding the final solution of 2×MEM with Cytochalasin B or mixing equal amounts of 50% Ficoll and 2×MEM with Cytochalasin B.

### Preparation of Enucleation Working Solution and Gradients

While the bubbles dissipate, 1×MEM with Cytochalasin B is added to 4 500 mL sterile bottles labeled 17%, 16%, 15%, and 12.5%, 102.4 mL of 1×MEM with Cytochalasin B is added to the 17% bottle; 28.8 mL of 1×MEM with Cytochalasin B is added to the 16% bottle; 32 mL of 1×MEM with Cytochalasin B is added to the 15% bottle; and 400 mL of 1×MEM with Cytochalasin B is added to the 12.5% bottle.

25% Ficoll is added to the 4 500 mL sterile bottles labeled 17% Ficoll, 16% Ficoll, 15% Ficoll, and 12.5% Ficoll. 217.6 mL of 25% Ficoll is added to the 17% bottle, 51.2 mL of 25% Ficoll is added to the 16% bottle, 48 mL of 25% Ficoll is added to the 15% bottle, and 400 mL of 25% Ficoll is added to the 12.5% bottle to make 17%, 16%, 15%, and 12.5% Ficoll layers.

Sterile bottles labeled 17%, 16%, 15%, and 12.5% with loosen caps are incubated overnight at 37° C. in a 5% CO₂ incubator.

### Enucleation of Cells

On the day of enucleation, the ultracentrifuge is preheated to 31° C. Serum-free αMEM is preheated in a 37° C. water bath. Cells detached from cell culturing surface are resuspend and mixed in 5 mL of 12.5% Ficoll. 452.6 mL of 12.5% Ficoll is added to the cell mixture for a total of 457.6 mL. Cells are strained through a 40 μM strainer. Cells are incubated for 30 minutes in a 37° C. incubator. To prepare Zonal centrifuge for acceleration to Zonal speed of 10000 relative centrifugal force (RCF), the rotor is filled with 1.6 L of 1×MEM using a peristaltic pump. After 30 minutes of incubation of cells, the layers are loaded into the centrifuge in the following order while the speed of the peristaltic pump is minimal: 457.6 mL of 12.5% Ficoll with cells; 286 mL of 12.5% Ficoll without cells; 71.5 mL of 15% Ficoll; 71.5 mL of 16% Ficoll; and 286 mL of 17% Ficoll, 286 mL of 25% Ficoll. The seal assembly is moved and capped and ran at 102000 RCF for one hour with the acceleration and deceleration set to minimum.

Once the run time is achieved, the rotor is decelerated to zonal speed (10000 RCF). The cap is removed and connected to the seal assembly. The extrusion solution is pushed from the outer wall and is collected in 10 mL or 50 mL fractions. Fractions are listed in Table 3. Once fractions are collected, the rotor is stopped and cleaned.

TABLE 3
Non-limited example of Ficoll fraction for enucleating
the cells with the method described herein

| Layer | | Volume | Fraction # | Fraction # |
|---|---|---|---|---|
| fractionation order | Layer | [mL] | [10 ml] | [50 ml] |
| 1 | 25% | 286 | 1-29 | 1-6 |
| 2 | 17% | 572 | 29-58 | 6-12 |
| 3 | 16% | 643.5 | 58-65 | 12-13 |
| 4 | 15% | 715 | 65-72 | 13-15 |
| 5 | 12.5% | 1458.6 | 72-146 | 15-30 |

Fractions containing the enucleated cells are identified and spun down at 200 RCF for 10 minutes. The enucleated cells are washed with pre-warmed serum-free αMEM and then resuspend with pre-warmed serum-free αMEM.

The enucleated cells are counted using Trypan Blue. About 20,000 of the enucleated cells from each layer are added to a 96-well plate. PBS is added to bring the total volume to 45 μL. 1:500 diluted Hoechst containing 20 μg/mL Hoechst is diluted to an additional 1:10 when added to the enucleated cells, creating a final Hoechst concentration of 2 μg/mL.

The enucleated cells are incubated at 37° C. with 5% CO₂ for 10 minutes. The enucleated cells are imaged using a fluorescence microscope in order to determine the enucleation efficiency measured as cells without Hoechst staining (e.g., the enucleated cells). The enucleated cells can be processed for downstream experimentation or freezing.

### Example 3. Manufacturing of Enucleated Cells with Continuous Flow Centrifugation

This example illustrates enucleated cells with continuous flow centrifugation with density gradients. Cells were thawed in a 5 vial intervals: taking out 5 vials out of the −80 C incubate for 45 seconds in 37° C. water bath; adding 1 ml of warm complete αMEM dropwise to each vial; transferring to a 50 ml tube; completing to a total of 50 ml complete αMEM; spinning down at 300 RCF for 5 minutes; and repeating with the next 5 vials. Supernatant was removed, and the cell pellet was resuspended in 1 ml of PBS. All 50 ml tubes were combined into two 50 ml tubes. PBS was added to reach a total of 50 ml in each tube. The cells were counted for each tube with Tube 1:3.21M/ml×50 ml=160 M, 91%, 14.42 μm; and Tube 2:2.68M/ml×50 ml=134M, 91%, 24.4 μm for a total of 294 M (millions) cells. About 200,000 cells were kept as control. Measure the refraction index of the 25% Ficoll was determined to be 1.3794. Cytochalasin B was added to each Ficoll gradient (Stock=10 mg/ml, final concentration=10 μg/ml) based on Table 4. Cells were resuspended with 50 ml of 12.5% Ficoll with Cytochalasin B and transferred through a 100 μm strainer for continuous flow centrifugation.

TABLE 4
Preparation of Ficoll gradient

| | Volume prepared | Volume loaded | Volume Cytochalasin |
|---|---|---|---|
| Layer | in this run [ml] | into centrifuge | B added [ul] |
| 12.5 Cells | 120 | 114 | 100 |
| 12.5 | 100 | 72 | 120 |
| 15 | 50 | 18 | 50 |
| 16 | 50 | 18 | 50 |
| 17 | 100 | 70 | 100 |
| 25 | 100 | 72 | 100 |

While centrifuge was running, the control nucleated cells were seeded as indicated in the bottom 96 well plate. 96 well plate and 6 well plates were coated with Fibronectin for 1 hour at 37° C. 10 ml fractions were collected into 15 ml tubes. Fractions (with visible cells) were chosen according to Table 5. Tubes from the same layer were merged into a single 50 ml tube and supplemented with warm serum free αMEM for a total of 50 ml. Supernatant was removed to the 10 ml line and wash again with 40 ml serum free αMEM. Cells were resuspended with warm complete αMEM and counted for seeding 20,000 cells per well. Cells were allowed to attach for 2 hours at 37° C. incubator. Unseeded cells were frozen with freezing media (90% serum+10% DMSO). Cells were then fixed with paraformaldehyde for Hoechst staining and imaging. Each field image was taken using bright field (total cells) and Hoechst channels (total nucleated cells). Table 6 illustrates that the enucleated cells were smaller as compared to nucleated cells. Table 6 also shows the yield (as determined by total number of nucleated cells divided by initial cell number and expressed in percentage) and enucleation efficacies.

TABLE 5
Fractions chosen for enucleated cells

| | | Fractions | |
|---|---|---|---|
| Fraction | | Volume | |
| elution | | (ml) | |
| order | Layer | [tubes #] | |
| 1 | 25% | 71.42 | [1-8] |
| 2 | 17% | 71.42 | [8-15] |
| 3 | 16% | 17.86 | [15-16] |
| 4 | 15% | 17.86 | [16-17] |
| 5 | 12.5% | 71.42 | [17-25] |
| 6 | 12.5% cells | 114.28 | [25-37] |
| 7 | 1X MEM | 25.71 | [37-40] |

| | | | | | | | Efficiency | Total | |
|---|---|---|---|---|---|---|---|---|---|
| Fraction | | Visible | Cell | Total | | Cell | [fixed | Enucleated | |
| elution | Tubes # | pellet | concentration | cell | Viability | size | Hoechst | cell | Yield |
| order | merged | ? | [10\hat{ }6/ml] | number | % | [um] | cells] | [10\hat{ }6] | [%] |
| 1 | 2-7 | Yes | 0.29 | 0.29 | 14 | 13.34 | 50% | 73.35 | 24.9489795918 |
| 2 | 8-11 | No | | | | | | | |
| 3 | 12-14 | No | | | | | | | |
| 4 | 15-17 | Yes | 2.95 | 14.75 | 90 | 11.81 | 95% | | |
| 5 | 18-20 | Yes | 2.78 | 27.8 | 83 | 11.83 | 95% | | |
| 6 | 21-22 | Yes | 2.09 | 10.45 | 89 | 12.29 | 95% | | |
| 7 | 23-25 | Yes | 2.67 | 13.35 | 90 | 12.75 | 95% | | |
| 8 | 26-29 | Yes | 1.4 | 7 | 82 | 10.74 | 95% | | |
| 9 | 30-33 | Yes | 4.47 | 4.47 | 84 | 10.39 | 50% | | |
| | | | | | | | debris | | |
| 10 | 34-39 | Yes | 1.11 | 5.55 | 74 | 10.7 | 60% | | |
| | | | | | | | debris | | |

TABLE 6
Yield of obtaining enucleated cells from continuous flow centrifugation

| | Initial | Initial | Total | | | |
|---|---|---|---|---|---|---|
| | cell | nucleated | enucleated | Enucleated | | Enucleation |
| Run | number | cell size | cell | cell size | Yield | efficiency |
| type | [10\hat{ }6] | [μm] | [10\hat{ }6] | [μm] | [%] | [%] |
| 5 Ficoll | 294 | 18 | 73.3 | 12 | 24.9 | >95 |
| layers | | | | | | |
| 3 Ficoll | 196.5 | 22.8 | 24.6 | 11.1 | 12.5 | >95 |
| layers | | | | | | |
| Continuous | | | | | | |
| flow with 5 | 298.5 | 21.7 | 39.8 | 12 | 13.3 | 50 |
| Ficoll layers | | | | | | |

### Example 4: Adherence of Cryopreserved Enucleated Cells

Enucleated cells (CAs) were generated by enucleating human telomerase reverse transcriptase (hTERT)-immortalized adipose-derived mesenchymal stromal cells by density gradient centrifugation using Ficoll gradients and high-speed centrifugation.

Following enucleation, 25,000 fresh enucleated cells were plated were plated per well in fibronectin-coated 24-well plates and incubated for 24 hours at 37° C. Separately, 5-10×10⁶ enucleated cells per milliliter (CAs/mL) were suspended in 90% fetal bovine serum (FBS)+10% dimethylsulfoxide (DMSO) and aliquoted into cryovials. The cryovials were transferred to a CoolBox and placed at −80° C. for at least 24 hours prior to being placed in liquid nitrogen for cryopreservation.

Cryopreserved vials of enucleated cells were thawed in a 37° C. water bath, washed with complete media, and 25,000 thawed cryopreserved enucleated cells were plated per well in fibronectin-coated 24-well plates and incubated at 37° C. for at least 24 hours. The workflow for this experiment is depicted in FIG. 3A.

Both the fresh enucleated cells and the cryopreserved enucleated cells were imaged 24 hours post plating in the fibronectin-coated plated. The fresh enucleated cells and the cryopreserved enucleated cells showed similar adherence to the fibronectin-coated plates and similar viabilities in in vitro cultures (FIG. 3B).

### Example 5: Functional Ability of Cryopreserved Enucleated Cells In Vitro

Enucleated cells were generated by the method described in Example 4. Following enucleation, the enucleated cells were transfected with mRNA encoding mouse IL-12α and IL-12β using Lipofectamine 3000. The transfected enucleated cells were then either plated at 25,000 enucleated cells per well in fibronectin-coated plates or cryopreserved in either 90% FBS+10% DMSO or CryoStor® CS10 as described in Example 4.

The cryopreserved enucleated cells were thawed in a 37° C. water bath and plated at 25,000 cells per well in fibronectin-coated plates. The plated fresh enucleated cells and plated cryopreserved enucleated cells were incubated at 37° C. for 24 hours.

After 24 hours, the supernatant from the plated fresh enucleated cells and the plated cryopreserved enucleated cells were collected and stored at −80° C. prior to analysis. The supernatant from each culture was thawed and tested for secreted IL-12p70 using an enzyme-linked immunoassay (ELISA). The workflow for this experiment is depicted in FIG. 4A.

Analysis of supernatants found that similar levels of IL-12p70 was secreted in cultures with fresh enucleated cells (solid bar in FIG. 4B), enucleated cells cryopreserved in 90% FBS+10% DMSO (stripped bar in FIG. 4B), and enucleated cells cryopreserved in CryoStor® CS10 (dotted bar in FIG. 4B).

### Example 6: Functional Ability of Cryopreserved Enucleated Cells In Vivo

### In Vitro IL-12 Secretion

Enucleated cells were generated by the method described in Example 4. Following enucleation, the enucleated cells were transfected with mRNA encoding mouse IL-12α and IL-12β and labeled with the lipophilic dye 1, 1′-dioctadecyl-3,3,3,3′-tetramethylindodicarbocyanine, 4-chlorobenzenesulfonate salt (DiD). The labeled enucleated cells were then cryopreserved in 90% FBS+10% DMSO.

The cryopreserved enucleated cells were thawed in a 37° C. water bath. On the same day the cryopreserved enucleated cells were thawed, fresh enucleated cells were generated by the method described in Example 4. The fresh enucleated cells were also transfected with mRNA encoding mouse IL-12α and IL-12β and labeled with DiD.

Both the fresh enucleated cells and the cryopreserved enucleated cells were suspended in phosphate buffer solution (PBS) at 1×107 cells/mL. 25,000 fresh and cryopreserved enucleated cells were then plated per well in complete media in separate fibronectin-coated wells. The plated fresh and cryopreserved enucleated cells were then incubated at 37° C. for 24 hours. After 24 hours, the supernatant from the fresh and cryopreserved enucleated cells wells were collected and tested for secreted IL-12p70 as described in Example 7. When the levels of IL-12p70 in the supernatant of the fresh enucleated cell culture and the cryopreserved enucleated cell culture were measured, the cryopreserved enucleated cells secreted more IL-12p70 compared to the fresh enucleated cells (FIG. 5B).

### In Vivo IL-12 Secretion and IFN-γ Induction

1×106 fresh enucleated cells or cryopreserved enucleated cells were suspended in 100 microliters (μL) of PBS and then injected intravenously into the tail vein of eight-week-old female Balb/C mice. A subset of mice injected with fresh enucleated cells or with the cryopreserved enucleated cells were sacrificed one day, two days, or three days post-injection. Mice sacrificed on each day underwent the following analyses: quantitation of IL-12p70 and IFN-γ levels in the plasma, quantitation of IL-12p70 and IFNγ mRNA levels in the lung and liver, and evaluation of migration of DiD-labeled enucleated cells to the lung. The level of IFN-γ was measured in order to evaluate the ability of the enucleated cells to induce transcription factors of known downstream factors of IL-12 (e.g., IFN-γ, PD-L1, and CXCL9). The entire workflow for this experiment is depicted in FIG. 5A. A third group of mice which was not injected with enucleated cells was included in the experiment as a negative control.

For quantitation of IL-12p70 and IFN-γ levels in the plasma, blood was collected from the heart of the sacrificed mice and transferred to 0.5M ethylenediaminetetraacetic acid (EDTA) tubes. The blood was centrifuged at 4° C. at 13,000 revolutions per minute (rpm) for 10 minutes. Following centrifugation, the plasma was separated out and stored at −80° C. prior to analysis. The level of IL-12p70 was analyzed via ELISA.

Mice injected with cryopreserved enucleated cells had higher levels of IL-12p70 (FIG. 5C) and IFN-γ (FIG. 5D) in the plasma on day 1 and day 2 post injection compared to mice injected with fresh enucleated cells. Levels of IL-12p70 and IFN-γ in mice on day 3 post injection were similar between mice injected with fresh enucleated cells and mice injected with cryopreserved enucleated cells.

For quantitation of IL-12p70 and IFN-γ mRNA expression levels in the lungs and liver, the lungs and livers of the mice sacrificed on day 1, day 2, and day 3 were collected, and a portion of the lungs and liver were snap-frozen and stored at −80° C. prior to analysis. The frozen tissue was thawed and homogenized in TRIzol using a bead-bug homogenizer. RNA from the lung homogenate and the liver homogenate were separated using chloroform extraction. cDNA was synthesized from the RNA and pRT-PCR was performed.

In the lungs, mice injected with fresh enucleated cells had higher levels of IL-12 mRNA expression on day 1 post injection compared to mice injected with cryopreserved enucleated cells. The level of IL-12 mRNA expression on day 2 and day 3 post injection were similar between mice injected with fresh enucleated cells and mice injected with cryopreserved enucleated cells (FIG. 5E). For IFN-γ, mice injected with cryopreserved enucleated cells had a higher level of IFN-γ mRNA expression on day 1 post injection compared to mice injected with fresh enucleated cells. The levels of IFN-γ mRNA expression on day 2 and day 3 post injection were similar between mice injected with fresh enucleated cells and mice injected with cryopreserved enucleated cells (FIG. 5F).

In the liver, mice injected with cryopreserved enucleated cells had higher levels of IL-12 mRNA expression on day 1 post injection compared to mice injected with fresh enucleated cells. The level of IL-12 mRNA expression on day 2 and day 3 post injection were similar between mice injected with fresh enucleated cells and mice injected with cryopreserved enucleated cells (FIG. 5G). For IFN-γ, mice injected with fresh enucleated cells had higher levels of IFN-γ mRNA expression on day 1 and day 2 post injection compared to mice injected with cryopreserved enucleated cells. The level of IFN-γ mRNA expression on day 3 post injection was similar between mice injected with fresh enucleated cells and mice injected with cryopreserved enucleated cells (FIG. 5H).

For evaluation of the migration of enucleated cells, lungs of mice sacrificed on day 1, day 2, and day 3 were collected and digested in PBS+Ca2++Mg2+ with 0.5 mg/mL collagenase and 5 U/mL DNase I. The lung digest was mechanically separated into single cells and subsequently analyzed on a FACS Canto cytometry for DiD labeling.

Mice injected with cryopreserved enucleated cells had a higher number of DiD-labeled enucleated cells in the lungs at day 1 and day 3 post injection compared to mice injected with fresh enucleated cells (FIG. 5I). The number of DiD-labeled enucleated cells in the lungs on day 2 was similar between mice injected with fresh enucleated cells and mice injected cryopreserved enucleated cells (FIG. 5I).

Overall, the secretion of IL-12, the induction of downstream factors of IL-12, and the migration in vivo of cryopreserved enucleated cells were similar to fresh enucleated cells.

### Example 7: Enucleated Cells Delivery of Oncolytic Virus

Enucleated cells were generated by the method described in Example 4. Following enucleation, the enucleated cells were infected with Vesicular Stomatitis Virus (VSV) encoding a mouse IFN-β mRNA at a multiplicity of infection (MOI) of 1.0. Following infection, the VSV-infected enucleated cells were cryopreserved.

Following cryopreservation, the cryopreserved enucleated cells were thawed. On the same day the cryopreserved enucleated cells were thawed, a fresh batch of enucleated cells were generated and infected with VSV encoding a mouse IFN-β mRNA at a MOI of 1.0.

### In Vivo Analysis

1×106 fresh VSV-infected enucleated cells or cryopreserved VSV-infected enucleated cells were injected intravenously into the tail vein of mice. 16 hours post injection, the mice were sacrificed and the VSV titers from lung homogenates were quantitated. The workflow for this experiment is depicted in FIG. 6A.

The lungs of mice injected with fresh enucleated cells and cryopreserved enucleated cells were harvested and homogenized. The lung homogenates were then used for a plaque assay. The lungs from mice injected with cryopreserved enucleated cells had similar titers of plaque-forming units (PFU) per gram of lung tissue as mice injected with fresh enucleated cells (FIG. 6B).

### In Vitro Analysis

Fresh and cryopreserved VSV-infected enucleated cells were plated in vitro and cultured for 48 hours. After 48 hours, the supernatants of the fresh enucleated cell culture and the cryopreserved enucleated cell culture were collected and used for a plaque assay. The cultures from mice injected with cryopreserved enucleated cells had similar titers of PFU per mL as the cultures from mice injected with fresh enucleated cells (FIG. 6C).

### Example 8. Therapeutic Efficacy of Previously Frozen Enucleated Cell

The vitality and therapeutic efficacy of the previously frozen enucleated cell was assessed. Human Wharton's Jelly MSCs and umbilical cord cells were transfected for expressing IL-12 and enucleated.

For the cell enucleation, the cells were gown to 80-90% confluency based on the cell type. Then, Ficoll gradients were prepared from 50% Ficoll as shown in Table 7. The 50% Ficoll, frozen or cryopreserved, was thawed in a heated 25° C. water bath for at least 10 minutes until the 50% Ficoll was completely thawed.

TABLE 7
Ficoll gradient composition

| | Volume of | Volume of | Volume of | Volume of |
|---|---|---|---|---|
| Number | 50% Ficoll | 2X MEM | 1X MEM | 25% Ficoll |
| of tubes | [ml] | [ml] | [ml] | [ml] |
| 1 | 30 | 55 | 50 | 60 |
| 2 | 60 | 120 | 100 | 120 |
| 3 | 84 | 160 | 140 | 168 |
| 4 | 108 | 210 | 180 | 216 |
| 5 | 132 | 250 | 220 | 264 |
| 6 | 156 | 300 | 260 | 312 |

2×MEM from the 4° C. refrigerator was removed and placed in 25° C. water-bath for at least 15 minutes. Cytochalasin B aliquots (10 mg/ml) were also thawed at room temperature. 120 μl Cytochalasin B (10 mg/ml) was transferred into a fresh Eppendorf with 480 μl of DMSO. The Cytochalasin B solution was added to the 2×MEM for a final concentration of 20 μg/ml (100 μl of 2 mg/ml solution per 9.9 ml of 2×MEM).

To prepare 1×MEM, equal volumes of ultra-pure water and 2×MEM were combined. To prepare 25% Ficoll, equal volume of 2×MEM With Cytochalasin B and 50% Ficoll were mixed by vortex for 30 seconds at 3000 rpm. To prepare additional Ficoll gradient, the 2×MEM and 50% Ficoll were mixed to generate the gradients as shown in Table 7. The quantity of the Ficoll gradient was assessed by refraction index to determine the correction centration of the Ficoll gradient. The Ficoll gradient could then be overlaid with the cells for enucleation. Table 8 illustrates an example of the number of tubes (e.g., Ultraclear tube) and their respective Ficoll gradients subjected to the centrifugation. First layer of the Ficoll gradient loaded in the tube was 25% Ficoll at 14.25 ml. Second layer of the Ficoll gradient in the tube was 17% Ficoll at 14.25 ml. Third layer of the Ficoll gradient in the tube was 16% Ficoll at 3.56 ml. Fourth layer of the Ficoll gradient in the tube was 15% Ficoll at 3.56 ml. Fifth layer of the Ficoll gradient in the tube was 12.5% Ficoll at 14.25 ml.

TABLE 8
Ficoll gradient layer

| Number of tubes | 25% | 17% | 16% | 15% | 12.5% |
|---|---|---|---|---|---|
| 1 | 16 | 10.2 | 3.2 | 3 | 20 |
| 2 | 32 | 20.4 | 6.4 | 6 | 40 |
| 3 | 46 | 29.3 | 9.2 | 8.6 | 57.5 |
| 4 | 60 | 38.25 | 12 | 11.25 | 75 |
| 5 | 75 | 47.81 | 15 | 14.06 | 93.75 |
| 6 | 90 | 57.375 | 18 | 16.875 | 112.5 |

After the Ficoll layers were loaded, the top of each Ultraclear tube was covered with parafilm. Each Ficoll layer was marked with a marker and incubated overnight at 37° C. in CO₂ incubator together with the 1×MEM, 12.5% Ficoll, any excess 25% Ficoll, and 2×MEM with loosened caps. Centrifuge rotor (e.g., Type 45Ti rotor and buckets) were placed in the 37° C. incubator without CO₂.

Cells (84-177×10⁶ MSC's per Ficoll Gradient Tube) were collected and resuspended with PBS to bring the concentration of the cells between 1-5×106 ml for determining the number and viability of the cells with Trypan blue assay. About 84-177 C 10⁶ cells were loaded into a 50 ml conical tube. The cells were washed and resuspended with 12.5% Ficoll. The cells were then strained drop-wise through a strainer into a new 50 ml conical tube. The number and viability of the cells that passed through the straining was determined again with Trypan blue assay.

Using a 3.2 ml syringe with 21G size needle, 3 ml of 12.5% Ficoll with cells was collected and overlaid on top of the Ficoll in the Ultraclear tube with the bevel against the side of the tube while the needle was about 1 cm above the liquid surface. Then, slowly release the 12.5% Ficoll with cells was released at a rate of 1 ml per 15 seconds. For each tube, a total of 6 ml of 12.5% Ficoll with cells was added). After loading the cells, 10.35 ml to a of 16.35 of 1×MEM was added to each tube to balance the weight of each tube to within +/−0.05 g for the centrifugation. The tubes containing the cells were then centrifuged in the ultracentrifuge for 33,000 rpm (or 85,000 RCF) for 60 minutes, acceleration to 7 and deceleration to 7.

Once the ultracentrifuge run was completed, each tube four layers: top of the 12.5% having some cells and a lot of cell debris; 12.5/15% interface having high number of enucleated cells; 17%-high or low number of enucleated cells; and bottom of 25% having nucleated cells and free nuclei. The layers having the enucleated cells were collected, washed, and pelleted in a conical tube. The enucleated cells were then resuspended for additional assay and for Hoechst assay to determine the enucleation efficiency. The enucleated cells were also cultured and allowed to attach to cell culture dish or plates for additional assays.

The enucleated cells were then either: directly assessed for their capability for secreting IL-12 to exert therapeutic efficacy; or first cryopreserved, thawed, and then assessed for the IL-12 secretion for exerting therapeutic efficacy. For in vitro IL-12 secretion assay, nucleated and enucleated MSCs (human Wharton's Jelly or human umbilical cord cells) were resuspended in antibiotic free media to 1×10⁶ cells/ml. Then, a total of 1 μg of mouse IL-12 mRNA was mixed together with MessengerMAX in OptiMEM according to manufacturer's protocol and incubated for five minutes at room temperature. The mRNA-MessengerMAX mixture was added to the cells and incubated for an additional 30 minutes in 37° C., 5% CO₂ incubator. Cells were washed once and seeded at 25,000 cells per well in a 24-well. The conditioned media was collected every 24 hours and analyzed.

For measurements of IL-12 expression and secretion by the enucleated cells, ELISA was used. Nucleated or enucleated mesenchymal stem cell cells (MSCs) were resuspended in antibiotic free media to 1×10⁶ cells/ml. Then, a total of 1 μg of mouse IL-12 mRNA was mixed together with MessengerMAX (ThermoFisher, cat #LMRNA015) in OptiMEM (Fisher Scientific, Cat #31985062) according to manufacturer's protocol and incubated for five minutes in room temperature. The mRNA-MessengerMAX mixture was added to the cells and incubated for an additional 30 minutes in 37° C., 5% CO₂ incubator. Cells were washed once and seeded at 25,000 cells per well in a 24-well plate. The conditioned media was collected every 24 hours and analyzed with a Biolegend ELISA (Cat #433604). Absorbance was measured using Tecan plate reader at 450 nm and 570 nm. n=3. FIG. 7A illustrates human Wharton's Jelly MSCs transfected with mouse IL-12 mRNA and seeded as fresh or after freezing and thawing process. The secreted mouse IL-12 was analyzed from the conditioned media of each condition by ELISA. n=3. “Nucleated cell fresh” denotes nucleated human Wharton's Jelly MSCs without cryopreserved before IL-12 secretion measurement. “Nucleated cell frozen” denotes nucleated human Wharton's Jelly MSCs cryopreserved and thawed before IL-12 secretion measurement. “Enucleated cell fresh” denotes enucleated human Wharton's Jelly MSCs without cryopreserved before IL-12 secretion measurement. “Enucleated cell frozen” denotes enucleated human Wharton's Jelly MSCs cryopreserved and thawed before IL-12 secretion measurement. As shown here, there was no significant difference in the amount of IL-12 secreted by the Wharton's Jelly MSCs (nucleated or enucleated) before or after cryopreservation. FIG. 7B illustrates human umbilical cord MSCs transfected with mouse IL-12 mRNA and seeded as fresh or after freezing and thawing process. The secreted mouse IL-12 was analyzed from the conditioned media of each condition by ELISA. n=3. “Nucleated cell fresh” denotes nucleated human umbilical cord MSCs without cryopreserved before IL-12 secretion measurement. “Enucleated cell fresh” denotes enucleated human umbilical cord MSCs without cryopreserved before IL-12 secretion measurement. “Nucleated cell frozen” denotes nucleated human umbilical cord MSCs without cryopreserved before IL-12 secretion measurement. “Enucleated cell frozen” denotes enucleated human umbilical cord MSCs cryopreserved and thawed for IL-12 secretion measurement. As shown here, there was no significant difference in the amount of IL-12 secreted by umbilical cord MSCs (nucleated or enucleated) before or after cryopreservation.

For the in vivo therapeutic efficacy assessment, C57BL/6 mice were inoculated subcutaneously with 1×10⁶ E0771 tumor cells. After 12 days, the mice were stratified to therapeutic groups by tumor volume. Every 3 days the mice were treated intratumorally (i.t.) with cryopreserved human bone marrow enucleated cells transfected or not transfected with mouse IL-12 mRNA alongside with i.p injections of PD-1 antibody. Tumor volumes were measured three times a week, and mice were sacrificed if not passing health criteria. FIG. 8 illustrate the in vivo assessment of therapeutic efficacy stemmed from secretion of IL-12 by the enucleated cell. The mice were injected with PD-1 antibody as part of the treatment. “CA” and “Ab” denote the administration of the enucleated cells described herein and the PD-1 antibody respectively. FIG. 8A and FIG. 8B illustrate four groups of C57BL/6 mice inoculated subcutaneously with 1×10⁶ EO771 tumor cells. After 12 days, the mice were stratified by tumor volume. Group 1 mice were injected: intratumorally (i.t.) with the freezing media containing 5% DMSO; and intraperitoneally (i.p.) with immunoglobulin G (IgG). Group 2 mice were injected: intratumorally (i.t.) with enucleated human bone marrow cells; and intraperitoneally (i.p.) with PD-1 antibody. Group 3 mice were injected: intratumorally (i.t.) with enucleated human bone marrow cells expressing IL-12; and intraperitoneally (i.p.) with IgG. Group 4 mice were injected: intratumorally (i.t.) with enucleated human bone marrow cells expressing IL-12; and intraperitoneally (i.p.) with PD-1 antibody. n indicated the number of mice for each group. Every 3 days the mice were treated intratumorally (i.t.) with cryopreserved human bone marrow enucleated cell transfected or not transfected with mouse IL-12 mRNA alongside intraperitoneal (i.p.) injections of anti PD-1 antibody. FIG. 8A: Tumor volumes were measured 3 times a week and mice were sacrificed if not passing health criteria. FIG. 8B: Kaplan-Meier curve of the same mice as in FIG. 8A. As shown in FIG. 8A and FIG. 8B, Group 3 and Group 4 mice (particularly Group 4) exhibited the most pronounced tumor volume decrease and survival increase.

While the foregoing disclosure has been described in some detail for purposes of clarity and understanding, it will be clear to one skilled in the art from a reading of this disclosure that various changes in form and detail may be made without departing from the true scope of the disclosure. For example, all the techniques and apparatus described above may be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

SEQUENCE LISTING

| SEQ ID | | Type of | | |
|---|---|---|---|---|
| NO | Sequence | Sequence | Organism | Name |
| 1 | MRHIICHGGVITEEMAASLLDQLIEEVL | Protein | Adenovirus | E1A |
| | ADNLPPPSHFEPPTLHELYDLDVTAPED | | | protein |
| | PNEEAVSQIFPDSVMLAVQEGIDLLTFPP | | | |
| | APGSPEPPHLSRQPEQPEQRALGPVSMP | | | |
| | NLVPEVIDLTCHEAGFPPSDDEDEEGEE | | | |
| | FVLDYVEHPGHGCRSCHYHRRNTGDPD | | | |
| | IMCSLCYMRTCGMFVYSK | | | |
| 2 | ATGAGACATATTATCTGCCACGGAGG | DNA | Adenovirus | E1A |
| | TGTTATTACCGAAGAAATGGCCGCCA | | | gene |
| | GTCTTTTGGACCAGCTGATCGAAGAG | | | |
| | GTACTGGCTGATAATCTTCCACCTCCT | | | |
| | AGCCATTTTGAACCACCTACCCTTCAC | | | |
| | GAACTGTATGATTTAGACGTGACGGC | | | |
| | CCCCGAAGATCCCAACGAGGAGGCGG | | | |
| | TTTCGCAGATTTTTCCCGACTCTGTAA | | | |
| | TGTTGGCGGTGCAGGAAGGGATTGAC | | | |
| | TTACTCACTTTTCCGCCGGCGCCCGGT | | | |
| | TCTCCGGAGCCGCCTCACCTTTCCCGG | | | |
| | CAGCCCGAGCAGCCGGAGCAGAGAG | | | |
| | CCTTGGGTCCGGTTTCTATGCCAAACC | | | |
| | TTGTACCGGAGGTGATCGATCTTACCT | | | |
| | GCCACGAGGCTGGCTTTCCACCCAGT | | | |
| | GACGACGAGGATGAAGAGGGTGAGG | | | |
| | AGTTTGTGTTAGATTATGTGGAGCACC | | | |
| | CCGGGCACGGTTGCAGGTCTTGTCATT | | | |
| | ATCACCGGAGGAATACGGGGGACCCA | | | |
| | GATATTATGTGTTCGCTTTGCTATATG | | | |
| | AGGACCTGTGGCATGTTTGTCTACAGT | | | |
| | AAGTGA | | | |
| 3 | CTTACCTGCCACGAGGCTGGCTTT | DNA | Adenovirus | E1A |
| | | | | gene |
| | | | | deletion |
| 4 | LTCHEAGF | Protein | Adenovirus | E1A |
| | | | | protein |
| 5 | CDCRGDCFC | Protein | Adenovirus | RGD- |
| | | | | 4C |
| | | | | peptide |
| 6 | CATCATCAATAATATACCTTATTTTGG | DNA | Adenovirus | Human |
| | ATTGAAGCCAATATGATAATGAGGGG | | | adenovirus |
| | GTGGAGTTTGTGACGTGGCGCGGGGC | | | 5 |
| | GTGGGAACGGGGGGGGTGACGTAGTA | | | |
| | GTGTGGCGGAAGTGTGATGTTGCAAG | | | |
| | TGTGGCGGAACACATGTAAGCGACGG | | | |
| | ATGTGGCAAAAGTGACGTTTTTGGTG | | | |
| | TGCGCCGGTGTACACAGGAAGTGACA | | | |
| | ATTTTCGCGCGGTTTTAGGCGGATGTT | | | |
| | GTAGTAAATTTGGGCGTAACCGAGTA | | | |
| | AGATTTGGCCATTTTCGCGGGAAAAC | | | |
| | TGAATAAGAGGAAGTGAAATCTGAAT | | | |
| | AATTTTGTGTTACTCATAGCGCGTAAT | | | |
| | ATTTGTCTAGGGCCGCGGGGACTTTG | | | |
| | ACCGTTTACGTGGAGACTCGCCCAGG | | | |
| | TGTTTTTCTCAGGTGTTTTCCGCGTTC | | | |
| | CGGGTCAAAGTTGGCGTTTTATTATTA | | | |
| | TAGTCAGCTGACGTGTAGTGTATTTAT | | | |
| | ACCCGGTGAGTTCCTCAAGAGGCCAC | | | |
| | TCTTGAGTGCCAGCGAGTAGAGTTTTC | | | |
| | TCCTCCGAGCCGCTCCGACACCGGGA | | | |
| | CTGAAAATGAGACATATTATCTGCCA | | | |
| | CGGAGGTGTTATTACCGAAGAAATGG | | | |
| | CCGCCAGTCTTTTGGACCAGCTGATCG | | | |
| | AAGAGGTACTGGCTGATAATCTTCCA | | | |
| | CCTCCTAGCCATTTTGAACCACCTACC | | | |
| | CTTCACGAACTGTATGATTTAGACGTG | | | |
| | ACGGCCCCCGAAGATCCCAACGAGGA | | | |
| | GGCGGTTTCGCAGATTTTTCCCGACTC | | | |
| | TGTAATGTTGGCGGTGCAGGAAGGGA | | | |
| | TTGACTTACTCACTTTTCCGCCGGCGC | | | |
| | CCGGTTCTCCGGAGCCGCCTCACCTTT | | | |
| | CCCGGCAGCCCGAGCAGCCGGAGCAG | | | |
| | AGAGCCTTGGGTCCGGTTTCTATGCCA | | | |
| | AACCTTGTACCGGAGGTGATCGATCT | | | |
| | TACCTGCCACGAGGCTGGCTTTCCACC | | | |
| | CAGTGACGACGAGGATGAAGAGGGT | | | |
| | GAGGAGTTTGTGTTAGATTATGTGGA | | | |
| | GCACCCCGGGCACGGTTGCAGGTCTT | | | |
| | GTCATTATCACCGGAGGAATACGGGG | | | |
| | GACCCAGATATTATGTGTTCGCTTTGC | | | |
| | TATATGAGGACCTGTGGCATGTTTGTC | | | |
| | TACAGTAAGTGAAAATTATGGGCAGT | | | |
| | GGGTGATAGAGTGGTGGGTTTGGTGT | | | |
| | GGTAATTTTTTTTTTAATTTTTACAGTT | | | |
| | TTGTGGTTTAAAGAATTTTGTATTGTG | | | |
| | ATTTTTTTAAAAGGTCCTGTGTCTGAA | | | |
| | CCTGAGCCTGAGCCCGAGCCAGAACC | | | |
| | GGAGCCTGCAAGACCTACCCGCCGTC | | | |
| | CTAAAATGGCGCCTGCTATCCTGAGA | | | |
| | CGCCCGACATCACCTGTGTCTAGAGA | | | |
| | ATGCAATAGTAGTACGGATAGCTGTG | | | |
| | ACTCCGGTCCTTCTAACACACCTCCTG | | | |
| | AGATACACCCGGTGGTCCCGCTGTGC | | | |
| | CCCATTAAACCAGTTGCCGTGAGAGT | | | |
| | TGGTGGGCGTCGCCAGGCTGTGGAAT | | | |
| | GTATCGAGGACTTGCTTAACGAGCCT | | | |
| | GGGCAACCTTTGGACTTGAGCTGTAA | | | |
| | ACGCCCCAGGCCATAAGGTGTAAACC | | | |
| | TGTGATTGCGTGTGTGGTTAACGCCTT | | | |
| | TGTTTGCTGAATGAGTTGATGTAAGTT | | | |
| | TAATAAAGGGTGAGATAATGTTTAAC | | | |
| | TTGCATGGCGTGTTAAATGGGGGGGG | | | |
| | GCTTAAAGGGTATATAATGCGCCGTG | | | |
| | GGCTAATCTTGGTTACATCTGACCTCA | | | |
| | TGGAGGCTTGGGAGTGTTTGGAAGAT | | | |
| | TTTTCTGCTGTGCGTAACTTGCTGGAA | | | |
| | CAGAGCTCTAACAGTACCTCTTGGTTT | | | |
| | TGGAGGTTTCTGTGGGGCTCATCCCA | | | |
| | GGCAAAGTTAGTCTGCAGAATTAAGG | | | |
| | AGGATTACAAGTGGGAATTTGAAGAG | | | |
| | CTTTTGAAATCCTGTGGTGAGCTGTTT | | | |
| | GATTCTTTGAATCTGGGTCACCAGGC | | | |
| | GCTTTTCCAAGAGAAGGTCATCAAGA | | | |
| | CTTTGGATTTTTCCACACCGGGGCGCG | | | |
| | CTGCGGCTGCTGTTGCTTTTTTGAGTT | | | |
| | TTATAAAGGATAAATGGAGCGAAGAA | | | |
| | ACCCATCTGAGCGGGGGGTACCTGCT | | | |
| | GGATTTTCTGGCCATGCATCTGTGGAG | | | |
| | AGCGGTTGTGAGACACAAGAATCGCC | | | |
| | TGCTACTGTTGTCTTCCGTCCGCCCGG | | | |
| | CGATAATACCGACGGAGGAGCAGCAG | | | |
| | CAGCAGCAGGAGGAAGCCAGGCGGC | | | |
| | GGCGGCAGGAGCAGAGCCCATGGAA | | | |
| | CCCGAGAGCCGGCCTGGACCCTCGGG | | | |
| | AATGAATGTTGTACAGGTGGCTGAAC | | | |
| | TGTATCCAGAACTGAGACGCATTTTG | | | |
| | ACAATTACAGAGGATGGGCAGGGGCT | | | |
| | AAAGGGGGTAAAGAGGGAGCGGGGG | | | |
| | GCTTGTGAGGCTACAGAGGAGGCTAG | | | |
| | GAATCTAGCTTTTAGCTTAATGACCAG | | | |
| | ACACCGTCCTGAGTGTATTACTTTTCA | | | |
| | ACAGATCAAGGATAATTGCGCTAATG | | | |
| | AGCTTGATCTGCTGGCGCAGAAGTAT | | | |
| | TCCATAGAGCAGCTGACCACTTACTG | | | |
| | GCTGCAGCCAGGGGATGATTTTGAGG | | | |
| | AGGCTATTAGGGTATATGCAAAGGTG | | | |
| | GCACTTAGGCCAGATTGCAAGTACAA | | | |
| | GATCAGCAAACTTGTAAATATCAGGA | | | |
| | ATTGTTGCTACATTTCTGGGAACGGG | | | |
| | GCCGAGGTGGAGATAGATACGGAGG | | | |
| | ATAGGGTGGCCTTTAGATGTAGCATG | | | |
| | ATAAATATGTGGCCGGGGGTGCTTGG | | | |
| | CATGGACGGGGTGGTTATTATGAATG | | | |
| | TAAGGTTTACTGGCCCCAATTTTAGCG | | | |
| | GTACGGTTTTCCTGGCCAATACCAACC | | | |
| | TTATCCTACACGGTGTAAGCTTCTATG | | | |
| | GGTTTAACAATACCTGTGTGGAAGCC | | | |
| | TGGACCGATGTAAGGGTTCGGGGCTG | | | |
| | TGCCTTTTACTGCTGCTGGAAGGGGGT | | | |
| | GGTGTGTCGCCCCAAAAGCAGGGCTT | | | |
| | CAATTAAGAAATGCCTCTTTGAAAGG | | | |
| | TGTACCTTGGGTATCCTGTCTGAGGGT | | | |
| | AACTCCAGGGTGCGCCACAATGTGGC | | | |
| | CTCCGACTGTGGTTGCTTCATGCTAGT | | | |
| | GAAAAGCGTGGCTGTGATTAAGCATA | | | |
| | ACATGGTATGTGGCAACTGCGAGGAC | | | |
| | AGGGCCTCTCAGATGCTGACCTGCTC | | | |
| | GGACGGCAACTGTCACCTGCTGAAGA | | | |
| | CCATTCACGTAGCCAGCCACTCTCGC | | | |
| | AAGGCCTGGCCAGTGTTTGAGCATAA | | | |
| | CATACTGACCCGCTGTTCCTTGCATTT | | | |
| | GGGTAACAGGAGGGGGGTGTTCCTAC | | | |
| | CTTACCAATGCAATTTGAGTCACACTA | | | |
| | AGATATTGCTTGAGCCCGAGAGCATG | | | |
| | TCCAAGGTGAACCTGAACGGGGTGTT | | | |
| | TGACATGACCATGAAGATCTGGAAGG | | | |
| | TGCTGAGGTACGATGAGACCCGCACC | | | |
| | AGGTGCAGACCCTGCGAGTGTGGCGG | | | |
| | TAAACATATTAGGAACCAGCCTGTGA | | | |
| | TGCTGGATGTGACCGAGGAGCTGAGG | | | |
| | CCCGATCACTTGGTGCTGGCCTGCACC | | | |
| | CGCGCTGAGTTTGGCTCTAGCGATGA | | | |
| | AGATACAGATTGAGGTACTGAAATGT | | | |
| | GTGGGCGTGGCTTAAGGGTGGGAAAG | | | |
| | AATATATAAGGTGGGGGTCTTATGTA | | | |
| | GTTTTGTATCTGTTTTGCAGCAGCCGC | | | |
| | CGCCGCCATGAGCACCAACTCGTTTG | | | |
| | ATGGAAGCATTGTGAGCTCATATTTG | | | |
| | ACAACGCGCATGCCCCCATGGGCCGG | | | |
| | GGTGCGTCAGAATGTGATGGGCTCCA | | | |
| | GCATTGATGGTCGCCCCGTCCTGCCCG | | | |
| | CAAACTCTACTACCTTGACCTACGAG | | | |
| | ACCGTGTCTGGAACGCCGTTGGAGAC | | | |
| | TGCAGCCTCCGCCGCCGCTTCAGCCG | | | |
| | CTGCAGCCACCGCCCGCGGGATTGTG | | | |
| | ACTGACTTTGCTTTCCTGAGCCCGCTT | | | |
| | GCAAGCAGTGCAGCTTCCCGTTCATC | | | |
| | CGCCCGCGATGACAAGTTGACGGCTC | | | |
| | TTTTGGCACAATTGGATTCTTTGACCC | | | |
| | GGGAACTTAATGTCGTTTCTCAGCAG | | | |
| | CTGTTGGATCTGCGCCAGCAGGTTTCT | | | |
| | GCCCTGAAGGCTTCCTCCCCTCCCAAT | | | |
| | GCGGTTTAAAACATAAATAAAAAACC | | | |
| | AGACTCTGTTTGGATTTGGATCAAGC | | | |
| | AAGTGTCTTGCTGTCTTTATTTAGGGG | | | |
| | TTTTGCGCGCGCGGTAGGCCCGGGAC | | | |
| | CAGCGGTCTCGGTCGTTGAGGGTCCT | | | |
| | GTGTATTTTTTCCAGGACGTGGTAAAG | | | |
| | GTGACTCTGGATGTTCAGATACATGG | | | |
| | GCATAAGCCCGTCTCTGGGGTGGAGG | | | |
| | TAGCACCACTGCAGAGCTTCATGCTG | | | |
| | CGGGGTGGTGTTGTAGATGATCCAGT | | | |
| | CGTAGCAGGAGCGCTGGGCGTGGTGC | | | |
| | CTAAAAATGTCTTTCAGTAGCAAGCT | | | |
| | GATTGCCAGGGGCAGGCCCTTGGTGT | | | |
| | AAGTGTTTACAAAGCGGTTAAGCTGG | | | |
| | GATGGGTGCATACGTGGGGATATGAG | | | |
| | ATGCATCTTGGACTGTATTTTTAGGTT | | | |
| | GGCTATGTTCCCAGCCATATCCCTCCG | | | |
| | GGGATTCATGTTGTGCAGAACCACCA | | | |
| | GCACAGTGTATCCGGTGCACTTGGGA | | | |
| | AATTTGTCATGTAGCTTAGAAGGAAA | | | |
| | TGCGTGGAAGAACTTGGAGACGCCCT | | | |
| | TGTGACCTCCAAGATTTTCCATGCATT | | | |
| | CGTCCATAATGATGGCAATGGGCCCA | | | |
| | CGGGCGGCGGCCTGGGCGAAGATATT | | | |
| | TCTGGGATCACTAACGTCATAGTTGTG | | | |
| | TTCCAGGATGAGATCGTCATAGGCCA | | | |
| | TTTTTACAAAGCGCGGGCGGAGGGTG | | | |
| | CCAGACTGCGGTATAATGGTTCCATC | | | |
| | CGGCCCAGGGGCGTAGTTACCCTCAC | | | |
| | AGATTTGCATTTCCCACGCTTTGAGTT | | | |
| | CAGATGGGGGGATCATGTCTACCTGC | | | |
| | GGGGCGATGAAGAAAACGGTTTCCGG | | | |
| | GGTAGGGGAGATCAGCTGGGAAGAA | | | |
| | AGCAGGTTCCTGAGCAGCTGCGACTT | | | |
| | ACCGCAGCCGGTGGGCCCGTAAATCA | | | |
| | CACCTATTACCGGGTGCAACTGGTAG | | | |
| | TTAAGAGAGCTGCAGCTGCCGTCATC | | | |
| | CCTGAGCAGGGGGGCCACTTCGTTAA | | | |
| | GCATGTCCCTGACTCGCATGTTTTCCC | | | |
| | TGACCAAATCCGCCAGAAGGCGCTCG | | | |
| | CCGCCCAGCGATAGCAGTTCTTGCAA | | | |
| | GGAAGCAAAGTTTTTCAACGGTTTGA | | | |
| | GACCGTCCGCCGTAGGCATGCTTTTG | | | |
| | AGCGTTTGACCAAGCAGTTCCAGGCG | | | |
| | GTCCCACAGCTCGGTCACCTGCTCTAC | | | |
| | GGCATCTCGATCCAGCATATCTCCTCG | | | |
| | TTTCGCGGGTTGGGGCGGCTTTCGCTG | | | |
| | TACGGCAGTAGTCGGTGCTCGTCCAG | | | |
| | ACGGGCCAGGGTCATGTCTTTCCACG | | | |
| | GGCGCAGGGTCCTCGTCAGCGTAGTC | | | |
| | TGGGTCACGGTGAAGGGGTGCGCTCC | | | |
| | GGGCTGCGCGCTGGCCAGGGTGCGCT | | | |
| | TGAGGCTGGTCCTGCTGGTGCTGAAG | | | |
| | CGCTGCCGGTCTTCGCCCTGCGCGTCG | | | |
| | GCCAGGTAGCATTTGACCATGGTGTC | | | |
| | ATAGTCCAGCCCCTCCGCGGCGTGGC | | | |
| | CCTTGGCGCGCAGCTTGCCCTTGGAG | | | |
| | GAGGCGCCGCACGAGGGGCAGTGCA | | | |
| | GACTTTTGAGGGCGTAGAGCTTGGGC | | | |
| | GCGAGAAATACCGATTCCGGGGAGTA | | | |
| | GGCATCCGCGCCGCAGGCCCCGCAGA | | | |
| | CGGTCTCGCATTCCACGAGCCAGGTG | | | |
| | AGCTCTGGCCGTTCGGGGTCAAAAAC | | | |
| | CAGGTTTCCCCCATGCTTTTTGATGCG | | | |
| | TTTCTTACCTCTGGTTTCCATGAGCCG | | | |
| | GTGTCCACGCTCGGTGACGAAAAGGC | | | |
| | TGTCCGTGTCCCCGTATACAGACTTGA | | | |
| | GAGGCCTGTCCTCGAGCGGTGTTCCG | | | |
| | CGGTCCTCCTCGTATAGAAACTCGGA | | | |
| | CCACTCTGAGACAAAGGCTCGCGTCC | | | |
| | AGGCCAGCACGAAGGAGGCTAAGTG | | | |
| | GGAGGGGTAGCGGTCGTTGTCCACTA | | | |
| | GGGGGTCCACTCGCTCCAGGGTGTGA | | | |
| | AGACACATGTCGCCCTCTTCGGCATC | | | |
| | AAGGAAGGTGATTGGTTTGTAGGTGT | | | |
| | AGGCCACGTGACCGGGTGTTCCTGAA | | | |
| | GGGGGGCTATAAAAGGGGGTGGGGG | | | |
| | CGCGTTCGTCCTCACTCTCTTCCGCAT | | | |
| | CGCTGTCTGCGAGGGCCAGCTGTTGG | | | |
| | GGTGAGTACTCCCTCTGAAAAGCGGG | | | |
| | CATGACTTCTGCGCTAAGATTGTCAGT | | | |
| | TTCCAAAAACGAGGAGGATTTGATAT | | | |
| | TCACCTGGCCCGCGGTGATGCCTTTGA | | | |
| | GGGTGGCCGCATCCATCTGGTCAGAA | | | |
| | AAGACAATCTTTTTGTTGTCAAGCTTG | | | |
| | GTGGCAAACGACCCGTAGAGGGCGTT | | | |
| | GGACAGCAACTTGGCGATGGAGCGCA | | | |
| | GGGTTTGGTTTTTGTCGCGATCGGCGC | | | |
| | GCTCCTTGGCCGCGATGTTTAGCTGCA | | | |
| | CGTATTCGCGCGCAACGCACCGCCAT | | | |
| | TCGGGAAAGACGGTGGTGCGCTCGTC | | | |
| | GGGCACCAGGTGCACGCGCCAACCGC | | | |
| | GGTTGTGCAGGGTGACAAGGTCAACG | | | |
| | CTGGTGGCTACCTCTCCGCGTAGGCG | | | |
| | CTCGTTGGTCCAGCAGAGGCGGCCGC | | | |
| | CCTTGCGCGAGCAGAATGGCGGTAGG | | | |
| | GGGTCTAGCTGCGTCTCGTCCGGGGG | | | |
| | GTCTGCGTCCACGGTAAAGACCCCGG | | | |
| | GCAGCAGGCGCGCGTCGAAGTAGTCT | | | |
| | ATCTTGCATCCTTGCAAGTCTAGCGCC | | | |
| | TGCTGCCATGCGCGGGCGGCAAGCGC | | | |
| | GCGCTCGTATGGGTTGAGTGGGGGAC | | | |
| | CCCATGGCATGGGGTGGGTGAGCGCG | | | |
| | GAGGCGTACATGCCGCAAATGTCGTA | | | |
| | AACGTAGAGGGGCTCTCTGAGTATTC | | | |
| | CAAGATATGTAGGGTAGCATCTTCCA | | | |
| | CCGCGGATGCTGGCGCGCACGTAATC | | | |
| | GTATAGTTCGTGCGAGGGAGCGAGGA | | | |
| | GGTCGGGACCGAGGTTGCTACGGGCG | | | |
| | GGCTGCTCTGCTCGGAAGACTATCTG | | | |
| | CCTGAAGATGGCATGTGAGTTGGATG | | | |
| | ATATGGTTGGACGCTGGAAGACGTTG | | | |
| | AAGCTGGCGTCTGTGAGACCTACCGC | | | |
| | GTCACGCACGAAGGAGGCGTAGGAGT | | | |
| | CGCGCAGCTTGTTGACCAGCTCGGCG | | | |
| | GTGACCTGCACGTCTAGGGCGCAGTA | | | |
| | GTCCAGGGTTTCCTTGATGATGTCATA | | | |
| | CTTATCCTGTCCCTTTTTTTTCCACAGC | | | |
| | TCGCGGTTGAGGACAAACTCTTCGCG | | | |
| | GTCTTTCCAGTACTCTTGGATCGGAAA | | | |
| | CCCGTCGGCCTCCGAACGGTAAGAGC | | | |
| | CTAGCATGTAGAACTGGTTGACGGCC | | | |
| | TGGTAGGCGCAGCATCCCTTTTCTACG | | | |
| | GGTAGCGCGTATGCCTGCGCGGCCTT | | | |
| | CCGGAGCGAGGTGTGGGTGAGCGCAA | | | |
| | AGGTGTCCCTGACCATGACTTTGAGG | | | |
| | TACTGGTATTTGAAGTCAGTGTCGTCG | | | |
| | CATCCGCCCTGCTCCCAGAGCAAAAA | | | |
| | GTCCGTGCGCTTTTTGGAACGCGGATT | | | |
| | TGGCAGGGCGAAGGTGACATCGTTGA | | | |
| | AGAGTATCTTTCCCGCGCGAGGCATA | | | |
| | AAGTTGCGTGTGATGCGGAAGGGTCC | | | |
| | CGGCACCTCGGAACGGTTGTTAATTA | | | |
| | CCTGGGCGGCGAGCACGATCTCGTCA | | | |
| | AAGCCGTTGATGTTGTGGCCCACAAT | | | |
| | GTAAAGTTCCAAGAAGCGCGGGATGC | | | |
| | CCTTGATGGAAGGCAATTTTTTAAGTT | | | |
| | CCTCGTAGGTGAGCTCTTCAGGGGAG | | | |
| | CTGAGCCCGTGCTCTGAAAGGGCCCA | | | |
| | GTCTGCAAGATGAGGGTTGGAAGCGA | | | |
| | CGAATGAGCTCCACAGGTCACGGGCC | | | |
| | ATTAGCATTTGCAGGTGGTCGCGAAA | | | |
| | GGTCCTAAACTGGCGACCTATGGCCA | | | |
| | TTTTTTCTGGGGTGATGCAGTAGAAG | | | |
| | GTAAGCGGGTCTTGTTCCCAGCGGTC | | | |
| | CCATCCAAGGTTCGCGGCTAGGTCTC | | | |
| | GCGCGGCAGTCACTAGAGGCTCATCT | | | |
| | CCGCCGAACTTCATGACCAGCATGAA | | | |
| | GGGCACGAGCTGCTTCCCAAAGGCCC | | | |
| | CCATCCAAGTATAGGTCTCTACATCGT | | | |
| | AGGTGACAAAGAGACGCTCGGTGCGA | | | |
| | GGATGCGAGCCGATCGGGAAGAACTG | | | |
| | GATCTCCCGCCACCAATTGGAGGAGT | | | |
| | GGCTATTGATGTGGTGAAAGTAGAAG | | | |
| | TCCCTGCGACGGGCCGAACACTCGTG | | | |
| | CTGGCTTTTGTAAAAACGTGCGCAGT | | | |
| | ACTGGCAGCGGTGCACGGGCTGTACA | | | |
| | TCCTGCACGAGGTTGACCTGACGACC | | | |
| | GCGCACAAGGAAGCAGAGTGGGAATT | | | |
| | TGAGCCCCTCGCCTGGCGGGTTTGGCT | | | |
| | GGTGGTCTTCTACTTCGGCTGCTTGTC | | | |
| | CTTGACCGTCTGGCTGCTCGAGGGGA | | | |
| | GTTACGGTGGATCGGACCACCACGCC | | | |
| | GCGCGAGCCCAAAGTCCAGATGTCCG | | | |
| | CGCGCGGCGGTCGGAGCTTGATGACA | | | |
| | ACATCGCGCAGATGGGAGCTGTCCAT | | | |
| | GGTCTGGAGCTCCCGCGGCGTCAGGT | | | |
| | CAGGCGGGAGCTCCTGCAGGTTTACC | | | |
| | TCGCATAGACGGGTCAGGGCGCGGGC | | | |
| | TAGATCCAGGTGATACCTAATTTCCA | | | |
| | GGGGCTGGTTGGTGGCGGCGTCGATG | | | |
| | GCTTGCAAGAGGCCGCATCCCCGCGG | | | |
| | CGCGACTACGGTACCGCGCGGCGGGC | | | |
| | GGTGGGCCGCGGGGGTGTCCTTGGAT | | | |
| | GATGCATCTAAAAGCGGTGACGCGGG | | | |
| | CGAGCCCCCGGAGGTAGGGGGGGCTC | | | |
| | CGGACCCGCCGGGAGAGGGGGCAGG | | | |
| | GGCACGTCGGCGCCGCGCGCGGGCAG | | | |
| | GAGCTGGTGCTGCGCGCGTAGGTTGC | | | |
| | TGGCGAACGCGACGACGCGGCGGTTG | | | |
| | ATCTCCTGAATCTGGCGCCTCTGCGTG | | | |
| | AAGACGACGGGCCCGGTGAGCTTGAG | | | |
| | CCTGAAAGAGAGTTCGACAGAATCAA | | | |
| | TTTCGGTGTCGTTGACGGCGGCCTGGC | | | |
| | GCAAAATCTCCTGCACGTCTCCTGAGT | | | |
| | TGTCTTGATAGGCGATCTCGGCCATG | | | |
| | AACTGCTCGATCTCTTCCTCCTGGAGA | | | |
| | TCTCCGCGTCCGGCTCGCTCCACGGTG | | | |
| | GCGGCGAGGTCGTTGGAAATGCGGGC | | | |
| | CATGAGCTGCGAGAAGGCGTTGAGGC | | | |
| | CTCCCTCGTTCCAGACGCGGCTGTAG | | | |
| | ACCACGCCCCCTTCGGCATCGCGGGC | | | |
| | GCGCATGACCACCTGCGCGAGATTGA | | | |
| | GCTCCACGTGCCGGGCGAAGACGGCG | | | |
| | TAGTTTCGCAGGCGCTGAAAGAGGTA | | | |
| | GTTGAGGGTGGTGGCGGTGTGTTCTG | | | |
| | CCACGAAGAAGTACATAACCCAGCGT | | | |
| | CGCAACGTGGATTCGTTGATATCCCCC | | | |
| | AAGGCCTCAAGGCGCTCCATGGCCTC | | | |
| | GTAGAAGTCCACGGCGAAGTTGAAAA | | | |
| | ACTGGGAGTTGCGCGCCGACACGGTT | | | |
| | AACTCCTCCTCCAGAAGACGGATGAG | | | |
| | CTCGGCGACAGTGTCGCGCACCTCGC | | | |
| | GCTCAAAGGCTACAGGGGCCTCTTCT | | | |
| | TCTTCTTCAATCTCCTCTTCCATAAGG | | | |
| | GCCTCCCCTTCTTCTTCTTCTGGCGGC | | | |
| | GGTGGGGGAGGGGGGACACGGCGGC | | | |
| | GACGACGGCGCACCGGGAGGCGGTCG | | | |
| | ACAAAGCGCTCGATCATCTCCCCGCG | | | |
| | GCGACGGCGCATGGTCTCGGTGACGG | | | |
| | CGCGGCCGTTCTCGCGGGGGCGCAGT | | | |
| | TGGAAGACGCCGCCCGTCATGTCCCG | | | |
| | GTTATGGGTTGGCGGGGGGCTGCCAT | | | |
| | GCGGCAGGGATACGGCGCTAACGATG | | | |
| | CATCTCAACAATTGTTGTGTAGGTACT | | | |
| | CCGCCGCCGAGGGACCTGAGCGAGTC | | | |
| | CGCATCGACCGGATCGGAAAACCTCT | | | |
| | CGAGAAAGGCGTCTAACCAGTCACAG | | | |
| | TCGCAAGGTAGGCTGAGCACCGTGGC | | | |
| | GGGCGGCAGCGGGCGGCGGTCGGGGT | | | |
| | TGTTTCTGGCGGAGGTGCTGCTGATG | | | |
| | ATGTAATTAAAGTAGGCGGTCTTGAG | | | |
| | ACGGCGGATGGTCGACAGAAGCACCA | | | |
| | TGTCCTTGGGTCCGGCCTGCTGAATGC | | | |
| | GCAGGCGGTCGGCCATGCCCCAGGCT | | | |
| | TCGTTTTGACATCGGCGCAGGTCTTTG | | | |
| | TAGTAGTCTTGCATGAGCCTTTCTACC | | | |
| | GGCACTTCTTCTTCTCCTTCCTCTTGTC | | | |
| | CTGCATCTCTTGCATCTATCGCTGCGG | | | |
| | CGGCGGCGGAGTTTGGCCGTAGGTGG | | | |
| | CGCCCTCTTCCTCCCATGCGTGTGACC | | | |
| | CCGAAGCCCCTCATCGGCTGAAGCAG | | | |
| | GGCTAGGTCGGCGACAACGCGCTCGG | | | |
| | CTAATATGGCCTGCTGCACCTGCGTG | | | |
| | AGGGTAGACTGGAAGTCATCCATGTC | | | |
| | CACAAAGCGGTGGTATGCGCCCGTGT | | | |
| | TGATGGTGTAAGTGCAGTTGGCCATA | | | |
| | ACGGACCAGTTAACGGTCTGGTGACC | | | |
| | CGGCTGCGAGAGCTCGGTGTACCTGA | | | |
| | GACGCGAGTAAGCCCTCGAGTCAAAT | | | |
| | ACGTAGTCGTTGCAAGTCCGCACCAG | | | |
| | GTACTGGTATCCCACCAAAAAGTGCG | | | |
| | GCGGCGGCTGGCGGTAGAGGGGCCAG | | | |
| | CGTAGGGTGGCCGGGGCTCCGGGGGC | | | |
| | GAGATCTTCCAACATAAGGCGATGAT | | | |
| | ATCCGTAGATGTACCTGGACATCCAG | | | |
| | GTGATGCCGGCGGCGGTGGTGGAGGC | | | |
| | GCGCGGAAAGTCGCGGACGCGGTTCC | | | |
| | AGATGTTGCGCAGCGGCAAAAAGTGC | | | |
| | TCCATGGTCGGGACGCTCTGGCCGGT | | | |
| | CAGGCGCGCGCAATCGTTGACGCTCT | | | |
| | AGACCGTGCAAAAGGAGAGCCTGTAA | | | |
| | GCGGGCACTCTTCCGTGGTCTGGTGG | | | |
| | ATAAATTCGCAAGGGTATCATGGCGG | | | |
| | ACGACCGGGGTTCGAGCCCCGTATCC | | | |
| | GGCCGTCCGCCGTGATCCATGCGGTT | | | |
| | ACCGCCCGCGTGTCGAACCCAGGTGT | | | |
| | GCGACGTCAGACAACGGGGGAGTGCT | | | |
| | CCTTTTGGCTTCCTTCCAGGCGCGGCG | | | |
| | GCTGCTGCGCTAGCTTTTTTGGCCACT | | | |
| | GGCCGCGCGCAGCGTAAGCGGTTAGG | | | |
| | CTGGAAAGCGAAAGCATTAAGTGGCT | | | |
| | CGCTCCCTGTAGCCGGAGGGTTATTTT | | | |
| | CCAAGGGTTGAGTCGCGGGACCCCCG | | | |
| | GTTCGAGTCTCGGACCGGCCGGACTG | | | |
| | CGGCGAACGGGGGTTTGCCTCCCCGT | | | |
| | CATGCAAGACCCCGCTTGCAAATTCC | | | |
| | TCCGGAAACAGGGACGAGCCCCTTTT | | | |
| | TTGCTTTTCCCAGATGCATCCGGTGCT | | | |
| | GCGGCAGATGCGCCCCCCTCCTCAGC | | | |
| | AGCGGCAAGAGCAAGAGCAGCGGCA | | | |
| | GACATGCAGGGCACCCTCCCCTCCTC | | | |
| | CTACCGCGTCAGGAGGGGCGACATCC | | | |
| | GCGGTTGACGCGGCAGCAGATGGTGA | | | |
| | TTACGAACCCCCGCGGCGCCGGGCCC | | | |
| | GGCACTACCTGGACTTGGAGGAGGGC | | | |
| | GAGGGCCTGGCGCGGCTAGGAGCGCC | | | |
| | CTCTCCTGAGCGGTACCCAAGGGTGC | | | |
| | AGCTGAAGCGTGATACGCGTGAGGCG | | | |
| | TACGTGCCGCGGCAGAACCTGTTTCG | | | |
| | CGACCGCGAGGGAGAGGAGCCCGAG | | | |
| | GAGATGCGGGATCGAAAGTTCCACGC | | | |
| | AGGGCGCGAGCTGCGGCATGGCCTGA | | | |
| | ATCGCGAGCGGTTGCTGCGCGAGGAG | | | |
| | GACTTTGAGCCCGACGCGCGAACCGG | | | |
| | GATTAGTCCCGCGCGCGCACACGTGG | | | |
| | CGGCCGCCGACCTGGTAACCGCATAC | | | |
| | GAGCAGACGGTGAACCAGGAGATTAA | | | |
| | CTTTCAAAAAAGCTTTAACAACCACG | | | |
| | TGCGTACGCTTGTGGCGCGCGAGGAG | | | |
| | GTGGCTATAGGACTGATGCATCTGTG | | | |
| | GGACTTTGTAAGCGCGCTGGAGCAAA | | | |
| | ACCCAAATAGCAAGCCGCTCATGGCG | | | |
| | CAGCTGTTCCTTATAGTGCAGCACAG | | | |
| | CAGGGACAACGAGGCATTCAGGGATG | | | |
| | CGCTGCTAAACATAGTAGAGCCCGAG | | | |
| | GGCCGCTGGCTGCTCGATTTGATAAA | | | |
| | CATCCTGCAGAGCATAGTGGTGCAGG | | | |
| | AGCGCAGCTTGAGCCTGGCTGACAAG | | | |
| | GTGGCCGCCATCAACTATTCCATGCTT | | | |
| | AGCCTGGGCAAGTTTTACGCCCGCAA | | | |
| | GATATACCATACCCCTTACGTTCCCAT | | | |
| | AGACAAGGAGGTAAAGATCGAGGGG | | | |
| | TTCTACATGCGCATGGCGCTGAAGGT | | | |
| | GCTTACCTTGAGCGACGACCTGGGCG | | | |
| | TTTATCGCAACGAGCGCATCCACAAG | | | |
| | GCCGTGAGCGTGAGCCGGCGGCGCGA | | | |
| | GCTCAGCGACCGCGAGCTGATGCACA | | | |
| | GCCTGCAAAGGGCCCTGGCTGGCACG | | | |
| | GGCAGCGGCGATAGAGAGGCCGAGTC | | | |
| | CTACTTTGACGCGGGCGCTGACCTGC | | | |
| | GCTGGGCCCCAAGCCGACGCGCCCTG | | | |
| | GAGGCAGCTGGGGCCGGACCTGGGCT | | | |
| | GGCGGTGGCACCCGCGCGCGCTGGCA | | | |
| | ACGTCGGCGGCGTGGAGGAATATGAC | | | |
| | GAGGACGATGAGTACGAGCCAGAGG | | | |
| | ACGGCGAGTACTAAGCGGTGATGTTT | | | |
| | CTGATCAGATGATGCAAGACGCAACG | | | |
| | GACCCGGCGGTGCGGGCGGCGCTGCA | | | |
| | GAGCCAGCCGTCCGGCCTTAACTCCA | | | |
| | CGGACGACTGGCGCCAGGTCATGGAC | | | |
| | CGCATCATGTCGCTGACTGCGCGCAA | | | |
| | TCCTGACGCGTTCCGGCAGCAGCCGC | | | |
| | AGGCCAACCGGCTCTCCGCAATTCTG | | | |
| | GAAGCGGTGGTCCCGGCGCGCGCAAA | | | |
| | CCCCACGCACGAGAAGGTGCTGGCGA | | | |
| | TCGTAAACGCGCTGGCCGAAAACAGG | | | |
| | GCCATCCGGCCCGACGAGGCCGGCCT | | | |
| | GGTCTACGACGCGCTGCTTCAGCGCG | | | |
| | TGGCTCGTTACAACAGCGGCAACGTG | | | |
| | CAGACCAACCTGGACCGGCTGGTGGG | | | |
| | GGATGTGCGCGAGGCCGTGGCGCAGC | | | |
| | GTGAGCGCGCGCAGCAGCAGGGCAAC | | | |
| | CTGGGCTCCATGGTTGCACTAAACGC | | | |
| | CTTCCTGAGTACACAGCCCGCCAACG | | | |
| | TGCCGCGGGGACAGGAGGACTACACC | | | |
| | AACTTTGTGAGCGCACTGCGGCTAAT | | | |
| | GGTGACTGAGACACCGCAAAGTGAGG | | | |
| | TGTACCAGTCTGGGCCAGACTATTTTT | | | |
| | TCCAGACCAGTAGACAAGGCCTGCAG | | | |
| | ACCGTAAACCTGAGCCAGGCTTTCAA | | | |
| | AAACTTGCAGGGGCTGTGGGGGGTGC | | | |
| | GGGCTCCCACAGGCGACCGCGCGACC | | | |
| | GTGTCTAGCTTGCTGACGCCCAACTCG | | | |
| | CGCCTGTTGCTGCTGCTAATAGCGCCC | | | |
| | TTCACGGACAGTGGCAGCGTGTCCCG | | | |
| | GGACACATACCTAGGTCACTTGCTGA | | | |
| | CACTGTACCGCGAGGCCATAGGTCAG | | | |
| | GCGCATGTGGACGAGCATACTTTCCA | | | |
| | GGAGATTACAAGTGTCAGCCGCGCGC | | | |
| | TGGGGCAGGAGGACACGGGCAGCCTG | | | |
| | GAGGCAACCCTAAACTACCTGCTGAC | | | |
| | CAACCGGCGGCAGAAGATCCCCTCGT | | | |
| | TGCACAGTTTAAACAGCGAGGAGGAG | | | |
| | CGCATTTTGCGCTACGTGCAGCAGAG | | | |
| | CGTGAGCCTTAACCTGATGCGCGACG | | | |
| | GGGTAACGCCCAGCGTGGCGCTGGAC | | | |
| | ATGACCGCGCGCAACATGGAACCGGG | | | |
| | CATGTATGCCTCAAACCGGCCGTTTAT | | | |
| | CAACCGCCTAATGGACTACTTGCATC | | | |
| | GCGCGGCCGCCGTGAACCCCGAGTAT | | | |
| | TTCACCAATGCCATCTTGAACCCGCAC | | | |
| | TGGCTACCGCCCCCTGGTTTCTACACC | | | |
| | GGGGGATTCGAGGTGCCCGAGGGTAA | | | |
| | CGATGGATTCCTCTGGGACGACATAG | | | |
| | ACGACAGCGTGTTTTCCCCGCAACCG | | | |
| | CAGACCCTGCTAGAGTTGCAACAGCG | | | |
| | CGAGCAGGCAGAGGCGGCGCTGCGA | | | |
| | AAGGAAAGCTTCCGCAGGCCAAGCAG | | | |
| | CTTGTCCGATCTAGGCGCTGCGGCCCC | | | |
| | GCGGTCAGATGCTAGTAGCCCATTTC | | | |
| | CAAGCTTGATAGGGTCTCTTACCAGC | | | |
| | ACTCGCACCACCCGCCCGCGCCTGCT | | | |
| | GGGCGAGGAGGAGTACCTAAACAACT | | | |
| | CGCTGCTGCAGCCGCAGCGCGAAAAA | | | |
| | AACCTGCCTCCGGCATTTCCCAACAA | | | |
| | CGGGATAGAGAGCCTAGTGGACAAGA | | | |
| | TGAGTAGATGGAAGACGTACGCGCAG | | | |
| | GAGCACAGGGACGTGCCAGGCCCGCG | | | |
| | CCCGCCCACCCGTCGTCAAAGGCACG | | | |
| | ACCGTCAGCGGGGTCTGGTGTGGGAG | | | |
| | GACGATGACTCGGCAGACGACAGCAG | | | |
| | CGTCCTGGATTTGGGAGGGAGTGGCA | | | |
| | ACCCGTTTGCGCACCTTCGCCCCAGGC | | | |
| | TGGGGAGAATGTTTTAAAAAAAAAAA | | | |
| | AGCATGATGCAAAATAAAAAACTCAC | | | |
| | CAAGGCCATGGCACCGAGCGTTGGTT | | | |
| | TTCTTGTATTCCCCTTAGTATGCGGCG | | | |
| | CGCGGCGATGTATGAGGAAGGTCCTC | | | |
| | CTCCCTCCTACGAGAGTGTGGTGAGC | | | |
| | GCGGCGCCAGTGGCGGCGGCGCTGGG | | | |
| | TTCTCCCTTCGATGCTCCCCTGGACCC | | | |
| | GCCGTTTGTGCCTCCGCGGTACCTGCG | | | |
| | GCCTACCGGGGGGAGAAACAGCATCC | | | |
| | GTTACTCTGAGTTGGCACCCCTATTCG | | | |
| | ACACCACCCGTGTGTACCTGGTGGAC | | | |
| | AACAAGTCAACGGATGTGGCATCCCT | | | |
| | GAACTACCAGAACGACCACAGCAACT | | | |
| | TTCTGACCACGGTCATTCAAAACAAT | | | |
| | GACTACAGCCCGGGGGAGGCAAGCAC | | | |
| | ACAGACCATCAATCTTGACGACCGGT | | | |
| | CGCACTGGGGCGGCGACCTGAAAACC | | | |
| | ATCCTGCATACCAACATGCCAAATGT | | | |
| | GAACGAGTTCATGTTTACCAATAAGT | | | |
| | TTAAGGCGCGGGTGATGGTGTCGCGC | | | |
| | TTGCCTACTAAGGACAATCAGGTGGA | | | |
| | GCTGAAATACGAGTGGGTGGAGTTCA | | | |
| | CGCTGCCCGAGGGCAACTACTCCGAG | | | |
| | ACCATGACCATAGACCTTATGAACAA | | | |
| | CGCGATCGTGGAGCACTACTTGAAAG | | | |
| | TGGGCAGACAGAACGGGGTTCTGGAA | | | |
| | AGCGACATCGGGGTAAAGTTTGACAC | | | |
| | CCGCAACTTCAGACTGGGGTTTGACC | | | |
| | CCGTCACTGGTCTTGTCATGCCTGGGG | | | |
| | TATATACAAACGAAGCCTTCCATCCA | | | |
| | GACATCATTTTGCTGCCAGGATGCGG | | | |
| | GGTGGACTTCACCCACAGCCGCCTGA | | | |
| | GCAACTTGTTGGGCATCCGCAAGCGG | | | |
| | CAACCCTTCCAGGAGGGCTTTAGGAT | | | |
| | CACCTACGATGATCTGGAGGGTGGTA | | | |
| | ACATTCCCGCACTGTTGGATGTGGAC | | | |
| | GCCTACCAGGCGAGCTTGAAAGATGA | | | |
| | CACCGAACAGGGCGGGGGTGGCGCA | | | |
| | GGCGGCAGCAACAGCAGTGGCAGCG | | | |
| | GCGCGGAAGAGAACTCCAACGCGGCA | | | |
| | GCCGCGGCAATGCAGCCGGTGGAGGA | | | |
| | CATGAACGATCATGCCATTCGCGGCG | | | |
| | ACACCTTTGCCACACGGGCTGAGGAG | | | |
| | AAGCGCGCTGAGGCCGAAGCAGCGGC | | | |
| | CGAAGCTGCCGCCCCCGCTGCGCAAC | | | |
| | CCGAGGTCGAGAAGCCTCAGAAGAAA | | | |
| | CCGGTGATCAAACCCCTGACAGAGGA | | | |
| | CAGCAAGAAACGCAGTTACAACCTAA | | | |
| | TAAGCAATGACAGCACCTTCACCCAG | | | |
| | TACCGCAGCTGGTACCTTGCATACAA | | | |
| | CTACGGCGACCCTCAGACCGGAATCC | | | |
| | GCTCATGGACCCTGCTTTGCACTCCTG | | | |
| | ACGTAACCTGCGGCTCGGAGCAGGTC | | | |
| | TACTGGTCGTTGCCAGACATGATGCA | | | |
| | AGACCCCGTGACCTTCCGCTCCACGC | | | |
| | GCCAGATCAGCAACTTTCCGGTGGTG | | | |
| | GGCGCCGAGCTGTTGCCCGTGCACTC | | | |
| | CAAGAGCTTCTACAACGACCAGGCCG | | | |
| | TCTACTCCCAACTCATCCGCCAGTTTA | | | |
| | CCTCTCTGACCCACGTGTTCAATCGCT | | | |
| | TTCCCGAGAACCAGATTTTGGCGCGC | | | |
| | CCGCCAGCCCCCACCATCACCACCGT | | | |
| | CAGTGAAAACGTTCCTGCTCTCACAG | | | |
| | ATCACGGGACGCTACCGCTGCGCAAC | | | |
| | AGCATCGGAGGAGTCCAGCGAGTGAC | | | |
| | CATTACTGACGCCAGACGCCGCACCT | | | |
| | GCCCCTACGTTTACAAGGCCCTGGGC | | | |
| | ATAGTCTCGCCGCGCGTCCTATCGAG | | | |
| | CCGCACTTTTTGAGCAAGCATGTCCAT | | | |
| | CCTTATATCGCCCAGCAATAACACAG | | | |
| | GCTGGGGCCTGCGCTTCCCAAGCAAG | | | |
| | ATGTTTGGCGGGGCCAAGAAGCGCTC | | | |
| | CGACCAACACCCAGTGCGCGTGCGCG | | | |
| | GGCACTACCGCGCGCCCTGGGGCGCG | | | |
| | CACAAACGCGGCCGCACTGGGCGCAC | | | |
| | CACCGTCGATGACGCCATCGACGCGG | | | |
| | TGGTGGAGGAGGCGCGCAACTACACG | | | |
| | CCCACGCCGCCACCAGTGTCCACAGT | | | |
| | GGACGCGGCCATTCAGACCGTGGTGC | | | |
| | GCGGAGCCCGGCGCTATGCTAAAATG | | | |
| | AAGAGACGGCGGAGGCGCGTAGCAC | | | |
| | GTCGCCACCGCCGCCGACCCGGCACT | | | |
| | GCCGCCCAACGCGCGGCGGCGGCCCT | | | |
| | GCTTAACCGCGCACGTCGCACCGGCC | | | |
| | GACGGGCGGCCATGCGGGCCGCTCGA | | | |
| | AGGCTGGCCGCGGGTATTGTCACTGT | | | |
| | GCCCCCCAGGTCCAGGCGACGAGCGG | | | |
| | CCGCCGCAGCAGCCGCGGCCATTAGT | | | |
| | GCTATGACTCAGGGTCGCAGGGGCAA | | | |
| | CGTGTATTGGGTGCGCGACTCGGTTA | | | |
| | GCGGCCTGCGCGTGCCCGTGCGCACC | | | |
| | CGCCCCCCGCGCAACTAGATTGCAAG | | | |
| | AAAAAACTACTTAGACTCGTACTGTT | | | |
| | GTATGTATCCAGCGGCGGCGGCGCGC | | | |
| | AACGAAGCTATGTCCAAGCGCAAAAT | | | |
| | CAAAGAAGAGATGCTCCAGGTCATCG | | | |
| | CGCCGGAGATCTATGGCCCCCCGAAG | | | |
| | AAGGAAGAGCAGGATTACAAGCCCCG | | | |
| | AAAGCTAAAGCGGGTCAAAAAGAAA | | | |
| | AAGAAAGATGATGATGATGAACTTGA | | | |
| | CGACGAGGTGGAACTGCTGCACGCTA | | | |
| | CCGCGCCCAGGCGACGGGTACAGTGG | | | |
| | AAAGGTCGACGCGTAAAACGTGTTTT | | | |
| | GCGACCCGGCACCACCGTAGTCTTTA | | | |
| | CGCCCGGTGAGCGCTCCACCCGCACC | | | |
| | TACAAGCGCGTGTATGATGAGGTGTA | | | |
| | CGGCGACGAGGACCTGCTTGAGCAGG | | | |
| | CCAACGAGCGCCTCGGGGAGTTTGCC | | | |
| | TACGGAAAGCGGCATAAGGACATGCT | | | |
| | GGCGTTGCCGCTGGACGAGGGCAACC | | | |
| | CAACACCTAGCCTAAAGCCCGTAACA | | | |
| | CTGCAGCAGGTGCTGCCCGCGCTTGC | | | |
| | ACCGTCCGAAGAAAAGCGCGGCCTAA | | | |
| | AGCGCGAGTCTGGTGACTTGGCACCC | | | |
| | ACCGTGCAGCTGATGGTACCCAAGCG | | | |
| | CCAGCGACTGGAAGATGTCTTGGAAA | | | |
| | AAATGACCGTGGAACCTGGGCTGGAG | | | |
| | CCCGAGGTCCGCGTGCGGCCAATCAA | | | |
| | GCAGGTGGCGCCGGGACTGGGCGTGC | | | |
| | AGACCGTGGACGTTCAGATACCCACT | | | |
| | ACCAGTAGCACCAGTATTGCCACCGC | | | |
| | CACAGAGGGCATGGAGACACAAACGT | | | |
| | CCCCGGTTGCCTCAGCGGTGGCGGAT | | | |
| | GCCGCGGTGCAGGCGGTCGCTGCGGC | | | |
| | CGCGTCCAAGACCTCTACGGAGGTGC | | | |
| | AAACGGACCCGTGGATGTTTCGCGTT | | | |
| | TCAGCCCCCCGGCGCCCGCGCGGTTC | | | |
| | GAGGAAGTACGGCGCCGCCAGCGCGC | | | |
| | TACTGCCCGAATATGCCCTACATCCTT | | | |
| | CCATTGCGCCTACCCCCGGCTATCGTG | | | |
| | GCTACACCTACCGCCCCAGAAGACGA | | | |
| | GCAACTACCCGACGCCGAACCACCAC | | | |
| | TGGAACCCGCCGCCGCCGTCGCCGTC | | | |
| | GCCAGCCCGTGCTGGCCCCGATTTCC | | | |
| | GTGCGCAGGGTGGCTCGCGAAGGAGG | | | |
| | CAGGACCCTGGTGCTGCCAACAGCGC | | | |
| | GCTACCACCCCAGCATCGTTTAAAAG | | | |
| | CCGGTCTTTGTGGTTCTTGCAGATATG | | | |
| | GCCCTCACCTGCCGCCTCCGTTTCCCG | | | |
| | GTGCCGGGATTCCGAGGAAGAATGCA | | | |
| | CCGTAGGAGGGGCATGGCCGGCCACG | | | |
| | GCCTGACGGGCGGCATGCGTCGTGCG | | | |
| | CACCACCGGCGGCGGCGCGCGTCGCA | | | |
| | CCGTCGCATGCGCGGCGGTATCCTGC | | | |
| | CCCTCCTTATTCCACTGATCGCCGCGG | | | |
| | CGATTGGCGCCGTGCCCGGAATTGCA | | | |
| | TCCGTGGCCTTGCAGGCGCAGAGACA | | | |
| | CTGATTAAAAACAAGTTGCATGTGGA | | | |
| | AAAATCAAAATAAAAAGTCTGGACTC | | | |
| | TCACGCTCGCTTGGTCCTGTAACTATT | | | |
| | TTGTAGAATGGAAGACATCAACTTTG | | | |
| | CGTCTCTGGCCCCGCGACACGGCTCG | | | |
| | CGCCCGTTCATGGGAAACTGGCAAGA | | | |
| | TATCGGCACCAGCAATATGAGCGGTG | | | |
| | GCGCCTTCAGCTGGGGCTCGCTGTGG | | | |
| | AGCGGCATTAAAAATTTCGGTTCCAC | | | |
| | CGTTAAGAACTATGGCAGCAAGGCCT | | | |
| | GGAACAGCAGCACAGGCCAGATGCTG | | | |
| | AGGGATAAGTTGAAAGAGCAAAATTT | | | |
| | CCAACAAAAGGTGGTAGATGGCCTGG | | | |
| | CCTCTGGCATTAGCGGGGTGGTGGAC | | | |
| | CTGGCCAACCAGGCAGTGCAAAATAA | | | |
| | GATTAACAGTAAGCTTGATCCCCGCC | | | |
| | CTCCCGTAGAGGAGCCTCCACCGGCC | | | |
| | GTGGAGACAGTGTCTCCAGAGGGGCG | | | |
| | TGGCGAAAAGCGTCCGCGCCCCGACA | | | |
| | GGGAAGAAACTCTGGTGACGCAAATA | | | |
| | GACGAGCCTCCCTCGTACGAGGAGGC | | | |
| | ACTAAAGCAAGGCCTGCCCACCACCC | | | |
| | GTCCCATCGCGCCCATGGCTACCGGA | | | |
| | GTGCTGGGCCAGCACACACCCGTAAC | | | |
| | GCTGGACCTGCCTCCCCCCGCCGACA | | | |
| | CCCAGCAGAAACCTGTGCTGCCAGGC | | | |
| | CCGACCGCCGTTGTTGTAACCCGTCCT | | | |
| | AGCCGCGCGTCCCTGCGCCGCGCCGC | | | |
| | CAGCGGTCCGCGATCGTTGCGGCCCG | | | |
| | TAGCCAGTGGCAACTGGCAAAGCACA | | | |
| | CTGAACAGCATCGTGGGTCTGGGGGT | | | |
| | GCAATCCCTGAAGCGCCGACGATGCT | | | |
| | TCTGAATAGCTAACGTGTCGTATGTGT | | | |
| | GTCATGTATGCGTCCATGTCGCCGCCA | | | |
| | GAGGAGCTGCTGAGCCGCCGCGCGCC | | | |
| | CGCTTTCCAAGATGGCTACCCCTTCGA | | | |
| | TGATGCCGCAGTGGTCTTACATGCAC | | | |
| | ATCTCGGGCCAGGACGCCTCGGAGTA | | | |
| | CCTGAGCCCCGGGCTGGTGCAGTTTG | | | |
| | CCCGCGCCACCGAGACGTACTTCAGC | | | |
| | CTGAATAACAAGTTTAGAAACCCCAC | | | |
| | GGTGGCGCCTACGCACGACGTGACCA | | | |
| | CAGACCGGTCCCAGCGTTTGACGCTG | | | |
| | CGGTTCATCCCTGTGGACCGTGAGGA | | | |
| | TACTGCGTACTCGTACAAGGCGCGGT | | | |
| | TCACCCTAGCTGTGGGTGATAACCGT | | | |
| | GTGCTGGACATGGCTTCCACGTACTTT | | | |
| | GACATCCGCGGCGTGCTGGACAGGGG | | | |
| | CCCTACTTTTAAGCCCTACTCTGGCAC | | | |
| | TGCCTACAACGCCCTGGCTCCCAAGG | | | |
| | GTGCCCCAAATCCTTGCGAATGGGAT | | | |
| | GAAGCTGCTACTGCTCTTGAAATAAA | | | |
| | CCTAGAAGAAGAGGACGATGACAAC | | | |
| | GAAGACGAAGTAGACGAGCAAGCTG | | | |
| | AGCAGCAAAAAACTCACGTATTTGGG | | | |
| | CAGGCGCCTTATTCTGGTATAAATATT | | | |
| | ACAAAGGAGGGTATTCAAATAGGTGT | | | |
| | CGAAGGTCAAACACCTAAATATGCCG | | | |
| | ATAAAACATTTCAACCTGAACCTCAA | | | |
| | ATAGGAGAATCTCAGTGGTACGAAAC | | | |
| | TGAAATTAATCATGCAGCTGGGAGAG | | | |
| | TCCTTAAAAAGACTACCCCAATGAAA | | | |
| | CCATGTTACGGTTCATATGCAAAACC | | | |
| | CACAAATGAAAATGGAGGGCAAGGC | | | |
| | ATTCTTGTAAAGCAACAAAATGGAAA | | | |
| | GCTAGAAAGTCAAGTGGAAATGCAAT | | | |
| | TTTTCTCAACTACTGAGGCGACCGCA | | | |
| | GGCAATGGTGATAACTTGACTCCTAA | | | |
| | AGTGGTATTGTACAGTGAAGATGTAG | | | |
| | ATATAGAAACCCCAGACACTCATATT | | | |
| | TCTTACATGCCCACTATTAAGGAAGG | | | |
| | TAACTCACGAGAACTAATGGGCCAAC | | | |
| | AATCTATGCCCAACAGGCCTAATTAC | | | |
| | ATTGCTTTTAGGGACAATTTTATTGGT | | | |
| | CTAATGTATTACAACAGCACGGGTAA | | | |
| | TATGGGTGTTCTGGCGGGCCAAGCAT | | | |
| | CGCAGTTGAATGCTGTTGTAGATTTGC | | | |
| | AAGACAGAAACACAGAGCTTTCATAC | | | |
| | CAGCTTTTGCTTGATTCCATTGGTGAT | | | |
| | AGAACCAGGTACTTTTCTATGTGGAA | | | |
| | TCAGGCTGTTGACAGCTATGATCCAG | | | |
| | ATGTTAGAATTATTGAAAATCATGGA | | | |
| | ACTGAAGATGAACTTCCAAATTACTG | | | |
| | CTTTCCACTGGGAGGTGTGATTAATAC | | | |
| | AGAGACTCTTACCAAGGTAAAACCTA | | | |
| | AAACAGGTCAGGAAAATGGATGGGA | | | |
| | AAAAGATGCTACAGAATTTTCAGATA | | | |
| | AAAATGAAATAAGAGTTGGAAATAAT | | | |
| | TTTGCCATGGAAATCAATCTAAATGC | | | |
| | CAACCTGTGGAGAAATTTCCTGTACTC | | | |
| | CAACATAGCGCTGTATTTGCCCGACA | | | |
| | AGCTAAAGTACAGTCCTTCCAACGTA | | | |
| | AAAATTTCTGATAACCCAAACACCTA | | | |
| | CGACTACATGAACAAGCGAGTGGTGG | | | |
| | CTCCCGGGTTAGTGGACTGCTACATTA | | | |
| | ACCTTGGAGCACGCTGGTCCCTTGACT | | | |
| | ATATGGACAACGTCAACCCATTTAAC | | | |
| | CACCACCGCAATGCTGGCCTGCGCTA | | | |
| | CCGCTCAATGTTGCTGGGCAATGGTC | | | |
| | GCTATGTGCCCTTCCACATCCAGGTGC | | | |
| | CTCAGAAGTTCTTTGCCATTAAAAACC | | | |
| | TCCTTCTCCTGCCGGGCTCATACACCT | | | |
| | ACGAGTGGAACTTCAGGAAGGATGTT | | | |
| | AACATGGTTCTGCAGAGCTCCCTAGG | | | |
| | AAATGACCTAAGGGTTGACGGAGCCA | | | |
| | GCATTAAGTTTGATAGCATTTGCCTTT | | | |
| | ACGCCACCTTCTTCCCCATGGCCCACA | | | |
| | ACACCGCCTCCACGCTTGAGGCCATG | | | |
| | CTTAGAAACGACACCAACGACCAGTC | | | |
| | CTTTAACGACTATCTCTCCGCCGCCAA | | | |
| | CATGCTCTACCCTATACCCGCCAACGC | | | |
| | TACCAACGTGCCCATATCCATCCCCTC | | | |
| | CCGCAACTGGGCGGCTTTCCGCGGCT | | | |
| | GGGCCTTCACGCGCCTTAAGACTAAG | | | |
| | GAAACCCCATCACTGGGCTCGGGCTA | | | |
| | CGACCCTTATTACACCTACTCTGGCTC | | | |
| | TATACCCTACCTAGATGGAACCTTTTA | | | |
| | CCTCAACCACACCTTTAAGAAGGTGG | | | |
| | CCATTACCTTTGACTCTTCTGTCAGCT | | | |
| | GGCCTGGCAATGACCGCCTGCTTACC | | | |
| | CCCAACGAGTTTGAAATTAAGCGCTC | | | |
| | AGTTGACGGGGAGGGTTACAACGTTG | | | |
| | CCCAGTGTAACATGACCAAAGACTGG | | | |
| | TTCCTGGTACAAATGCTAGCTAACTAC | | | |
| | AACATTGGCTACCAGGGCTTCTATATC | | | |
| | CCAGAGAGCTACAAGGACCGCATGTA | | | |
| | CTCCTTCTTTAGAAACTTCCAGCCCAT | | | |
| | GAGCCGTCAGGTGGTGGATGATACTA | | | |
| | AATACAAGGACTACCAACAGGTGGGC | | | |
| | ATCCTACACCAACACAACAACTCTGG | | | |
| | ATTTGTTGGCTACCTTGCCCCCACCAT | | | |
| | GCGCGAAGGACAGGCCTACCCTGCTA | | | |
| | ACTTCCCCTATCCGCTTATAGGCAAGA | | | |
| | CCGCAGTTGACAGCATTACCCAGAAA | | | |
| | AAGTTTCTTTGCGATCGCACCCTTTGG | | | |
| | CGCATCCCATTCTCCAGTAACTTTATG | | | |
| | TCCATGGGCGCACTCACAGACCTGGG | | | |
| | CCAAAACCTTCTCTACGCCAACTCCGC | | | |
| | CCACGCGCTAGACATGACTTTTGAGG | | | |
| | TGGATCCCATGGACGAGCCCACCCTT | | | |
| | CTTTATGTTTTGTTTGAAGTCTTTGAC | | | |
| | GTGGTCCGTGTGCACCGGCCGCACCG | | | |
| | CGGCGTCATCGAAACCGTGTACCTGC | | | |
| | GCACGCCCTTCTCGGCCGGCAACGCC | | | |
| | ACAACATAAAGAAGCAAGCAACATCA | | | |
| | ACAACAGCTGCCGCCATGGGCTCCAG | | | |
| | TGAGCAGGAACTGAAAGCCATTGTCA | | | |
| | AAGATCTTGGTTGTGGGCCATATTTTT | | | |
| | TGGGCACCTATGACAAGCGCTTTCCA | | | |
| | GGCTTTGTTTCTCCACACAAGCTCGCC | | | |
| | TGCGCCATAGTCAATACGGCCGGTCG | | | |
| | CGAGACTGGGGGCGTACACTGGATGG | | | |
| | CCTTTGCCTGGAACCCGCACTCAAAA | | | |
| | ACATGCTACCTCTTTGAGCCCTTTGGC | | | |
| | TTTTCTGACCAGCGACTCAAGCAGGTT | | | |
| | TACCAGTTTGAGTACGAGTCACTCCTG | | | |
| | CGCCGTAGCGCCATTGCTTCTTCCCCC | | | |
| | GACCGCTGTATAACGCTGGAAAAGTC | | | |
| | CACCCAAAGCGTACAGGGGCCCAACT | | | |
| | CGGCCGCCTGTGGACTATTCTGCTGCA | | | |
| | TGTTTCTCCACGCCTTTGCCAACTGGC | | | |
| | CCCAAACTCCCATGGATCACAACCCC | | | |
| | ACCATGAACCTTATTACCGGGGTACC | | | |
| | CAACTCCATGCTCAACAGTCCCCAGG | | | |
| | TACAGCCCACCCTGCGTCGCAACCAG | | | |
| | GAACAGCTCTACAGCTTCCTGGAGCG | | | |
| | CCACTCGCCCTACTTCCGCAGCCACA | | | |
| | GTGCGCAGATTAGGAGCGCCACTTCT | | | |
| | TTTTGTCACTTGAAAAACATGTAAAA | | | |
| | ATAATGTACTAGAGACACTTTCAATA | | | |
| | AAGGCAAATGCTTTTATTTGTACACTC | | | |
| | TCGGGTGATTATTTACCCCCACCCTTG | | | |
| | CCGTCTGCGCCGTTTAAAAATCAAAG | | | |
| | GGGTTCTGCCGCGCATCGCTATGCGC | | | |
| | CACTGGCAGGGACACGTTGCGATACT | | | |
| | GGTGTTTAGTGCTCCACTTAAACTCAG | | | |
| | GCACAACCATCCGCGGCAGCTCGGTG | | | |
| | AAGTTTTCACTCCACAGGCTGCGCAC | | | |
| | CATCACCAACGCGTTTAGCAGGTCGG | | | |
| | GCGCCGATATCTTGAAGTCGCAGTTG | | | |
| | GGGCCTCCGCCCTGCGCGCGCGAGTT | | | |
| | GCGATACACAGGGTTGCAGCACTGGA | | | |
| | ACACTATCAGCGCCGGGTGGTGCACG | | | |
| | CTGGCCAGCACGCTCTTGTCGGAGAT | | | |
| | CAGATCCGCGTCCAGGTCCTCCGCGTT | | | |
| | GCTCAGGGCGAACGGAGTCAACTTTG | | | |
| | GTAGCTGCCTTCCCAAAAAGGGCGCG | | | |
| | TGCCCAGGCTTTGAGTTGCACTCGCAC | | | |
| | CGTAGTGGCATCAAAAGGTGACCGTG | | | |
| | CCCGGTCTGGGCGTTAGGATACAGCG | | | |
| | CCTGCATAAAAGCCTTGATCTGCTTAA | | | |
| | AAGCCACCTGAGCCTTTGCGCCTTCA | | | |
| | GAGAAGAACATGCCGCAAGACTTGCC | | | |
| | GGAAAACTGATTGGCCGGACAGGCCG | | | |
| | CGTCGTGCACGCAGCACCTTGCGTCG | | | |
| | GTGTTGGAGATCTGCACCACATTTCG | | | |
| | GCCCCACCGGTTCTTCACGATCTTGGC | | | |
| | CTTGCTAGACTGCTCCTTCAGCGCGCG | | | |
| | CTGCCCGTTTTCGCTCGTCACATCCAT | | | |
| | TTCAATCACGTGCTCCTTATTTATCAT | | | |
| | AATGCTTCCGTGTAGACACTTAAGCTC | | | |
| | GCCTTCGATCTCAGCGCAGCGGTGCA | | | |
| | GCCACAACGCGCAGCCCGTGGGCTCG | | | |
| | TGATGCTTGTAGGTCACCTCTGCAAAC | | | |
| | GACTGCAGGTACGCCTGCAGGAATCG | | | |
| | CCCCATCATCGTCACAAAGGTCTTGTT | | | |
| | GCTGGTGAAGGTCAGCTGCAACCCGC | | | |
| | GGTGCTCCTCGTTCAGCCAGGTCTTGC | | | |
| | ATACGGCCGCCAGAGCTTCCACTTGG | | | |
| | TCAGGCAGTAGTTTGAAGTTCGCCTTT | | | |
| | AGATCGTTATCCACGTGGTACTTGTCC | | | |
| | ATCAGCGCGCGCGCAGCCTCCATGCC | | | |
| | CTTCTCCCACGCAGACACGATCGGCA | | | |
| | CACTCAGCGGGTTCATCACCGTAATTT | | | |
| | CACTTTCCGCTTCGCTGGGCTCTTCCT | | | |
| | CTTCCTCTTGCGTCCGCATACCACGCG | | | |
| | CCACTGGGTCGTCTTCATTCAGCCGCC | | | |
| | GCACTGTGCGCTTACCTCCTTTGCCAT | | | |
| | GCTTGATTAGCACCGGTGGGTTGCTG | | | |
| | AAACCCACCATTTGTAGCGCCACATC | | | |
| | TTCTCTTTCTTCCTCGCTGTCCACGATT | | | |
| | ACCTCTGGTGATGGCGGGCGCTCGGG | | | |
| | CTTGGGAGAAGGGCGCTTCTTTTTCTT | | | |
| | CTTGGGCGCAATGGCCAAATCCGCCG | | | |
| | CCGAGGTCGATGGCCGCGGGCTGGGT | | | |
| | GTGCGCGGCACCAGCGCGTCTTGTGA | | | |
| | TGAGTCTTCCTCGTCCTCGGACTCGAT | | | |
| | ACGCCGCCTCATCCGCTTTTTTGGGGG | | | |
| | CGCCCGGGGAGGCGGCGGCGACGGG | | | |
| | GACGGGGACGACACGTCCTCCATGGT | | | |
| | TGGGGGACGTCGCGCCGCACCGCGTC | | | |
| | CGCGCTCGGGGGTGGTTTCGCGCTGC | | | |
| | TCCTCTTCCCGACTGGCCATTTCCTTC | | | |
| | TCCTATAGGCAGAAAAAGATCATGGA | | | |
| | GTCAGTCGAGAAGAAGGACAGCCTAA | | | |
| | CCGCCCCCTCTGAGTTCGCCACCACC | | | |
| | GCCTCCACCGATGCCGCCAACGCGCC | | | |
| | TACCACCTTCCCCGTCGAGGCACCCC | | | |
| | CGCTTGAGGAGGAGGAAGTGATTATC | | | |
| | GAGCAGGACCCAGGTTTTGTAAGCGA | | | |
| | AGACGACGAGGACCGCTCAGTACCAA | | | |
| | CAGAGGATAAAAAGCAAGACCAGGA | | | |
| | CAACGCAGAGGCAAACGAGGAACAA | | | |
| | GTCGGGCGGGGGGACGAAAGGCATG | | | |
| | GCGACTACCTAGATGTGGGAGACGAC | | | |
| | GTGCTGTTGAAGCATCTGCAGCGCCA | | | |
| | GTGCGCCATTATCTGCGACGCGTTGC | | | |
| | AAGAGCGCAGCGATGTGCCCCTCGCC | | | |
| | ATAGCGGATGTCAGCCTTGCCTACGA | | | |
| | ACGCCACCTATTCTCACCGCGCGTACC | | | |
| | CCCCAAACGCCAAGAAAACGGCACAT | | | |
| | GCGAGCCCAACCCGCGCCTCAACTTC | | | |
| | TACCCCGTATTTGCCGTGCCAGAGGT | | | |
| | GCTTGCCACCTATCACATCTTTTTCCA | | | |
| | AAACTGCAAGATACCCCTATCCTGCC | | | |
| | GTGCCAACCGCAGCCGAGCGGACAAG | | | |
| | CAGCTGGCCTTGCGGCAGGGCGCTGT | | | |
| | CATACCTGATATCGCCTCGCTCAACG | | | |
| | AAGTGCCAAAAATCTTTGAGGGTCTT | | | |
| | GGACGCGACGAGAAGCGCGCGGCAA | | | |
| | ACGCTCTGCAACAGGAAAACAGCGAA | | | |
| | AATGAAAGTCACTCTGGAGTGTTGGT | | | |
| | GGAACTCGAGGGTGACAACGCGCGCC | | | |
| | TAGCCGTACTAAAACGCAGCATCGAG | | | |
| | GTCACCCACTTTGCCTACCCGGCACTT | | | |
| | AACCTACCCCCCAAGGTCATGAGCAC | | | |
| | AGTCATGAGTGAGCTGATCGTGCGCC | | | |
| | GTGCGCAGCCCCTGGAGAGGGATGCA | | | |
| | AATTTGCAAGAACAAACAGAGGAGG | | | |
| | GCCTACCCGCAGTTGGCGACGAGCAG | | | |
| | CTAGCGCGCTGGCTTCAAACGCGCGA | | | |
| | GCCTGCCGACTTGGAGGAGCGACGCA | | | |
| | AACTAATGATGGCCGCAGTGCTCGTT | | | |
| | ACCGTGGAGCTTGAGTGCATGCAGCG | | | |
| | GTTCTTTGCTGACCCGGAGATGCAGC | | | |
| | GCAAGCTAGAGGAAACATTGCACTAC | | | |
| | ACCTTTCGACAGGGCTACGTACGCCA | | | |
| | GGCCTGCAAGATCTCCAACGTGGAGC | | | |
| | TCTGCAACCTGGTCTCCTACCTTGGAA | | | |
| | TTTTGCACGAAAACCGCCTTGGGCAA | | | |
| | AACGTGCTTCATTCCACGCTCAAGGG | | | |
| | CGAGGCGCGCCGCGACTACGTCCGCG | | | |
| | ACTGCGTTTACTTATTTCTATGCTACA | | | |
| | CCTGGCAGACGGCCATGGGCGTTTGG | | | |
| | CAGCAGTGCTTGGAGGAGTGCAACCT | | | |
| | CAAGGAGCTGCAGAAACTGCTAAAGC | | | |
| | AAAACTTGAAGGACCTATGGACGGCC | | | |
| | TTCAACGAGCGCTCCGTGGCCGCGCA | | | |
| | CCTGGCGGACATCATTTTCCCCGAAC | | | |
| | GCCTGCTTAAAACCCTGCAACAGGGT | | | |
| | CTGCCAGACTTCACCAGTCAAAGCAT | | | |
| | GTTGCAGAACTTTAGGAACTTTATCCT | | | |
| | AGAGCGCTCAGGAATCTTGCCCGCCA | | | |
| | CCTGCTGTGCACTTCCTAGCGACTTTG | | | |
| | TGCCCATTAAGTACCGCGAATGCCCT | | | |
| | CCGCCGCTTTGGGGCCACTGCTACCTT | | | |
| | CTGCAGCTAGCCAACTACCTTGCCTAC | | | |
| | CACTCTGACATAATGGAAGACGTGAG | | | |
| | CGGTGACGGTCTACTGGAGTGTCACT | | | |
| | GTCGCTGCAACCTATGCACCCCGCAC | | | |
| | CGCTCCCTGGTTTGCAATTCGCAGCTG | | | |
| | CTTAACGAAAGTCAAATTATCGGTAC | | | |
| | CTTTGAGCTGCAGGGTCCCTCGCCTGA | | | |
| | CGAAAAGTCCGCGGCTCCGGGGTTGA | | | |
| | AACTCACTCCGGGGCTGTGGACGTCG | | | |
| | GCTTACCTTCGCAAATTTGTACCTGAG | | | |
| | GACTACCACGCCCACGAGATTAGGTT | | | |
| | CTACGAAGACCAATCCCGCCCGCCAA | | | |
| | ATGCGGAGCTTACCGCCTGCGTCATT | | | |
| | ACCCAGGGCCACATTCTTGGCCAATT | | | |
| | GCAAGCCATCAACAAAGCCCGCCAAG | | | |
| | AGTTTCTGCTACGAAAGGGACGGGGG | | | |
| | GTTTACTTGGACCCCCAGTCCGGCGA | | | |
| | GGAGCTCAACCCAATCCCCCCGCCGC | | | |
| | CGCAGCCCTATCAGCAGCAGCCGCGG | | | |
| | GCCCTTGCTTCCCAGGATGGCACCCA | | | |
| | AAAAGAAGCTGCAGCTGCCGCCGCCA | | | |
| | CCCACGGACGAGGAGGAATACTGGGA | | | |
| | CAGTCAGGCAGAGGAGGTTTTGGACG | | | |
| | AGGAGGAGGAGGACATGATGGAAGA | | | |
| | CTGGGAGAGCCTAGACGAGGAAGCTT | | | |
| | CCGAGGTCGAAGAGGTGTCAGACGAA | | | |
| | ACACCGTCACCCTCGGTCGCATTCCCC | | | |
| | TCGCCGGCGCCCCAGAAATCGGCAAC | | | |
| | CGGTTCCAGCATGGCTACAACCTCCG | | | |
| | CTCCTCAGGCGCCGCCGGCACTGCCC | | | |
| | GTTCGCCGACCCAACCGTAGATGGGA | | | |
| | CACCACTGGAACCAGGGCCGGTAAGT | | | |
| | CCAAGCAGCCGCCGCCGTTAGCCCAA | | | |
| | GAGCAACAACAGCGCCAAGGCTACCG | | | |
| | CTCATGGCGCGGGCACAAGAACGCCA | | | |
| | TAGTTGCTTGCTTGCAAGACTGTGGG | | | |
| | GGCAACATCTCCTTCGCCCGCCGCTTT | | | |
| | CTTCTCTACCATCACGGCGTGGCCTTC | | | |
| | CCCCGTAACATCCTGCATTACTACCGT | | | |
| | CATCTCTACAGCCCATACTGCACCGG | | | |
| | CGGCAGCGGCAGCGGCAGCAACAGC | | | |
| | AGCGGCCACACAGAAGCAAAGGCGA | | | |
| | CCGGATAGCAAGACTCTGACAAAGCC | | | |
| | CAAGAAATCCACAGCGGCGGCAGCAG | | | |
| | CAGGAGGAGGAGCGCTGCGTCTGGCG | | | |
| | CCCAACGAACCCGTATCGACCCGCGA | | | |
| | GCTTAGAAACAGGATTTTTCCCACTCT | | | |
| | GTATGCTATATTTCAACAGAGCAGGG | | | |
| | GCCAAGAACAAGAGCTGAAAATAAA | | | |
| | AAACAGGTCTCTGCGATCCCTCACCC | | | |
| | GCAGCTGCCTGTATCACAAAAGCGAA | | | |
| | GATCAGCTTCGGCGCACGCTGGAAGA | | | |
| | CGCGGAGGCTCTCTTCAGTAAATACT | | | |
| | GCGCGCTGACTCTTAAGGACTAGTTTC | | | |
| | GCGCCCTTTCTCAAATTTAAGCGCGA | | | |
| | AAACTACGTCATCTCCAGCGGCCACA | | | |
| | CCCGGCGCCAGCACCTGTCGTCAGCG | | | |
| | CCATTATGAGCAAGGAAATTCCCACG | | | |
| | CCCTACATGTGGAGTTACCAGCCACA | | | |
| | AATGGGACTTGCGGCTGGAGCTGCCC | | | |
| | AAGACTACTCAACCCGAATAAACTAC | | | |
| | ATGAGCGCGGGACCCCACATGATATC | | | |
| | CCGGGTCAACGGAATCCGCGCCCACC | | | |
| | GAAACCGAATTCTCTTGGAACAGGCG | | | |
| | GCTATTACCACCACACCTCGTAATAA | | | |
| | CCTTAATCCCCGTAGTTGGCCCGCTGC | | | |
| | CCTGGTGTACCAGGAAAGTCCCGCTC | | | |
| | CCACCACTGTGGTACTTCCCAGAGAC | | | |
| | GCCCAGGCCGAAGTTCAGATGACTAA | | | |
| | CTCAGGGGCGCAGCTTGCGGGCGGCT | | | |
| | TTCGTCACAGGGTGCGGTCGCCCGGG | | | |
| | CAGGGTATAACTCACCTGACAATCAG | | | |
| | AGGGCGAGGTATTCAGCTCAACGACG | | | |
| | AGTCGGTGAGCTCCTCGCTTGGTCTCC | | | |
| | GTCCGGACGGGACATTTCAGATCGGC | | | |
| | GGCGCCGGCCGTCCTTCATTCACGCCT | | | |
| | CGTCAGGCAATCCTAACTCTGCAGAC | | | |
| | CTCGTCCTCTGAGCCGCGCTCTGGAG | | | |
| | GCATTGGAACTCTGCAATTTATTGAG | | | |
| | GAGTTTGTGCCATCGGTCTACTTTAAC | | | |
| | CCCTTCTCGGGACCTCCCGGCCACTAT | | | |
| | CCGGATCAATTTATTCCTAACTTTGAC | | | |
| | GCGGTAAAGGACTCGGCGGACGGCTA | | | |
| | CGACTGAATGTTAAGTGGAGAGGCAG | | | |
| | AGCAACTGCGCCTGAAACACCTGGTC | | | |
| | CACTGTCGCCGCCACAAGTGCTTTGCC | | | |
| | CGCGACTCCGGTGAGTTTTGCTACTTT | | | |
| | GAATTGCCCGAGGATCATATCGAGGG | | | |
| | CCCGGCGCACGGCGTCCGGCTTACCG | | | |
| | CCCAGGGAGAGCTTGCCCGTAGCCTG | | | |
| | ATTCGGGAGTTTACCCAGCGCCCCCT | | | |
| | GCTAGTTGAGCGGGACAGGGGACCCT | | | |
| | GTGTTCTCACTGTGATTTGCAACTGTC | | | |
| | CTAACCTTGGATTACATCAAGATCTTT | | | |
| | GTTGCCATCTCTGTGCTGAGTATAATA | | | |
| | AATACAGAAATTAAAATATACTGGGG | | | |
| | CTCCTATCGCCATCCTGTAAACGCCAC | | | |
| | CGTCTTCACCCGCCCAAGCAAACCAA | | | |
| | GGCGAACCTTACCTGGTACTTTTAACA | | | |
| | TCTCTCCCTCTGTGATTTACAACAGTT | | | |
| | TCAACCCAGACGGAGTGAGTCTACGA | | | |
| | GAGAACCTCTCCGAGCTCAGCTACTC | | | |
| | CATCAGAAAAAACACCACCCTCCTTA | | | |
| | CCTGCCGGGAACGTACGAGTGCGTCA | | | |
| | CCGGCCGCTGCACCACACCTACCGCC | | | |
| | TGACCGTAAACCAGACTTTTTCCGGA | | | |
| | CAGACCTCAATAACTCTGTTTACCAG | | | |
| | AACAGGAGGTGAGCTTAGAAAACCCT | | | |
| | TAGGGTATTAGGCCAAAGGCGCAGCT | | | |
| | ACTGTGGGGTTTATGAACAATTCAAG | | | |
| | CAACTCTACGGGCTATTCTAATTCAGG | | | |
| | TTTCTCTAGAATCGGGGTTGGGGTTAT | | | |
| | TCTCTGTCTTGTGATTCTCTTTATTCTT | | | |
| | ATACTAACGCTTCTCTGCCTAAGGCTC | | | |
| | GCCGCCTGCTGTGTGCACATTTGCATT | | | |
| | TATTGTCAGCTTTTTAAACGCTGGGGT | | | |
| | CGCCACCCAAGATGATTAGGTACATA | | | |
| | ATCCTAGGTTTACTCACCCTTGCGTCA | | | |
| | GCCCACGGTACCACCCAAAAGGTGGA | | | |
| | TTTTAAGGAGCCAGCCTGTAATGTTAC | | | |
| | ATTCGCAGCTGAAGCTAATGAGTGCA | | | |
| | CCACTCTTATAAAATGCACCACAGAA | | | |
| | CATGAAAAGCTGCTTATTCGCCACAA | | | |
| | AAACAAAATTGGCAAGTATGCTGTTT | | | |
| | ATGCTATTTGGCAGCCAGGTGACACT | | | |
| | ACAGAGTATAATGTTACAGTTTTCCA | | | |
| | GGGTAAAAGTCATAAAACTTTTATGT | | | |
| | ATACTTTTCCATTTTATGAAATGTGCG | | | |
| | ACATTACCATGTACATGAGCAAACAG | | | |
| | TATAAGTTGTGGCCCCCACAAAATTG | | | |
| | TGTGGAAAACACTGGCACTTTCTGCT | | | |
| | GCACTGCTATGCTAATTACAGTGCTCG | | | |
| | CTTTGGTCTGTACCCTACTCTATATTA | | | |
| | AATACAAAAGCAGACGCAGCTTTATT | | | |
| | GAGGAAAAGAAAATGCCTTAATTTAC | | | |
| | TAAGTTACAAAGCTAATGTCACCACT | | | |
| | AACTGCTTTACTCGCTGCTTGCAAAAC | | | |
| | AAATTCAAAAAGTTAGCATTATAATT | | | |
| | AGAATAGGATTTAAACCCCCCGGTCA | | | |
| | TTTCCTGCTCAATACCATTCCCCTGAA | | | |
| | CAATTGACTCTATGTGGGATATGCTCC | | | |
| | AGCGCTACAACCTTGAAGTCAGGCTT | | | |
| | CCTGGATGTCAGCATCTGACTTTGGCC | | | |
| | AGCACCTGTCCCGCGGATTTGTTCCAG | | | |
| | TCCAACTACAGCGACCCACCCTAACA | | | |
| | GAGATGACCAACACAACCAACGCGGC | | | |
| | CGCCGCTACCGGACTTACATCTACCA | | | |
| | CAAATACACCCCAAGTTTCTGCCTTTG | | | |
| | TCAATAACTGGGATAACTTGGGCATG | | | |
| | TGGTGGTTCTCCATAGCGCTTATGTTT | | | |
| | GTATGCCTTATTATTATGTGGCTCATC | | | |
| | TGCTGCCTAAAGCGCAAACGCGCCCG | | | |
| | ACCACCCATCTATAGTCCCATCATTGT | | | |
| | GCTACACCCAAACAATGATGGAATCC | | | |
| | ATAGATTGGACGGACTGAAACACATG | | | |
| | TTCTTTTCTCTTACAGTATGATTAAAT | | | |
| | GAGACATGATTCCTCGAGTTTTTATAT | | | |
| | TACTGACCCTTGTTGCGCTTTTTTGTG | | | |
| | CGTGCTCCACATTGGCTGCGGTTTCTC | | | |
| | ACATCGAAGTAGACTGCATTCCAGCC | | | |
| | TTCACAGTCTATTTGCTTTACGGATTT | | | |
| | GTCACCCTCACGCTCATCTGCAGCCTC | | | |
| | ATCACTGTGGTCATCGCCTTTATCCAG | | | |
| | TGCATTGACTGGGTCTGTGTGCGCTTT | | | |
| | GCATATCTCAGACACCATCCCCAGTA | | | |
| | CAGGGACAGGACTATAGCTGAGCTTC | | | |
| | TTAGAATTCTTTAATTATGAAATTTAC | | | |
| | TGTGACTTTTCTGCTGATTATTTGCAC | | | |
| | CCTATCTGCGTTTTGTTCCCCGACCTC | | | |
| | CAAGCCTCAAAGACATATATCATGCA | | | |
| | GATTCACTCGTATATGGAATATTCCAA | | | |
| | GTTGCTACAATGAAAAAAGCGATCTT | | | |
| | TCCGAAGCCTGGTTATATGCAATCATC | | | |
| | TCTGTTATGGTGTTCTGCAGTACCATC | | | |
| | TTAGCCCTAGCTATATATCCCTACCTT | | | |
| | GACATTGGCTGGAAACGAATAGATGC | | | |
| | CATGAACCACCCAACTTTCCCCGCGC | | | |
| | CCGCTATGCTTCCACTGCAACAAGTTG | | | |
| | TTGCCGGCGGCTTTGTCCCAGCCAATC | | | |
| | AGCCTCGCCCCACTTCTCCCACCCCCA | | | |
| | CTGAAATCAGCTACTTTAATCTAACA | | | |
| | GGAGGAGATGACTGACACCCTAGATC | | | |
| | TAGAAATGGACGGAATTATTACAGAG | | | |
| | CAGCGCCTGCTAGAAAGACGCAGGGC | | | |
| | AGCGGCCGAGCAACAGCGCATGAATC | | | |
| | AAGAGCTCCAAGACATGGTTAACTTG | | | |
| | CACCAGTGCAAAAGGGGTATCTTTTG | | | |
| | TCTGGTAAAGCAGGCCAAAGTCACCT | | | |
| | ACGACAGTAATACCACCGGACACCGC | | | |
| | CTTAGCTACAAGTTGCCAACCAAGCG | | | |
| | TCAGAAATTGGTGGTCATGGTGGGAG | | | |
| | AAAAGCCCATTACCATAACTCAGCAC | | | |
| | TCGGTAGAAACCGAAGGCTGCATTCA | | | |
| | CTCACCTTGTCAAGGACCTGAGGATC | | | |
| | TCTGCACCCTTATTAAGACCCTGTGCG | | | |
| | GTCTCAAAGATCTTATTCCCTTTAACT | | | |
| | AATAAAAAAAAATAATAAAGCATCAC | | | |
| | TTACTTAAAATCAGTTAGCAAATTTCT | | | |
| | GTCCAGTTTATTCAGCAGCACCTCCTT | | | |
| | GCCCTCCTCCCAGCTCTGGTATTGCAG | | | |
| | CTTCCTCCTGGCTGCAAACTTTCTCCA | | | |
| | CAATCTAAATGGAATGTCAGTTTCCTC | | | |
| | CTGTTCCTGTCCATCCGCACCCACTAT | | | |
| | CTTCATGTTGTTGCAGATGAAGCGCG | | | |
| | CAAGACCGTCTGAAGATACCTTCAAC | | | |
| | CCCGTGTATCCATATGACACGGAAAC | | | |
| | CGGTCCTCCAACTGTGCCTTTTCTTAC | | | |
| | TCCTCCCTTTGTATCCCCCAATGGGTT | | | |
| | TCAAGAGAGTCCCCCTGGGGTACTCT | | | |
| | CTTTGCGCCTATCCGAACCTCTAGTTA | | | |
| | CCTCCAATGGCATGCTTGCGCTCAAA | | | |
| | ATGGGCAACGGCCTCTCTCTGGACGA | | | |
| | GGCCGGCAACCTTACCTCCCAAAATG | | | |
| | TAACCACTGTGAGCCCACCTCTCAAA | | | |
| | AAAACCAAGTCAAACATAAACCTGGA | | | |
| | AATATCTGCACCCCTCACAGTTACCTC | | | |
| | AGAAGCCCTAACTGTGGCTGCCGCCG | | | |
| | CACCTCTAATGGTCGCGGGCAACACA | | | |
| | CTCACCATGCAATCACAGGCCCCGCT | | | |
| | AACCGTGCACGACTCCAAACTTAGCA | | | |
| | TTGCCACCCAAGGACCCCTCACAGTG | | | |
| | TCAGAAGGAAAGCTAGCCCTGCAAAC | | | |
| | ATCAGGCCCCCTCACCACCACCGATA | | | |
| | GCAGTACCCTTACTATCACTGCCTCAC | | | |
| | CCCCTCTAACTACTGCCACTGGTAGCT | | | |
| | TGGGCATTGACTTGAAAGAGCCCATT | | | |
| | TATACACAAAATGGAAAACTAGGACT | | | |
| | AAAGTACGGGGCTCCTTTGCATGTAA | | | |
| | CAGACGACCTAAACACTTTGACCGTA | | | |
| | GCAACTGGTCCAGGTGTGACTATTAA | | | |
| | TAATACTTCCTTGCAAACTAAAGTTAC | | | |
| | TGGAGCCTTGGGTTTTGATTCACAAG | | | |
| | GCAATATGCAACTTAATGTAGCAGGA | | | |
| | GGACTAAGGATTGATTCTCAAAACAG | | | |
| | ACGCCTTATACTTGATGTTAGTTATCC | | | |
| | GTTTGATGCTCAAAACCAACTAAATC | | | |
| | TAAGACTAGGACAGGGCCCTCTTTTT | | | |
| | ATAAACTCAGCCCACAACTTGGATAT | | | |
| | TAACTACAACAAAGGCCTTTACTTGTT | | | |
| | TACAGCTTCAAACAATTCCAAAAAGC | | | |
| | TTGAGGTTAACCTAAGCACTGCCAAG | | | |
| | GGGTTGATGTTTGACGCTACAGCCAT | | | |
| | AGCCATTAATGCAGGAGATGGGCTTG | | | |
| | AATTTGGTTCACCTAATGCACCAAAC | | | |
| | ACAAATCCCCTCAAAACAAAAATTGG | | | |
| | CCATGGCCTAGAATTTGATTCAAACA | | | |
| | AGGCTATGGTTCCTAAACTAGGAACT | | | |
| | GGCCTTAGTTTTGACAGCACAGGTGC | | | |
| | CATTACAGTAGGAAACAAAAATAATG | | | |
| | ATAAGCTAACTTTGTGGACCACACCA | | | |
| | GCTCCATCTCCTAACTGTAGACTAAAT | | | |
| | GCAGAGAAAGATGCTAAACTCACTTT | | | |
| | GGTCTTAACAAAATGTGGCAGTCAAA | | | |
| | TACTTGCTACAGTTTCAGTTTTGGCTG | | | |
| | TTAAAGGCAGTTTGGCTCCAATATCTG | | | |
| | GAACAGTTCAAAGTGCTCATCTTATTA | | | |
| | TAAGATTTGACGAAAATGGAGTGCTA | | | |
| | CTAAACAATTCCTTCCTGGACCCAGA | | | |
| | ATATTGGAACTTTAGAAATGGAGATC | | | |
| | TTACTGAAGGCACAGCCTATACAAAC | | | |
| | GCTGTTGGATTTATGCCTAACCTATCA | | | |
| | GCTTATCCAAAATCTCACGGTAAAAC | | | |
| | TGCCAAAAGTAACATTGTCAGTCAAG | | | |
| | TTTACTTAAACGGAGACAAAACTAAA | | | |
| | CCTGTAACACTAACCATTACACTAAA | | | |
| | CGGTACACAGGAAACAGGAGACACA | | | |
| | ACTCCAAGTGCATACTCTATGTCATTT | | | |
| | TCATGGGACTGGTCTGGCCACAACTA | | | |
| | CATTAATGAAATATTTGCCACATCCTC | | | |
| | TTACACTTTTTCATACATTGCCCAAGA | | | |
| | ATAAAGAATCGTTTGTGTTATGTTTCA | | | |
| | ACGTGTTTATTTTTCAATTGCAGAAAA | | | |
| | TTTCAAGTCATTTTTCATTCAGTAGTA | | | |
| | TAGCCCCACCACCACATAGCTTATAC | | | |
| | AGATCACCGTACCTTAATCAAACTCA | | | |
| | CAGAACCCTAGTATTCAACCTGCCAC | | | |
| | CTCCCTCCCAACACACAGAGTACACA | | | |
| | GTCCTTTCTCCCCGGCTGGCCTTAAAA | | | |
| | AGCATCATATCATGGGTAACAGACAT | | | |
| | ATTCTTAGGTGTTATATTCCACACGGT | | | |
| | TTCCTGTCGAGCCAAACGCTCATCAGT | | | |
| | GATATTAATAAACTCCCCGGGCAGCT | | | |
| | CACTTAAGTTCATGTCGCTGTCCAGCT | | | |
| | GCTGAGCCACAGGCTGCTGTCCAACT | | | |
| | TGCGGTTGCTTAACGGGCGGCGAAGG | | | |
| | AGAAGTCCACGCCTACATGGGGGTAG | | | |
| | AGTCATAATCGTGCATCAGGATAGGG | | | |
| | CGGTGGTGCTGCAGCAGCGCGCGAAT | | | |
| | AAACTGCTGCCGCCGCCGCTCCGTCCT | | | |
| | GCAGGAATACAACATGGCAGTGGTCT | | | |
| | CCTCAGCGATGATTCGCACCGCCCGC | | | |
| | AGCATAAGGCGCCTTGTCCTCCGGGC | | | |
| | ACAGCAGCGCACCCTGATCTCACTTA | | | |
| | AATCAGCACAGTAACTGCAGCACAGC | | | |
| | ACCACAATATTGTTCAAAATCCCACA | | | |
| | GTGCAAGGCGCTGTATCCAAAGCTCA | | | |
| | TGGCGGGGACCACAGAACCCACGTGG | | | |
| | CCATCATACCACAAGCGCAGGTAGAT | | | |
| | TAAGTGGCGACCCCTCATAAACACGC | | | |
| | TGGACATAAACATTACCTCTTTTGGCA | | | |
| | TGTTGTAATTCACCACCTCCCGGTACC | | | |
| | ATATAAACCTCTGATTAAACATGGCG | | | |
| | CCATCCACCACCATCCTAAACCAGCT | | | |
| | GGCCAAAACCTGCCCGCCGGCTATAC | | | |
| | ACTGCAGGGAACCGGGACTGGAACAA | | | |
| | TGACAGTGGAGAGCCCAGGACTCGTA | | | |
| | ACCATGGATCATCATGCTCGTCATGAT | | | |
| | ATCAATGTTGGCACAACACAGGCACA | | | |
| | CGTGCATACACTTCCTCAGGATTACA | | | |
| | AGCTCCTCCCGCGTTAGAACCATATCC | | | |
| | CAGGGAACAACCCATTCCTGAATCAG | | | |
| | CGTAAATCCCACACTGCAGGGAAGAC | | | |
| | CTCGCACGTAACTCACGTTGTGCATTG | | | |
| | TCAAAGTGTTACATTCGGGCAGCAGC | | | |
| | GGATGATCCTCCAGTATGGTAGCGCG | | | |
| | GGTTTCTGTCTCAAAAGGAGGTAGAC | | | |
| | GATCCCTACTGTACGGAGTGCGCCGA | | | |
| | GACAACCGAGATCGTGTTGGTCGTAG | | | |
| | TGTCATGCCAAATGGAACGCCGGACG | | | |
| | TAGTCATATTTCCTGAAGCAAAACCA | | | |
| | GGTGCGGGCGTGACAAACAGATCTGC | | | |
| | GTCTCCGGTCTCGCCGCTTAGATCGCT | | | |
| | CTGTGTAGTAGTTGTAGTATATCCACT | | | |
| | CTCTCAAAGCATCCAGGCGCCCCCTG | | | |
| | GCTTCGGGTTCTATGTAAACTCCTTCA | | | |
| | TGCGCCGCTGCCCTGATAACATCCAC | | | |
| | CACCGCAGAATAAGCCACACCCAGCC | | | |
| | AACCTACACATTCGTTCTGCGAGTCAC | | | |
| | ACACGGGAGGAGCGGGAAGAGCTGG | | | |
| | AAGAACCATGTTTTTTTTTTTATTCCA | | | |
| | AAAGATTATCCAAAACCTCAAAATGA | | | |
| | AGATCTATTAAGTGAACGCGCTCCCC | | | |
| | TCCGGTGGCGTGGTCAAACTCTACAG | | | |
| | CCAAAGAACAGATAATGGCATTTGTA | | | |
| | AGATGTTGCACAATGGCTTCCAAAAG | | | |
| | GCAAACGGCCCTCACGTCCAAGTGGA | | | |
| | CGTAAAGGCTAAACCCTTCAGGGTGA | | | |
| | ATCTCCTCTATAAACATTCCAGCACCT | | | |
| | TCAACCATGCCCAAATAATTCTCATCT | | | |
| | CGCCACCTTCTCAATATATCTCTAAGC | | | |
| | AAATCCCGAATATTAAGTCCGGCCAT | | | |
| | TGTAAAAATCTGCTCCAGAGCGCCCT | | | |
| | CCACCTTCAGCCTCAAGCAGCGAATC | | | |
| | ATGATTGCAAAAATTCAGGTTCCTCA | | | |
| | CAGACCTGTATAAGATTCAAAAGCGG | | | |
| | AACATTAACAAAAATACCGCGATCCC | | | |
| | GTAGGTCCCTTCGCAGGGCCAGCTGA | | | |
| | ACATAATCGTGCAGGTCTGCACGGAC | | | |
| | CAGCGCGGCCACTTCCCCGCCAGGAA | | | |
| | CCATGACAAAAGAACCCACACTGATT | | | |
| | ATGACACGCATACTCGGAGCTATGCT | | | |
| | AACCAGCGTAGCCCCGATGTAAGCTT | | | |
| | GTTGCATGGGCGGCGATATAAAATGC | | | |
| | AAGGTGCTGCTCAAAAAATCAGGCAA | | | |
| | AGCCTCGCGCAAAAAAGAAAGCACAT | | | |
| | CGTAGTCATGCTCATGCAGATAAAGG | | | |
| | CAGGTAAGCTCCGGAACCACCACAGA | | | |
| | AAAAGACACCATTTTTCTCTCAAACAT | | | |
| | GTCTGCGGGTTTCTGCATAAACACAA | | | |
| | AATAAAATAACAAAAAAACATTTAAA | | | |
| | CATTAGAAGCCTGTCTTACAACAGGA | | | |
| | AAAACAACCCTTATAAGCATAAGACG | | | |
| | GACTACGGCCATGCCGGCGTGACCGT | | | |
| | AAAAAAACTGGTCACCGTGATTAAAA | | | |
| | AGCACCACCGACAGCTCCTCGGTCAT | | | |
| | GTCCGGAGTCATAATGTAAGACTCGG | | | |
| | TAAACACATCAGGTTGATTCACATCG | | | |
| | GTCAGTGCTAAAAAGCGACCGAAATA | | | |
| | GCCCGGGGGAATACATACCCGCAGGC | | | |
| | GTAGAGACAACATTACAGCCCCCATA | | | |
| | GGAGGTATAACAAAATTAATAGGAGA | | | |
| | GAAAAACACATAAACACCTGAAAAAC | | | |
| | CCTCCTGCCTAGGCAAAATAGCACCC | | | |
| | TCCCGCTCCAGAACAACATACAGCGC | | | |
| | TTCCACAGCGGCAGCCATAACAGTCA | | | |
| | GCCTTACCAGTAAAAAAGAAAACCTA | | | |
| | TTAAAAAAACACCACTCGACACGGCA | | | |
| | CCAGCTCAATCAGTCACAGTGTAAAA | | | |
| | AAGGGCCAAGTGCAGAGCGAGTATAT | | | |
| | ATAGGACTAAAAAATGACGTAACGGT | | | |
| | TAAAGTCCACAAAAAACACCCAGAAA | | | |
| | ACCGCACGCGAACCTACGCCCAGAAA | | | |
| | CGAAAGCCAAAAAACCCACAACTTCC | | | |
| | TCAAATCGTCACTTCCGTTTTCCCACG | | | |
| | TTACGTAACTTCCCATTTTAAGAAAAC | | | |
| | TACAATTCCCAACACATACAAGTTAC | | | |
| | TCCGCCCTAAAACCTACGTCACCCGC | | | |
| | CCCGTTCCCACGCCCCGCGCCACGTC | | | |
| | ACAAACTCCACCCCCTCATTATCATAT | | | |
| | TGGCTTCAATCCAAAATAAGGTATAT | | | |
| | TATTGATGATG | | | |

What is claimed:

1. A method of delivering a therapeutic agent to a target cell of a subject, the method comprising introducing a plurality of enucleated cells comprising the therapeutic agent to the subject or a sample of the subject in vivo or ex vivo under conditions sufficient to deliver the therapeutic agent to the target cell of the subject, wherein the plurality of enucleated cells is obtained from a cryopreserved composition or a cryohibernated composition, and wherein the therapeutic agent is delivered to the target cell in an amount that is greater than or equal to about an amount of the therapeutic agent delivered to an otherwise comparable target cell of the subject by otherwise comparable enucleated cells that were not cryopreserved or not cryohibernated.

2. The method of claim 1, further comprising preparing a fluid composition comprising the plurality of enucleated cells from the cryopreserved composition.

3. The method of claim 1 or claim 2, wherein the cryopreserved composition is cryopreserved in liquid nitrogen.

4. The method of any one of claims 1-3, wherein the cryopreserved composition is cryopreserved for at least about 24 hours, for at least about 48 hours, at least about 72 hours, at least 96 about hours, at least about 5 days, at least about 6 days, at least about 7 days, at least about 10 days, at least about 15 days, at least about one month, at least about one month, or at least about one year.

5. The method of any one of claims 1-4, wherein the cryopreserved composition is stored at at most about −80° C. prior to cryopreserving the cryopreserved composition.

6. The method of claim 5, wherein the cryopreserved composition is stored at a temperature no higher than about −80° C. for at least about 24 hours.

7. The method of claim 1, further comprising preparing a fluid composition comprising the plurality of enucleated cells from the cryohibernated composition.

8. The method of claim 1 or claim 7, wherein the cryohibernated composition is stored at a temperature no higher than about 4° C.

9. The method of claim 1, claim 7, or claim 8, wherein the cryohibernated composition is cryohibernated for at least about 24 hours, for at least about 48 hours, at least about 72 hours, at least 96 about hours, at least about 5 days, at least about 6 days, at least about 7 days, at least about 10 days, at least about 15 days, at least about one month, at least about one month, or at least about one year.

10. The method of any one of claims 1-9, wherein the plurality of enucleated cells from the cryopreserved composition are suspended in a xeno-free media.

11. The method of any one of claims 1-10, wherein the plurality of enucleated cells from the cryopreserved composition are suspended in a freezing media.

12. The method of claim 11, wherein the freezing media comprises at least 2%, at least 5%, or at least 10% DMSO.

13. The method of claim 11 or claim 12, wherein the freezing media comprises CryoStor® media.

14. The method of claim 13, wherein the CryoStor® media is CryoStor® CS5 or CryoStor® CS10.

15. The method of claim 11, wherein the freezing media comprises DMSO, sucrose, sodium hydroxide, potassium hydroxide, or a combination thereof.

16. The method of claim 15, wherein the freezing media comprises about 2% to about 15% DMSO.

17. The method of claim 15, wherein the freezing media comprises about 0.5% to about 2% sucrose.

18. The method of claim 17, wherein the freezing media comprises about 1% sucrose.

19. The method of claim 15, wherein the freezing media comprises about 0.5% to about 1% sodium hydroxide.

20. The method of claim 19, wherein the freezing media comprises about 0.6% sodium hydroxide.

21. The method of claim 15, wherein the freezing media comprises about 0.05% to about 0.5% potassium hydroxide.

22. The method of claim 21, wherein the freezing media comprises about 0.1% potassium hydroxide.

23. The method of claim 2, wherein the preparing the fluid composition comprises thawing the cryopreserved composition.

24. The method of claim 23, wherein the thawing the cryopreserved composition is performed at room temperature or at 37° C.

25. The method of claim 23 or claim 24, further comprising reconstituting the plurality of enucleated cells from the cryopreserved composition subsequent to the thawing.

26. The method of claim 25, wherein the reconstituting the plurality of enucleated cells from the cryopreserved composition uses phosphate buffer solution (PBS).

27. The method of claim 25, wherein the reconstituting the plurality of enucleated cells from the cryopreserved composition uses sodium lactate solution.

28. The method of claim 25, wherein the reconstituting the plurality of enucleated cells from the cryopreserved composition uses saline solution.

29. The method of any one of claims 1-28, wherein the therapeutic agent comprises a virus, an exogenous DNA molecule, an exogenous RNA molecule, an exogenous protein, an exogenous peptide, or any combination thereof.

30. The method of claim 29, wherein the therapeutic agent comprises the virus.

31. The method of claim 30, wherein the virus is an adeno-associated virus (AAV), an adenovirus, a reovirus, a coxsackie virus, a retrovirus, a poxvirus, a baculovirus, or a herpes virus.

32. The method of claim 30, wherein the virus comprises an oncolytic virus.

33. The method of claim 32, wherein the oncolytic virus is an adenovirus, a human immunodeficiency virus, a Maraba virus, a Measles virus, a Newcastle disease virus, a poliovirus, a Seneca Valley virus, a parvovirus, a Semliki Forest virus, a Vesicular Stomatitis virus, a Sindbis virus, or any combination thereof.

34. The method of any one of claims 30-33, wherein the amount of the virus delivered to the subject is measured in viral titers in the target cell.

35. The method of claim 34, wherein the viral titers measured in the target cell are greater than the viral titers measured in the otherwise comparable target cell.

36. The method of claim 34, wherein the viral titers measured in the target cell are equal to about the viral titers measured in the otherwise comparable target cell.

37. The method of claim 29, wherein the exogenous protein comprises a cytokine or a cytokine receptor-binding fragment thereof.

38. The method of claim 37, wherein the amount of the cytokine or the cytokine receptor-binding fragment thereof delivered to the subject is measured by the secretion of the cytokine or the cytokine receptor-binding fragment thereof from the plurality of enucleated cells.

39. The method of claim 38, wherein the secretion of the cytokine or the cytokine receptor-binding fragment thereof measured is greater than or equal to about the secretion of the cytokine or the cytokine receptor-binding fragment thereof by an otherwise comparable enucleated cell that was not cryopreserved.

40. The method of claim 38, wherein the secretion of the cytokine or the cytokine receptor-binding fragment thereof measured is greater than or equal to about the secretion of the cytokine or the cytokine receptor-binding fragment thereof by an otherwise comparable nucleated cell that was cryopreserved.

41. The method of claim 29, wherein the exogenous protein comprises an immune checkpoint inhibitor.

42. The method of claim 41, wherein the immune checkpoint inhibitor comprises an inhibitor specific to PD-L1, PD-1, or a combination thereof.

43. The method of claim 29, wherein the exogenous protein comprises an antigen.

44. The method of claim 29, wherein the exogenous protein comprises an immunomodulatory protein.

45. The method of claim 29, wherein the therapeutic agent comprises the exogenous RNA molecule.

46. The method of claim 45, wherein the exogenous RNA molecule encodes a cytokine or the cytokine receptor-binding fragment thereof, a chemokine, or any combination thereof.

47. The method of claim 46, wherein the exogenous RNA molecule encodes the cytokine or the cytokine receptor-binding fragment thereof.

48. The method of claim 47, wherein the cytokine or the cytokine receptor-binding fragment thereof comprises interleukin-12 (IL-12), interferon-α (IFN-α), interferon-β (IFN-β), interferon-γ (IFN-γ), interleukin-7 (IL-7), interleukin-21 (IL-21), tumor necrosis factor α (TNF-α), granulocyte-macrophage colony-stimulating factor (GM-CSF), interleukin-15 (IL-15), or any combination thereof.

49. The method of claim 46, wherein the exogenous RNA molecule encodes the chemokine.

50. The method of claim 49, wherein the chemokine comprises stromal cell-derived factor-1α (SDF1α), C-C motif chemokine ligand 2 (CCL2), C-C motif chemokine ligand 3 (CCL3), C-C motif chemokine ligand 5 (CCL5), C-C motif chemokine ligand 8 (CCL8), C-C motif chemokine ligand 1 (CCL1), CXC motif chemokine ligand 9 (CXCL9), CXC motif chemokine ligand 10 (CXCL10), C-C motif chemokine ligand 11 (CCL11), CXC motif chemokine ligand 12 (CXCL12), or any combination thereof.

51. The method of claim 45, wherein the exogenous RNA molecule encodes an immune checkpoint inhibitor, an antigen, or an immunomodulatory protein.

52. The method of claim 51, wherein the immune checkpoint inhibitor comprises an inhibitor specific to PD-L1, PD-1, or a combination thereof.

53. The method of any one of claims 1-52, further comprising treating a disease or a condition in the subject.

54. The method of claim 53, wherein the disease is a cancer.

55. The method of claim 54, wherein the cancer comprises a solid tumor.

56. The method of claim 54, wherein the cancer is a lung cancer, a cancer metastasis in lung tissue, a liver cancer, or a cancer metastases in liver tissue.

57. The method of claim 56, wherein the liver cancer is a hepatocellular carcinoma or a cholangiocarcinoma.

58. The method of claim 54, wherein the cancer is the lung cancer.

59. The method of claim 58, wherein the lung cancer is a small cell lung cancer, a non-small lung cancer, or a bronchial carcinoid.

60. The method of claim 58, wherein the lung cancer is the small cell lung cancer.

61. The method of claim 58, wherein the lung cancer is the bronchial carcinoids.

62. The method of claim 58, wherein the lung cancer is the non-small cell lung cancer.

63. The method of claim 62, wherein the non-small cell lung cancer is an adenocarcinoma, squamous cell carcinoma, or large cell carcinoma.

64. The method of any one of claims 1-63, further comprising administering the plurality of enucleated cells to the subject intravenously.

65. The method of any one of claims 1-64, wherein the target cell of the subject comprises a cancer cell.

66. The method of any one of claims 1-64, wherein the target cell of the subject comprises a solid tumor cell.

67. The method of any one of claims 1-64, wherein the target cell of the subject comprises a lung cell.

68. The method of any one of claims 1-64, wherein the target cell of the subject comprises a liver cell.

69. A composition, comprising: a plurality of enucleated cells formulated from a cryopreserved composition or a cryohibernated composition, wherein the cryopreserved composition or the cryohibernated composition comprises the plurality of enucleated cells that are cryopreserved or cryohibernated, wherein at least a subset of the plurality of enucleated cells comprises (i) a therapeutic agent, and (ii) intracellular organelles sufficient to release the therapeutic agent in vivo or ex vivo in an amount that is greater than or equal to about an amount of the therapeutic agent released by otherwise identical enucleated cells that were not cryopreserved or not cryohibernated.

70. The composition of claim 69, wherein the plurality of enucleated cells comprises a diameter comprising less than or equal to about 70% of an average diameter of a nucleated parent cell.

71. The composition of claim 69 or claim 70, wherein the plurality of enucleated cells comprises a diameter comprising between about 1 micrometer (μm) to about 100 μm.

72. The composition of claim 71, wherein the plurality of enucleated cells comprises a diameter comprising between about 5 μm to about 25 μm.

73. The composition of claim 72, wherein the plurality of enucleated cells comprises a diameter comprising about 8 μm.

74. The composition of any one of claims 69-73, wherein the therapeutic agent comprises a virus, an exogenous DNA molecule, an exogenous RNA molecule, an exogenous protein, or an exogenous peptide, or any combination thereof.

75. The composition of claim 74, wherein the therapeutic agent comprises the virus.

76. The composition of claim 75, wherein the virus is an adeno-associated virus (AAV), an adenovirus, a reovirus, a coxsackie virus, a retrovirus, a poxvirus, a baculovirus, or a herpes virus.

77. The composition of claim 75, wherein the virus comprises an oncolytic virus.

78. The composition of claim 77, wherein the oncolytic virus is an adenovirus, a human immunodeficiency disease, a Maraba virus, a Measles virus, a Newcastle disease virus, a poliovirus, a Seneca Valley virus, a parvovirus, a Semliki Forest virus, a Vesicular Stomatitis virus, a Sindbis virus, or any combination thereof.

79. The composition of claim 75, wherein the amount of the virus released is measured in viral titers in a target cell.

80. The composition of claim 79, wherein the viral titers measured in the target cell are greater than the viral titers measured in an otherwise comparable target cell.

81. The composition of claim 79, wherein the viral titers measured in the target cell are equal to about the viral titers measured in an otherwise comparable target cell.

82. The composition of claim 74, wherein the therapeutic agent comprises a cytokine or cytokine receptor-binding fragment thereof.

83. The composition of claim 82, wherein the amount of the cytokine or the cytokine receptor-binding fragment released in vivo or ex vivo is a measurement of the secretion of the cytokine or the cytokine receptor-binding fragment thereof from the plurality of enucleated cells.

84. The composition of claim 83, wherein the amount of the cytokine or the cytokine receptor-binding fragment thereof measured is greater than or equal to about the secretion of the cytokine or the cytokine receptor-binding fragment thereof by the otherwise comparable enucleated cells that were not cryopreserved or not cryohibernated.

85. The composition of claim 83, wherein the amount of the cytokine or the cytokine receptor-binding fragment thereof measured is greater than or equal to about the secretion of the cytokine or the cytokine receptor-binding fragment thereof by the otherwise comparable nucleated cells that were cryopreserved or cryohibernated.

86. The composition of claim 74, wherein the exogenous protein comprises an immune checkpoint inhibitor.

87. The composition of claim 74, wherein the exogenous protein comprises an antigen.

88. The composition of claim 74, wherein the exogenous protein comprises an immunomodulatory protein.

89. The composition of claim 86, wherein the immune checkpoint inhibitor comprises an inhibitor specific to PD-L1, PD-1, or a combination thereof.

90. The composition of claim 74, wherein the therapeutic agent comprises an exogenous RNA molecule.

91. The composition of claim 90, wherein the exogenous RNA molecule encodes a cytokine or the cytokine receptor-binding fragment thereof, a chemokine, or any combination thereof.

92. The composition of claim 90, wherein the exogenous RNA molecule encodes a cytokine or the cytokine receptor-binding fragment thereof.

93. The composition of claim 92, wherein the cytokine or the cytokine receptor-binding fragment thereof comprises IL-12, IFN-α, IFN-β, IFN-γ, IL-7, IL-21, TNF-α, GM-CSF, IL-15, or any combination thereof.

94. The composition of claim 90, wherein the exogenous RNA molecule encodes a chemokine.

95. The composition of claim 94, wherein the chemokine comprises SDF1α, CCL2, CCL3, CCL5, CCL8, CCL1, CXCL9, CXCL10, CCL11, CXCL12, or combination thereof.

96. The composition of claim 90, wherein the exogenous RNA molecule encodes an immune checkpoint inhibitor, an antigen, or an immunomodulatory protein.

97. The composition of claim 96, wherein the immune checkpoint inhibitor comprises an inhibitor specific to PD-L1, PD-1, or a combination thereof.

98. The composition of any one of claims 69-97, wherein each enucleated cell of the plurality of enucleated cells lacks a nucleus and comprises one or more structural features of a nucleated cell.

99. The composition of claim 98, wherein the one or more structural features comprises one or more tunneling nanotubes.

100. The composition of claim 69, wherein the intracellular organelles comprise a Golgi apparatus, an endoplasmic reticulum, or any combination thereof.

101. A pharmaceutical composition, comprising:
(a) the composition of any one of claims 69-100, and
(b) a pharmaceutically acceptable: excipient, diluent, or carrier.

102. The pharmaceutical composition of claim 101, wherein the pharmaceutical composition is in a unit dose form.

103. The pharmaceutical composition of claim 101 or claim 102, wherein the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesicaly, intravaginally, intrarectally, orally, sublingually, transdermally, by inhalation, by inhaled nebulized form, by intraluminal-GI route, or any combination thereof, to a subject.

104. The pharmaceutical composition of claim 103, wherein the pharmaceutical composition is formulated for administering intravenously.

105. The pharmaceutical composition of any one of claims 101-104, further comprising at least one additional active agent.

106. The pharmaceutical composition of claim 105, wherein the at least one additional active agent comprises a cytokine, a growth factor, a hormone, an enzyme, a small molecule, a compound, or any combination thereof.

107. A kit, comprising:
(a) the composition of claims 69-100; or
(b) the pharmaceutical composition of any one of claim 101-106; and
(c) a container storing the composition or the pharmaceutical composition.

108. The kit of claim 107, further comprising a resuspension buffer.

109. The kit of claim 108, wherein the resuspension buffer comprises PBS.

110. The kit of claim 108, wherein the resuspension buffer comprises saline solution.

111. The kit of claim 108, wherein the resuspension buffer comprises sodium lactate solution.

112. The kit of any one of claims 107-111, further comprising instructions comprising a method for delivering the composition or the pharmaceutical composition to a target cell of a subject, wherein the method comprises: introducing the composition or the pharmaceutical composition to the target cell of a subject in vivo or ex vivo under conditions sufficient to deliver the therapeutic agent to the target cell.

113. The kit of claim 112, wherein the method further comprises treating a disease or a condition of the subject by administering the therapeutic agent to the target cell of the subject.

114. The kit of claim 113, wherein the disease or the condition comprises cancer.

115. The kit of claim 113, wherein the cancer comprises solid tumor.

116. The kit of claim 114, wherein the cancer is a lung cancer, a cancer metastases in lung tissue, a liver cancer, or a cancer metastases in liver tissue.

117. The kit of any one of claims 107-116, wherein the introducing the composition or the pharmaceutical composition to the target cell of a subject comprises administering the composition or the pharmaceutical composition to the subject intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesicaly, intravaginally, intrarectally, orally, sublingually, transdermally, by inhalation, by inhaled nebulized form, by intraluminal-GI route, or any combination thereof.

118. The kit of any one of claims 107-117, further comprising at least one additional active agent, wherein the at least one additional active agent comprises a cytokine, a growth factor, a hormone, an enzyme, a small molecule, a compound, or any combination thereof.


SEQUENCE LISTING (6 sequences)

SEQ ID NO: 1 - protein, 186 aa, synthetic construct
MRHIICHGGVITEEMAASLLDQLIEEVLADNLPPPSHFEPPTLHELYDLDVTAPEDPNEEAVSQIFPDSVMLAVQEGIDLLTFPPAPGSPEPPHLSRQPEQPEQRALGPVSMPNLVPEVIDLTCHEAGFPPSDDEDEEGEEFVLDYVEHPGHGCRSCHYHRRNTGDPDIMCSLCYMRTCGMFVYSK

SEQ ID NO: 2 - DNA, 561 bp, synthetic construct
  source 1..561: mol_type = other DNA
atgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtga

SEQ ID NO: 3 - DNA, 24 bp, synthetic construct
  source 1..24: mol_type = other DNA
cttacctgccacgaggctggcttt

SEQ ID NO: 4 - protein, 8 aa, synthetic construct
LTCHEAGF

SEQ ID NO: 5 - protein, 9 aa, synthetic construct
CDCRGDCFC

SEQ ID NO: 6 - DNA, 35938 bp, synthetic construct
  source 1..35938: mol_type = other DNA
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg